(19) United States
(12) Patent Application Publication
(10) Pub. No.: US 2008/0051419 A1
(43) Pub. Date: Feb. 28, 2008

(54) AMINE DERIVATIVES USEFUL AS ANTICANCER AGENTS

(75) Inventors: Matthew Stephen Corbett, Norwich, CT (US); Kevin Daniel Freeman-Cook, Clinton, CT (US); Goss Stryker Kauffman, Ledyard, CT (US); Blaise S. Lippa, Mystic, CT (US); Michael Joseph Luzzio, Noank, CT (US); Joel Morris, East Lyme, CT (US)

Correspondence Address: PFIZER INC, 10555 SCIENCE CENTER DRIVE, SAN DIEGO, CA, 92121, US

(73) Assignee: PFIZER INC.

(21) Appl. No.: 11/780,654

(22) Filed: Jul. 20, 2007

Related U.S. Application Data
(60) Provisional application No. 60/820,441, filed on Jul. 26, 2006.

(51) Int. Cl. A61K 31/497; A61P 35/04; C07D 487/02

(52) U.S. Cl. 514/258.1; 544/280

(57) ABSTRACT

The invention relates to compounds of formula I: 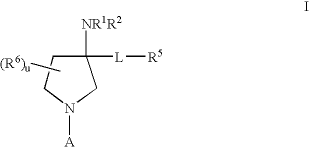 or a pharmaceutically acceptable salt thereof, wherein:
A is a moiety of formula: 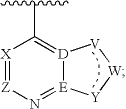
and to pharmaceutically acceptable salts and solvates thereof, wherein X, Z, D, E, V, W, Y, R¹, R², R⁵, R⁶, L, and u are as defined herein. The invention also relates to methods of treating abnormal cell growth in mammals by administering the compounds of formula I to a patient in need thereof, and to compositions for treating such disorders which contain the compounds of formula I. The invention also relates to methods of making the compounds of formula I.

### CROSS-REFERENCE TO RELATED APPLICATIONS

This application claims the benefit of U.S. Provisional Application No. 60/820,441, filed Jul. 26, 2006, the content of which is incorporated herein by reference in its entirety.

### BACKGROUND OF THE INVENTION

This invention relates to novel amine derivatives that are useful in the treatment of abnormal cell growth, such as cancer, in mammals. This invention also relates to a method of using such compounds in the treatment of abnormal cell growth in mammals, especially humans, and to compositions containing such compounds.

It is known that a cell may become cancerous by virtue of the transformation of a portion of its DNA into an oncogene (i.e., a gene which, on activation, leads to the formation of malignant tumor cells). Many oncogenes encode proteins that are aberrant kinases capable of causing cell transformation. Alternatively, the overexpression of a normal proto-oncogenic kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Other receptor tyrosine kinases include c-erbB-2, c-met, tie-2, PDGFr, FGFr, VEGF and TGF-β. When activated, these receptor kinases reportedly induce intracellular events such as intracellular signaling (see J. Dancer et al., Nature Reviews, 2:296-313, 2003).

The targeted angiogenesis inhibitor Avastin® (Genentech) that prevents the formation of blood vessels by binding to the vascular endothelial growth factor (VEGF) has been approved in the United States for the treatment of colon cancer with a combination chemotherapy regimen that includes 5-fluorouracil (5-FU) and Camptosar® (Irinotecan). Additionally, a second targeted monoclonal antibody Erbitux® (cetuximab) (Imclone) that is believed to bind to the epidermal growth factor receptor (EGFR) was also recently approved for the treatment of colon cancer. A large number of other targeted agents are in clinical development for a variety of cancers.

Intracellular protein kinases such as serine/threonine kinases are reportedly involved in intracellular signaling pathways (see Nature Reviews, 2:296-313, 2003). These serine/threonine kinases are also reported to play a role in cancer. For example, it is reported that serine/threonine kinases are involved in uncontrolled cell proliferation and reduced cell death in tumor cells (see C. Sachsenmaier, Onkologie, 24:346-355, 2001). Examples of serine/threonine kinases reported to be involved in human cancer include protein kinase B (Akt), cyclin-dependent kinases (CDKs), mammalian target of rapamycin (mTor), mitogen-activated protein kinase (MEK), P70s6K kinase and protein kinase C (PKC) (see Nature Reviews, 2:296-313, 2003).

Akt is a serine/threonine, intracellular kinase, which is reported to be a component of multiple signal transduction pathways involving cell proliferation, apoptosis, angiogenesis, and diabetes. It is reported that the Akt activation pathway may be activated by receptor tyrosine kinases, Ras, G protein-coupled receptors (GPCRs), or inactivation of the tumor suppressor phosphatase and tensin homolog deleted on chromosome ten (PTEN) (see, e.g., West et al., Drug Resist. Updates, 5:234-248, 2002). It is also reported that Akt can be activated by cellular stress including heat shock, administration of ultraviolet light, ischemia, hypoxia, hypoglycemia, and oxidative stress (see West et al., Drug Resist. Updates, 5:234-248, 2002).

It is also reported that Akt is deregulated in tumor cells (see, e.g., E. S. Kandel et. al., Exp. Cell Res., 253:21-229, 1999; Nicholson et. al., Cellular Signalling 14:381-395, 2002; and West et. al., Drug Resist. Updates, 5:234-248, 2002). Thus, high Akt activity in tumor cells provides an attractive target for drug intervention and the potential for a significant opportunity for controlling cell division in many types of cancer, and in particular for lung cancer, prostate cancer, colon cancer and breast cancer (see, e.g., International Appl. No. PCT/IB2006/000406).

S6 kinase (e.g. P70S6K1 and P70S6K2) is a key effector of mTOR, and has attracted attention as a possible anticancer drug discovery target. S6K phosphorylates ribosomal protein S6, which promotes the synthesis of proteins involved in ribosome biogenesis and translation initiation. As such, S6K regulates both the rate at which cells grow, and their subsequent commitment to cell cycle entry. Increased levels of S6K activity have been associated with cell transformation and elevated proliferation rates in tumors. For example, over-expression of S6K1 has been observed in human papillary thyroid tumors and meningiomas (Miyakawa et al., 2003, Endocrine J., 50:77; Surace at al., 2004, Ann Neurol 56:295). Moreover, amplification and overexpression of S6K1 has been observed in 10% of breast carcinomas, and was associated with poor prognosis and an increased risk of local recurrence (van der Hage, 2004, Br J Cancer 90: 1543). Studies in cultured cells have confirmed that S6K activity is enhanced by mechanisms which activate the PI 3-kinase/Akt/mTOR pathway (e.g., loss of the tumor suppressor PTEN (see Neshat et al., 2001, PNAS 98: 10314), and have shown a positive correlation between S6K activity and tumor growth.

Applicants have identified novel amines which are inhibitors of Akt kinase and/or P70S6K1 such that the compounds are able to modulate (reduce) the activity of the Akt kinase and/or the P70S6K1 directly and in cancer cells. Accordingly, such agents are useful in affecting tumor growth.

### SUMMARY OF THE INVENTION

The present invention relates to novel amine derivatives that are useful in the treatment of abnormal cell growth, such as cancer, in mammals. In particular, the present invention relates to a compound of formula I: 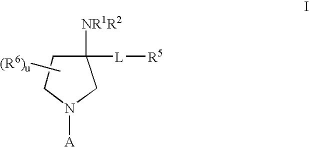
or a pharmaceutically acceptable salt thereof, wherein:

A is a moiety of formula:

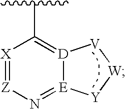

u is an integer from 0 to 3;

V is selected from the group consisting of N and CR⁷;

W and X are each independently selected from the group consisting of N and CR⁸;

Y is selected from the group consisting of CH, N and NH;

Z is selected from the group consisting of CH and N;

D and E are each selected from the group consisting of C and N, and wherein at least one of D and E is C;

L is a linker selected from the group consisting of —(CR³R⁴)ₘ— and —C(O)—, wherein one of said —(CR³R⁴)— moieties may optionally be replaced by a —CR³═CR⁴— group;

m is an integer from 1 to 6;

R¹ and R² are each independently selected from the group consisting of —H, —(C₁-C₆)alkyl, —(C₃-C₁₀)cycloalkyl, —(C₅-C₁₀)cycloalkenyl, —(C₂-C₉)heterocycloalkyl and —(C₄-C₉)heterocycloalkenyl; wherein each of the foregoing —(C₁-C₆)alkyl, —(C₃-C₁₀)cycloalkyl, —(C₅-C₁₀)cycloalkenyl, —(C₂-C₉)heterocycloalkyl and —(C₄-C₉)heterocycloalkenyl moieties is optionally substituted with one to five substituents independently selected from the group consisting of -halo, -cyano, —CF₃, —OR⁹, —C(O)R¹⁰, —NR¹¹R¹², —(C₁-C₆)alkyl and —(C₃-C₁₀)cycloalkyl;

R³ and R⁴ are each independently selected from the group consisting of —H, —(C₁-C₆)alkyl and —CF₃;

R⁵ is selected from the group consisting of:

(a) —OR¹³, —NR¹⁴R¹⁵, —NR¹¹C(O)R¹⁰, —NR¹¹C(O)OR⁹, —NR¹¹C(O)NR¹¹R¹², —NR¹¹S(O)ⱼR¹⁶, —NR¹¹C(═N—R¹⁷)NR¹¹R¹², —C(O)OR¹⁸, —OC(O)OR⁹, —OC(O)R⁹, and —S(O)ⱼR²⁰;

(b) —(C₁-C₆)alkyl substituted with one to five R²¹ groups, —(C₂-C₆)alkenyl, —(C₂-C₆)alkynyl, —(C₃-C₁₀)cycloalkyl substituted with one to five R²¹ groups, —(C₅-C₁₀)cycloalkenyl, —(C₆-C₁₀)bicycloalkyl, and —(C₆-C₁₀)bicycloalkenyl;

(c) —(C₂-C₉)heterocycloalkyl substituted with one to five R²² groups, —(C₄-C₉)heterocycloalkenyl, —(C₆-C₉)heterobicycloalkyl, and —(C₆-C₉)heterobicycloalkenyl;

(d) —(C₆-C₁₀)aryl substituted with one to five R²³ groups; wherein two R²³ groups when attached to adjacent carbon atoms may be taken together with the carbon atoms to which they are attached to form a moiety selected from the group consisting of —(C₃-C₇)cycloalkyl, —(C₅-C₁₀)cycloalkenyl, —(C₂-C₉)heterocyclyl, and —(C₂-C₁₀)heterocycloalkenyl; and wherein each of the foregoing —(C₃-C₇)cycloalkyl, —(C₅-C₁₀)cycloalkenyl, —(C₂-C₉)heterocyclyl, and —(C₂-C₁₀)heterocycloalkenyl moieties formed by the joinder of two R²³ groups may optionally be fused to a —(C₆-C₁₀)aryl or —(C₁-C₉)heteroaryl moiety;

(e) —(C₁-C₉)heteroaryl substituted with one to five R²⁴ groups; wherein two R²⁴ groups when attached to adjacent carbon atoms may be taken together with the carbon atoms to which they are attached to form a moiety selected from the group consisting of —(C₃-C₇)cycloalkyl, —(C₅-C₁₀)cycloalkenyl, —(C₂-C₉)heterocyclyl and —(C₂-C₁₀)heterocycloalkenyl; and wherein each of the foregoing —(C₃-C₇)cycloalkyl, —(C₅-C₁₀)cycloalkenyl, —(C₂-C₉)heterocyclyl, and —(C₂-C₁₀)heterocycloalkenyl moieties formed by the joinder of two R²⁴ groups is optionally fused to a —(C₆-C₁₀)aryl or —(C₁-C₉)heteroaryl moiety;

wherein each of the foregoing —(C₂-C₆)alkenyl, —(C₂-C₆)alkynyl, —(C₅-C₁₀)cycloalkenyl, —(C₆-C₁₀)bicycloalkyl, —(C₆-C₁₀)bicycloalkenyl, —(C₂-C₉)heterocycloalkenyl, —(C₆-C₉)heterobicycloalkyl and —(C₆-C₉)heterobicycloalkenyl R⁵ moieties in (b), (c), (d) and (e) above may optionally be substituted with one to five substituents independently selected from the group consisting of -halo, —OH, -cyano, —CF₃, —OCF₃, —OR⁹, —C(O)R¹⁰, —C(O)OR⁹, —OC(O)R¹⁰, —NR¹¹R¹², —NR¹¹C(O)R¹⁰, —C(O)NR¹¹R¹², —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl;

R⁶ is selected from the group consisting of —(C₁-C₆)alkyl and —(C₃-C₁₀)cycloalkyl;

R⁷ is selected from the group consisting of -halo, —OH, —CF₃, —NR¹¹R¹², -cyano, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein

each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R⁸ is independently selected from the group consisting of —H, -halo, -cyano, —OH and —(C₁-C₆)alkyl;

each R⁹ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R¹⁰ is independently selected from the group consisting of —H, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₆-C₁₀)bicycloalkyl, —(C₆-C₁₀)bicycloalkenyl, —(C₂-C₉)heterocycloalkyl, —(C₄-C₉)heterocycloalkenyl, —(C₆-C₉)heterobicycloalkyl, —(C₆-C₉)heterobicycloalkenyl, —(C₆-C₁₀)aryl, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₆-C₁₀)bicycloalkyl, —(C₆-C₁₀)bicycloalkenyl, —(C₂-C₉)heterocycloalkyl, —(C₄-C₉)heterocycloalkenyl, —(C₆-C₉)heterobicycloalkyl, —(C₆-C₉)heterobicycloalkenyl, —(C₆-C₁₀)aryl, and —(C₁-C₉)heteroaryl moieties is optionally substituted with one to five R²⁴ groups;

R¹¹ and R¹² are each independently selected from the group consisting of —H, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

R¹¹ and R¹² when attached to the same N atom may be taken together with the N atom to which they are attached to form a 3- to 11-membered mono or bicyclic ring optionally containing one to two additional heteroatoms independently selected from the group consisting of N, O and S(O)ⱼ; wherein said 3- to 11-membered mono or bicyclic ring may be saturated, unsaturated or aromatic; wherein each ring carbon atom of said 3- to 11-membered mono or bicyclic ring is optionally substituted with an oxo moiety; and wherein each N atom of said 3- to 11-membered mono or bicyclic ring is optionally substituted with a —(C₁-C₆)alkyl;

each R¹³ is independently selected from the group consisting of —(C₁-C₆)alkyl substituted with one to five R²⁵ groups, —(C₃-C₁₀)cycloalkyl substituted with one to five R²⁵ groups, —(C₅-C₁₀)cycloalkenyl, —(C₂-C₆)alkenyl, —(C₂-C₆)alkynyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl moieties is optionally substituted with one to five R²⁴ groups;

each R¹⁴ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R¹⁵ is independently selected from the group consisting of —(C₁-C₆)alkyl substituted with one to five R²² groups, —(C₂-C₆)alkenyl, —(C₂-C₆)alkynyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₂-C₆)alkenyl, —(C₂-C₆)alkynyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R¹⁶ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R¹⁷ is independently selected from the group consisting of —H, —CF₃, -nitro, -cyano, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R¹⁸ is independently selected from the group consisting of —(C₁-C₆)alkyl substituted with one to five R²⁶ groups, —(C₂-C₆)alkenyl, —(C₂-C₆)alkynyl, —(C₃-C₁₀)cycloalkyl substituted with substituted with one to five R²⁶ groups, —(C₅-C₁₀)cycloalkenyl, —(C₆-C₁₀)aryl and —(C₁-C₉)heteroaryl; wherein each of the foregoing —(C₂-C₆)alkenyl, —(C₂-C₆)alkynyl, —(C₅-C₁₀)cycloalkenyl, —(C₆-C₁₀)aryl and —(C₁-C₉)heteroaryl moieties is optionally substituted with one to five R²⁴ groups;

each R¹⁹ is independently selected from the group consisting of —H, —NR²⁸R²⁹, —(C₁-C₆)alkyl substituted with one to five R²⁴ groups, —(C₂-C₆)alkenyl, —(C₂-C₆)alkynyl, —(C₃-C₁₀)cycloalkyl substituted with one to five R²⁴ groups, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl moieties is optionally substituted with one to five R²⁴ groups;

each R²⁰ is independently selected from the group consisting of —H, —NR²⁸R²⁹, —(C₁-C₆)alkyl substituted with one to five R²² groups, —(C₂-C₆)alkenyl, —(C₂-C₆)alkynyl, —(C₃-C₁₀)cycloalkyl substituted with one to five R²² groups, —(C₅-C₁₀)cycloalkenyl, —(C₂-C₉)heterocycloalkyl, —(C₄-C₉)heterocycloalkenyl, —(C₆-C₁₀)aryl substituted with one to five R²² groups, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, and —(C₁-C₉)heteroaryl moieties is optionally substituted with one to five R²⁴ groups;

each R²¹ is independently selected from the group consisting of —CN, —NO₂, —SO₂NR²⁸R²⁹, —(C₂-C₆)alkenyl, and —(C₂-C₆)alkynyl; wherein each of the foregoing —(C₂-C₆)alkenyl and —(C₂-C₆)alkynyl moieties is optionally substituted with one to five R²⁴ groups;

each R²² is independently selected from the group consisting of -halo, —CF₃, —CN, —NO₂, —OR²⁸, —C(O)OR²⁸, —OC(O)R²⁸, —OC(O)OR²⁸, —NR²⁸R²⁹, —NR²⁸C(O)R²⁹, —S(O)₂R²⁸, —SO₂NR²⁸R²⁹ and —NR²⁸SO₂R²⁹, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl, wherein each of the foregoing —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;

each R²³ is independently selected from the group consisting of -halo, —CF₃, —CN, —NO₂, —OR²⁸, —C(O)R²⁸, —C(O)OR²⁸, —OC(O)R²⁸, —OC(O)OR²⁸, —NR²⁸R²⁹, —NR²⁸C(O)R²⁹, —S(O)₂R²⁸, —SO₂NR²⁸R²⁹, —NR²⁸SO₂R²⁹, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl substituted with one to five R²⁷ groups, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl and —(C₁-C₉)heteroaryl moieties is optionally substituted with one to five R²⁴ groups;

each R²⁴ is independently selected from the group consisting of -halo, —OH, —CF₃, —CN, —OCF₃, —NR²⁸R²⁹, —NR²⁸C(O)R²⁹, —C(O)R²⁸, —C(O)OR²⁸, —C(O)NR²⁸R²⁹, —C(O)NR²⁸C(O)R²⁹, —C(O)NR²⁸C(O)NR²⁹, —SO₂R²⁸, —SO₂NR²⁸R²⁹, —(C₁-C₆)alkyl, —O(C₁-C₆)alkyl, —(C₃-C₁₀)cycloalkyl, —O(C₃-C₁₀)cycloalkyl, —(C₅-C₁₀)cycloalkenyl, —O(C₅-C₁₀)cycloalkenyl, —(C₂-C₉)heterocycloalkyl, —O(C₂-C₉)heterocycloalkyl, —C₄-C₉)heterocycloalkenyl, —O(C₄-C₉)heterocycloalkenyl, —(C₆-C₁₀)aryl, —O(C₆-C₁₀)aryl, —(C₁-C₉)heteroaryl and —O(C₁-C₉)heteroaryl; wherein each of the foregoing —(C₁-C₆)alkyl, —O(C₁-C₆)alkyl, —(C₃-C₁₀)cycloalkyl, —O(C₃-C₁₀)cycloalkyl, —(C₅-C₁₀)cycloalkenyl, —O(C₅-C₁₀)cycloalkenyl, —(C₂-C₉)heterocycloalkyl, —O(C₂-C₉)heterocycloalkyl, —(C₄-C₉)heterocycloalkenyl, —O(C₄-C₉)heterocycloalkenyl, —(C₆-C₁₀)aryl, —O(C₆-C₁₀)aryl, —(C₁-C₉)heteroaryl and —O(C₁-C₉)heteroaryl moieties is optionally substituted by one to three moieties independently selected from the group consisting of -halo, —CF₃, —CN, —NO₂, —OR²⁸, —C(O)OR²⁸, —OC(O)R²⁹, —OC(O)OR²⁸, —NR²⁸R²⁹, —NR²⁸C(O)R²⁹, —S(O)₂R²⁸, —SO₂NR²⁸R²⁹, —NR²⁸SO₂R²⁹, —(C₁-C₆)alkyl, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl and —(C₁-C₉)heteroaryl;

each R²⁵ is independently selected from the group consisting of -halo, —CF₃, —CN, —NO₂, —OR²⁸, —C(O)OR²⁸, —OC(O)R²⁸, —OC(O)OR²⁸, —NR²⁸R²⁹, —NR²⁸C(O)R²⁹, —S(O)₂R²⁸, —SO₂NR²⁸R²⁹ and —NR²⁸SO₂R²⁸, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl substituted with one to five R²⁷ groups, and —(C₁-C₉)heteroaryl substituted with one to five R²⁶ groups;

each R²⁶ is independently selected from the group consisting of -halo, —CN, —NO₂, —OC(O)R²⁸, —OC(O)OR²⁸, —NR²⁸C(O)R²⁹, —SO₂NR²⁸R²⁹, —(C₂-C₆)alkenyl, —(C₂-C₆)alkynyl, —(C₃-C₁₀)cycloalkyl, —(C₆-C₁₀)aryl substituted with one to five R²⁷ groups, and —(C₁-C₉)heteroaryl substituted with one to five R²⁷ groups;

each R²⁷ is independently selected from the group consisting of -halo, —CF₃, —CN, —NO₂, —C(O)OR²⁸—OC(O)OR²⁸, —SO₂NR²⁸R²⁹, —NR²⁸SO₂R²⁹, —(C₁-C₆)alkyl, —(C₂-C₆)alkenyl, and —(C₂-C₆)alkynyl;

R²⁸ and R²⁹ are each independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; and

each j is independently an integer from 0 to 2.

The compounds of the invention may also exist in unsolvated and solvated forms. Accordingly, the invention also relates to the hydrates and solvates of the compounds of the invention. Thus, it will be understood that the compounds of formula I, and pharmaceutically acceptable salts thereof also include hydrates and solvates of said compounds of formula I, and pharmaceutically acceptable salts thereof, as discussed below.

The term “solvate” is used herein to describe a noncovelent or easily reversible combination between solvent and solute, or dispersion means and disperse phase. It will be understood that the solvate can be in the form of a solid, slurry (e.g., a suspension or dispersoid), or solution. Non-limiting examples of solvents include ethanol, methanol, propanol, acetonitrile, dimethyl ether, diethyl ether, tetrahydrofuan, methylene chloride, and water. The term ‘hydrate’ is employed when said solvent is water.

The term ‘hydrate’ is employed when said solvent is water. A currently accepted classification system for organic hydrates is one that defines isolated site, channel, or metal-ion coordinated hydrates (see Polymorphism in Pharmaceutical Solids by K. R. Morris (Ed. H. G. Brittain, Marcel Dekker, 1995)). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The invention also relates to prodrugs of the compounds of formula I. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985).

Some non-limiting examples of prodrugs in accordance with the invention include

(i) where the compound of formula I contains a carboxylic acid functionality (—COOH), an ester thereof, for example, a compound wherein the hydrogen of the carboxylic acid functionality of the compound of formula I is replaced by (C₁-C₆)alkyl;

(ii) where the compound of formula I contains an alcohol functionality (—OH), an ether thereof, for example, a compound wherein the hydrogen of the alcohol functionality of the compound of the invention is replaced by (C₁-C₆)alkanoyloxymethyl; and

(iii) where the compound of formula I contains a primary or secondary amino functionality (—NH₂ or —NHR where R≠H), an amide thereof, for example, a compound wherein, as the case may be, one or both hydrogens of the amino functionality of the compound of the invention is/are replaced by (C₁-C₆)alkanoyl.

Further examples of replacement groups in accordance with the foregoing examples and examples of other prodrug types may be found in the aforementioned references.

Moreover, certain compounds of the invention may themselves act as prodrugs of other compounds of formula I.

Also included within the scope of the invention are metabolites of compounds of formula I, that is, compounds formed in vivo upon administration of the drug. Some examples of metabolites in accordance with the invention include:

(i) where the compound of formula I contains a methyl group, an hydroxymethyl derivative thereof (e.g., —CH₃->-CH₂OH):

(ii) where the compound of formula I contains an alkoxy group, an hydroxy derivative thereof (e.g., —OR¹¹->-OH);

(iii) where the compound of formula I contains a tertiary amino group, a secondary amino derivative thereof (e.g., —NR¹³R¹⁴->-NHR¹³ or —NHR¹⁴);

(iv) where the compound of formula I contains a secondary amino group, a primary derivative thereof (e.g., —NHR¹³->-NH₂);

(v) where the compound of formula I contains a phenyl moiety, a phenol derivative thereof (e.g., -Ph ->-PhOH); and

(vi) where the compound of formula I contains an amide group, a carboxylic acid derivative thereof (e.g., —CONH₂->COOH).

This invention also encompasses compounds of formula I containing protective groups. One skilled in the art will also appreciate that compounds of formula I may also be prepared with certain protecting groups that are useful for purification or storage and may be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G. M. Wuts, Wiley-Interscience (1999). Non-limiting examples of useful protecting groups include, e.g., —C(O)—O-benzyl and —N(H)—C(O)—O-tert-butyl.

Included within the scope of the present invention are all stereoisomers, e.g., cis and trans isomers, and optical isomers such as R and S enantiomers, racemic, diastereomeric and other mixtures of such optical isomers; geometric isomers; and tautomeric forms of the compounds of formula I, including compounds exhibiting more than one type of isomerism; and mixtures of one or more thereof. Also included are acid addition or base addition salts wherein the counterion is optically active, for example, d-lactate or 1-lysine, or racemic, for example, dl-tartrate or dl-arginine.

For example, the compounds, salts and prodrugs of formula I may exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds.

In one embodiment, the compounds of formula I may contain olefin-like double bonds. When such bonds are present, the compounds of the invention exist as cis and trans configurations and as mixtures thereof. Cis/trans isomers may be separated by conventional techniques well known to those skilled in the art, for example, chromatography and fractional crystallization.

The present invention also includes atropisomers of compounds of formula I. Atropisomers refer to compounds of the invention that may be separated into rotationally restricted isomers.

Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

The invention also includes stereoisomers of the compounds of formula I. It will be understood that compounds of formula I containing one or more asymmetric carbon atoms may exist as two or more stereoisomers. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC) as described herein.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of an alcoholic solvent such as isopropanol, typically from 2% to 20%, and from 0 to 5% by volume of an alkylamine, typically 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to one skilled in the art.

As noted above, the invention also relates to salt forms of the compound of formula I, wherein the counter ion can be optically active or racemic, e.g., d-lactate or I-lysine, or racemic, for example, dI-tartrate or dI-arginine.

In one embodiment, the invention relates to a compound of formula I wherein u is 1.

In another embodiment, the invention relates to a compound of formula I wherein u is 1 and R⁶ is —(C₁-C₆)alkyl.

In another embodiment, the invention relates to a compound of formula I wherein u is 0.

In one embodiment, the invention relates to a compound of formula I wherein V is CR⁷.

In another embodiment, the invention relates to a compound of formula I wherein V is CR⁷ and R⁷ is selected from the group consisting of -halo, —OH, —CF₃, —NR¹¹R¹², and -cyano.

In another embodiment, the invention relates to a compound of formula I wherein V is CR⁷, and R⁷ is selected from the group consisting of —(C₁-C₆)alkyl, —(C₂-C₆)alkenyl and —(C₂-C₆)alkynyl; wherein each of the foregoing —(C₁-C₆)alkyl, —(C₂-C₆)alkenyl and —(C₂-C₆)alkynyl moieties is optionally substituted with one to five R²⁴ groups.

In another embodiment, the invention relates to a compound of formula I wherein V is CR⁷ and R⁷ is —(C₁-C₆)alkyl optionally substituted with one to five R²⁶ groups.

In another embodiment, the invention relates to a compound of formula I wherein V is CR⁷ and R⁷ is —(C₁-C₆)alkyl.

In another embodiment, the invention relates to a compound of formula I wherein V is CR⁷ and R⁷ is selected from the group consisting of -methyl, -ethyl and -propyl.

In another embodiment, the invention relates to a compound of formula I wherein V is CR⁷ and R⁷ is -ethyl.

In one embodiment, the invention relates to a compound of formula I wherein V is CR⁷ and R⁷ is selected from the group consisting of —(C₃-C₁₀)cycloalkyl, —(C₅-C₁₀)cycloalkenyl, —(C₂-C₉)heterocycloalkyl, and —(C₄-C₉)heterocycloalkenyl; and wherein each of the foregoing —(C₃-C₁₀)cycloalkyl, —(C₅-C₁₀)cycloalkenyl, —(C₂-C₉)heterocycloalkyl and —(C₄-C₉)heterocycloalkenyl moieties is optionally substituted with one to five R²⁴ groups.

In another embodiment, the invention relates to a compound of formula I wherein V is CR⁷, and R⁷ is selected from the group consisting of —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups.

In one embodiment, the invention relates to a compound of formula I wherein V is N.

In one embodiment, the invention relates to a compound of formula I wherein W is CH.

In another embodiment, the invention relates to a compound of formula I, wherein W is CR⁸, and R⁸ is —(C₁-C₆)alkyl.

In another embodiment, the invention relates to a compound of formula I, wherein W is CR⁸, and R⁸ is selected from the group consisting of -methyl, -ethyl and -propyl.

In one embodiment, the invention relates to a compound of formula I wherein W is N.

In one embodiment, the invention relates to a compound of formula I wherein X is CR⁸.

In one embodiment, the invention relates to a compound of formula I wherein X is CH.

In another embodiment, the invention relates to a compound of formula I, wherein X is CR⁸ and R⁸ is —(C₁-C₆)alkyl.

In another embodiment, the invention relates to a compound of formula I, wherein X is CR⁸ and R⁸ is selected from the group consisting of -methyl, -ethyl and -propyl.

In another embodiment, the invention relates to a compound of formula I, wherein X is N.

In one embodiment, the invention relates to a compound of formula I wherein Y is CH.

In another embodiment, the invention relates to a compound of formula I wherein Y is NH.

In another embodiment, the invention relates to a compound of formula I wherein Y is N.

In one embodiment, the invention relates to a compound of formula I wherein Z is CH.

In another embodiment, the invention relates to a compound of formula I wherein Z is N.

In one embodiment, the invention relates to a compound of formula I wherein D is N.

In one embodiment, the invention relates to a compound of formula I wherein E is N.

In one embodiment, the invention relates to a compound of formula I wherein D is C and E is C.

In another embodiment, the invention relates to a compound of formula I where X is N, Z is CH, D is C, and E is C.

In another embodiment, the invention relates to a compound of formula I where X is N, Z is CH, D is C, E is C, and V is CR⁷. In another embodiment, the invention relates to a compound of formula I where X is N, Z is CH, D is C, E is C, and W is CR⁸.

In another embodiment, the invention relates to a compound of formula I where X is N, Z is CH, D is C, E is C, and Y is NH.

In another embodiment, the invention relates to a compound of formula I where X is N, Z is CH, D is C, E is C, W is CR⁸, Y is NH, and V is CR⁷.

In another embodiment, the invention relates to a compound of formula I wherein the moiety A is selected from the group consisting of:

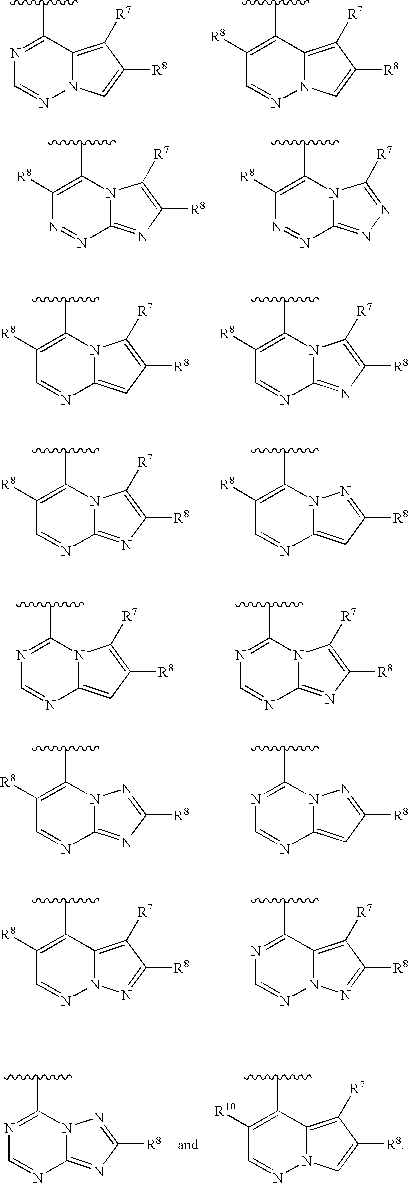

In another embodiment, the invention relates to a compound of formula I wherein the moiety A is selected from the group consisting of:

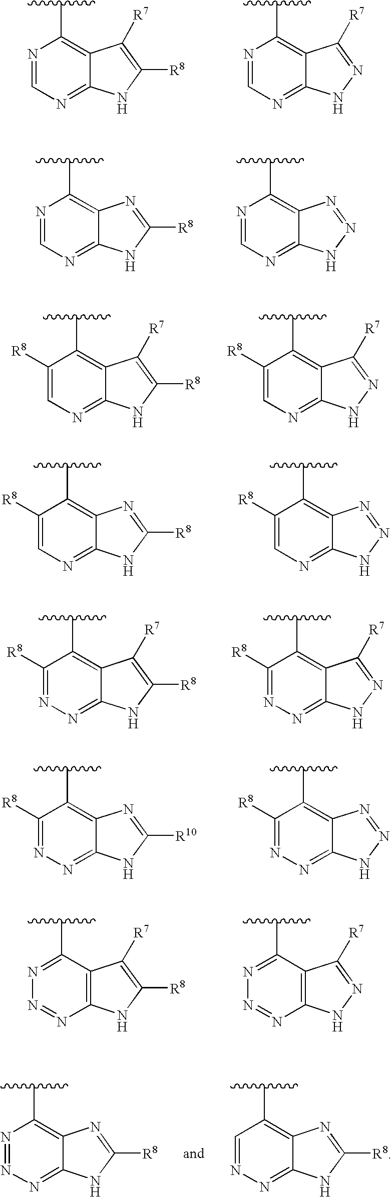

In another embodiment, the invention relates to a compound of formula I wherein the moiety A is selected from the group consisting of:

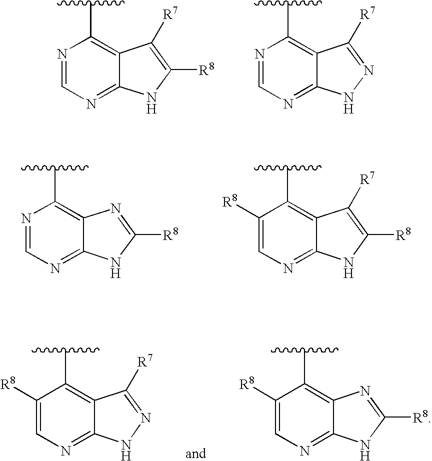

In one embodiment, the invention relates to a compound of formula I wherein R¹ and R² are each independently selected from the group consisting of —H and —(C₁-C₆)alkyl; wherein said —(C₁-C₆)alkyl may optionally be substituted with one to five substituents independently selected from the group consisting of -halo, -cyano, —CF₃, —OR⁹, —C(O)R¹⁰, —NR¹¹R¹², —(C₁-C₆)alkyl and —(C₃-C₁₀)cycloalkyl.

In another embodiment, the invention relates to a compound of formula I wherein R¹ and R² are each —H.

In one embodiment, the invention relates to a compound of formula I wherein L is —C(O)—.

In another embodiment, the invention relates to a compound of formula I wherein L is —(CR³R⁴)ₘ—; and wherein one of said —(CR³R⁴)— moieties may optionally be replaced by a —CR³═CR⁴— moiety.

In another embodiment, the invention relates to a compound of formula I wherein L is —(CR³R⁴)ₘ—; and wherein one of said —(CR³R⁴)— moieties is —CR³═CR⁴—.

In another embodiment, the invention relates to a compound of formula I wherein L is —(CR³R⁴)ₘ—; wherein one of said —(CR³R⁴)— moieties may optionally be replaced by a —CR³═CR⁴— moiety; and wherein m is an integer from 1 to 3.

In another embodiment, the invention relates to a compound of formula I wherein L is —(CR³R⁴)ₘ—; wherein one of said —(CR³R⁴)— moieties may optionally be replaced by a —CR³═CR⁴— moiety; wherein m is an integer from 1 to 3; and wherein R³ and R⁴ are each —H.

In another embodiment, the invention relates to a compound of formula I wherein L is selected from the group consisting of —CH₂—, —CH═CH—, —CH₂CH═CH— and —CH═CHCH₂—.

In another embodiment, the invention relates to a compound of formula I wherein L is —CH₂—.

In another embodiment, the invention relates to a compound of formula I wherein L is —CH═CH—.

In one embodiment, the invention relates to a compound of formula I wherein R⁵ is selected from the group consisting of —OR¹³, —NR¹⁴R¹⁵, —NR¹¹C(O)R¹⁰, —NR¹¹C(O)OR⁹, —NR¹¹C(O)NR¹¹R¹², —NR¹¹S(O)ⱼR¹⁶, —NR¹¹C(═N—R¹⁷)NR¹¹R¹², —C(O)R¹⁸, —OC(O)OR⁹, —OC(O)R¹⁹, and —S(O)ⱼR²⁰.

In another embodiment, the invention relates to a compound of formula I wherein R⁵ is selected from the group consisting of —NR¹⁴R¹⁵, —NR¹¹C(O)R¹⁰, —NR¹¹C(O)NR¹¹R¹², —NR¹¹S(O)ⱼR¹⁶ and —NR¹¹C(═N—R¹⁷)NR¹¹R¹².

In another embodiment, the invention relates to a compound of formula I wherein R⁵ is —NR¹¹C(O)R¹⁰.

In another embodiment, the invention relates to a compound of formula I wherein R⁵ is —NR¹¹C(O)R¹⁰, and R¹⁰ and R¹¹ are each independently selected from the group consisting of —H, —(C₁-C₆)alkyl, —(C₃-C₁₀)cycloalkyl, —(C₂-C₉)heterocycloalkyl, —(C₆-C₁₀)aryl, and —(C₁-C₉)heteroaryl; wherein each of the foregoing —(C₁-C₆)alkyl, —(C₃-C₁₀)cycloalkyl, —(C₂-C₉)heterocycloalkyl, —(C₆-C₁₀)aryl and —(C₁-C₉)heteroaryl moieties may optionally be substituted with one to five R²⁴ groups.

In another embodiment, the invention relates to a compound of formula I wherein R⁵ is —NR¹¹C(O)R¹⁰; wherein R¹⁰ is selected from the group consisting of —(C₆-C₁₀)aryl, and —(C₁-C₉)heteroaryl; wherein R¹¹ is selected from the group consisting of —H and —(C₁-C₆)alkyl; and wherein each of the foregoing —(C₁-C₆)alkyl, —(C₃-C₁₀)cycloalkyl, —(C₂-C₉)heterocycloalkyl, —(C₆-C₁₀)aryl and —(C₁-C₉)heteroaryl moieties of said R¹⁰ and R¹¹ groups may optionally be substituted with one to five R²⁴ groups.

In another embodiment, the invention relates to a compound of formula I wherein R⁵ is —NR¹¹C(O)R¹⁰; wherein R¹⁰ is —(C₆-C₁₀)aryl and R¹¹ is —H; wherein said —(C₆-C₁₀)aryl of said R¹⁰ group may optionally be substituted with one to five groups selected from the group consisting of -halo, —OH, —CF₃, —CN, —OCF₃, —NR¹¹R¹², —NR¹¹C(O)R¹⁰, —C(O)R¹¹, —C(O)OR¹¹, —C(O)NR¹¹R¹², —C(O)NR¹¹C(O)R¹⁰, —C(O)NR¹¹C(O)NR¹², —SO₂R¹¹, —SO₂NR¹¹R¹², —(C₁-C₆)alkyl and —O(C₁-C₆)alkyl; wherein each of the foregoing —(C₁-C₆)alkyl and —O(C₁-C₆)alkyl moieties may optionally be substituted by one to three moieties independently selected from the group consisting of -halo, —CF₃, —CN, —NO₂, —OR¹¹, —C(O)OR¹¹—OC(O)R¹¹, —OC(O)OR¹¹, —NR¹¹R¹², NR¹¹C(O)R¹⁰, —S(O)₂R¹¹, —SO₂NR¹¹R¹², —NR¹¹SO₂R¹², —(C₁-C₆)alkyl, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl and —(C₁-C₉)heteroaryl.

In another embodiment, the invention relates to a compound of formula I wherein R⁵ is —NR¹¹C(O)R¹⁰; wherein R¹⁰ is —(C₆-C₁₀)aryl and R¹¹ is —H; wherein said —(C₆-C₁₀)aryl of said R¹⁰ group is substituted with one to five groups selected from the group consisting of -halo, —OH, —CF₃, —CN, —OCF₃, —(C₁-C₆)alkyl and —(C₃-C₁₀)cycloalkyl.

In one embodiment, the invention relates to a compound of formula I wherein R⁵ is —C(O)OR¹⁸.

In another embodiment, the invention relates to a compound of formula I wherein R⁵ is —C(O)OR¹⁸; wherein R¹⁸ is selected from the group consisting of —(C₁-C₆)alkyl substituted with one to five R²⁶ groups, —(C₂-C₆)alkenyl, —(C₂-C₆)alkynyl, —(C₃-C₁₀)cycloalkyl substituted with substituted with one to five R²⁶ groups, and —(C₅-C₁₀)cycloalkenyl; wherein each of the foregoing —(C₂-C₆)alkenyl, —(C₂-C₆)alkynyl, —(C₅-C₁₀)cycloalkenyl moieties may optionally be substituted with one to five R²⁴ groups.

In one embodiment, the invention relates to a compound of formula I wherein R⁵ is —OR¹³.

In another embodiment, the invention relates to a compound of formula I wherein R⁵ is —OR¹³; and wherein R¹³ is selected from the group consisting of —(C₁-C₆)alkyl substituted with one to five R²⁵ groups and —(C₃-C₁₀)cycloalkyl substituted with one to five R²⁵ groups.

In another embodiment, the invention relates to a compound of formula I wherein R⁵ is —OR¹³; wherein R¹³ is selected from the group consisting of —(C₆-C₁₀)aryl and —(C₁-C₉)heteroaryl; and wherein each of the foregoing —(C₆-C₁₀)aryl and —(C₁-C₉)heteroaryl moieties may optionally be substituted with one to five R²⁴ groups.

In one embodiment, the invention relates to a compound of formula I, wherein R⁵ is selected from the group consisting —(C₁-C₆)alkyl substituted with one to five R²¹ groups, —(C₂-C₆)alkenyl, —(C₂-C₆)alkynyl, —(C₃-C₁₀)cycloalkyl substituted with one to five R²¹ groups, —(C₅-C₁₀)cycloalkenyl, —(C₆-C₁₀)bicycloalkyl and —(C₆-C₁₀)bicycloalkenyl; wherein each of the foregoing —(C₂-C₆)alkenyl, —(C₂-C₆)alkynyl, —(C₅-C₁₀)cycloalkenyl, —(C₆-C₁₀)bicycloalkyl and —(C₆-C₁₀)bicycloalkenyl moieties may optionally be substituted with one to five substituents independently selected from the group consisting of -halo, —OH, -cyano, —CF₃, —OCF₃, —OR⁹, —C(O)R¹⁰, —C(O)OR⁹, —OC(O)R¹⁰, —NR¹¹R¹², —NR¹¹C(O)R¹⁰, —C(O)NR¹¹R¹², —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl.

In another embodiment, the invention relates to a compound of formula I, wherein R⁵ is selected from the group consisting —(C₁-C₆)alkyl substituted with one to five R²¹ groups and —(C₃-C₁₀)cycloalkyl substituted with one to five R²¹ groups.

In another embodiment, the invention relates to a compound of formula I, wherein R⁵ is selected from the group consisting —(C₂-C₉)heterocycloalkyl substituted with one to five R²² groups, —(C₄-C₉)heterocycloalkenyl, —(C₆-C₉)heterobicycloalkyl and —(C₆-C₉)heterobicycloalkenyl; and wherein each of the foregoing —(C₂-C₉)heterocycloalkenyl, —(C₆-C₉)heterobicycloalkyl and —(C₆-C₉)heterobicycloalkenyl moieties may optionally be substituted with one to five substituents independently selected from the group consisting of -halo, —OH, -cyano, —CF₃, —OCF₃, —OR⁹, —C(O)R¹⁰, —C(O)OR⁹, —OC(O)R¹⁰, —NR¹¹R¹², —NR¹¹C(O)R¹⁰, —C(O)NR¹¹R¹², —(C₁-C₆)alkyl, —(C₂-C₆)alkenyl, —(C₂-C₆)alkynyl, —(C₃-C₁₀)cycloalkyl —C₁₀)cycloalkenyl, —(C₂-C₉)heterocycloalkyl, —(C₄-C₉)heterocycloalkenyl, —(C₆-C₁₀)aryl and —(C₁-C₉)heteroaryl.

In another embodiment, the invention relates to a compound of formula I, wherein R⁵ is a —(C₂-C₉)heterocycloalkyl substituted with one to five R²² groups.

In another embodiment, the invention relates to a compound of formula I, wherein R⁵ is a —(C₂-C₉)heterocycloalkyl substituted with one to five R²² groups; wherein R²² is selected from the group consisting of -halo, —CF₃, —CN, —NO₂, —OR², —C(O)OR², —OC(O)R²⁸—OC(O)OR²⁸, —NR²⁸R²⁹, —NR²⁸C(O)R²⁹, —S(O)₂R²⁸, —SO₂NR²⁸R²⁹ and —NR²⁸SO₂R²⁹, —(C₃-C₁₀)cycloalkyl, —(C₂-C₉)heterocycloalkyl, —(C₆-C₁₀)aryl and —(C₁-C₉)heteroaryl, wherein each of the foregoing —(C₃-C₁₀)cycloalkyl, —(C₂-C₉)heterocycloalkyl, —(C₆-C₁₀)aryl and —(C₁-C₉)heteroaryl moieties is optionally substituted with one to five R²⁴ groups.

In another embodiment, the invention relates to a compound of formula I, wherein R⁵ is —(C₆-C₁₀)aryl substituted with one to five R²³ groups; wherein two R²³ groups when attached to adjacent carbon atoms may optionally be taken together with the carbon atoms to which they are attached to form a moiety selected from the group consisting of —(C₃-C₁₀)cycloalkyl, —(C₅-C₁₀)cycloalkenyl, —(C₂-C₉)heterocyclyl, and —(C₂-C₁₀)heterocycloalkenyl; and wherein each of the foregoing —(C₃-C₁₀)cycloalkyl, —(C₅-C₁₀)cycloalkenyl, —(C₂-C₉)heterocyclyl, and —(C₂-C₁₀)heterocycloalkenyl moieties formed by the joinder of two R²³ groups may optionally be fused to a —(C₆-C₁₀)aryl or —(C₁-C₉)heteroaryl moiety.

In another embodiment, the invention relates to a compound of formula I, wherein R⁵ is —(C₆-C₁₀)aryl substituted with one to five groups selected from the group consisting of -halo, —OH, —CF₃, —CN, —OCF₃, —NR²⁸R²⁹, —NR²⁸C(O)R²⁹, —C(O)R²⁸, —C(O)OR²⁸, —C(O)NR²⁸R²⁹, —C(O)NR²⁸C(O)R²⁹, —C(O)NR²⁸C(O)NR²⁹, —SO₂R²⁸, —SO₂NR²⁸R²⁹, —(C₁-C₆)alkyl and —O(C₁-C₆)alkyl; wherein each of the foregoing —(C₁-C₆)alkyl and —O(C₁-C₆)alkyl moieties is optionally substituted by one to three moieties independently selected from the group consisting of -halo, —CF₃, —CN, —NO₂, —OR²⁸, —C(O)OR²⁸, —OC(O)R²⁹, —OC(O)OR²⁸, —NR²⁸R²⁹, —NR²⁸C(O)R²⁹—S(O)₂R²⁸, —SO₂NR²⁸R²⁹, —NR²⁸SO₂R²⁹, —(C₁-C₆)alkyl, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl and —(C₁-C₉)heteroaryl.

In another embodiment, the invention relates to a compound of formula I, wherein R⁵ is —(C₆-C₁₀)aryl substituted with one to five groups selected from the group consisting of -halo, —OH, —CF₃, —CN, —OCF₃, —(C₁-C₆)alkyl and —(C₃-C₁₀)cycloalkyl.

In another embodiment, the invention relates to a compound of formula I, wherein R⁵ is —(C₁-C₉)heteroaryl substituted with one to five groups is independently selected from the group consisting of -halo, —OH, —CF₃, —CN, —OCF₃, —NR¹¹R¹², —NR¹¹C(O)R¹⁰, —C(O)R¹¹, —C(O)OR¹¹, —C(O)NR¹¹R¹², —C(O)NR¹¹C(O)R¹⁰, —C(O)NR¹¹C(O)NR¹², —SO₂R¹¹, —SO₂NR¹¹R¹², —(C₁-C₆)alkyl, —O(C₁-C₆)alkyl, —(C₃-C₁₀)cycloalkyl, —O(C₃-C₁₀)cycloalkyl, —(C₂-C₉)heterocycloalkyl, —O(C₂-C₉)heterocycloalkyl, —(C₆-C₁₀)aryl, —O(C₆-C₁₀)aryl, —(C₁-C₉)heteroaryl and —O(C₁-C₉)heteroaryl; wherein each of the foregoing —(C₁-C₆)alkyl, —O(C₁-C₆)alkyl, —(C₃-C₁₀)cycloalkyl, —O(C₃-C₁₀)cycloalkyl, —(C₅-C₁₀)cycloalkenyl, —O(C₅-C₁₀)cycloalkenyl, —(C₂-C₉)heterocycloalkyl, —O(C₂-C₉)heterocycloalkyl, —O(C₄-C₉)heterocycloalkenyl, —O(C₄-C₉)heterocycloalkenyl, —(C₆-C₁₀)aryl, —O(C₆-C₁₀)aryl, —(C₁-C₉)heteroaryl and —O(C₁-C₉)heteroaryl moieties may optionally be substituted by one to three moieties independently selected from the group consisting of -halo, —CF₃, —CN, —NO₂, —OR¹¹, —C(O)OR¹¹, —OC(O)R¹¹, —OC(O)OR¹¹, —NR¹¹R¹², —NR¹¹C(O)R¹⁰, —S(O)₂R¹¹, —SO₂NR¹¹R¹² and —NR¹¹SO₂R¹², —(C₁-C₆)alkyl, —(C₂-C₆)alkenyl, —(C₂-C₆)alkynyl, —(C₃-C₇)cycloalkyl, —(C₂-C₉)heterocycloalkyl, —(C₄-C₉)heterocycloalkenyl, —(C₆-C₁₀)aryl and —(C₁-C₉)heteroaryl.

In another embodiment, the invention relates to a compound of formula I, wherein R⁵ is —(C₁-C₉)heteroaryl substituted with one to five groups selected from the group consisting of -halo, —OH, —CF₃, —CN, —OCF₃, —(C₁-C₆)alkyl and —(C₃-C₁₀)cycloalkyl.

In one embodiment, the invention relates to a compound of formula I wherein R⁵ is —NR¹¹S(O)ⱼR¹⁶.

In another embodiment, the invention relates to a compound of formula I wherein R⁵ is —NR¹¹S(O)ⱼR¹⁶; wherein R¹¹ is selected from the group consisting of —H, —(C₁-C₆)alkyl; and wherein said —(C₁-C₆)alkyl is optionally substituted with one to five R²⁴ groups.

In another embodiment, the invention relates to a compound of formula I wherein R⁵ is —NR¹¹S(O)ⱼR¹⁶; wherein R¹⁶ is selected from the group consisting of —(C₆-C₁₀)aryl and —(C₁-C₉)heteroaryl; and wherein each of the foregoing —(C₆-C₁₀)aryl and —(C₁-C₉)heteroaryl moieties is optionally substituted with one to five R²⁴ groups.

In another embodiment, the invention relates to a compound of formula I wherein R⁵ is —NR¹¹S(O)ⱼR¹⁶; and wherein j is 0.

In another embodiment, the invention relates to a compound of formula I wherein R⁵ is —NR¹¹S(O)ⱼR⁶; and wherein j is 1.

In another embodiment, the invention relates to a compound of formula I wherein R⁵ is —NR¹¹S(O)ⱼR¹⁶; and wherein j is 2.

In one embodiment, the invention relates to a compound of formula I wherein R⁵ is —NR¹¹C(O)NR¹¹R¹².

In another embodiment, the invention relates to a compound of formula I wherein R⁵ is —NR¹¹C(O)NR¹¹R¹²; wherein each R¹¹ is selected from the group consisting of —H, —(C₁-C₆)alkyl; and wherein said —(C₁-C₆)alkyl is optionally substituted with one to five R²⁴ groups.

In another embodiment, the invention relates to a compound of formula I wherein R⁵ is —NR¹¹C(O)NR¹¹R¹²; wherein R¹² is selected from the group consisting of —(C₆-C₁₀)aryl and —(C₁-C₉)heteroaryl; and wherein each of the foregoing —(C₆-C₁₀)aryl and —(C₁-C₉)heteroaryl moieties is optionally substituted with one to five R²⁴ groups.

In one embodiment, the invention relates to a compound of formula I wherein R⁵ is —NR¹⁴R¹⁵.

In another embodiment, the invention relates to a compound of formula I wherein R⁵ is —NR¹⁴R¹⁵; wherein R¹⁴ is selected from the group consisting of —H and —(C₁-C₆)alkyl; and wherein said —(C₁-C₆)alkyl is optionally substituted with one to five R²⁴ groups.

In another embodiment, the invention relates to a compound of formula I wherein R⁵ is —NR¹⁴R¹⁵; and wherein R¹⁵ is selected from the group consisting of —(C₁-C₆)alkyl substituted with one to five R²⁴ groups.

In another embodiment, the invention relates to a compound of formula I wherein R⁵ is —NR¹⁴R¹⁵; wherein R¹⁵ is selected from the group consisting of —(C₆-C₁₀)aryl and —(C₁-C₉)heteroaryl; and wherein each of the foregoing —(C₆-C₁₀)aryl and —(C₁-C₉)heteroaryl moieties is optionally substituted with one to five R²⁴ groups.

The invention also relates to a compound of formula I selected from the group consisting of:
(3S)-3-{[(4-chlorophenyl)amino]methyl}-1-(5-methyl-7H-pyrrolo[2,3-d]pyrimidin-4-yl)pyrrolidin-3-amine;
3-({[2-fluoro-3-(trifluoromethyl)phenyl]amino}methyl)-1-(5-methyl-7H-pyrrolo[2,3-d]pyrimidin-4-yl)pyrrolidin-3-amine;
(3S)-1-(5-methyl-7H-pyrrolo[2,3-d]pyrimidin-4-yl)-3-({[3-(trifluoromethyl)phenyl]amino}methyl)pyrrolidin-3-amine;
(3S)-3-({[2-fluoro-3-(trifluoromethyl)phenyl]amino}methyl)-1-(5-methyl-7H-pyrrolo[2,3-d]pyrimidin-4-yl)pyrrolidin-3-amine;
N-{[(3S)-3-amino-1-(5-methyl-7H-pyrrolo[2,3-d]pyrimidin-4-yl)pyrrolidin-3-yl]methyl}-5-tert-butylisoxazol-3-amine;
(3S)-3-{[(3-fluorophenyl)amino]methyl}-1-(5-methyl-7H-pyrrolo[2,3-d]pyrimidin-4-yl)pyrrolidin-3-amine;
N-{[(3S)-3-amino-1-(5-chloro-7H-pyrrolo[2,3-d]pyrimidin-4-yl)pyrrolidin-3-yl]methyl}-4-methylpyridin-3-amine;
N-{[(3S)-3-amino-1-(5-chloro-7H-pyrrolo[2,3-d]pyrimidin-4-yl)pyrrolidin-3-yl]methyl}-5-isopropyl-1H-pyrazol-3-amine;
N-{[(3S)-3-amino-1-(5-methyl-7H-pyrrolo[2,3-d]pyrimidin-4-yl)pyrrolidin-3-yl]methyl}-6-(trifluoromethyl)pyridin-2-amine;
N-{[(3S)-3-amino-1-(5-chloro-7H-pyrrolo[2,3-d]pyrimidin-4-yl)pyrrolidin-3-yl]methyl}pyridin-3-amine;
N-{[(3S)-3-amino-1-(5-chloro-7H-pyrrolo[2,3-d]pyrimidin-4-yl)pyrrolidin-3-yl]methyl}-5-methylisoxazol-3-amine;
N-{[(3S)-3-amino-1-(5-chloro-7H-pyrrolo[2,3-d]pyrimidin-4-yl)pyrrolidin-3-yl]methyl}-4-chloropyridin-2-amine;
N-{[(3S)-3-amino-1-(5-chloro-7H-pyrrolo[2,3-d]pyrimidin-4-yl)-pyrrolidin-3-yl]methyl}-5-chloropyridin-2-amine;
N-{[(3S)-3-amino-1-(5-chloro-7H-pyrrolo[2,3-d]pyrimidin-4-yl)pyrrolidin-3-yl]methyl}-2,5-difluorobenzamide;
N-{[(3S)-3-amino-1-(5-chloro-7H-pyrrolo[2,3-d]pyrimidin-4-yl)pyrrolidin-3-yl]methyl}-2-chloro-4-fluorobenzamide;
N-{[(3S)-3-amino-1-(5-chloro-7H-pyrrolo[2,3-d]pyrimidin-4-yl)pyrrolidin-3-yl]methyl}benzamide;
N-{[(3S)-3-amino-1-(5-chloro-7H-pyrrolo[2,3-d]pyrimidin-4-yl)pyrrolidin-3-yl]methyl}-3-chlorobenzamide;
N-{[(3S)-3-amino-1-(5-chloro-7H-pyrrolo[2,3-d]pyrimidin-4-yl)pyrrolidin-3-yl]methyl}-3,4-difluorobenzamide; and
N-{[(3S)-3-amino-1-(5-ethyl-7H-pyrrolo[2,3-d]pyrimidin-4-yl)pyrrolidin-3-yl]methyl}-3-chlorobenzamide

In another embodiment, the invention also relates to a compound of formula I selected from the group consisting of:
N-{[(3S)-3-amino-1-(5-chloro-7H-pyrrolo[2,3-d]pyrimidin-4-yl)pyrrolidin-3-yl]methyl}-4-fluorobenzamide;
N-{[(3S)-3-amino-1-(5-ethyl-7H-pyrrolo[2,3-d]pyrimidin-4-yl)pyrrolidin-3-yl]methyl}-5-methylisoxazol-3-amine;
N-{[(3S)-3-amino-1-(5-chloro-7H-pyrrolo[2,3-d]pyrimidin-4-yl)pyrrolidin-3-yl]methyl}-4-chlorobenzamide;
N-{[(3S)-3-amino-1-(5-chloro-7H-pyrrolo[2,3-d]pyrimidin-4-yl)pyrrolidin-3-yl]methyl}-2-fluorobenzamide;
N-{[(3S)-3-amino-1-(5-chloro-7H-pyrrolo[2,3-d]pyrimidin-4-yl)pyrrolidin-3-yl]methyl}-2,3-difluorobenzamide;
N-{[(3S)-3-amino-1-(5-chloro-7H-pyrrolo[2,3-d]pyrimidin-4-yl)pyrrolidin-3-yl]methyl}-2,4-difluorobenzamide;
N-{[(3S)-3-amino-1-(5-chloro-7H-pyrrolo[2,3-d]pyrimidin-4-yl)pyrrolidin-3-yl]methyl}-4-chloro-2-fluorobenzamide;
N-{[(3S)-3-amino-1-(5-chloro-7H-pyrrolo[2,3-d]pyrimidin-4-yl)pyrrolidin-3-yl]methyl}-2-chlorobenzamide;
N-{[(3S)-3-amino-1-(5-ethyl-7H-pyrrolo[2,3-d]pyrimidin-4-yl)pyrrolidin-3-yl]methyl}-4-chlorobenzamide;
N-{[(3S)-3-amino-1-(5-methyl-7H-pyrrolo[2,3-d]pyrimidin-4-yl)pyrrolidin-3-yl]methyl}-4-fluorobenzamide;
N-{[(3S)-3-amino-1-(5-ethyl-7H-pyrrolo[2,3-d]pyrimidin-4-yl)pyrrolidin-3-yl]methyl}-4-fluorobenzamide;
N-{[(3R)-3-amino-1-(5-propyl-7H-pyrrolo[2,3-d]pyrimidin-4-yl)pyrrolidin-3-yl]methyl}-5-methyl isoxazole-3-carboxamide;
N-{[(3S)-3-amino-1-(5-methyl-7H-pyrrolo[2,3-d]pyrimidin-4-yl)pyrrolidin-3-yl]methyl}-2,4-difluorobenzamide;
N-{[(3S)-3-amino-1-(5-ethyl-7H-pyrrolo[2,3-d]pyrimidin-4-yl)pyrrolidin-3-yl]methyl}-2-fluorobenzamide;
N-{[(3S)-3-amino-1-(5-methyl-7H-pyrrolo[2,3-d]pyrimidin-4-yl)pyrrolidin-3-yl]methyl}-2-chloro-4-fluorobenzamide;
N-{[(3S)-3-amino-1-(5-propyl-7H-pyrrolo[2,3-d]pyrimidin-4-yl)pyrrolidin-3-yl]methyl}pyridin-3-amine;
N-{[(3S)-3-amino-1-(3-chloro-1H-pyrrolo[2,3-b]pyridin-4-yl)pyrrolidin-3-yl]methyl}-4-methylpyridin-2-amine;
N-{[(3S)-3-amino-1-(5-ethyl-7H-pyrrolo[2,3-d]pyrimidin-4-yl)pyrrolidin-3-yl]methyl}-2,4-difluorobenzamide;
N-{[(3S)-3-amino-1-(5-methyl-7H-pyrrolo[2,3-d]pyrimidin-4-yl)pyrrolidin-3-yl]methyl}-4-chloro-2-fluorobenzamide; and
N-{[(3S)-3-amino-1-(5-ethyl-7H-pyrrolo[2,3-d]pyrimidin-4-yl)pyrrolidin-3-yl]methyl}-4-chloro-2-fluorobenzamide.

In another embodiment, the invention also relates to a compound of formula I selected from the group consisting of:
4-{[(4-chlorobenzyl)amino]methyl}-1-(5-methyl-7H-pyrrolo[2,3-d]pyrimidin-4-yl)piperidin-4-amine;
3-(2-fluoro-3-(trifluoromethyl)phenylamino)methyl)-1-(5-methyl-7H-pyrrolo[2,3-d]pyrimidin-4-yl)pyrrolidin-3-amine;
4-{3-amino-3-[(4-chloro-phenylamino)-methyl]-pyrrolidin-1-yl}-7H-pyrrolo[2,3-d]pyrimidine-5-carbonitrile;
2-{[3-Amino-1-(3-methyl-1H-pyrazolo[3,4-d]pyrimidin-4-yl)-pyrrolidin-3-ylmethyl]-amino}-benzoic acid methyl ester;
1-(5-Chloro-7H-pyrrolo[2,3-d]pyrimidin-4-yl)-3-[(3-phenoxy-phenylamino)-methyl]-pyrrolidin-3-ylamine;
4-(3-amino-3-((3-chloro-2-fluorophenylamino)methyl)pyrrolidin-1-yl)-1H-pyrazolo[3,4-d]pyrimidine-3-carbonitrile;
4-{3-Amino-3-[(3-chloro-phenylamino)-methyl]-pyrrolidin-1-yl}-1H-pyrazolo[3,4-d]pyrimidine-3-carbonitrile;
3-{[(2,3-dichlorophenyl)amino]methyl}-1-(5-methylpyrrolo[2,1-f][1,2,4]triazin-4-yl)pyrrolidin-3-amine;
4-(3-amino-3-{[(2-phenoxyphenyl)amino]methyl}pyrrolidin-1-yl)-1H-pyrrolo[2,3-b]pyridine-3-carbonitrile;
4-({[3-amino-1-(5-methyl-7H-pyrrolo[2,3-d]pyrimidin-4-yl)pyrrolidin-3-yl]methyl}amino)-N-benzylbenzenesulfonamide;
1-(9H-purin-6-yl)-3-({[3-(trifluoromethyl)phenyl]amino}methyl)pyrrolidin-3-amine;
3-{[(2-{[(3R)-3-fluoropyrrolidin-1-yl]sulfonyl}phenyl)amino]methyl}-1-(5-methyl-7H-pyrrolo[2,3-d]pyrimidin-4-yl)pyrrolidin-3-amine;
2-[4-({[3-amino-1-(5-methyl-7H-pyrrolo[2,3-d]pyrimidin-4-yl)pyrrolidin-3-yl]methyl}amino)phenyl]acetamide;
3-{[(4-chlorophenyl)amino]methyl}-1-(1H-pyrrolo[2,3-b]pyridin-4-yl)pyrrolidin-3-amine;
methyl 2-({[3-amino-1-(5-methyl-7H-pyrrolo[2,3-d]pyrimidin-4-yl)pyrrolidin-3-yl]methyl}-amino)benzoate;
1-(5-methyl-7H-pyrrolo[2,3-d]pyrimidin-4-yl)-3-{[(2-phenoxyphenyl)amino]methyl}-pyrrolidin-3-amine;
3-{[(3-chloro-2-fluorophenyl)amino]methyl}-1-(5-methyl-7H-pyrrolo[2,3-d]pyrimidin-4-yl)pyrrolidin-3-amine;
4-({[3-amino-1-(5-methyl-7H-pyrrolo[2,3-d]pyrimidin-4-yl)pyrrolidin-3-yl]methyl}amino)-2-chlorobenzonitrile; and
1-(5-methyl-7H-pyrrolo[2,3-d]pyrimidin-4-yl)-3-({[3-(trifluoromethyl)phenyl]-amino}methyl)pyrrolidin-3-amine.

In another embodiment, the invention also relates to a compound of formula I selected from the group consisting of:
1-(5-methyl-7H-pyrrolo[2,3-d]pyrimidin-4-yl)-3-{[(2-piperidin-1-ylphenyl)amino]methyl}-pyrrolidin-3-amine;
1-(5-methyl-7H-pyrrolo[2,3-d]pyrimidin-4-yl)-3-({[3-(trifluoromethoxy)phenyl]amino}-methyl)pyrrolidin-3-amine;
3-{[(2,3-dichlorophenyl)amino]methyl}-1-(5-methyl-7H-pyrrolo[2,3-d]pyrimidin-4-yl)pyrrolidin-3-amine;
2-({[3-amino-1-(5-methyl-7H-pyrrolo[2,3-d]pyrimidin-4-yl)pyrrolidin-3-yl]methyl}amino)-N-benzylbenzamide;
3-{[(3-fluoro-2-methylphenyl)amino]methyl}-1-(5-methyl-7H-pyrrolo[2,3-d]pyrimidin-4-yl)pyrrolidin-3-amine;
1-(5-methyl-7H-pyrrolo[2,3-d]pyrimidin-4-yl)-3-{[(2-morpholin-4-ylphenyl)amino]-methyl}pyrrolidin-3-amine;
3-({[3-methoxy-5-(trifluoromethyl)phenyl]amino}methyl)-1-(5-methyl-7H-pyrrolo[2,3-d]pyrimidin-4-yl)pyrrolidin-3-amine;
3-{[(4-chloro-2-fluorophenyl)amino]methyl}-1-(5-methyl-7H-pyrrolo[2,3-d]pyrimidin-4-yl)pyrrolidin-3-amine;
3-{[(2,3-difluorophenyl)amino]methyl}-1-(5-methyl-7H-pyrrolo[2,3-d]pyrimidin-4-yl)pyrrolidin-3-amine;
3-({[2-(4-chlorophenoxy)-5-(trifluoromethyl)phenyl]amino}methyl)-1-(5-methyl-7H-pyrrolo[2,3-d]pyrimidin-4-yl)pyrrolidin-3-amine;
3-{[(2,4-difluorophenyl)amino]methyl}-1-(5-methyl-7H-pyrrolo[2,3-d]pyrimidin-4-yl)pyrrolidin-3-amine;
1-(5-methyl-7H-pyrrolo[2,3-d]pyrimidin-4-yl)-3-[(5,6,7,8-tetrahydronaphthalen-1-ylamino)methyl]pyrrolidin-3-amine;
4-(3-amino-3-{[(4-chlorophenyl)amino]methyl}pyrrolidin-1-yl)-7H-pyrrolo[2,3-d]pyrimidine-5-carbonitrile;
4-{3-amino-3-[(5,6,7,8-tetrahydronaphthalen-1-ylamino)methyl]pyrrolidin-1-yl}-7H-pyrrolo[2,3-d]pyrimidine-5-carbonitrile;
4-{3-amino-3-[(1,2,3,4-tetrahydroisoquinolin-7-ylamino)methyl]pyrrolidin-1-yl}-7H-pyrrolo[2,3-d]pyrimidine-5-carbonitrile;
4-[3-amino-3-({[2-fluoro-3-(trifluoromethyl)phenyl]amino}methyl)pyrrolidin-1-yl]-7H-pyrrolo[2,3-d]pyrimidine-5-carbonitrile;
methyl-2-({[3-amino-1-(3-methyl-1H-pyrazolo[3,4-d]pyrimidin-4-yl)pyrrolidin-3-yl]methyl}amino)benzoate;
3-{[(3-chloro-2-fluorophenyl)amino]methyl}-1-(3-methyl-1H-pyrazolo[3,4-d]pyrimidin-4-yl)pyrrolidin-3-amine;
1-(1H-pyrrolo[2,3-b]pyridin-4-yl)-3-({[3-(trifluoromethyl)phenyl]amino}methyl)pyrrolidin-3-amine; and
1-(5-chloro-7H-pyrrolo[2,3-d]pyrimidin-4-yl)-3-{[(3-phenoxyphenyl)amino]methyl}-pyrrolidin-3-amine.

In another embodiment, the invention also relates to a compound of formula I selected from the group consisting of:
3-{[(3-chloro-2-methylphenyl)amino]methyl}-1-(5-chloro-7H-pyrrolo[2,3-d]pyrimidin-4-yl)pyrrolidin-3-amine;
4-(3-amino-3-{[(3-chloro-2-fluorophenyl)amino]methyl}pyrrolidin-1-yl)-1H-pyrazolo[3,4-d]pyrimidine-3-carbonitrile;
4-(3-amino-3-{[(3-chlorophenyl)amino]methyl}pyrrolidin-1-yl)-1H-pyrazolo[3,4-d]pyrimidine-3-carbonitrile;
1-(5-ethyl-7H-pyrrolo[2,3-d]pyrimidin-4-yl)-3-({[3-(trifluoromethyl)phenyl]amino}-methyl)pyrrolidin-3-amine;
3-[(benzylamino)methyl]-1-(5-methyl-7H-pyrrolo[2,3-d]pyrimidin-4-yl)piperidin-3-amine;
3-(anilinomethyl)-1-(5-methyl-7H-pyrrolo[2,3-d]pyrimidin-4-yl)piperidin-3-amine;
N-{[3-amino-1-(5-methyl-7H-pyrrolo[2,3-d]pyrimidin-4-yl)piperidin-3-yl]methyl}pyridin-3-amine;
1-(5-methyl-7H-pyrrolo[2,3-d]pyrimidin-4-yl)-3-({[3-(1,3-oxazol-5-yl)phenyl]amino}-methyl)pyrrolidin-3-amine;
4-(anilinomethyl)-1-(5-methyl-7H-pyrrolo[2,3-d]pyrimidin-4-yl)piperidin-4-amine;
4-({[(1S)-1-(4-chlorophenyl)ethyl]amino}methyl)-1-(5-methyl-7H-pyrrolo[2,3-d]pyrimidin-4-yl)piperidin-4-amine;
3-{[(4-chlorobenzyl)(methyl)amino]methyl}-1-(5-methyl-7H-pyrrolo[2,3-d]pyrimidin-4-yl)pyrrolidin-3-amine;
N′-{[3-amino-1-(5-methyl-7H-pyrrolo[2,3-d]pyrimidin-4-yl)pyrrolidin-3-yl]methyl}-N,N-dimethylethane-1,2-diamine;
N-{[3-amino-1-(5-methyl-7H-pyrrolo[2,3-d]pyrimidin-4-yl)pyrrolidin-3-yl]methyl}-N-benzyl-N′, N′-dimethylethane-1,2-diamine;
3-{[methyl(phenyl)amino]methyl}-1-(5-methyl-7H-pyrrolo[2,3-d]pyrimidin-4-yl)pyrrolidin-3-amine;
3-(3,4-dihydroquinolin-1(2H)-ylmethyl)-1-(5-methyl-7H-pyrrolo[2,3-d]pyrimidin-4-yl)pyrrolidin-3-amine;
3-{[ethyl(phenyl)amino]methyl}-1-(5-methyl-7H-pyrrolo[2,3-d]pyrimidin-4-yl)pyrrolidin-3-amine;
3-(2,3-dihydro-1H-indol-1-ylmethyl)-1-(5-methyl-7H-pyrrolo[2,3-d]pyrimidin-4-yl)pyrrolidin-3-amine; and
N-{[3-amino-1-(5-methyl-7H-pyrrolo[2,3-d]pyrimidin-4-yl)pyrrolidin-3-yl]methyl}-6-chloropyridazin-3-amine.

In another embodiment, the invention also relates to a compound of formula I selected from the group consisting of:
(3S)-3-({[(5-methylisoxazol-3-yl)methyl]amino}methyl)-1-(5-methyl-7H-pyrrolo[2,3-d]pyrimidin-4-yl)pyrrolidin-3-amine;
3-{[(cyclopropyl methyl)amino]methyl}-1-(5-methyl-7H-pyrrolo[2,3-d]pyrimidin-4-yl)pyrrolidin-3-amine;
3-{[(4-chlorophenyl)(methyl)amino]methyl}-1-(5-chloro-7H-pyrrolo[2,3-d]pyrimidin-4-yl)pyrrolidin-3-amine;
6-({[(3S)-3-amino-1-(5-methyl-7H-pyrrolo[2,3-d]pyrimidin-4-yl)pyrrolidin-3-yl]methyl}amino)pyridin-2(3H)-one;
N-{[3-amino-1-(5-methyl-7H-pyrrolo[2,3-d]pyrimidin-4-yl)pyrrolidin-3-yl]methyl}-4-(trifluoromethyl)pyrimidin-2-amine;
N-{[3-amino-1-(5-methyl-7H-pyrrolo[2,3-d]pyrimidin-4-yl)pyrrolidin-3-yl]methyl}pyrimidin-2-amine;
N-{[3-amino-1-(1H-pyrrolo[2,3-b]pyridin-4-yl)pyrrolidin-3-yl]methyl}-5-chloropyrimidin-2-amine;
1-(5-Methyl-7H-pyrrolo[2,3-d]pyrimidin-4-yl)-3-(3-trifluoromethyl-phenoxymethyl)-pyrrolidin -3-ylamine;
1-(5-methyl-7H-pyrrolo[2,3-d]pyrimidin-4-yl)-3-{[3-(trifluoromethyl)phenoxy]-methyl}pyrrolidin-3-amine;
(3R)-1-(5-methyl-7H-pyrrolo[2,3-d]pyrimidin-4-yl)-3-{[3-(trifluoromethyl)phenoxy]-methyl}pyrrolidin-3-amine;
(3S)-1-(5-methyl-7H-pyrrolo[2,3-d]pyrimidin-4-yl)-3-{[3-(trifluoromethyl)phenoxy]-methyl}pyrrolidin-3-amine;
4-(3-amino-3-{[3-(trifluoromethyl)phenoxy]methyl}pyrrolidin-1-yl)-7H-pyrrolo[2,3-d]pyrimidine-5-carbonitrile;
1-(5-ethyl-7H-pyrrolo[2,3-d]pyrimidin-4-yl)-3-{[3-(trifluoromethyl)phenoxy]-methyl}pyrrolidin-3-amine;
1-(5-ethyl-7H-pyrrolo[2,3-d]pyrimidin-4-yl)-3-[(3-methyl phenoxy)methyl]pyrrolidin-3-amine;
1-(5-methyl-7H-pyrrolo[2,3-d]pyrimidin-4-yl)-3-((3-(trifluoromethyl)phenylthio)methyl)-pyrrolidin-3-amine;
1-(5-methyl-7H-pyrrolo[2,3-d]pyrimidin-4-yl)-3-{2-[3-(trifluoromethyl)phenyl]-ethyl}pyrrolidin-3-amine;
(E)-3-(3-trifluoromethyl)styryl)-1-(5-methyl-7H-pyrrolo[2,3-d]pyrimidin-4-yl)-3-amine;
N-((3-amino-1-(5-methyl-7H-pyrrolo[2,3-d]pyrimidin-4-yl)pyrrolidin-3-yl)methyl)benzamide;
N—(((S)-3-amino-1-(5-methyl-7H-pyrrolo[2,3-d]pyrimidin-4-yl)pyrrolidin-3-yl)methyl)-2-chlorobenzamide; and
N-{[(3S)-3-amino-1-(5-ethyl-7H-pyrrolo[2,3-d]pyrimidin-4-yl)pyrrolidin-3-yl]methyl}-2-methylpropanamide.

In another embodiment, the invention also relates to a compound of formula I selected from the group consisting of:
(S)—N-((3-amino-1-(5-methyl-7H-pyrrolo[2,3-d]pyrimidin-4-yl)pyrrolidin-3-yl)methyl)-4-chlorobenzamide;
1-{[3-amino-1-(5-methyl-7H-pyrrolo[2,3-d]pyrimidin-4-yl)pyrrolidin-3-yl]methyl}-3-(2,3-dimethylphenyl)urea;
1-{[3-amino-1-(5-methyl-7H-pyrrolo[2,3-d]pyrimidin-4-yl)pyrrolidin-3-yl]methyl}-1-(2-methoxyethyl)-3-phenylurea;
N-{[3-amino-1-(5-methyl-7H-pyrrolo[2,3-d]pyrimidin-4-yl)pyrrolidin-3-yl]methyl}benzenesulfonamide;
N-{[(3R)-3-amino-1-(5-chloro-7H-pyrrolo[2,3-d]pyrimidin-4-yl)pyrrolidin-3-yl]methyl}-5-methyl isoxazole-3-carboxamide;
N-{[(3S)-3-amino-1-(5-chloro-7H-pyrrolo[2,3-d]pyrimidin-4-yl)pyrrolidin-3-yl]methyl}-4-chlorobenzamide;
N-{[(3S)-3-amino-1-(5-chloro-7H-pyrrolo[2,3-d]pyrimidin-4-yl)pyrrolidin-3-yl]methyl}-4-chloro-3-fluorobenzamide;
N-{[(3S)-3-amino-1-(5-cyano-7H-pyrrolo[2,3-d]pyrimidin-4-yl)pyrrolidin-3-yl]methyl}-4-chlorobenzamide;
N-{[(3S)-3-amino-1-(5-methyl-7H-pyrrolo[2,3-d]pyrimidin-4-yl)pyrrolidin-3-yl]methyl}-2,3-difluorobenzamide;
N-{[(3S)-3-amino-1-(5-methyl-7H-pyrrolo[2,3-d]pyrimidin-4-yl)pyrrolidin-3-yl]methyl}-4-chlorobenzamide;
N-{[(3S)-3-amino-1-(5-ethyl-7H-pyrrolo[2,3-d]pyrimidin-4-yl)pyrrolidin-3-yl]methyl}-2,3-difluorobenzamide;
N-{[(3S)-3-amino-1-(5-cyano-7H-pyrrolo[2,3-d]pyrimidin-4-yl)pyrrolidin-3-yl]methyl}-2,3-difluorobenzamide;
N-{[(3S)-3-amino-1-(5-cyano-7H-pyrrolo[2,3-d]pyrimidin-4-yl)pyrrolidin-3-yl]methyl}-4-fluorobenzamide;
N-{[(3S)-3-amino-1-(5-cyano-7H-pyrrolo[2,3-d]pyrimidin-4-yl)pyrrolidin-3-yl]methyl}-2,4-difluorobenzamide;
N-{[(3S)-3-amino-1-(5-cyano-7H-pyrrolo[2,3-d]pyrimidin-4-yl)pyrrolidin-3-yl]methyl}-4-chloro-2-fluorobenzamide;
N-{[(3S)-3-amino-1-(5-methyl-7H-pyrrolo[2,3-d]pyrimidin-4-yl)pyrrolidin-3-yl]methyl}-2,5-difluorobenzamide;
N-{[(3S)-3-amino-1-(5-ethyl-7H-pyrrolo[2,3-d]pyrimidin-4-yl)pyrrolidin-3-yl]methyl}-2,4-difluorobenzamide; and
N-{[(3S)-3-amino-1-(5-chloro-7H-pyrrolo[2,3-d]pyrimidin-4-yl)pyrrolidin-3-yl]methyl}-3-fluorobenzamide.

In another embodiment, the invention also relates to a compound of formula I selected from the group consisting of:
N-{[(3S)-3-amino-1-(5-ethyl-7H-pyrrolo[2,3-d]pyrimidin-4-yl)pyrrolidin-3-yl]methyl}-3-fluorobenzamide;
N-{[(3S)-3-amino-1-(5-methyl-7H-pyrrolo[2,3-d]pyrimidin-4-yl)pyrrolidin-3-yl]methyl}benzamide;
N-{[(3S)-3-amino-1-(5-ethyl-7H-pyrrolo[2,3-d]pyrimidin-4-yl)pyrrolidin-3-yl]methyl}benzamide;
N-{[(3S)-3-amino-1-(5-cyano-7H-pyrrolo[2,3-d]pyrimidin-4-yl)pyrrolidin-3-yl]methyl}benzamide;
N-{[(3S)-3-amino-1-(5-chloro-7H-pyrrolo[2,3-d]pyrimidin-4-yl)pyrrolidin-3-yl]methyl}-2-chloro-6-fluorobenzamide;
N-{[(3S)-3-amino-1-(5-methyl-7H-pyrrolo[2,3-d]pyrimidin-4-yl)pyrrolidin-3-yl]methyl}-3,4-difluorobenzamide;
N-{[(3S)-3-amino-1-(5-methyl-7H-pyrrolo[2,3-d]pyrimidin-4-yl)pyrrolidin-3-yl]methyl}-3-chlorobenzamide;
N-{[(3S)-3-amino-1-(5-methyl-7H-pyrrolo[2,3-d]pyrimidin-4-yl)pyrrolidin-3-yl]methyl}-2-chloro-6-fluorobenzamide;
N-{[(3S)-3-amino-1-(5-methyl-7H-pyrrolo[2,3-d]pyrimidin-4-yl)pyrrolidin-3-yl]methyl}-2-chloro-3-methylbenzamide;
N-{[(3S)-3-amino-1-(5-ethyl-7H-pyrrolo[2,3-d]pyrimidin-4-yl)pyrrolidin-3-yl]methyl}-2-chloro-3-methylbenzamide;
N-{[(3S)-3-amino-1-(5-chloro-7H-pyrrolo[2,3-d]pyrimidin-4-yl)pyrrolidin-3-yl]methyl}-2-chloro-3-methylbenzamide;
N-{[(3S)-3-amino-1-(5-methyl-7H-pyrrolo[2,3-d]pyrimidin-4-yl)pyrrolidin-3-yl]methyl}-2,3,6-trifluorobenzamide;
N-{[(3S)-3-amino-1-(5-methyl-7H-pyrrolo[2,3-d]pyrimidin-4-yl)pyrrolidin-3-yl]methyl}-2-fluoro-6-methylbenzamide;
N-{[(3S)-3-amino-1-(5-chloro-7H-pyrrolo[2,3-d]pyrimidin-4-yl)pyrrolidin-3-yl]methyl}-2-fluoro-6-methylbenzamide;
N-{[(3S)-3-amino-1-(5-methyl-7H-pyrrolo[2,3-d]pyrimidin-4-yl)pyrrolidin-3-yl]methyl}-2-chloro-3-fluorobenzamide;
N-{[(3S)-3-amino-1-(5-chloro-7H-pyrrolo[2,3-d]pyrimidin-4-yl)pyrrolidin-3-yl]methyl}-2-chloro-3-fluorobenzamide;
N-{[(3S)-3-amino-1-(5-ethyl-7H-pyrrolo[2,3-d]pyrimidin-4-yl)pyrrolidin-3-yl]methyl}-2-chloro-3-fluorobenzamide;
N-{[(3S)-3-amino-1-(5-methyl-7H-pyrrolo[2,3-d]pyrimidin-4-yl)pyrrolidin-3-yl]methyl}-2,6-difluorobenzamide;
N-{[(3S)-3-amino-1-(5-methyl-7H-pyrrolo[2,3-d]pyrimidin-4-yl)pyrrolidin-3-yl]methyl}-2-methylbenzamide; and
N-{[(3S)-3-amino-1-(5-methyl-7H-pyrrolo[2,3-d]pyrimidin-4-yl)pyrrolidin-3-yl]methyl}-2-chlorobenzamide.

In another embodiment, the invention also relates to a compound of formula I selected from the group consisting of:
N-{[(3S)-3-amino-1-(5-ethyl-7H-pyrrolo[2,3-d]pyrimidin-4-yl)pyrrolidin-3-yl]methyl}-2-chlorobenzamide;
N-{[(3S)-3-amino-1-(5-chloro-7H-pyrrolo[2,3-d]pyrimidin-4-yl)pyrrolidin-3-yl]methyl}-2,6-difluorobenzamide;
N-{[(3S)-3-amino-1-(5-chloro-7H-pyrrolo[2,3-d]pyrimidin-4-yl)pyrrolidin-3-yl]methyl}-2-methylbenzamide;
N-{[(3S)-3-amino-1-(5-ethyl-7H-pyrrolo[2,3-d]pyrimidin-4-yl)pyrrolidin-3-yl]methyl}-2,3,6-trifluorobenzamide;
N-{[(3S)-3-amino-1-(5-cyano-7H-pyrrolo[2,3-d]pyrimidin-4-yl)pyrrolidin-3-yl]methyl}-2-chloro-4-fluorobenzamide;
N-{[(3S)-3-amino-1-(5-cyano-7H-pyrrolo[2,3-d]pyrimidin-4-yl)pyrrolidin-3-yl]methyl}-2,5-difluorobenzamide;
N-{[(3S)-3-amino-1-(5-ethyl-7H-pyrrolo[2,3-d]pyrimidin-4-yl)pyrrolidin-3-yl]methyl}-2-chloro-4-fluorobenzamide;
N-{[(3S)-3-amino-1-(5-ethyl-7H-pyrrolo[2,3-d]pyrimidin-4-yl)pyrrolidin-3-yl]methyl}-2,5-difluorobenzamide;
N-{[(3S)-3-amino-1-(7H-pyrrolo[2,3-d]pyrimidin-4-yl)pyrrolidin-3-yl]methyl}-2-chloro-4-fluorobenzamide;
N-{[(3S)-3-amino-1-(7H-pyrrolo[2,3-d]pyrimidin-4-yl)pyrrolidin-3-yl]methyl}-2,5-difluorobenzamide;
N-{[(3S)-3-amino-1-(5-ethyl-7H-pyrrolo[2,3-d]pyrimidin-4-yl)pyrrolidin-3-yl]methyl}-2-chloro-6-fluorobenzamide;
N-{[(3S)-3-amino-1-(5-ethyl-7H-pyrrolo[2,3-d]pyrimidin-4-yl)pyrrolidin-3-yl]methyl}-2,6-difluorobenzamide;
N-{[(3S)-3-amino-1-(5-ethyl-7H-pyrrolo[2,3-d]pyrimidin-4-yl)pyrrolidin-3-yl]methyl}-2-methylbenzamide;
N-{[(3S)-3-amino-1-(5-methyl-7H-pyrrolo[2,3-d]pyrimidin-4-yl)pyrrolidin-3-yl]methyl}-3-fluoro-2-methylbenzamide;
N-{[(3S)-3-amino-1-(5-methyl-7H-pyrrolo[2,3-d]pyrimidin-4-yl)pyrrolidin-3-yl]methyl}-2-chloro-4,5-difluorobenzamide;
N-{[(3S)-3-amino-1-(5-cyano-7H-pyrrolo[2,3-d]pyrimidin-4-yl)pyrrolidin-3-yl]methyl}-2-chloro-6-fluorobenzamide;
N-{[(3S)-3-amino-1-(5-cyano-7H-pyrrolo[2,3-d]pyrimidin-4-yl)pyrrolidin-3-yl]methyl}-2,3,6-trifluorobenzamide;
N-{[(3S)-3-amino-1-(5-chloro-7H-pyrrolo[2,3-d]pyrimidin-4-yl)pyrrolidin-3-yl]methyl}-2-chloro-4,5-difluorobenzamide;
N-{[(3S)-3-amino-1-(5-ethyl-7H-pyrrolo[2,3-d]pyrimidin-4-yl)pyrrolidin-3-yl]methyl}-3-fluoro-2-methylbenzamide; and
N-{[(3S)-3-amino-1-(5-cyano-7H-pyrrolo[2,3-d]pyrimidin-4-yl)pyrrolidin-3-yl]methyl}-3-fluoro-2-methylbenzamide.

In another embodiment, the invention also relates to a compound of formula I selected from the group consisting of:
N-{[(3S)-3-amino-1-(5-cyano-7H-pyrrolo[2,3-d]pyrimidin-4-yl)pyrrolidin-3-yl]methyl}-2-chloro-4,5-difluorobenzamide;
N-{[(3S)-3-amino-1-(5-cyano-7H-pyrrolo[2,3-d]pyrimidin-4-yl)pyrrolidin-3-yl]methyl}-3-chlorobenzamide;
N-{[(3S)-3-amino-1-(5-ethyl-7H-pyrrolo[2,3-d]pyrimidin-4-yl)pyrrolidin-3-yl]methyl}-2-chloro-4,5-difluorobenzamide;
N-{[(3S)-3-amino-1-(5-ethyl-7H-pyrrolo[2,3-d]pyrimidin-4-yl)pyrrolidin-3-yl]methyl}-3-fluoro-2-methylbenzamide;
N-{[(3S)-3-amino-1-(5-ethyl-7H-pyrrolo[2,3-d]pyrimidin-4-yl)pyrrolidin-3-yl]methyl}cyclopentanecarboxamide;
N-{[(3S)-3-amino-1-(5-chloro-7H-pyrrolo[2,3-d]pyrimidin-4-yl)pyrrolidin-3-yl]methyl}-1-methylcyclopropanecarboxamide;
N-{[(3S)-3-amino-1-(5-cyano-7H-pyrrolo[2,3-d]pyrimidin-4-yl)pyrrolidin-3-yl]methyl}-3-chloro-2-fluorobenzamide;
N-{[(3S)-3-amino-1-(5-methyl-7H-pyrrolo[2,3-d]pyrimidin-4-yl)pyrrolidin-3-yl]methyl}-4-methoxybenzamide;
N-{[(3S)-3-amino-1-(5-ethyl-7H-pyrrolo[2,3-d]pyrimidin-4-yl)pyrrolidin-3-yl]methyl}-4-methoxybenzamide;
N-{[(3S)-3-amino-1-(5-chloro-7H-pyrrolo[2,3-d]pyrimidin-4-yl)pyrrolidin-3-yl]methyl}-4-methoxybenzamide;
N-{[(3S)-3-amino-1-(5-cyano-7H-pyrrolo[2,3-d]pyrimidin-4-yl)pyrrolidin-3-yl]methyl}-4-methoxybenzamide;
N-{[(3S)-3-amino-1-(5-methyl-7H-pyrrolo[2,3-d]pyrimidin-4-yl)pyrrolidin-3-yl]methyl}-2-chloro-3-(trifluoromethyl)benzamide;
N-{[(3S)-3-amino-1-(5-chloro-7H-pyrrolo[2,3-d]pyrimidin-4-yl)pyrrolidin-3-yl]methyl}-2-chloro-3-(trifluoromethyl)benzamide;
N-{[(3S)-3-amino-1-(5-ethyl-7H-pyrrolo[2,3-d]pyrimidin-4-yl)pyrrolidin-3-yl]methyl}-2-chloro-3-(trifluoromethyl)benzamide;
N-{[(3S)-3-amino-1-(5-methyl-7H-pyrrolo[2,3-d]pyrimidin-4-yl)pyrrolidin-3-yl]methyl}-2-fluoro-4-methylbenzamide;
N-{[(3S)-3-amino-1-(5-chloro-7H-pyrrolo[2,3-d]pyrimidin-4-yl)pyrrolidin-3-yl]methyl}-2-fluoro-4-methylbenzamide;
N-{[(3S)-3-amino-1-(5-ethyl-7H-pyrrolo[2,3-d]pyrimidin-4-yl)pyrrolidin-3-yl]methyl}-2-fluoro-4-methylbenzamide;
N-{[(3S)-3-amino-1-(5-methyl-7H-pyrrolo[2,3-d]pyrimidin-4-yl)pyrrolidin-3-yl]methyl}-4-fluoro-3-(trifluoromethyl)benzamide; and
N-{[(3S)-3-amino-1-(5-ethyl-7H-pyrrolo[2,3-d]pyrimidin-4-yl)pyrrolidin-3-yl]methyl}-4-fluoro-3-(trifluoromethyl)benzamide.

In another embodiment, the invention also relates to a compound of formula I selected from the group consisting of:
N-{[(3S)-3-amino-1-(5-cyano-7H-pyrrolo[2,3-d]pyrimidin-4-yl)pyrrolidin-3-yl]methyl}-4-fluoro-3-(trifluoromethyl)benzamide;
N-{[(3S)-3-amino-1-(5-chloro-7H-pyrrolo[2,3-d]pyrimidin-4-yl)pyrrolidin-3-yl]methyl}-4-fluoro-3-(trifluoromethyl)benzamide;
N-{[(3S)-3-amino-1-thieno[2,3-d]pyrimidin-4-ylpyrrolidin-3-yl]methyl}-2,4-difluorobenzamide;
N-{[(3S)-3-amino-1-(5-ethyl-7H-pyrrolo[2,3-d]pyrimidin-4-yl)pyrrolidin-3-yl]methyl}-2-cyclopropylacetamide;
N-{[(3S)-3-amino-1-(5-ethyl-7H-pyrrolo[2,3-d]pyrimidin-4-yl)pyrrolidin-3-yl]methyl}cyclopropanecarboxamide;
N-{[(3S)-3-amino-1-(5-ethyl-7H-pyrrolo[2,3-d]pyrimidin-4-yl)pyrrolidin-3-yl]methyl}-3-chloro-2-fluorobenzamide;
N-{[(3S)-3-amino-1-(5-ethyl-7H-pyrrolo[2,3-d]pyrimidin-4-yl)pyrrolidin-3-yl]methyl}-2-fluoro-6-methylbenzamide;
N-{[(3S)-3-amino-1-(5-ethyl-7H-pyrrolo[2,3-d]pyrimidin-4-yl)pyrrolidin-3-yl]methyl}-4-methoxybutanamide;
N-{[(3S)-3-amino-1-(5-ethyl-7H-pyrrolo[2,3-d]pyrimidin-4-yl)pyrrolidin-3-yl]methyl}-3-methylbutanamide;
N-{[(3S)-3-amino-1-(5-ethyl-7H-pyrrolo[2,3-d]pyrimidin-4-yl)pyrrolidin-3-yl]methyl}-3-ethyl-1-methyl-1H-pyrazole-5-carboxamide;
N-{[(3S)-3-amino-1-(5-ethyl-7H-pyrrolo[2,3-d]pyrimidin-4-yl)pyrrolidin-3-yl]methyl}-5-fluoro-2-methylbenzamide;
N-{[(3S)-3-amino-1-(5-ethyl-7H-pyrrolo[2,3-d]pyrimidin-4-yl)pyrrolidin-3-yl]methyl}-3-fluoro-4-methoxybenzamide;
N-{[(3S)-3-amino-1-(5-ethyl-7H-pyrrolo[2,3-d]pyrimidin-4-yl)pyrrolidin-3-yl]methyl}tetrahydrofuran-3-carboxamide;
N-{[(3S)-3-amino-1-(5-ethyl-7H-pyrrolo[2,3-d]pyrimidin-4-yl)pyrrolidin-3-yl]methyl}-2-(4-methoxyphenyl)acetamide;
N-{[(3S)-3-amino-1-(5-ethyl-7H-pyrrolo[2,3-d]pyrimidin-4-yl)pyrrolidin-3-yl]methyl}cyclobutanecarboxamide;
N-{[(3S)-3-amino-1-(5-ethyl-7H-pyrrolo[2,3-d]pyrimidin-4-yl)pyrrolidin-3-yl]methyl}-1-ethyl-3-methyl-1H-pyrazole-5-carboxamide;
N-{[(3S)-3-amino-1-(5-ethyl-7H-pyrrolo[2,3-d]pyrimidin-4-yl)pyrrolidin-3-yl]methyl}tetrahydrofuran-2-carboxamide;
N-{[(3S)-3-amino-1-(5-ethyl-7H-pyrrolo[2,3-d]pyrimidin-4-yl)pyrrolidin-3-yl]methyl}-3,3-dimethylbutanamide;
N-{[(3S)-3-amino-1-(5-ethyl-7H-pyrrolo[2,3-d]pyrimidin-4-yl)pyrrolidin-3-yl]methyl}-3-methoxybenzamide; and
N-{[(3S)-3-amino-1-(5-ethyl-7H-pyrrolo[2,3-d]pyrimidin-4-yl)pyrrolidin-3-yl]methyl}-3,5-difluorobenzamide.

In another embodiment, the invention also relates to a compound of formula I selected from the group consisting of:
N-{[(3S)-3-amino-1-(5-ethyl-7H-pyrrolo[2,3-d]pyrimidin-4-yl)pyrrolidin-3-yl]methyl}-4-fluoro-2-(trifluoromethyl)benzamide;
N-{[(3S)-3-amino-1-(5-ethyl-7H-pyrrolo[2,3-d]pyrimidin-4-yl)pyrrolidin-3-yl]methyl}-3-isopropyl-1-methyl-1H-pyrazole-5-carboxamide;
N-{[(3S)-3-amino-1-(5-ethyl-7H-pyrrolo[2,3-d]pyrimidin-4-yl)pyrrolidin-3-yl]methyl}-2-methylpropanamide;
N-{[(3S)-3-amino-1-(5-ethyl-7H-pyrrolo[2,3-d]pyrimidin-4-yl)pyrrolidin-3-yl]methyl}-2-ethoxyacetamide;
N-{[(3S)-3-amino-1-(5-ethyl-7H-pyrrolo[2,3-d]pyrimidin-4-yl)pyrrolidin-3-yl]methyl}tetrahydro-2H-pyran-4-carboxamide;
N-{[(3S)-3-amino-1-(5-ethyl-7H-pyrrolo[2,3-d]pyrimidin-4-yl)pyrrolidin-3-yl]methyl}-2-(4-fluorophenyl)acetamide;
N-{[1-(5-chloro-7H-pyrrolo[2,3-d]pyrimidin-4-yl)-3-(methylamino)pyrrolidin-3-yl]methyl}benzamide;
N-{[3-amino-1-(5-methyl-7H-pyrrolo[2,3-d]pyrimidin-4-yl)azetidin-3-yl]methyl}benzamide;
N-{[3-amino-1-(5-methyl-7H-pyrrolo[2,3-d]pyrimidin-4-yl)azetidin-3-yl]methyl}propanamide;
N-{[3-amino-1-(5-methyl-7H-pyrrolo[2,3-d]pyrimidin-4-yl)azetidin-3-yl]methyl}-3-chlorobenzamide;
N-{[3-amino-1-(5-methyl-7H-pyrrolo[2,3-d]pyrimidin-4-yl)azetidin-3-yl]methyl}propanamide;
1-{[3-amino-1-(5-methyl-7H-pyrrolo[2,3-d]pyrimidin-4-yl)pyrrolidin-3-yl]methyl}-1-[2-(dimethylamino)ethyl]-3-phenylurea;
1-{[3-amino-1-(5-methyl-7H-pyrrolo[2,3-d]pyrimidin-4-yl)pyrrolidin-3-yl]methyl}-3-(2,3-dimethylphenyl)urea;
1-{[3-amino-1-(5-methyl-7H-pyrrolo[2,3-d]pyrimidin-4-yl)pyrrolidin-3-yl]methyl}-3-(2,3-dimethylphenyl)urea;
N-((3-amino-1-(5-methyl-7H-pyrrolo[2,3-d]pyrimidin-4-yl)pyrrolidin-3-yl)methyl)-4-chloro-N-methylbenzamide;
N-((3-amino-1-(5-methyl-7H-pyrrolo[2,3-d]pyrimidin-4-yl)pyrrolidin-3-yl)methyl)-2-fluoro-N-methylbenzamide;
N-{[3-amino-1-(5-methyl-7H-pyrrolo[2,3-d]pyrimidin-4-yl)pyrrolidin-3-yl]methyl}-2,6-difluorobenzenesulfonamide; and
N-{[1-(5-chloro-7H-pyrrolo[2,3-d]pyrimidin-4-yl)-3-(methylamino)pyrrolidin-3-yl]methyl}benzamide.

In one embodiment, the invention relates to a compound of formula I, wherein said compound contains about an equimolar amount of the 3S-pyrrolidinyl enantiomer and the 3R-pyrrolidinyl enantiomer.

In another embodiment, the invention relates to a 3S-pyrrolidinyl enantiomer of the compound of formula I.

In another embodiment, the invention relates to a 3R-pyrrolidinyl enantiomer of the compound of formula I.

The present invention also includes isotopically-labeled compounds, which are identical to those recited in formula I above, but for the fact that one or more atoms are replaced by an atom having an atomic mass or mass number different from the atomic mass or mass number usually found in nature. Examples of isotopes that may be incorporated into compounds of the invention include isotopes of hydrogen, carbon, nitrogen, oxygen, phosphorous, fluorine and chlorine, such as, but not limited to, ²H, ³H, ¹³C, ¹⁴C, ¹⁵N, ¹⁸O, ¹⁷O, ³¹P, ³²P, ³⁵S, ¹³F, and ³⁶Cl, respectively. Compounds of formula I, prodrugs thereof, and pharmaceutically acceptable salts of said compounds or of said prodrugs which contain the aforementioned isotopes and/or other isotopes of other atoms are within the scope of this invention. Certain isotopically-labeled compounds of formula I, for example those into which radioactive isotopes such as 3H and 14C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of this invention and prodrugs thereof may generally be prepared by carrying out the procedures disclosed in the Schemes and/or in the Examples and Preparations below, by substituting a readily available isotopically-labeled reagent for a non-isotopically-labeled reagent.

The present invention also relates to the pharmaceutically acceptable acid addition salts and base addition salts of the compounds of formula I. The phrase “pharmaceutically acceptable salt(s)”, as used herein, unless otherwise indicated, includes salts of acidic or basic groups which may be present in the compounds of formula I.

The invention relates to acid addition salts of the compounds of formula I. For example, the compounds of formula I that are basic in nature are capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)]salts. The compounds of formula I that include a basic moiety, such as an amino group, may form pharmaceutically acceptable salts with various amino acids, in addition to the acids mentioned above.

The invention also relates to base addition salts of the compounds of formula I. The chemical bases that may be used as reagents to prepare pharmaceutically acceptable base salts of the compounds of the invention that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

As used herein, the moiety:

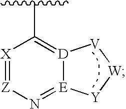

refers to either a group of formula:

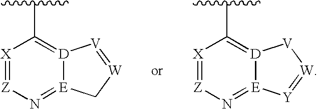

The term “replaced by” refers to embodiments where an element of a straight-chain, branch-chain or cyclic group is replaced by a different element such as, for example, —O—, —S(O)ⱼ—, —C(O)—, —NR¹³— and —CR⁵═CR⁶—, where R⁵, R⁶ and R¹³ are as defined above. For example, if a substituent is a carbocyclic group, such as a cyclobutane group: 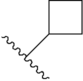
a —(CH₂)— element of the ring may be replaced by, e.g, a —C(O)— to form a cyclobutanone group:

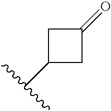

such that two ring atoms of the cyclobutane group are interrupted by the —C(O)— group. Compounds of the invention may accommodate up to three such replacements or interruptions.

As used herein, the term “(C₁-C₆)alkyl,” as well as the alkyl moieties of other groups referred to herein (e.g., (C₁-C₆)alkoxy), refers to linear or branched (e.g., methyl, ethyl, n-propyl, isopropyl, n-butyl, iso-butyl, secondary-butyl, tertiary-butyl) radicals of 1 to 6 carbon atoms; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said (C₁-C₆)alkyl” as used herein refers to any of the preceding alkyl moieties within a group such as alkoxy, alkenyl or alkylamino. Preferred alkyls include (C₁-C₆)alkyl, more preferred are (C₁-C₄)alkyl, and most preferred are methyl and ethyl.

As used herein, the term “halo” includes fluoro, chloro, bromo or iodo.

As used herein, the term “(C₂-C₆)alkenyl” means straight or branched chain unsaturated radicals of 2 to 6 carbon atoms, including, but not limited to ethenyl, 1-propenyl, 2-propenyl(allyl), iso-propenyl, 2-methyl-1-propenyl, 1-butenyl, 2-butenyl, and the like; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. When the compounds of the invention contain a (C₂-C₆)alkenyl group, the compound may exist as the pure E (entgegen) form, the pure Z (zusammen) form, or any mixture thereof.

As used herein, the term “(C₂-C₆)alkynyl” is used herein to mean straight or branched hydrocarbon chain radicals having 2 to 6 carbon atoms and one triple bond including, but not limited to, ethynyl, propynyl, butynyl, and the like; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “(C₃-C₁₀)cycloalkyl” refers to a mono-carbocyclic ring having from 3 to 10 carbon atoms (e.g., cyclopropyl, cyclobutyl, cyclopentyl, cyclohexyl, cycloheptyl, cyclooctyl, cyclononyl); optionally substituted by 1 to 5 suitable substituents as defined above such as, e.g., fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “(C₅-C₁₀)cycloalkenyl” refers to a mono-carbocyclic ring having from 5 to 10 carbon atoms and further containing 1 or 2 double bonds (e.g., cyclopentenyl, cyclohexenyl); optionally substituted by 1 to 5 suitable substituents as defined above.

As used herein, the term “(C₆-C₁₀)bicycloalkyl” refers to a cycloalkyl as defined above which is bridged to a second carbocyclic ring (e.g., bicyclo[2.2.1]heptanyl, bicyclo[3.2.1]octanyl and bicyclo[5.2.0]nonanyl, etc.).

As used herein, the term “(C₆-C₁₀)bicycloalkenyl” refers to a bicycloalkyl as defined above and further containing 1 or 2 double bonds.

As used herein, the term “(C₆-C₁₀)aryl” means aromatic radicals such as phenyl, naphthyl, tetrahydronaphthyl, indanyl and the like; optionally substituted by 1 to 5 suitable substituents as defined above.

As used herein, the term “(C₁-C₉)heteroaryl” refers to an aromatic heterocyclic group usually with one heteroatom selected from O, S and N in the ring. In addition to said heteroatom, the aromatic group may optionally have up to four additional heteroatoms atoms in the ring. For example, heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; optionally substituted by 1 to 5 suitable substituents as defined above such as, e.g., fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term heteroatom refers to an atom or group selected from N, O, S(O)ⱼ or NR¹³, where j is an integer from 0 to 2 and R¹³ is a substituent group as defined above.

The term “(C₂-C₉)heterocycloalkyl” as used herein refers to a cyclic group containing 2-9 carbon atoms and 1 to 4 hetero atoms. Examples of such rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, and the like. Examples of said monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholin-yl, 1,2-tetrahydrothiazin-2-yl, 1,3-tetrahydrothiazin-3-yl, tetrahydrothiadiazin-yl, morpholin-yl, 1,2-tetrahydrodiazin-2-yl, 1,3-tetrahydrodiazin-1-yl, 1,4-oxazin-2-yl, 1,2,5-oxathiazin-4-yl and the like; optionally containing 1 or 2 double bonds and optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “(C₆-C₉)heterobicycloalkyl” refers to a bicycloalkyl as defined above, wherein at least one but not more than four of the carbon ring atoms has been replaced by at least one heteroatom (e.g. tropane).

As used herein, the term “(C₆-C₉)heterobicycloalkenyl” refers to a heterobicycloalkyl as defined above and further containing 1 or 2 double bonds.

As used herein, the term “BOC” refers to —C(O)—O-t-butyl.

As used herein, the term “CBZ” refers to —C(O)—O—CH₂—C₆H₅.

As used herein, the term “DMB” refers to -2,4-dimethoxybenzyl.

“Embodiment” as used herein refers to specific groupings of compounds or uses into discrete subgenera. Such subgenera may be cognizable according to one particular substituent such as, e.g., a specific R¹ or R² group. Other subgenera are cognizable according to combinations of various substituents, such as all compounds wherein R¹ is (C₁-C₆)alkyl and R² is hydrogen.

The invention also relates to methods of making the compounds of formula I. In one embodiment, the invention relates to a method of making a compound of formula I comprising,

reacting a cyclic amine of formula:

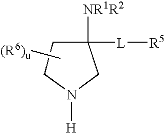

with a heterobicyclic compound of formula:

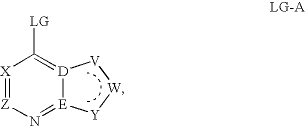

to provide a compound of formula I, wherein

D, E, V, W, X, Y, Z, u, R¹, R², R⁵ and R⁶ are as defined above; and

LG is a leaving group.

In another embodiment, the invention relates to a method for making a compound of formula I comprising,

reacting a cyclic amine of formula:

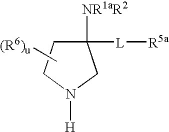

with a heterobicyclic compound of formula:

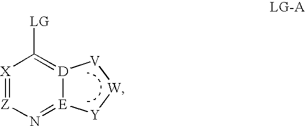

to provide a protected intermediate compound, and

deprotecting the protected intermediate compound to provide a compound of formula I, wherein

D, E, V, W, X, Y, Z, u, R² and R⁶ are as defined above;

R^{1a} is as defined above for R¹ or a protecting group;

R^{5a} is as defined above for R⁵ or a protecting group;

LG is a leaving group; and wherein

at least one of R^{1a} and R^{5a} is a protecting group.

In one embodiment, the protecting group (PG) used in the method for making compounds of formula I is a selected from the group consisting of -benzyl, —C(O)O-benzyl, -2,4-dimethoxybenzyl, and —C(O)-tert-butyl.

In one embodiment, the leaving group (LG) used in the method for making compounds of formula I is selected from the group consisting of —F, —Cl, —Br, —I, -mesylate and -tosylate.

When preparing compounds of formula I in accordance with the invention, it is open to a person skilled in the art to routinely select the form of the intermediate compound(s) which provides the best combination of features for this purpose. Such features include the melting point, solubility, processability and yield of the intermediate form and the resulting ease with which the product may be purified on isolation.

The invention also relates to novel intermediate compounds that are useful for making the compounds of formula I.

In one embodiment, the novel intermediate compound of the invention is selected from the group consisting of: 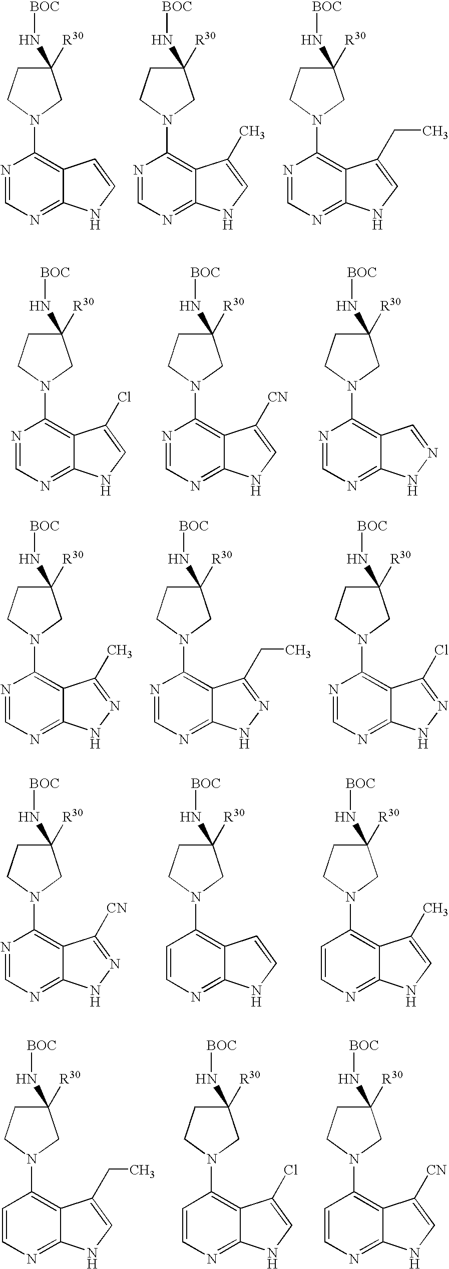 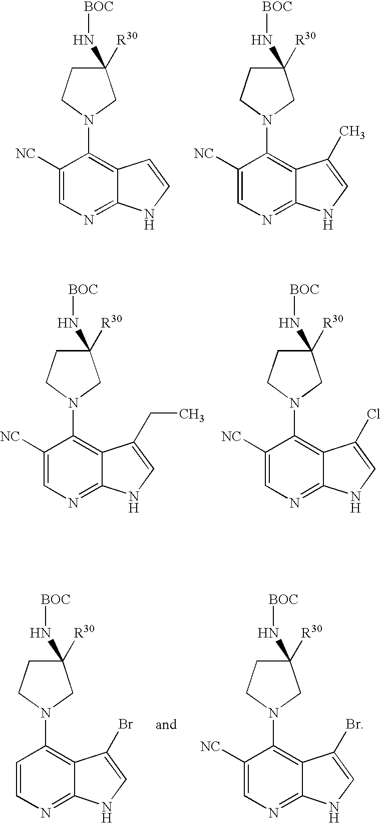
wherein R³⁰ is selected from the group consisting of —CH₂OH, —CH₂NH₂, —C(O)H

In yet another embodiment of the invention, the novel intermediate compound of the invention is selected from the group consisting of:

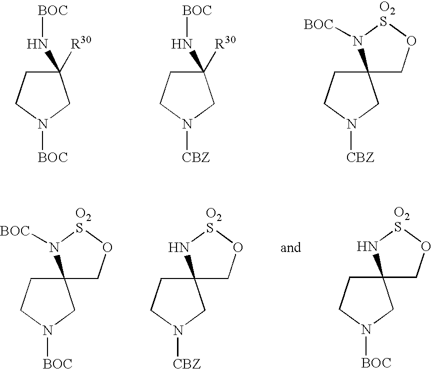

where R³⁴ is as defined above.

In yet another embodiment of the invention, the novel intermediate compound of the invention is selected from the group consisting of: 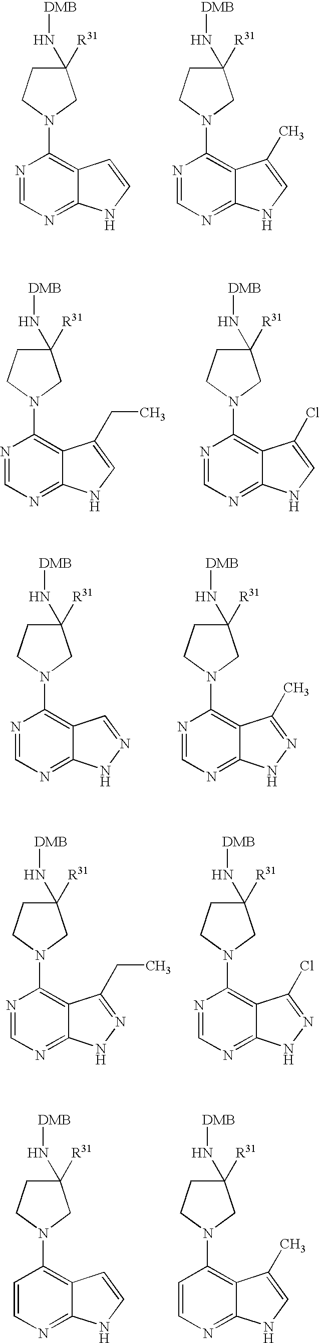 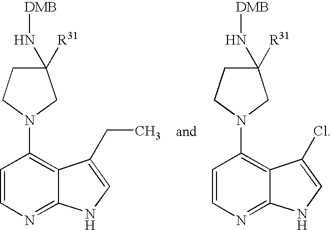
wherein R³¹ is selected from the group consisting of —CN and —CH₂NH₂.

It will be understood that the intermediate compounds of the invention depicted above are not limited to the particular enantiomer shown, but also include all stereoisomers and mixtures thereof.

This invention also relates to a method for the treatment of abnormal cell growth in a mammal, preferably a human, comprising administering to said mammal an amount of a compound of the formula I, or a pharmaceutically acceptable salt thereof (including hydrates, solvates and polymorphs of said compound of formula I or pharmaceutically acceptable salts thereof), that is effective in treating abnormal cell growth.

The present invention also relates to methods of administering the compositions described above to an animal in need thereof.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor kinase; (2) benign and malignant cells of other proliferative diseases in which aberrant tyrosine kinase activation occurs; (3) tumor cells (tumors) that proliferate through the deletion of protein(s) involved in regulating cell growth and apoptosis, such as a PTEN deletion; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

In one embodiment of this method, the abnormal cell growth is cancer, including, but not limited to, mesothelioma, hepatobilliary cancers (hepatic and billiary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

Another more specific embodiment of the present invention is directed to a cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, prostate cancer, neoplasms of the central nervous system (CNS), primary CNS lymphoma, non-Hodgkins's lymphoma, spinal axis tumors, or a combination of one or more of the foregoing cancers.

In another more specific embodiment of the present invention the cancer is selected from lung cancer (NSCLC and SCLC), breast cancer, ovarian cancer, colon cancer, rectal cancer, prostate cancer, cancer of the anal region, or a combination of one or more of the foregoing cancers.

In another embodiment of the present invention, said abnormal cell growth is a benign proliferative disease, including, but not limited to, psoriasis, benign prostatic hypertrophy, restinosis, synovial proliferation disorder, retinopathy or other neovascular disorders of the eye, pulmonary hypertension or mobilization of TIE-2 positive stem cells from bone marrow for use in reconstituting normal cells of any tissue.

This invention also relates to a method for the treatment of abnormal cell growth in a mammal in need of such treatment, which comprises administering to said mammal an amount of a compound of formula I (including hydrates, solvates and polymorphs of said compound of formula I or pharmaceutically acceptable salts thereof), in combination with one or more (preferable one to three) anti-cancer agents selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), anti-hormones, anti-androgens, gene silencing agents, gene activating agents and anti-vascular agents, wherein the amounts of the compound of formula I together with the amounts of the combination anticancer agents is effective in treating abnormal cell growth.

The invention also relates to a method for the treatment of a hyperproliferative disorder in a mammal in need of such treatment, comprising administering to said mammal an amount of a compound of formula I (including hydrates, solvates and polymorphs of said compound of formula I or pharmaceutically acceptable salts thereof), in combination with an anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), hormones, anti-hormones, anti-androgens, gene silencing agents, gene activating agents and anti-vascular agents, wherein the amounts of the compound of formula I together with the amounts of the combination anticancer agents is effective in treating said hyperproliferative disorder.

In one embodiment, the invention relates to compositions comprising a compound of the invention and at least one additional ingredient (hereinafter “the compositions of the invention”). It will be understood that the compositions of the invention will encompass any combination of the compound of the invention and the at least one additional ingredient. Non-limiting examples of the at least one additional ingredient include impurities (e.g., intermediates present in the unrefined compounds of the invention), active ingredients as discussed herein (e.g., an additional anti-tumor agent), pharmaceutically acceptable excipients, or one or more solvents (e.g., a pharmaceutically acceptable carrier as discussed herein).

The term “solvent” as it relates to the compositions of the invention includes organic solvents (e.g., methanol, ethanol, isopropanol, ethyl acetate, methylene chloride, and tetrahydrofuran) and water. The one or more solvents may be present in a non-stoichiometric amount, e.g., as a trace impurity, or in sufficient excess to dissolve the compound of the invention. Alternatively, the one or more solvents may be present in a stoichiometric amount, e.g., 0.5:1, 1:1, or 2:1 molar ratio, based on the amount of compound of the invention.

In one embodiment, the at least one additional ingredient that is present in the composition of the invention is an organic solvent.

In another embodiment, the at least one additional ingredient that is present in the composition of the invention is water.

In one embodiment, the at least one additional ingredient that is present in the composition of the invention is a pharmaceutically acceptable carrier.

In another embodiment, the at least one additional ingredient that is present in the composition of the invention is a pharmaceutically acceptable excipient.

In one embodiment, the composition of the invention is a solution.

In another embodiment, the composition of the invention is a suspension.

In another embodiment, the composition of the invention is a solid.

In another embodiment, the composition of the invention comprises an amount of the compound of the invention effective for treating abnormal cell growth.

In yet another embodiment, the invention relates to a composition comprising an effective amount of the compound of the invention, and a pharmaceutically acceptable carrier.

In another embodiment, the invention relates to a composition comprising a therapeutically effective amount of the compound the invention as defined above, a pharmaceutically acceptable carrier and, optionally, at least one additional medicinal or pharmaceutical agents (hereinafter “the pharmaceutical compositions of the invention”). In a preferred embodiment, the at least one additional medicinal or pharmaceutical agent is an anti-cancer agent.

This invention also relates to a pharmaceutical composition for the treatment of abnormal cell growth in a mammal, including a human, comprising an amount of a compound of the formula I, as defined above (including hydrates, solvates and polymorphs of said compound of formula I or pharmaceutically acceptable salts thereof), that is effective in treating abnormal cell growth, and a pharmaceutically acceptable carrier. In one embodiment of this composition, the abnormal cell growth is cancer, including, but not limited to, mesothelioma, hepatobilliary cancer (hepatic and billiary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

The invention also relates to a pharmaceutical composition for the treatment of abnormal cell growth in a mammal, including a human, which comprises an amount of a compound of formula I, as defined above (including hydrates, solvates and polymorphs of said compound of formula I or pharmaceutically acceptable salts thereof), in combination with one or more (preferably one to three) anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers, hormones, anti-hormones, anti-androgens gene silencing agents, gene activating agents and anti-vascular agents and a pharmaceutically acceptable carrier, wherein the amounts of the compound of formula I and the combination anti-cancer agents when taken as a whole is therapeutically effective for treating said abnormal cell growth.

In one embodiment of the present invention the anti-cancer agent used in conjunction with a compound of formula I and pharmaceutical compositions described herein is an anti-angiogenesis agent.

A more specific embodiment of the present invention includes combinations of the compounds of formula I with anti-angiogenesis agents selected from VEGF inhibitors, VEGFR inhibitors, TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), Vandetanib (Zactima), sorafenib (Bayer/Onyx), AEE788 (Novartis), AZD-2171, VEGF Trap (Regeneron/Aventis), vatalanib (also known as PTK-787, ZK-222584: Novartis & Schering AG as described in U.S. Pat. No. 6,258,812), Macugen (pegaptanib octasodium, NX-1838, EYE-001, Pfizer Inc./Gilead/Eyetech), IM862 (Cytran Inc. of Kirkland, Wash., USA); Neovastat (Aeterna); and Angiozyme (a synthetic ribozyme that cleaves mRNA producing VEGF1) and combinations thereof. VEGF inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG-13736, AG-28262, Vatalanib, sorafenib, Macugen and combinations thereof.

Additional VEGF inhibitors are described in, for example in U.S. Pat. No. 6,492,383, issued Dec. 10, 2002, U.S. Pat. No. 6,235,764 issued May 22, 2001, U.S. Pat. No. 6,177,401 issued Jan. 23, 2001, U.S. Pat. No. 6,395,734 issued May 28, 2002, U.S. Pat. No. 6,534,524 (discloses AG13736) issued Mar. 18, 2003, U.S. Pat. No. 5,834,504 issued Nov. 10, 1998, U.S. Pat. No. 6,316,429 issued Nov. 13, 2001, U.S. Pat. No. 5,883,113 issued Mar. 16, 1999, U.S. Pat. No. 5,886,020 issued Mar. 23, 1999, U.S. Pat. No. 5,792,783 issued Aug. 11, 1998, U.S. Pat. No. 6,653,308 issued Nov. 25, 2003, WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

PDGFr inhibitors include but not limited to those disclosed in International Patent Publication number WO 01/40217, published Jun. 7, 2001 and International Patent Publication number WO 2004/020431, published Mar. 11, 2004, the contents of which are incorporated in their entirety for all purposes.

Preferred PDGFr inhibitors include Pfizer's CP-673,451 and CP-868,596 and their pharmaceutically acceptable salts.

TIE-2 inhibitors include GlaxoSmithKline's benzimidazoles and pyridines including GW-697465A such as described in International Patent Publications WO 02/044156 published Jun. 6, 2002, WO 03/066601 published Aug. 14, 2003, WO 03/074515 published Sep. 12, 2003, WO 03/022852 published Mar. 20, 2003 and WO 01/37835 published May 31, 2001. Other TIE-2 inhibitors include Regeneron's biologicals such as those described in International Patent Publication WO 09/611,269 published Apr. 18, 1996, Amgen's AMG-386, and Abbott's pyrrolopyrimidines such as A-422885 and BSF-466895 described in International Patent Publications WO 09/955,335, WO 09/917,770, WO 00/075139, WO 00/027822, WO 00/017203 and WO 00/017202.

In another more specific embodiment of the present invention the anti-cancer agent used in conjunction with a compound of formula I and pharmaceutical compositions described herein is where the anti-angiogenesis agent is a protein kinase C β inhibitor such as enzastaurin, midostaurin, perifosine, a staurosporine derivative (such as RO 318425, R0317549, R0318830 or RO 318220 (Roche)), teprenone (Selbex) and UCN-01 (Kyowa Hakko).

Examples of useful COX-II inhibitors which may be used in conjunction with a compound of formula I and pharmaceutical compositions described herein include Celebrex (celecoxib), parecoxib, deracoxib, ABT-963, COX-189 (Lumiracoxib), BMS 347070, RS 57067, NS-398, Bextra (valdecoxib), Vioxx (rofecoxib), SD-8381, 4-methyl-2-(3,4-dimethylphenyl)-1-(4-sulfamoyl-phenyl)-1H-pyrrole, 2-(4-ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one specific embodiment of particular interest the anti-tumor agent is celecoxib as disclosed in U.S. Pat. No. 5,466,823, the contents of which are incorporated by reference in its entirety for all purposes.

In another embodiment the anti-tumor agent is deracoxib as disclosed in U.S. Pat. No. 5,521,207, the contents of which are incorporated by reference in its entirety for all purposes.

Other useful anti-angiogenic inhibitors used in conjunction with a compound of formula I and pharmaceutical compositions described herein include aspirin, and non-steroidal anti-inflammatory drugs (NSAIDs) which nonselectively inhibit the enzymes that make prostaglandins (cyclooxygenase I and II), resulting in lower levels of prostaglandins. Such agents include, but are not limited to, Aposyn (exisulind), Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred nonselective cyclooxygenase inhibitors include ibuprofen (Motrin), nuprin, naproxen (Aleve), indomethacin (Indocin), nabumetone (Relafen) and combinations thereof.

MMP inhibitors include ABT-510 (Abbott), ABT 518 (Abbott), Apratastat (Amgen), AZD 8955 (AstraZeneca), Neovostat (AE-941), COL 3 (CollaGenex Pharmaceuticals), doxycycline hyclate, MPC 2130 (Myriad) and PCK 3145 (Procyon).

Other anti-angiogenic compounds include acitretin, angiostatin, aplidine, cilengtide, COL-3, combretastatin A-4, endostatin, fenretinide, halofuginone, Panzem (2-methoxyestradiol), PF03446962 (ALK-1 inhibitor), rebimastat, removab, Revlimid, squalamine, thalidomide, ukrain, Vitaxin (alpha-v/beta-3 integrin), and zoledronic acid.

In another embodiment the anti-cancer agent is a so called signal transduction inhibitor. Such inhibitors include small molecules, antibodies, and antisense molecules. Signal transduction inhibitors include kinase inhibitors, such as tyrosine kinase inhibitors, serine/threonine kinase inhibitors. Such inhibitors may be antibodies or small molecule inhibitors. More specifically signal transduction inhibitors include farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, FAK inhibitors, PLK inhibitors, ALK inhibitors, Src inhibitors, PAR inhibitors, NEK inhibitors, and inhibitors of the WNT pathway and so called multi-targeted kinase inhibitors

In another embodiment the anti-cancer signal transduction inhibitor is a farnesyl protein transferase inhibitor. Farnesyl protein transferase inhibitors include the compounds disclosed and claimed in U.S. Pat. No. 6,194,438, issued Feb. 27, 2002; U.S. Pat. No. 6,258,824, issued Jul. 10, 2001; U.S. Pat. No. 6,586,447, issued Jul. 1, 2003; U.S. Pat. No. 6,071,935, issued Jun. 6, 2000; and U.S. Pat. No. 6,150,377, issued Nov. 21, 2000. Other farnesyl protein transferase inhibitors include AZD-3409 (AstraZeneca), BMS-214662 (Bristol-Myers Squibb), Lonafarnib (Sarasar) and RPR-115135 (Sanofi-Aventis). Each of the foregoing patent applications and provisional patent applications is herein incorporated by reference in their entirety.

In another embodiment the anti-cancer signal transduction inhibitor is a GARF inhibitor. Preferred GARF inhibitors (glycinamide ribonucleotide formyltransferse inhibitors) include Pfizer's AG-2037 (pelitrexol) and its pharmaceutically acceptable salts. GARF inhibitors useful in the practice of formula I are disclosed in U.S. Pat. No. 5,608,082 which is incorporated in its entirety for all purposed.

In another embodiment the anti-cancer signal transduction inhibitors used in conjunction with a compound of formula I and pharmaceutical compositions described herein include ErbB-1 (EGFr) inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), Matuzumab (Merck AG), Nimotuzumab, Panitumumab (Abgenix/Amgen), Vandetanib, hR3 (York Medical and Center for Molecular Immunology), TP-38 (IVAX), EGFR fusion protein, EGF-vaccine, anti-EGFr immunoliposomes (Hermes Biosciences Inc.) and combinations thereof.

Preferred EGFr inhibitors include Iressa (gefitinib), Erbitux, Tarceva and combinations thereof.

In another embodiment the anti-cancer signal transduction inhibitor is selected from pan erb receptor inhibitors or ErbB2 receptor inhibitors, such as CP-724,714, PF-299804, CI-1033 (canertinib, Pfizer, Inc.), Herceptin (trastuzumab, Genentech Inc.), Omnitarg (2C4, pertuzumab, Genentech Inc.), TAK-165 (Takeda), GW-572016 (lapatinib, GlaxoSmithKline), Pelitinib (EKB-569 Wyeth), BMS-599626, PKI-166 (Novartis), dHER2 (HER2Vaccine, Corixa and GlaxoSmithKline), Osidem (IDM-1), APC8024 (HER2Vaccine, Dendreon), anti-HER2/neu bispecific antibody (Decof Cancer Center), B7.her2.1gG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erb receptor inhibitors include GW572016, PF-299804, Pelitinib, and Omnitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277, each of which is herein incorporated by reference in its entirety.

Various other compounds, such as styrene derivatives, have also been shown to possess tyrosine kinase inhibitory properties, and some of tyrosine kinase inhibitors have been identified as erbB2 receptor inhibitors. Other erbB2 Inhibitors are described in European patent publications EP 566,226 A1 (published Oct. 20, 1993), EP 602,851 A1 (published Jun. 22, 1994), EP 635,507 A1 (published Jan. 25, 1995), EP 635,498 A1 (published Jan. 25, 1995), and EP 520,722 A1 (published Dec. 30, 1992). These publications refer to certain bicyclic derivatives, in particular quinazoline derivatives possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO0/44728 (published Aug. 3, 2000), EP 1029853A1 (published Aug. 23, 2000), and WO01/98277 (published Dec. 12, 2001) all of which are incorporated herein by reference in their entirety.

In another embodiment the anti-cancer signal transduction inhibitor is an IGF1R inhibitor. Specific IGF1R antibodies (such as CP-751871) that may be used in the present invention include those described in International Patent Application No. WO 2002/053596, which is herein incorporated by reference in its entirety.

In another embodiment the anti-cancer signal transduction inhibitor is a MEK inhibitor. MEK inhibitors include Pfizer's MEK1/2 inhibitor PD325901, Array Biopharm's MEK inhibitor ARRY-142886, and combinations thereof.

In another embodiment the anti-cancer signal transduction inhibitor is an mTOR inhibitor. mTOR inhibitors include everolimus (RAD001, Novartis), zotarolimus, temsirolimus (CCI-779, Wyeth), AP 23573 (Ariad), AP23675, Ap23841, TAFA 93, rapamycin (sirolimus) and combinations thereof.

In another embodiment the anti-cancer signal transduction inhibitor is an Aurora 2 inhibitor such as VX-680 and derivatives thereof (Vertex), R 763 and derivatives thereof (Rigel) and ZM 447-439 and AZD 1152 (AstraZeneca), or a Checkpoint kinase ½ inhibitors such as XL844 (Exelixis).

In another embodiment the anti-cancer signal transduction inhibitor is an Akt inhibitor (Protein Kinase B) such as API-2, perifosine and RX-0201.

Preferred multitargeted kinase inhibitors include Sutent, (sunitinib, SU-11248), described in U.S. Pat. No. 6,573,293 (Pfizer, Inc, NY, USA) and imatinib mesylate (Gleevec).

Additionally, other targeted anti-cancer agents include the raf inhibitors sorafenib (BAY-43-9006, Bayer/Onyx), GV-1002, ISIS-2503, LE-AON and GI-4000.

The invention also relates to the use of the compounds of formula I together with cell cycle inhibitors such as the CDK2 inhibitors ABT-751 (Abbott), AZD-5438 (AstraZeneca), Alvocidib (flavopiridol, Aventis), BMS-387,032 (SNS 032 Bristol Myers), EM-1421 (Erimos), indisulam (Esai), seliciclib (Cyclacel), BIO 112 (One Bio), UCN-01 (Kyowa Hakko), and AT7519 (Astex Therapeutics) and Pfizer's multitargeted CDK inhibitors PD0332991 and AG24322.

The invention also relates to the use of the compounds of formula I together with telomerase inhibitors such as transgenic B lymphocyte immunotherapy (Cosmo Bioscience), GRN 163L (Geron), GV1001 (Pharmexa), RO 254020 (and derivatives thereof), and diazaphilonic acid.

Biological response modifiers (such as antibodies, immunotherapeutics and peptide mimics), are agents that modulate defense mechanisms of living organisms or biological responses, such as survival, growth, or differentiation of tissue cells to direct them to have anti-tumor activity.

Immunologicals including interferons and numerous other immune enhancing agents that may be used in combination therapy with compounds of formula I, optionally with one or more other agent include, but are not limited to interferon alpha, interferon alpha-2a, interferon, alpha-2b, interferon beta, interferon gamma-1a, interferon gamma-1b (Actimmune), or interferon gamma-n1, PEG Intron A, and combinations thereof. Other agents include interleukin 2 agonists (such as aldesleukin, BAY-50-4798, Ceplene (histamine dihydrochloride), EMD-273063, MVA-HPV-IL2, HVA-Muc-1-IL2, interleukin 2, teceleukin and Virulizin), Ampligen, Canvaxin, CeaVac (CEA), denileukin, filgrastim, Gastrimmune (G17DT), gemtuzumab ozogamicin, Glutoxim (BAM-002), GMK vaccine (Progenics), Hsp 90 inhibitors (such as HspE7 from Stressgen, AG-858, KOS-953, MVJ-1-1 and STA-4783), imiquimod, krestin (polysaccharide K), lentinan, Melacine (Corixa), MelVax (mitumomab), molgramostim, Oncophage (HSPPC-96), OncoVAX (including OncoVAX-CL and OncoVAX-Pr), oregovomab, sargramostim, sizofuran, tasonermin, TheraCys, thymalfasin, pemtumomab (Y-muHMFG1), picibanil, Provenge (Dendreon), ubenimex, WF-10 (Immunokine), Z-100 (Ancer-20 from Zeria), Lenalidomide (REVIMID, Celegene), thalomid (Thalidomide), and combinations thereof.

Anti-cancer agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736. Additional, specific CTLA4 antibodies that may be used in the present invention include those described in U.S. Provisional Application 60/113,647 (filed Dec. 23, 1998), U.S. Pat. No. 6,682,736 both of which are herein incorporated by reference in their entirety.

In another embodiment of the present invention the anti-cancer agent used in conjunction with a compound of formula I and pharmaceutical compositions described herein is a CD20 antagonist. Specific CD20 antibody antagonists that may be used in the present invention include rituximab (Rituxan), Zevalin (Ibritumomab tiuxetan), Bexxar (131-I -tositumomab), Belimumab (LymphoStat-B), HuMax-CD20 (HuMax, Genmab), R 1594 (Roche Genentech), TRU-015 (Trubion Pharmaceuticals) and Ocrelizumab (PRO 70769).

In another embodiment of the present invention the anti-cancer agent used in conjunction with a compound of formula I and pharmaceutical compositions described herein is a CD40 antagonist. Specific CD40 antibody antagonists that may be used in the present invention include CP-870893, CE-35593 and those described in International Patent Application No. WO 2003/040170 which is herein incorporated by reference in its entirety. Other CD40 antagonists include ISF-154 (Ad-CD154, Tragen), toralizumab, CHIR 12.12 (Chiron), SGN 40 (Seattle Genetics) and ABI-793 (Novartis).

In another embodiment of the present invention the anti-cancer agent used in conjunction with a compound of formula I and pharmaceutical compositions described herein is a hepatocyte growth factor receptor antagonist (HGFr or c-MET).

Immunosuppressant agents useful in combination with the compounds of formula I include epratuzumab, alemtuzumab, daclizumab, lenograstim and pentostatin (Nipent or Coforin).

The invention also relates to the use of the compounds of formula I together with hormonal, anti-hormonal, anti-androgenal therapeutic agents such as anti-estrogens including, but not limited to fulvestrant, toremifene, raloxifene, lasofoxifene, letrozole (Femara, Novartis), anti-androgens such as bicalutamide, finasteride, flutamide, mifepristone, nilutamide, Casodex® (4′-cyano-3-(4-fluorophenylsulphonyl)-2-hydroxy-2-methyl-3′-(trifluoromethyl)-propionanilide, bicalutamide) and combinations thereof.

The invention also relates to the use of the compounds of formula I together with hormonal therapy, including but not limited to, exemestane (Aromasin, Pfizer Inc.), Abarelix (Praecis), Trelstar, anastrozole (Arimidex, Astrazeneca), Atamestane (Biomed-777), Atrasentan (Xinlay), Bosentan, Casodex (AstraZeneca), doxercalciferol, fadrozole, formestane, gosrelin (Zoladex, AstraZeneca), Histrelin (histrelin acetate), letrozole, leuprorelin (Lupron or Leuplin, TAP/Abbott/Takeda), tamoxifen citrate (tamoxifen, Nolvadex, AstraZeneca), and combinations thereof.

The invention also relates to the use of the compounds of formula I together with gene silencing agents or gene activating agents such as histone deacetylase (HDAC) inhibitors such as suberolanilide hydroxamic acid (SAHA, Merck Inc./Aton Pharmaceuticals), depsipeptide (FR901228 or FK228), G2M-777, MS-275, pivaloyloxymethyl butyrate and PXD-101.

The invention also relates to the use of the compounds of formula I together with gene therapeutic agents such as Advexin (ING 201), TNFerade (GeneVec, a compound which express TNFalpha in response to radiotherapy), RB94 (Baylor College of Medicine).

The invention also relates to the use of the compounds of formula I together with ribonucleases such as Onconase (ranpirnase).

The invention also relates to the use of the compounds of formula I together with antisense oligonucleotides such as bcl-2 antisense inhibitor Genasense (Oblimersen, Genta,

The invention also relates to the use of the compounds of formula I together with proteosomics such as PS-341 (MLN-341) and Velcade (bortezomib).

The invention also relates to the use of the compounds of formula I together with anti-vascular agents such as Combretastatin A4P (Oxigene).

The invention also relates to the use of the compounds of formula I together with traditional cytotoxic agents including DNA binding agents, mitotic inhibitors, alkylating agents, anti-metabolites, intercalating antibiotics, topoisomerase inhibitors and microtubulin inhibitors.

Topoisomerase I inhibitors useful in the combination embodiments of the present invention include 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, and combinations thereof.

Camptothecin derivatives are of particular interest in the combination embodiments of the invention and include camptothecin, 10-hydroxycamptothecin, 9-aminocamptothecin, irinotecan, SN-38, edotecarin, topotecan and combinations thereof.

A particularly preferred toposimerase I inhibitor is irinotecan HCl (Camptosar®)

Topoisomerase II inhibitors useful in the combination embodiments of the present invention include aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, and Zinecard® (dexrazoxane).

Particularly preferred toposimerase II inhibitors include epirubicin (Ellence®), doxorubicin, daunorubicin, idarubicin and etoposide.

Alkylating agents that may be used in combination therapy with compounds of formula I, optionally with one or more other agents include, but are not limited to, nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin® (carboplatin), eptaplatin, lobaplatin, nedaplatin, Eloxatin® (oxaliplatin, Sanofi), streptozocin, or satrplatin and combinations thereof.

Particularly preferred alkylating agents include Eloxatin® (oxaliplatin).

Antimetabolites that may be used in combination therapy with compounds of formula I, optionally with one or more other agents include, but are not limited to dihydrofolate reductase inhibitors (such as methotrexate and NeuTrexin® (trimetresate glucuronate)), purine antagonists (such as 6-mercaptopurine riboside, mercaptopurine, 6-thioguanine, cladribine, clofarabine (Clolar™), fludarabine, nelarabine, and raltitrexed), pyrimidine antagonists (such as 5-fluorouracil (5-FU), Alimta® (premetrexed disodium, LY231514, MTA), capecitabine (Xeloda), cytosine arabinoside, Gemzar® (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

In another embodiment the anti-cancer agent is a poly(ADP-ribose) polymerase-1 (PARP-1) inhibitor such as AG-014699, ABT-472, INO-1001, KU-0687 and GPI 18180.

Microtubulin inhibitors that may be used in combination therapy with compounds of formula I, optionally with one or more other agents include, but are not limited to ABI-007, Albendazole, Batabulin, CPH-82, EPO 906 (Novartis), discodermolide (XAA-296), Vinfunine and ZD-6126 (AstraZeneca).

Antibiotics that may be used in combination therapy with compounds of formula I, optionally with one or more other agent including, but are not limited to, intercalating antibiotics such as actinomycin D, bleomycin, mitomycin C, neocarzinostatin (Zinostatin), peplomycin, and combinations thereof.

Plant derived anti-tumor substances (also known as spindle inhibitors) that may be used in combination therapy with compounds of formula I, optionally with one or more other agent include, but are not limited to, mitotic inhibitors, for example vinblastine, vincristine, vindesine, vinorelbine (Navelbine), docetaxel (Taxotere), Ortataxel, paclitaxel (including Taxoprexin a DHA/paciltaxel conjugate) and combinations thereof.

Platinum-coordinated compounds include but are not limited to, cisplatin, carboplatin, nedaplatin, oxaliplatin (Eloxatin), Satraplatin (JM-216), and combinations thereof.

Particularly preferred cytotoxic agents include Camptosar, capecitabine (Xeloda), oxaliplatin (Eloxatin), Taxotere and combinations thereof.

Other antitumor agents include alitretinoin, I-asparaginase, AVE-8062 (Aventis), calcitriol (Vitamin D derivative), Canfosfamide (Telcyta, TLK-286), Cotara (131I chTNT 1/b), DMXAA (Antisoma), exisulind, ibandronic acid, Miltefosine, NBI-3001 (IL-4), pegaspargase, RSR13 (efaproxiral), Targretin (bexarotene), tazarotne (Vitamin A derivative), Tesmilifene (DPPE), Theratope, tretinoin, Trizaone (tirapazamine), Xcytrin (motexafin gadolinium) and Xyotax (polyglutamate paclitaxel), and combinations thereof.

In another embodiment of the present invention statins may be used in conjunction with a compound of formula I and pharmaceutical compositions. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet, Lipitor and torcetrapib.

Another embodiment of the present invention of particular interest relates to a method for the treatment of breast cancer in a human in need of such treatment, comprising administering to said human an amount of a compound of formula I (including hydrates, solvates and polymorphs of said compound of formula I or pharmaceutically acceptable salts thereof), in combination with one or more (preferably one to three) anti-cancer agents selected from the group consisting of trastuzumab (Herceptin), docetaxel (Taxotere), paclitaxel, capecitabine (Xeloda), gemcitabine (Gemzar), vinorelbine (Navelbine), exemestane (Aromasin), letrozole (Femara) and anastrozole (Arimidex).

Another embodiment of the present invention of particular interest relates to a method for the treatment of colorectal cancer in a human in need of such treatment, comprising administering to said human an amount of a compound of formula I (including hydrates, solvates and polymorphs of said compound of formula I or pharmaceutically acceptable salts thereof), in combination with one or more (preferably one to three) anti-cancer agents selected from the group consisting of capecitabine (Xeloda), irinotecan HCl (Camptosar), bevacizumab (Avastin), cetuximab (Erbitux), oxaliplatin (Eloxatin), premetrexed disodium (Alimta), vatalanib (PTK-787), Sutent (sunitinib), AG-13736 (axitinib), SU-14843, PF-0337210, PD-325901, PF-2341066, Tarceva, Iressa, Pelitinib, Lapatinib, Mapatumumab, Gleevec, BMS184476, CCl 779, ISIS 2503, ONYX 015 and Flavopyridol, wherein the amounts of the compound of formula I together with the amounts of the combination anticancer agents is effective in treating colorectal cancer.

Another embodiment of the present invention of particular interest relates to a method for the treatment of renal cell carcinoma in a human in need of such treatment, comprising administering to said human an amount of a compound of formula I (including hydrates, solvates and polymorphs of said compound of formula I or pharmaceutically acceptable salts thereof), in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the compound of formula I together with the amounts of the combination anticancer agents is effective in treating renal cell carcinoma.

Another embodiment of the present invention of particular interest relates to a method for the treatment of melanoma in a human in need of such treatment, comprising administering to said human an amount of a compound of formula I (including hydrates, solvates and polymorphs of said compound of formula I or pharmaceutically acceptable salts thereof), in combination with one or more (preferably one to three) anti-cancer agents selected from the group consisting of interferon alpha, interleukin-2, temozolomide, docetaxel (Taxotere), paclitaxel, DTIC, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the compound of formula I together with the amounts of the combination anticancer agents is effective in treating melanoma.

Another embodiment of the present invention of particular interest relates to a method for the treatment of Lung cancer in a human in need of such treatment, comprising administering to said human an amount of a compound of formula I (including hydrates, solvates and polymorphs of said compound of formula I or pharmaceutically acceptable salts thereof), in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, and Paraplatin (carboplatin), wherein the amounts of the compound of formula I together with the amounts of the combination anticancer agents is effective in treating Lung cancer.

In one preferred embodiment radiation may be used in conjunction with a compound of formula I and pharmaceutical compositions described herein. Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays α-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with a compound of formula I and pharmaceutical compositions described herein.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

Further, the invention provides a compound of formula I alone or in combination with one or more supportive care products, e.g., a product selected from the group consisting of Filgrastim (Neupogen), ondansetron (Zofran), Fragmin, Procrit, Aloxi, Emend, or combinations thereof.

### DETAILED DESCRIPTION OF THE INVENTION

Non-limiting methods for making the compounds of formula I are described in detail in the Experimental Section and below in Schemes 1 to 10. Schemes 1 to 10 depict methods for making the pyrrolidinyl-derivative compounds of formula I, i.e., where r is 1 and s is 1, or r is 2 and s is 0. However it will be understood that one of skill in the art could make the corresponding azetidinyl-, piperidinyl- and hexamethyleneimine-derivatives of the compounds of formula I using methods similar to those described in the Schemes, the Examples, or the literature. It will be understood that the compounds depicted in the Schemes below are not limited to the particular enantiomer(s) shown, but also include all stereoisomers and mixtures thereof.

Scheme 1 shows a non-limiting method for making amide-derivatives of the compounds of formula I where a cyclic amine group is derivatized followed by reaction with a heterobicyclic compound to form the amide derivative of a compound of formula I. 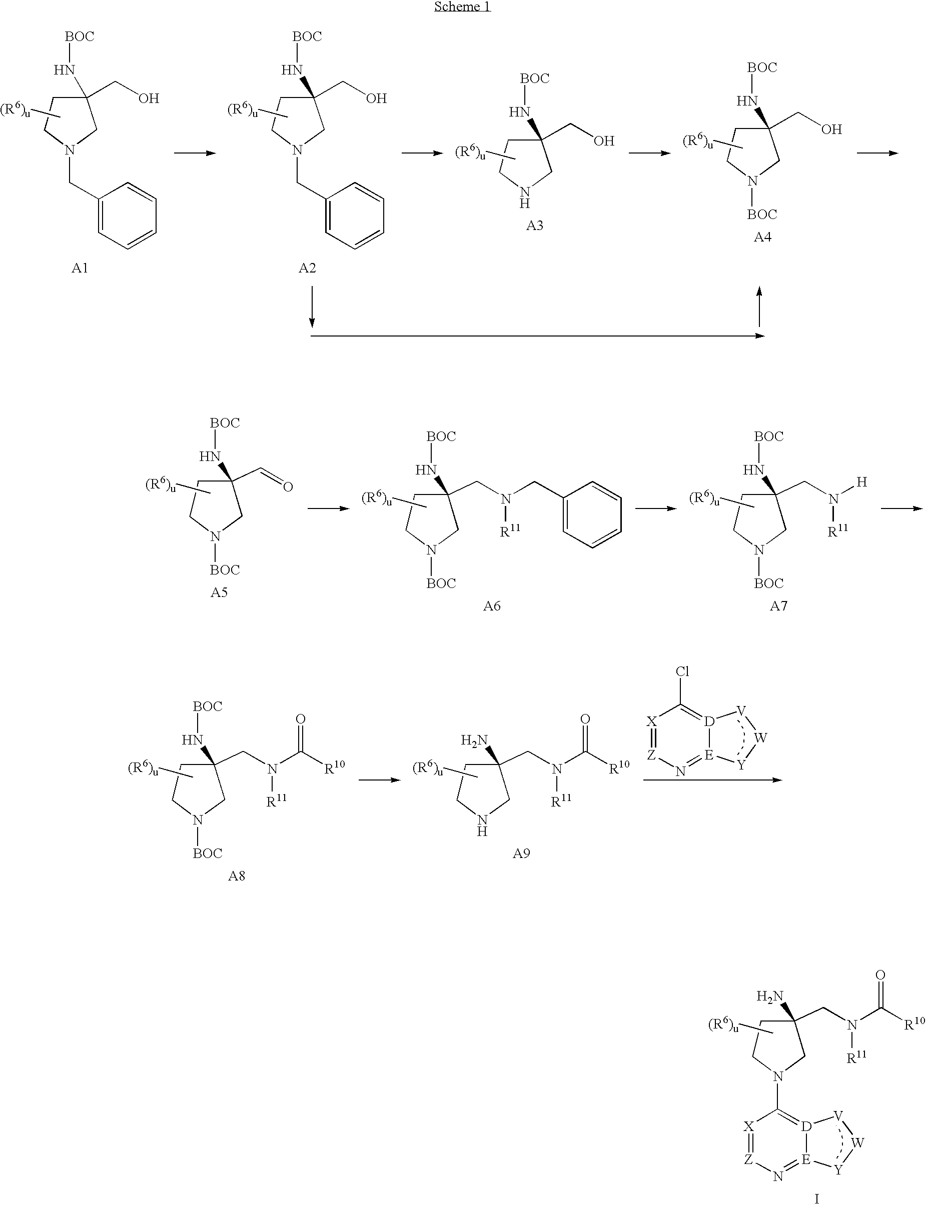
Compound A1 is commercially available or may be prepared by methods described herein or known to those skilled in the art. A1 may be resolved either by chiral HPLC chromatography or by salt formation with a chiral acid such that crystallization with a chiral acid purifies A1 to a single diasteriomeric salt. Typical chiral acids that may be used include, but are not limited to, (+) or (−) tartaric acid, (+) or (−) lactic acid, or (+) or (−) mandelic acid. The amine salt may either be converted to the free amine or used directly. A2 may be converted to A3 by selective removal of the N1 benzyl group followed by selective protecting N1 with BOC functionalization of N1 to give A4. Alternatively, A2 may be converted directly to A4 by a protocol which removes the N1 benzyl group under catalytic hydrogenation conditions in the presence of a protecting group reagent such as (BOC)₂O which concomitantly protects N1. Alcohol A4 may be converted to the aldehyde A5 using known oxidizing reagents such as, e.g., SO₃-pyridine complex. A5 may be reacted with benzyl amine, a substituted aryl methyl amine (Aryl-CH₂—NH₂) or secondary substituted aryl methyl amine (e.g., Aryl-CH₂—NH—R¹³) under reducing conditions such as those described for reductive aminations to form the amine A6. The benzyl moiety may be selectively removed under reducing conditions to give amine A7. A7 may be acylated with a suitable R¹⁰-carboxylic acid group such as, e.g., R¹⁰-carboxylic acid chloride, activated R¹⁰-carboxylic acid or R¹⁰-carboxylic anhydride to form A8. The protecting groups of A8 may be removed globally if the same or, if different protecting groups are used, selectively by choice in a stepwise manner to form A9. Reaction of A9 with a heterobicyclic compound of formula LG-A as defined above provides the amide derivative of a compound of formula I.

Scheme 2 shows a non-limiting method for making the amide-derivatives of the compound of formula I where a cyclic amine group is first combined with a bicyclic compound of formula LG-A followed by derivatization of the cyclic amine portion of the formed compound to provide the amide derivative. 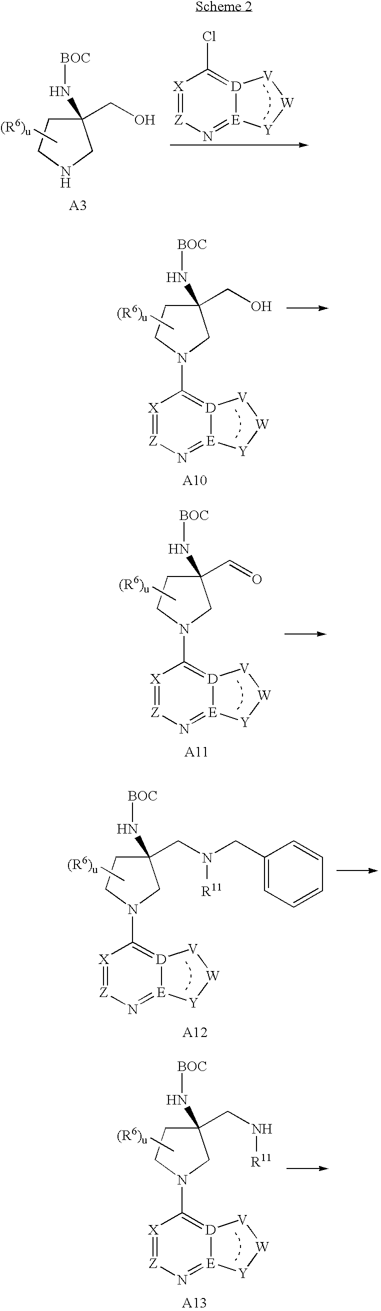 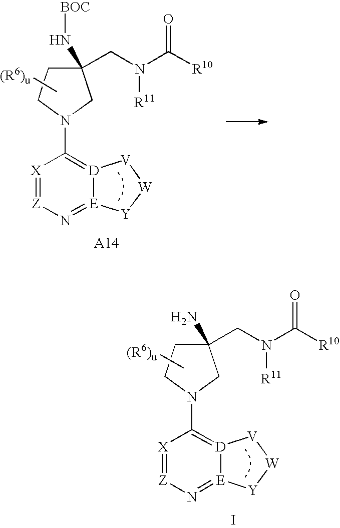
In Scheme 2, A3 (prepared as described in Scheme 1) may be combined with a heterobicyclic group of formula LG-A as defined above to form A10. Using methods similar to those described in Scheme I, alcohol A10 may be oxidized to aldehyde A11. A11 may then be converted to A12 through a reductive amination, followed by a de-benzylation (removal of the Aryl-CH₂— protecting group) to form A13. A13 may then be acylated with a suitable R¹⁰-carboxylic acid group such as, e.g., R¹⁰-carboxylic acid chloride, activated R¹⁰-carboxylic acid or R¹⁰-carboxylic anhydride to form A14. Removal of the amine protecting group on A14 by standard procedure provides the amide-derivatives of the compound of formula I.

Scheme 3 shows a non-limiting method for making amine-derivatives of the compound of formula I where a cyclic amine compound is derivatized followed by reaction with a bicyclic compound to form the amine derivative.

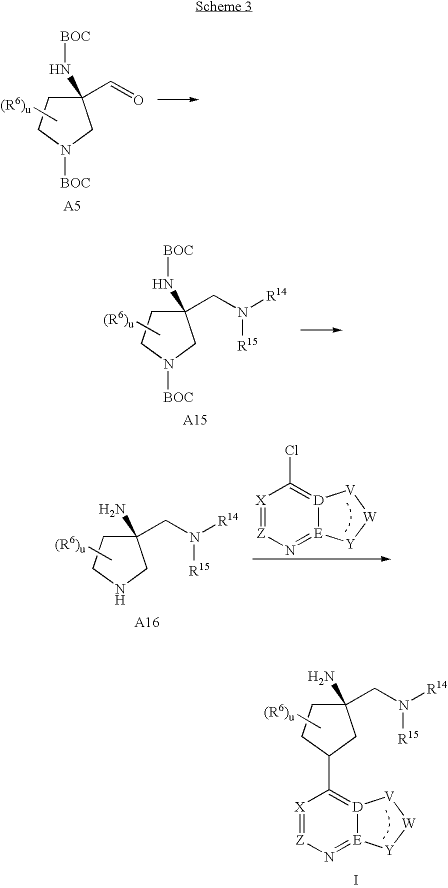

In Scheme 3, A5 (prepared as previously described in Scheme 1) may be reacted with an amine of the formula R¹⁴R¹⁵NH under reducing conditions such as those described for reductive aminations to provide the amine A15. The protecting groups of A15 may be removed globally if the same or, if different protecting groups are used, selectively by choice in a stepwise manner to give an intermediate of formula A16. A16 may then be allowed to react with a heterobicyclic compound of formula LG-A as defined above to provide the amine-derivatives of the compound of formula I.

Scheme 4 also shows a method for making amine-derivatives of the compound of formula I where the cyclic amine moiety is first combined with a heterobicyclic group of formula LG-A, followed by derivatization of the cyclic amine to form the amide derivative.

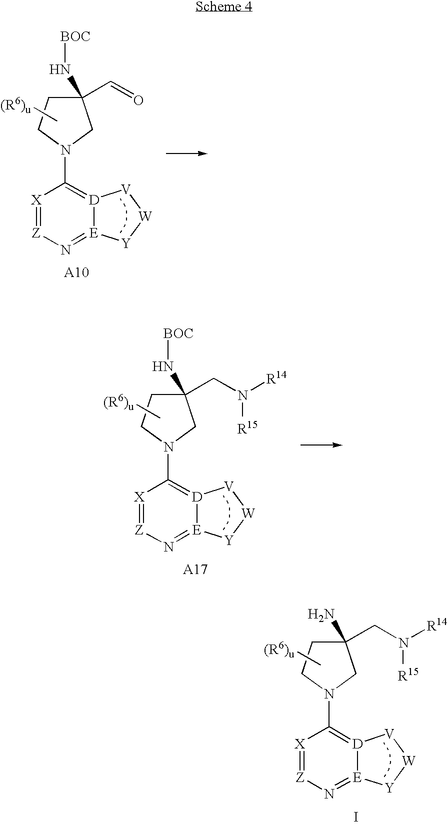

In Scheme 4, A10 (prepared as previously described in Scheme 2) may be reacted with an amine of the formula R¹⁴R¹⁵NH under reducing conditions such as described for reductive aminations to form the amine A17. Removal of the protecting group on A17 by standard procedures provides the amine-derivatives of the compound of formula I.

Scheme 5 shows a non-limiting method for making urea derivatives of the compound of formula I. 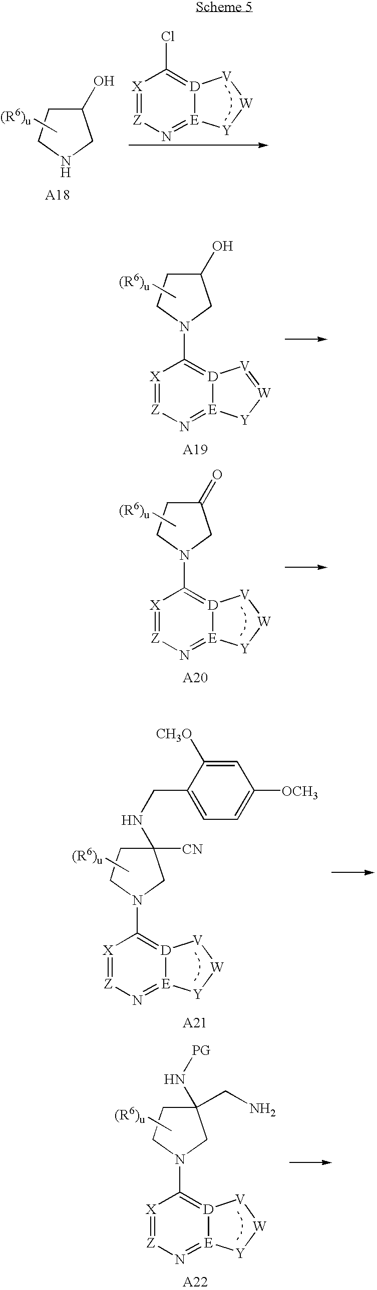 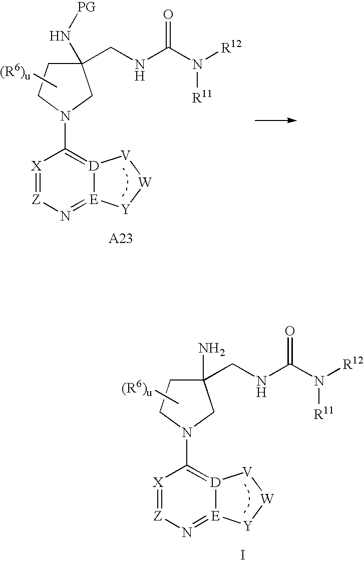
Compound A18 is commercially available or may be prepared by methods described herein or known to those skilled in the art. In Scheme 5, A18 may be combined with a heterobicyclic compound of formula LG-A as defined above to form alcohol A19. A19 may be converted to the ketone A20 using known oxidizing reagents such as, e.g., the SO₃-pyridine complex. A20 may be reacted with any benzyl amine or substituted aryl methyl amine (Aryl-CH₂—NH₂) or secondary amine (Aryl-CH₂—NH—R^{1′}) or any amine of the formula R^{1′}R^{2′}NH (where R^{1′} and R^{2′} are as defined as R¹ and R² above and may further include protecting groups) in the presence of a cyanide source such as, but not limited to, sodium or potassium cyanide or trimethylsilyl cyanide to form A21. A21 may be converted under reducing conditions to provide the amino methyl intermediate A22. As depicted in Scheme 5, A22 can be reacted with a suitable R¹⁰-isocyanate, an activated urethane such as R¹⁰R¹¹N—C(O)—O-p-nitrophenyl, or an activated R¹⁰-carboxylic acid group such as, e.g., R¹⁰-carboxylic acid halide, R¹⁰-carboxylic acid mixed anhydride or a symmetrical R¹⁰-carboxylic anhydride to provide A23. Alternatively, A22 can be reacted with an R¹⁰-sulfonyl halide to form a protected intermediate substituted with a -sulfonyl-R¹⁰ group. Removal of the amine protecting group on A23 using standard procedures provides a urea derivative of the compound of formula I.

Schemes 6 and 7 also illustrate methods for making the compounds of formula I, where a cyclic amine group is derivatized followed by reaction with a heterobicyclic compound of formula LG-A as defined above to form an amide derivative. In Scheme 6, A3 (prepared as described in Scheme 1) is protected at the N1 pyrrolidine nitrogen with a suitable protecting group such as, but not limited to, benzyloxycarbonyl (CBZ) or the tert-butyloxycarbonyl (BOC) group. As depicted in Scheme 3, A3 is converted to the CBZ-protected compound A24. A24 is then reacted with thionyl chloride followed by oxidation to provide the spirocyclic oxy-sultam A25. A25 may be reacted with a number of groups including, but not limited to, to sodium azide, sodium cyanide, sulfur nucleophiles such as R²⁰—S(O)ⱼ and nucleophilic heterocycles such as, e.g., pyrazole and triazole. As depicted in Scheme 6, A25 is reacted with sodium azide under reducing conditions to form the azide A26, which may be converted to the amine A27. 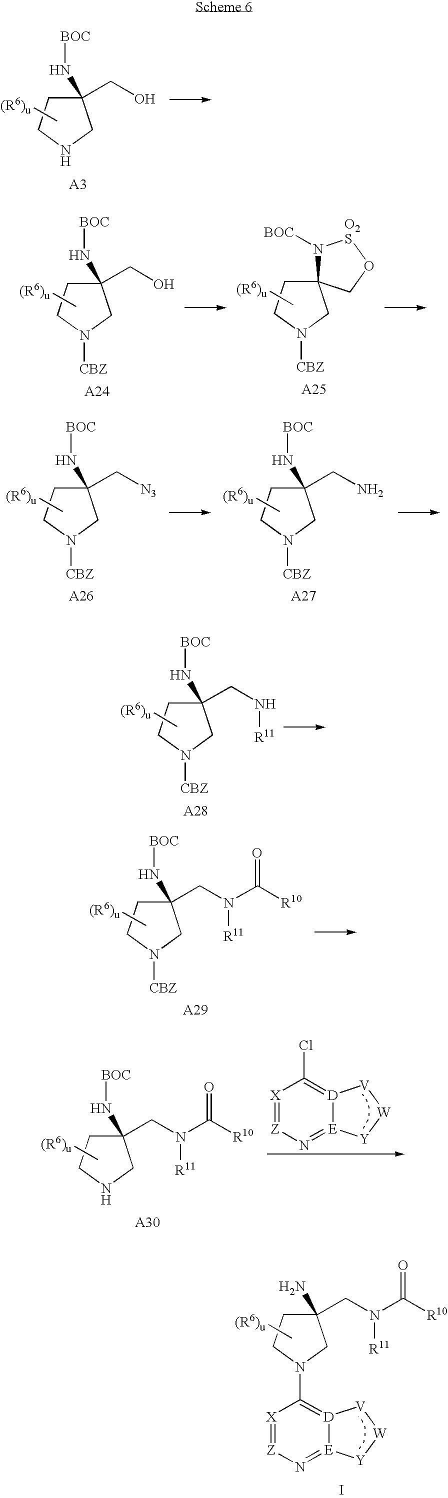
Compound A27 formed in Scheme 6 be used directly in a variety of transformations including, but not limited to, acylations, alkylations, and additions to activated heterocycles such as, e.g., 2-chloropyrimidines. As shown in Scheme 6, A27 is alkylated by reductive amination with R¹¹—CHO to incorporate a R¹¹ group and provide A28, which is then acylated to form A29. The protecting groups of A29 may be removed globally in one step or, if different protecting groups are used, then selectively by choice in a stepwise manner to form A30. As depicted in Scheme 6, A30 may be combined with a heterobicyclic compound of formula LG-A as defined above to provide a compound of formula I or, if needed, the product of the reaction of A30 and LG-A may undergo an additional de-protection step to provide a compound of formula I.

In Scheme 7, the spirocyclic oxy-sultam A325 (prepared as previously described in Scheme 6) may be reacted with a suitable reagent such as ruthenium(III) chloride and sodium periodate followed by reaction with thiophenol to provide the thio compound A31. A31 may then be deprotected to form A32 followed by reaction with a heterobicyclic compound of formula LG-A as defined above to provide a compound of formula I.

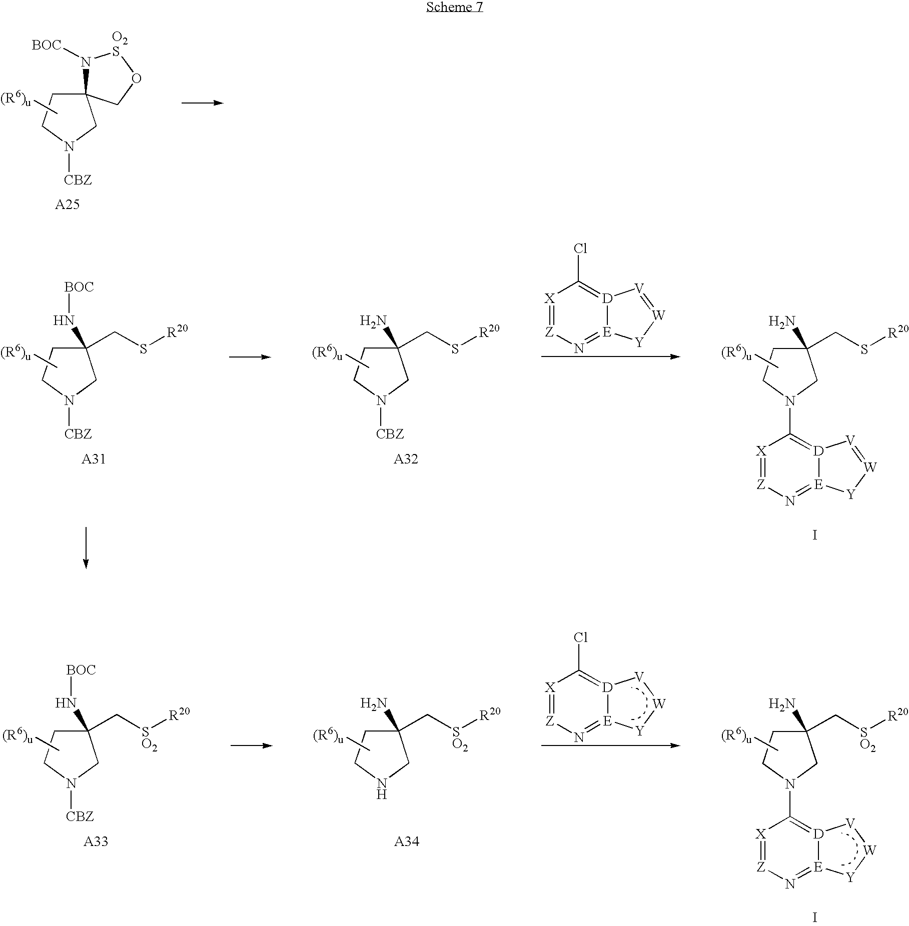

Alternatively, Scheme 7 also shows that compound A31 may be oxidized by known methods to form a sulfone or sulfoxide such as A32. A32 may then be deprotected to form the compound A33 followed by reaction with a heterobicyclic compound of formula LG-A as defined above to provide a compound of formula I.

Scheme 8 depicts a non-limiting method for making pyrrolidinyl ether derivatives of the compounds of formula I having an R⁶ group attached to the 4-position of the pyrrolidinyl moiety.

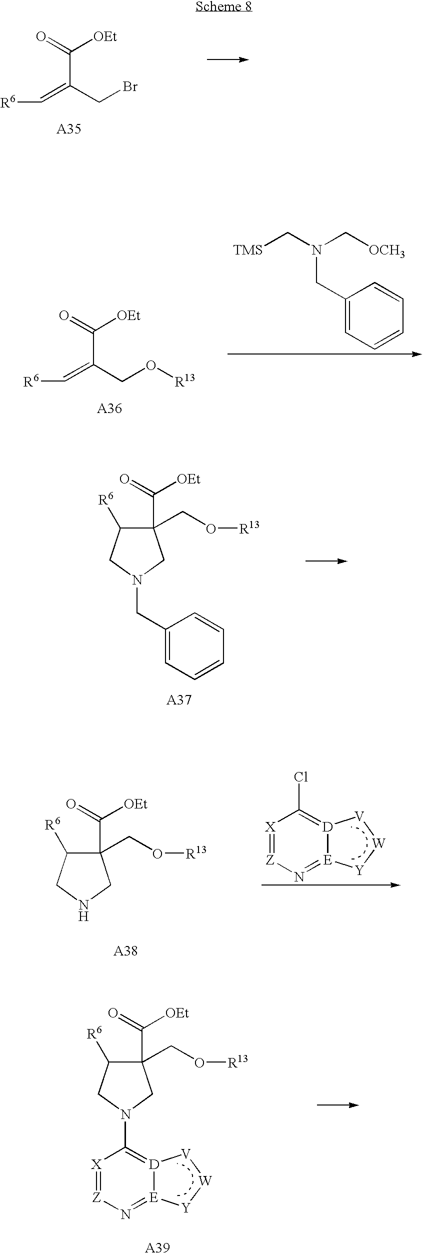

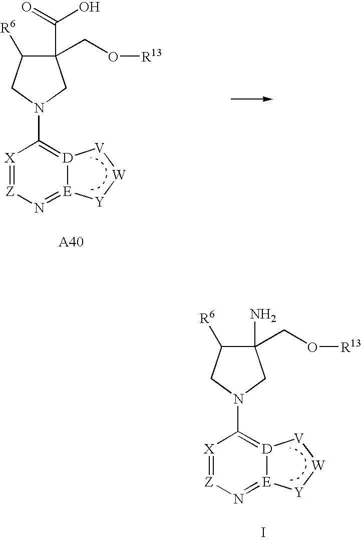

Compound A35 is commercially available or may be prepared by methods known to those skilled in the art. A35 is converted to the ether analog A36. It will be understood that appended ester group of A35 may optionally have a chiral auxiliary in place of the -ethyl group such as, e.g., (+) or (−) 8-phenyl menthol ester. A36 may be combined with N-benzyl-N-(methoxymethyl)-N-((trimethylsilane)methyl)-amine, or a compound of similar structure, to provide A37. The benzyl group of A37 may be selectively removed under reducing conditions to provide A38. A38 may be allowed to react with a heterobicyclic compound of formula LG-A as defined above to provide the ester compound A39. The ester of A39 may be cleaved to provide the corresponding carboxylic acid A40 and converted by acid rearrangement (e.g., a Curtius reaction) to provide a compound of formula I.

In a variation of the method described in Scheme 8, Scheme 9 depicts a method for making pyrrolidinyl ether derivatives of the compounds of formula I having an R⁶ group attached to the 4-position of the pyrrolidinyl moiety where the cyclic amine is formed by reaction of a chiral alkene. 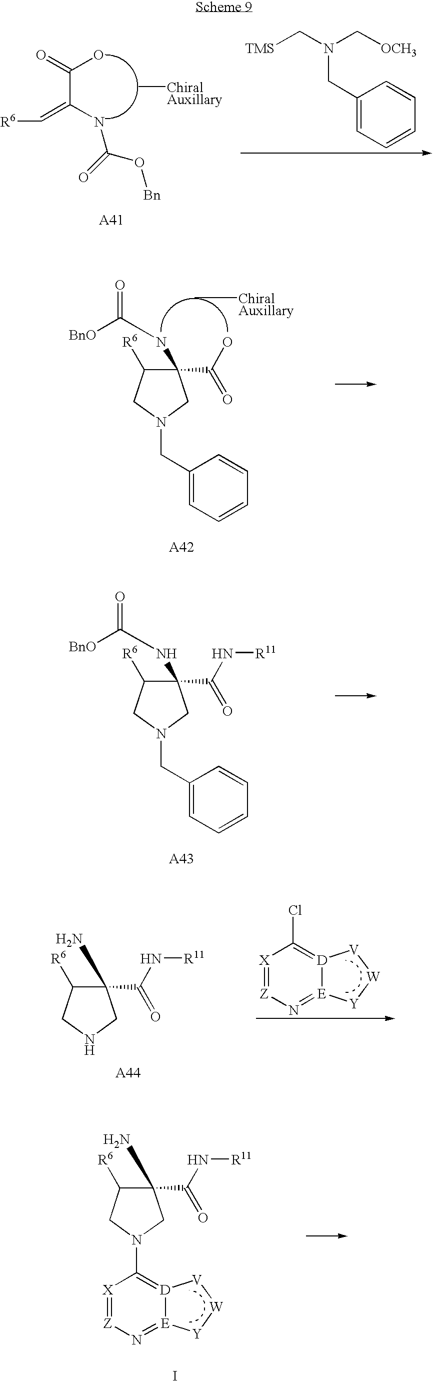 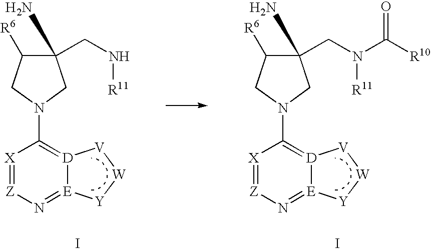
In Scheme 9, the chiral alkene A41 (which may be prepared by known methods) is allowed to react with the commercially available reagent N-benzyl-N-(methoxymethyl)-N-((trimethylsilane)methyl)amine, or compound of similar structure, to provide the compound A42. A42 may be combined with an amine of formula R¹¹—NH₂ or R¹⁴R¹⁵—NH₂ to provide compound A43. The protecting groups of A43 may be removed globally in one step or, if different protecting groups are used, then selectively by choice in a stepwise manner to provide A44. A44 may be combined with a heterobicyclic compound of formula LG-A as defined above to form an amide compound of formula I. The carbonyl group of amide compound of formula I may then be reduced using known methods to the corresponding alkane compound of formula I. Also as depicted in Scheme 9, alkane compound of formula I may be acylated with a suitable carbonyl compound such as, but not limited to, an isocyanate or a R¹⁰-carboxylic acid group such, e.g., R¹⁰-carboxylic acid chloride, activated R¹⁰-carboxylic acid, or R¹⁰-carboxylic anhydride to form an amide compound of formula I.

Scheme 10 depicts a non-limiting method for making an intermediate cyclic amine having an R⁸ group attached to the 5-position of the pyrrolidinyl moiety.

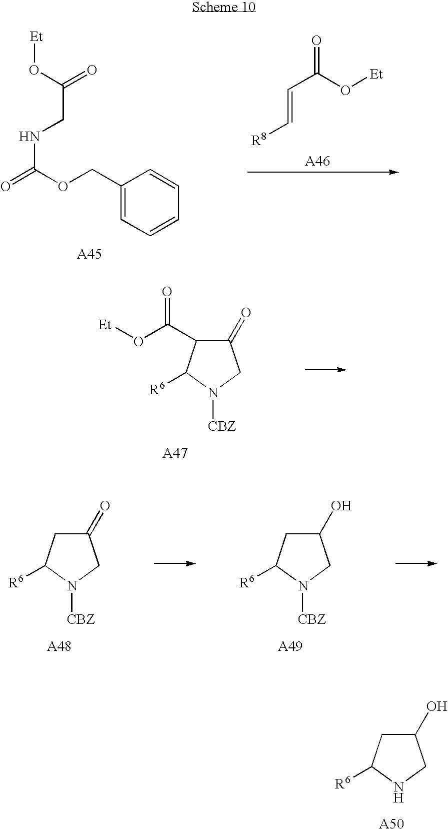

In Scheme 10, compounds A45 and A46 may be combined under basic conditions (see, e.g., Kende et al., J. Org. Chem. (1990), 55(3), 918-24 and references cited therein) to provide A47. A47 can then be decarboxylated under Krapcho conditions to produce the ketone A48, which can then be treated with a reducing agent such as sodium borohydride to produce the alcohol A49. The CBZ group of A49 can then be removed using known conditions to give the amine A50, which is the 5-substituted cyclic amine analog of A18 described in Scheme 6. A50 can then be reacted using procedures similar to those described in Scheme 6 to provide the urea derivatives of the compound of formula I where the 5-position of the pyrrolidinyl moiety is substituted with an R⁶ group.

Other methods for making intermediates useful for making compounds of the invention are known in the art (see, e.g., Tomiita et al., Synthesis and Structure-Activity Relationships of Novel 7-Substituted 1,4-Dihydro-4-oxo-1-(2-thiazolyl)-1,8-naphthyridine-3-carboxylic Acids as Antitumor Agents. Part 1. Journal of Medicinal Chemistry (2002), 45(25), 5564-5575 and references cited therein).

As noted above, the compounds of the invention are useful for treating abnormal cell growth such, e.g., cancer. Also as noted above, the serine/threonine kinases Akt and P70S6k are implicated in human cancer. For example, Akt is known to be highly active certain human cancers. Without being limited by theory, Applicants believe that the compounds of the invention are useful for treating or preventing abnormal cell growth by inhibiting the Akt and/or the P70S6K1 kinases.

Applicants have also found that the compounds of formula I are more selective in targeting Akt and S6 kinases than are analogous compounds in which the pyrrolidinyl ring of the compound of formula I is replaced by a piperidinyl ring.

In one embodiment, the invention relates to a method of using the compound of formula I to regulate the expression of at least one serine/threonine kinase.

In another embodiment, the invention relates to a method of using the compound of formula I to regulate the expression of at least one serine/threonine kinase wherein the at least one serine/threonine kinase is selected from the group consisting of Akt and P70S6K1.

In another embodiment, the invention relates to a method of using the compound of formula I to regulate the expression of Akt.

In another embodiment, the invention relates to a method of using the compound of formula I to regulate the expression of P70S6K1.

The in vitro activity of the compounds of formula I may be determined by the following procedures.

Akt:

The Akt1 kinase assay is based on the measurement of fluorescence polarization using IMAP technology (Molecular Devices Corporation #R8062). Two microliters of inhibitor compounds diluted to a concentration of 10 millimolar are added to column 2 of a polypropylene 96-well plate containing 98 microliters of 100% DMSO; the wells in columns 3-12 contain 60 microliters of 100% DMSO. The various test compounds are serially diluted 1:3 across the plate by pipetting 30 microliters of compounds into wells containing 60 microliters of 100% DMSO. Column 12 receives DMSO only and is used as a negative control for inhibition. The components of the wells are mixed and 15 microliters of each well are transferred to another 96-well plate already containing 60 microliters of reaction buffer (RB: 10 mM Tris-HCl, pH 7.5, 10 mM MgCl₂, 0.1 mM EGTA, 0.01% Triton-X100 (Sigma #X-100), freshly added 1 mM DTT). After mixing, the S6K1 reactions are assembled. First, five microliters of the above compound/reaction buffer mixture is transferred to the bottom of a 96-well black polystyrene reaction plate (Costar, #3694). Next, ten microliters of a solution containing 4 micromolar ATP and 40 nanomolar fluorescent-labeled Crosstide (Tamara-labeled GRPRTSSFAEG peptide) are added. Then, 5 microliters (1.5 nanograms) of Akt protein in RB are added. The version of Akt used in these studies lacks the pleckstrin homology (PH) domain, and contains an aspartic acid residue in place of a serine residue in position 473 within the Akt1 hydrophobic motif. The Akt1 protein contains a polyhistidine tag at the amino terminus and is prephosphorylated on threonine at position 308 in order to activate latent kinase activity. Once the reaction components and inhibitors are assembled, the plates are gently tapped, covered with foil, and then incubated at ambient temperature for 30 minutes. IMAP beads (Molecular Devices) are then added (60 microliters of a 1:400 dilution of beads in RB). Plates are read on a Victor Plate Reader with the following settings: CW lamp filter: 544 nm, emission filter: 615 nm. Control values from wells lacking Akt protein are subtracted from the gross readings, and IC50 values are calculated using XLDA.

The compounds of formula I produce inhibition of Akt kinase activity at concentrations of less than 10 uM. For example, Table 1 shows the concentrations at which exemplary compounds of formula I inhibit Akt kinase activity:

TABLE 1
Concentration (uM) at which exemplary compounds of formula I
inhibit Akt kinase activity.

| Concentration | |
|---|---|
| Range, uM | Compound of formula I* |
| <0.00025-0.0025 | 3, 9, 13, 18, 26, 36, 43, 73, 82, 88, 93, 97, 98, |
| | 106, 107 |
| 0.00251-0.010 | 10, 11, 19, 21, 23, 28, 31, 33, 34, 35, 37, 38, |
| | 40, 59, 70, 71, 78, 79, 80, 81, 83, 85, 87, 89, |
| | 91, 92, 94, 95, 96, 100, 103, 104, 108, 109, |
| | 110, 112, 115, 119, 123, 124, 129, 130, 131, |
| | 133 |
| 0.0101-0.10 | 1, 4, 6, 7, 12, 15, 16, 20, 22, 24, 25, 29, 30, |
| | 32, 41, 42, 51, 52, 53, 55, 56, 57, 60, 61, 63, |
| | 64, 65, 69, 74, 84, 86, 90, 99, 101, 102, 105, |
| | 111, 113, 114, 116, 117, 118, 120, 121, 122, |
| | 125, 126, 127, 128, 132, 194, 198 |
| 0.101-10 | 5, 8, 14, 17, 27, 39, 44, 45, 46, 47, 48, 49, 50, |
| | 54, 58, 62, 66, 67, 68, 75, 76, 77, 195, 196, |
| | 197 |

*See the Examples Section for the structures corresponding to the numbered compounds.

Accordingly, in one embodiment, the compounds of formula I produce inhibition of Akt kinase activity at concentrations of less than 10 uM. In another embodiment, the compounds of formula I produce inhibition of Akt kinase activity at concentrations of less than 5 uM. In another embodiment, the compounds of formula I produce inhibition of Akt kinase activity at concentrations of less than 1 uM. In another embodiment, the compounds of formula I produce inhibition of Akt kinase activity at concentrations of less than 0.01 uM. In another embodiment, the compounds of formula I produce inhibition of Akt kinase activity at concentrations of less than 0.001 uM.

P70S6K1:

The P70S6K1 kinase assay is based on the measurement of fluorescence polarization using IMAP technology (Molecular Devices Corporation #R8062). Two microliters of inhibitor compounds diluted to a concentration of 10 millimolar are added to column 2 of a polypropylene 96-well plate containing 98 microliters of 100% DMSO; the wells in columns 3-11 contain 60 microliters of 100% DMSO. The various test compounds are serially diluted 1:3 across the plate by pipetting 30 microliters of compounds into wells containing 60 microliters of 100% DMSO. Column 12 receives DMSO only and is used as a negative control for inhibition. The components of the wells are mixed and 15 microliters of each well are transferred to another 96-well plate already containing 60 microliters of reaction buffer (RB: 10 mM Tris-HCl, pH 7.5, 10 mM MgCl₂, 0.1 mM EGTA, 0.01% Triton-X100 (Sigma #X-100), freshly added 1 mM DTT). After mixing, the P70S6K1 reactions are assembled. First, five microliters of the above compound/reaction buffer mixture is transferred to the bottom of a 96-well black polystyrene reaction plate (Costar, #3694). Next, ten microliters of a solution containing 4 micromolar ATP and 200 nanomolar fluorescent-labeled peptide substrate (TAM-labeled AKRRRLSSLRA peptide) are added. Then, 5 microliters (26 nanograms) of S6 Kinase (T412E; Upstate #14-486) in RB are added. The version of P70S6K1 used in these studies contains a glutamic acid residue in place of a threonine residue in position 412 within P70S6K1. Once the reaction components and inhibitors are assembled, the plates are gently tapped, covered with foil, and then incubated at ambient temperature for 30 minutes. IMAP beads (Molecular Devices) are then added (60 microliters of a 1:400 dilution of beads in RB). Plates are read on a Victor Plate Reader with the following settings: CW lamp filter: 485 nm, emission filter: 535 nm. Control values from wells lacking S6K protein are subtracted from the gross readings, and IC50 values are calculated using XLDA.

S6K1 Cell Assay: Cells of interest (NIH/3T3, U87) are seeded in complete medium containing 10-15% fetal bovine serum at 20,000 cells/well into 48-well plates, and grown at 37 degrees in a humidified tissue culture incubator. Compounds are serially diluted in cell growth medium (Dulbecco's Modified Eagle Medium [DMEM, Gibco] supplemented with L-glutamine and penicillin-streptomycin (Gibco)). For most studies, fetal bovine serum is omitted from the compound dilution plates. Compounds are diluted 1:2 to provide a range of final concentrations from 10 to 0.15 micromolar. Cells are allowed to grow in complete medium until they reach 60-95% confluence in the wells, at which point the medium is carefully removed and replaced with serum-free medium containing the diluted compounds. After two hours incubation, medium and compounds are carefully removed, and the monolayers are washed once with PBS. A cell lysis buffer (100 microliters of a buffer containing 1 mM EDTA, 1% (v/v) glycerol, 50 mM HEPES buffer, 1.5 mM MgCl₂, 1.6 mM Na₃VO4, 150 mM NaCl, 10 mM NaF, 1% (v/v) Triton X-100 and protease inhibitor cocktail tablets (Roche Diagnostics; 1 tablet per 25 mL buffer)) is added to each well, and the plates are left to incubate on ice for 30 minutes. Cell lysates are harvested and protein concentrations are determined. Polyacrylamide gels are loaded using 2.5 micrograms of clarified cell lysate per lane. After gel electrophoresis and western blotting, nitrocellulose filters are probed using antibodies specific for the phosphorylated form of ribosomal S6 protein (S6 residues S240/244; Cell Signaling Technology # 2215). Filters are simultaneously probed using antibodies to β-actin as a normalization control, if needed. After image development, the results are captured on a Lumi-Imager F1 (Roche). Normalized signal intensities, expressed as a percentage of DMSO-only control cultures, are plotted and IC50 values are calculated using XLDA.

The compounds of formula I produce inhibition of S6 kinase activity at concentrations of less than 10 uM. In another embodiment, the compounds of formula I produce inhibition of S6 kinase activity at concentrations of less than 5 uM. In another embodiment, the compounds of formula I produce inhibition of S6 kinase activity at concentrations of less than 1 uM. In another embodiment, the compounds of formula I produce inhibition of S6 kinase activity at concentrations of less than 0.1 uM. In another embodiment, the compounds of formula I produce inhibition of Akt kinase activity at concentrations of less than 0.01 uM.

PKA Kinase Assay

The PKA Kinase assay is used to measure the selectivity of the compounds of formula I for PKA kinase as discussed below. The PKA activity of the compounds of formula I was determined using the PKA IMAP® Kinase Assay (G7096A).

Materials:
1) Kinase Reaction Buffer (RB):_{—}10 mM Tris-HCl, pH 7.5, 10 mM MgCl₂, 0.1 mM EGTA, 0.01% Triton-X 100 (Sigma #X-100); 1 mM DTT (N.E. Biolabs #B7705S) added fresh
2) ATP (Sigma #5394) 10 mM stock in H₂O, −20° C.
3) 20 μM working stock TAMRA Kemptide (TK. Molecular Devices #R7332): [N-Term]TMRS-Leu-Arg-Arg-Ala-Ser-Leu-Gly-OH[C-Term]
4) PKA, catalytic subunit, recombinant (Upstate #14-440). 5 ug/25 ul lot 22361AU
5) DMSO (Baker #9224-01)
6) IMAP® Progressive Express Screening Kit for IPP (Molecular Devices #R8124) Includes IMAP® Progressive Beads and Binding Buffer
7) 96-well ½ area plates, black polystyrene, Costar #3694
8) 96-well round bottom polypropylene plates, Costar #9667

Assay:

NOTE: Protect all Tamra-Containing Reagents from Light.

1) Prepare the 1× Reaction Buffer (RB) by fresh addition of DTT to 1 mM
2) For single point testing, make up compound dilution plate as follows: “96 well plates; Row Empty: empty; 84 cmpds” are added to the remaining wells at 10 nM.
3) For 150 determination: To column 2 of polypropylene plate add 100 ul 100% DMSO; to all other wells add 60 ul 100% DMSO.
4) Pipet 2 ul 10 mM stock compound to column 2; mix well
5) Serially dilute 1:3 across from column 2-11, transferring 30 ul to next well and mixing well.
6) Leave column 12 for zero compound (control signal)
7) For Single Point testing (10 uM final) add 1 ul 10 mM compound to 100 ul DMSO; mix; transfer 5 ul to 120 ul RB; mix; transfer 5 ul to Assay Plate
8) For 150 determination: Make up compound RB dilution plate: 60 μl/well RB in 96-well polypropylene round bottom plate.
9) Transfer compounds to assay plate as follows: Mix DMSO dilutions; transfer 15 μl to 60 μl RB; transfer 5 μl/well to assay plates (single points, triplicate plates), delivering carefully to the bottom of the well; repeat for each dilution up to 10 μM.
10) To run PKA Reactions: Make up ATP/KT solution; 4 uM ATP, 200 nM KT (4 uM ATP, 100 nM KTfinal)
11) Pipet 10 μl KT/ATP to wells containing compound.
12) Make up protein solution in RB and determine the protein concentration.
13) Deliver 5 μl protein solution to all wells except A1-H1, delivering to the upper region of the side of the well; Add 5 μl RB to A1-H1 (these are the 0 protein background controls)
14) Gently tap the plate to ensure all volumes get to the bottom. Cover with foil lid.
15) Incubate RT on bench top 30 minutes.
16) Add 60 μl IMAP® beads in IMAP® Progressive binding buffer (100% A) diluted 1:1000
17) Read on VICTOR™ plate reader (protocol LissieTAMRA2) with the following settings: CW lamp filter 544 nm; emission filter 615 nm. Some adjustments may be made to the plate reader protocol depending on the reader and available filters.
18) Use XLDA to calculate IC50s.

As noted above, the compounds of formula I are selective in targeting Akt and S6 kinases. The selectivity can be determined by measuring the PKA activity and/or the Akt or S6 activity. The ratio of the (PKA activity)/(Akt activity) or (PKA activity)/(S6 activity) is then calculated using the IC50 values. Compounds which are selective for Akt (or S6) will have ratios greater than one. Typically, the compounds of formula I exhibit PKA/Akt selectivity ratios of at least about 2. Accordingly, in one embodiment, the compounds of formula I exhibit PKA/Akt selectivity ratios of at least about 2; in another embodiment, the compounds of formula I exhibit PKA/Akt selectivity ratios of at least about 10. And in another embodiment, the compounds of formula I exhibit PKA/Akt selectivity ratios of at least about 20.

Administration of the compounds of the present invention (hereinafter the “active compound(s)”) may be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

As noted above, the active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

As noted above, the invention also relates to a pharmaceutical composition comprising a compound of formula I. The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms may be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of formula I and methods of preparing such compounds. It is to be understood that the scope of the present invention is not limited in any way by the scope of the following examples and preparations. In the following examples, “Ac” means acetyl, “Et” means ethyl, “Me” means methyl, and “Bu” means butyl.

### EXPERIMENTAL

HPLC: Where HPLC chromatography is referred to in the preparations and examples below, the general conditions used, unless otherwise indicated, are as detailed by HPLC methods A through L as shown in Table 2:

TABLE 2
HPLC conditions used in the Examples.

| HPLC methods | Column | Gradient |
|---|---|---|
| A | Symmetry C18 (4.6 × 50 mm; 3.5 um), | H₂O/CH₃CN/1% TFA in H₂O (85:10:5 |
| | 2.0 mL/min | at 0 min. and 0:95:5 at 5 min) |
| B | Symmetry C8 (4.6 × 50 mm; 3.5 um), | H₂O/CH₃CN/1% TFA in H₂O (90:5:5 |
| | 2.0 mL/min | at 0 min. and 0:95:5 at 5 min) |
| C | Symmetry C18 (4.6 × 50 mm; 3.5 um), | H₂O/CH₃CN/1% TFA in H₂O (75:20:5 |
| | 2.0 mL/min | at 0 min. and 0:95:5 at 5 min) |
| D | Symmetry C18 (4.6 × 100 mm, 5 um), | H₂O/CH₃CN/1% TFA in H₂O (90:5:5 |
| | 2.0 mL/min | at 0 min. and 0:95:5 at 7.5 min) |
| E | XTerra MS C8 (4.6 × 50 mm; 3.5 um), | H₂O/CH₃CN/2% NH₃ in H₂O (90:5:5 |
| | 2.0 mL/min | at 0 min. and 0:95:5 at 5 min) |
| F | Symmetry C8 (4.6 × 50 mm; 3.5 um), | H₂O/CH₃CN/1% TFA in H₂O (90:5:5 |
| | 2.0 mL/min | at 0 min. and 15:80:5 at 5 min) |
| G | Xterra MS C18 (4.6 × 50 mm; 3.5 um), | H₂O/CH₃CN/2% NH₃ in H₂O (90:5:5 |
| | 2.0 mL/min | at 0 min. and 0:95:5 at 5 min) |
| H | Zorbax 5B-C18, 5 micron, 4.6 × 150 mm, | 0.2 M ammonium acetate/acetic |
| | 3.00 mL/min | acid aqueous buffer/acetonitrile |
| | | (100:0 at 0 min., 0:100 at 10 min) |
| I | Polaris 5 micron C18-A 20 × 2.0 mm, | 94.952% water, 4.998% acetonitrile, |
| (LCMS STD) | 1 mL/min | 0.05% formic acid/0.05% formic |
| | | acid in acetonitrile (95:5 at 0 min., |
| | | 80:20 at 1.05 min., 50:50 at 2.30 min., |
| | | 0:100 at 3.55 min) |
| J | Polaris 5 micron C18-A, 20 × 2.0 mm, | 94.952% water, 4.998% acetonitrile, |
| (LCMS Polar) | 1 mL/min | 0.05% formic acid/0.05% formic |
| | | acid in acetonitrile (95:5 at 0 min., |
| | | 80:20 at 2.00 min., 50:50 at 2.30 min., |
| | | 0:100 at 3.50 min) |
| K | Waters Xterra MS C18, 5 mm, | 0.1% TFA/acetonitrile (100:0 at 0 min., |
| | 3.0 × 50 mm, 1.5 mL/min | 100:0 at 1 min., 0:100 at 6 min) |
| L | Waters Xterra MS C18, 3.5 μm, | water, acetonitrile, 0.6% ammonium |
| (LCMS Basic) | 4.6 × 50 mm, 2.0 mL/min | hydroxide in water (80:15:5 at 0 min., |
| | | 0:95:5 at 3.50 min., 0:95:5 at 4.00 min) |

NMR Data: Analytical data for compounds I-198 described in Examples 1-198 can be found in Tables 3-5.
Akt Kinase cell activity: The Akt kinase activity (IC50) of the compound described in Examples 1-198 can be found in Tables 3-5.

### Example 1

### Preparation of N—(((S)-3-amino-1-(5-ethyl-7H-pyrrolo[2,3-d]pyrimidin-4-yl)pyrrolidin-3-yl)methyl)-5-methylisoxazol-3-amine (1)

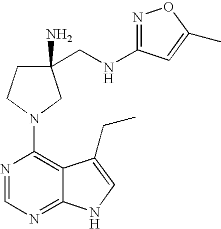

Step 1: 3-hydroxymethyl-pyrrolidin-3-yl)-carbamic acid tert-butyl ester (C1) (2.00 g; 9.25 mmol) (see Tomita et al., J. Med. Chem. 2002, 45, 5564) was reacted with 1 equivalent 15 of benzyl bromide in the presence of N,N-diisopropylamine (DIIPEA) to provide the racemate of tert-butyl (R)-1-benzyl-3-(hydroxymethyl)pyrrolidin-3-ylcarbamate (C2), which was resolved using chiral HPLC to provide C2.

Step 2. A solution of C2 (11.00 g, 34.8 mmol) in THF (110 mL) was charged to a Parr vessel, and the resultant solution was sparged with nitrogen. di-t-Butyl carbonate (Boc₂O) (7.83 g, 34.8 mmol) and Pearlman's Catalyst (2.0 g, 50% H₂O) were added to the Parr vessel, and the contents of the Parr Vessel were hydrogenated at 50 psig for 16 hr. The resultant mixture was filtered over Celite and the solids washed with methanol. The combined filtrates were then concentrated to provide 11.52 g of (R)-1-(tert-butoxycarbonyl)-3-(hydroxymethyl)pyrrolidin-3-ylcarbamate (C3) as a white foam in quantitative yield. LRMS (M+): 317.3; t_{R} (LCMS standard): 2.03 min.

Step 3: A solution of a SO₃-Pyridine complex (17.2 g; 104 mmol) in dimethylsulfoxide (DMSO-d₆) (100 mL) was cooled to 12° C. and treated with TEA (15.4 mL, 104 mmol). A solution of C3 (11.52 g; 34.8 mmol) in DMSO (32 mL) was slowly added to the reaction mixture via syringe. The reaction mixture was warmed to ambient temperature and stirred for 2 hr. The mixture was then treated with ethyl acetate (800 mL) and washed in this order with brine (2×175 mL), H₂O (175 mL), 5% aq Na₂HCO₃ (175 mL) and saturated aq. CuSO₄. The organic phase was collected, dried over Na₂SO₄, filtered, and concentrated to provide tert-butyl (R)-1-(tert-butoxycarbonyl)-3-formylpyrrolidin-3-ylcarbamate (C4) as a white foam. Yield: 8.04 g, 74%. ¹H NMR (300 MHz methanol-d₄) δ: 9.46 (s, 1H), 3.64-3.50 (m, 2H), 3.48-3.32 (m, 4H), 2.17-2.06 (m, 1H), 2.04-1.93 (m, 1H), 1.383 (s, 9H), 1.365 (s, 9H).

Step 4: A mixture of C4 (8.04 g), anhydrous acetonitrile (30 mL), 5-methylisoxazol-3-amine (0.53 g, 5.41 mmol) and molecular sieves (1 g) was treated with sodium triacetoxyborohydride (1.72 g, 8.11 mmol). The reaction mixture was cooled to 0° C. and treated drop-wise with trifluoroacetic acid (TFA) (1.5 mL). The reaction mixture was stirred over night while warming to 25° C. The mixture was then quenched with saturated NaHCO₃, extracted with ethyl acetate (3×45 mL), and the combined organic phases were concentrated. The resultant residue was purified via Isco Combiflash using MeOH/Chloroform gradient, and the fractions containing produced were combined and concentrated to provide tert-butyl (S)-1-(tert-butoxycarbonyl)-3-((5-methylisoxazol-3-ylamino)methyl)pyrrolidin-3-ylcarbamate (C5). Yield: 1.78 g, 83%. LRMS (M+): 397.0; t_{R} (LCMS standard): 2.55 min.

Step 5: A solution of C5 (1.78 g) in dichloromethane (DCM) (20 mL) was treated with TFA (20 mL) and stirred at 25° C. for 2 hr. The mixture was then concentrated to provide 2.91 g of the TFA salt of N—(((R)-3-aminopyrrolidin-3-yl)methyl)-5-methylisoxazol-3-amine (C6) in quantitative yield. The crude material was freebased using Water's Oasis MCX cartridge by placing 600 mg of the crude material on a 6 g cartridge. APCI (M+): 197.3; t_{R} (Agilent AKT1.M): 3.38 min.

Step 6: A solution of 4-Chloro-5-ethyl-7H-pyrrolo[2,3-d]pyrimidine (C7) (200 mg, 1.06 mmol) (see Townsend et al., J. Med. Chem. 1990, 33 (7), 1984) in 2-propanol (2 mL) was treated with DIIPEA (0.21 mL; 1.17 mmol) followed by C6 (193 mg; 1.06 mmol). The mixture was stirred at 80° C. overnight then concentrated. The resultant residue was dissolved in DMSO (2 mL) and filtered, and the filtrate was purified by reverse phase HPLC using 1% ammonium hydroxide in water/acetonitrile system. The pure aqueous fractions were concentrated to provide (1). Yield: 106.4 mg, 27%.

### Example 2

### Preparation of N—(((S)-3-amino-1-(3-chloro-1H-pyrrolo[2,3-b]pyridin-4-yl)pyrrolidin-3-yl)methyl)-2-methylpyridin-3-amine (2)

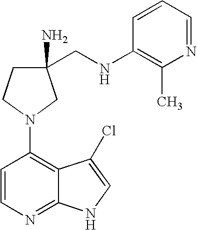

Step 1: A solution of C4 (1.6 g, 5.09 mmol) in anhydrous acetonitrile (32 mL) was treated with 2-methylpyridin-3-amine (0.55 g, 5.09 mmol) and molecular sieves (1 g). The resultant mixture was then treated with sodium triacetoxyborohydride (1.62 g, 7.63 mmol), cooled to 0° C., and treated drop-wise with TFA (1.5 mL). The mixture was allowed to warm to 25° C. and stirred overnight. The mixture was then quenched with saturated NaHCO₃ and extracted with ethyl acetate (3×45 mL). The combined organic extracts were concentrated, and the resultant residue was purified via Isco Combiflash using MeOH/Chloroform gradient. The fractions containing produce were concentrated to provide (S)-tert-butyl 3-(tert-butoxycarbonyl)-3-((2-methylpyridin-3-ylamino)methyl)pyrrolidine-1-carboxylate (C8). Yield: 1.31 g, 63%. LRMS (M+): 407.5; tR (LCMS standard): 1.46 min.

Step 2: A solution of C8 in DCM (20 mL) was treated with TFA (20 mL) and stirred at 25° C. for 2 hr. The mixture was then concentrated to provide the TFA salt of (R)—N-((3-aminopyrrolidin-3-yl)methyl)-2-methylpyridin-3-amine (C9) in quantitative yield (710 mg). The free base form of C9 was obtained by placing 600 mg of the TFA salt form on a 6 g Water's Oasis MCX cartridge and eluting with methanol. LRMS (M+): 207.4; tR (LCMS polar): 0.19 min.

Step 3: A solution of m-chloro-perbenzoic acid (m-CPBA) (102 g, 0.457 mM) in DCM (100 mL) at 0° C. was treated over 1 hr. with a solution of 7-azaindole (20 g, 0.1692 mM) in DCM (120 mL). The mixture was allowed to warm to 25° C., stirred for 2 hr., and concentrated. The resultant residue was dissolved in MeOH (200 mL) and saturated aqueous K₂CO₃ (50 mL), mixed for 30 min., and filtered. The filtrate was concentrated, and the resultant residue was purified by silica gel column using 10% MeOH/CHCl₃ as eluting solvent. The pure fractions were combined and concentrated to provide 7-azaindole-7-oxide (C10) as pale brown solid. Yield: 20 g, 85%. (The pure product still contained traces of benzoic acid as an impurity). ¹HNMR (CDCl₃) δ: 8.2-8.3 (d, 1H), 7.6-7.8 (d, 1H), 7.42-7.46 (d, 1H), 7.06-7.14 (t, 1H), and 6.6 (d, 1H). Mass: (M+1) 135.2 calculated for mol. C₇H₈N₂O.

Step 4: C10 (18 g, 136 mm) was slowly added to a POCl₃ solution (90 mL) solution at 0° C. The mixture was then slowly heated to 75-80° C., stirred for 16 hr., and cooled to 25° C. The reaction mixture was treated with petroleum ether (50 mL) and stirred for 15 min. The petroleum ether layer was decanted from the reaction mixture. The reaction mixture was treated again with petroleum ether (50 mL) and stirred as described above. The petroleum ether layer was decanted off and the resultant thick residue was slowly poured into ice. Solid K₂CO₃ was to achieve a pH of 8 the mixture was 8 using solid K₂CO₃. The resultant solids were collected by filtration and dried under reduced pressure to provide 4-chloro-1H-pyrrolo[2,3-b]pyridine (C11) as a pale pink solid. Yield: 12 g, 46%. ¹HNMR (CDCl₃) δ: 10.2 (b, 1H), 8.2 (d, 1H), 7.4 (d, 1H), 7.0-7.09 (d, 1H), 6.6 (d, 1H). Mass: (M+1) 153 calculated for mol. form. C₇H₅ClN₂.

Step 5: A neat sample of C9 (190 mg, 0.92 mmol) and C11 (112 mg, 0.74 mmol) was combined with Hunig's base (0.129 mL, 0.74 mmol). The resultant mixture was heated for 10 hr. at 120° C. on a shaker plate and DMSO (2 mL) was added to dissolve the solids. The mixture was filtered and purified by preparative chromatography using 0.1% trifluoroacetic acid in acetonitrile/water to provide (S)—N-((3-amino-1-(1H-pyrrolo[2,3-b]pyridin-4-yl)pyrrolidin-3-yl)methyl)-2-methylpyridin-3-amine (C12). Yield: 440 mg, 0.66 mmol, 89%. LRMS (M+): 323.5; t_{R} (LCMS polar): 0.15 min.

Step 6: In a manner similar to that described in Eldrup et al., J. Med. Chem. 2004, 47 (21), 5287), a solution of C12 (340 mg, 0.51 mmol) in methanol (2 mL) was treated on a Waters Oasis MCX cartridge (6 g) to provide (165.9 mg, 0.51 mmol) of freebased material. This material was dissolved in DMF (1 mL), and cooled to 0° C., and treated drop-wise with a solution of N-chlorosuccinimide (103 mg; 0.77 mmol) in DMF (0.5 mL). The reaction was slowly warmed slowly to 25°, stirred for 2 hr., treated with aqueous NaHSO₃, and concentrated. The resultant residue was dissolved in DMSO (1 mL) and filtered, and the resultant filtrate was purified by reverse phase HPLC using 0.1% trifluoroacetic acid in water/acetonitrile system. The pure aqueous fractions were dried in a vacuum centrifuge to provide 2. Yield: 12.8 mg, 3.6%.

### Example 3

### Preparation of 3-(2-fluoro-3-(trifluoromethyl)phenylamino)methyl)-1-(5-methyl-7H-pyrrolo[2,3-d]pyrimidin-4-yl)pyrrolidin-3-amine (3)

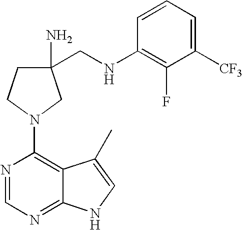

Step 1: A solution of 4-chloro-5-methyl-7H-pyrrolo[2,3-d]pyrimidine (C13) (1.51 g; 9.25 mmol) (see Townsend et al. J. Med. Chem. 1990, 33 (7), 1984) was treated with DIIPEA (5 mL; 27.25 mmol) followed by (3-hydroxymethyl-pyrrolidin-3-yl)-carbamic acid tert-butyl ester (C14) (2.00 g; 9.25 mmol) (see Tomita et al., J. Med. Chem. 2002, 45, 5564). The reaction mixture was stirred at 80° C. overnight. The reaction mixture was then concentrated, treated with ethyl acetate (100 mL), and filtered. The solids were rinsed with ethyl acetate (2×75 mL) and dried to provide tert-butyl [3-(hydroxymethyl)-1-(5-methyl-7H-pyrrolo[2,3-d]pyrimidin-4-yl)pyrrolidin-3-yl]carbamate (C15). Yield: 2.31 g, 72%. LRMS (M+): 348.1; t_{R} (LCMS standard): 1.11 min.

Step 2: A solution of C15 (1.40 g; 4.0 mmol) in DMSO (19 mL) was cooled to 0° C., treated with TEA (1.65 mL; 12.04 mmol), and stirred at 0° C. for 10 min. The reaction mixture was then treated with a solution of a SO₃-Pyridine complex (1.90 g; 12.04 mmol) in DMSO (6 mL). The mixture was stirred at 25° C. for 2 hr., treated with ethyl acetate (300 mL), and washed in this order with brine (75 mL), H₂O (75 mL), 5% aq Na₂HCO₃ (75 mL) and saturated aq. CuSO₄. The organic phase was collected, dried over Na₂SO₄, filtered, and concentrated to provide tert-butyl 3-formyl-1-(5-methyl-7H-pyrrolo[2,3-d]pyrimidin-4-yl)pyrrolidin-3-ylcarbamate (C16). Yield: 1.40 g, 93%. LRMS (M+): 346.1; t_{R} (LCMS standard): 1.09 min.

Step 3: A mixture of 2-fluoro-3-(trifluoromethyl)aniline (228 ul; 1.77 mmol) and 3 Å molecular sieves (0.5 g) was added to a solution of C16 (0.5 g; 1.45 mmol), acetic acid (1 mL; 10% in V) in MeOH (9 mL). The resulting reaction mixture was stirred at 25° C. overnight, treated with MP-Cyanoborohydride (1.45 g, 2.5 mmol/g, 3.65 mmol), and stirred for an additional 5 hr. The mixture was filtered and the solids rinsed with MeOH. The combined organic phases were concentrated to provide tert-butyl 3-((2-fluoro-3-(trifluoromethyl)phenylamino)methyl)-1-(5-methyl-7H-pyrrolo[2,3-d]pyrimidin-4-yl)pyrrolidin-3-ylcarbamate (C17). LRMS (M+): 509.3; t_{R} (LCMS standard): 2.3 min.

Step 4: C17 was treated with DCM (10 mL) and TFA (10 mL), and the resultant reaction mixture was stirred at 25° C. for 3 hr. The mixture was then concentrated, and the resultant residue was purified by chromatography on silica gel (eluting with aq 30-40% NH₄OH/MeOH/DCM) to provide 3. Yield: 320 mg, 69%.

### Example 4

### Preparation of 4-{3-amino-3-[(4-chloro-phenylamino)-methyl]-pyrrolidin-1-yl}-7H-pyrrolo[2,3-d]pyrimidine-5-carbonitrile (4)

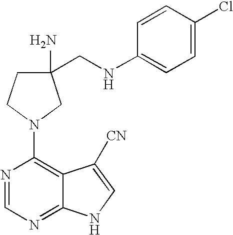

Step 1: A solution of 5-bromo-4-chloro-7H-pyrrolo[2,3-d]pyrimidine (4 g, 17.2 mmol) (see Townsend et al., J. Med. Chem. 1990, 33 (7), 1984) in 170 mL of anhydrous tetrahydrofuran (THF) was cooled to −78° C., and a solution of n-BuLi (15.14 mL, 37.8 mmol, 2.2 eq.) in hexanes was added slowly over 10 min. The reaction mixture was stirred for 1 hr. at −78° C., and resultant yellow suspension was treated drop-wise with dimethylformamide (DMF) (1.465 mL, 18.9 mmol, 1.1 eq) over 10 min. The reaction mixture was stirred at −78° C. for 30 min., warmed to 25° C., and stirred at 25° C. for 1 hr. The reaction mixture was then quenched with 2 mL of water, and the THF was removed under reduced pressure. The resultant slurry was treated with ethyl acetate, water and saturated NH₄Cl. The resultant organic layer was removed, and the aqueous layer was extracted four times with ethyl acetate. After the last extraction, a precipitate formed in the water layer. The precipitate was filtered, washed with water and dried under reduced pressure to provide 4-chloro-7H-pyrrolo[2,3-d]pyrimidine-5-carbaldehyde (C18). Yield: 2.44 g (78%). ¹H NMR (DMSOd₆) δ: 10.23 (s, 1H), 8.75 (s, 1H), 8.61 (s, 1H) ppm.

Step 2: A portion of C18 (1.6755 g, 9.22 mmol) was crushed by mortar and pestle and suspended in EtOH (25 mL). The resultant mixture was treated with solid hydroxylamine hydrochloride (0.7694 g, 11.1 mmol, 1.2 eq) and a solution of aqueous 2M NaOH (5.45 mL, 10.9 mmol, 1.18 eq). The mixture was stirred for 3 hr. at 25° C. and diluted with a sufficient amount of EtOH to allow stirring. The mixture was then heated at 50° C. for 2 hr. and filtered. The solids were washed with water and dried to provide 4-chloro-7H-pyrrolo[2,3-d]pyrimidine-5-carbaldehyde oxime (C19) as a mixture of isomers. Yield: 1.7160 g, 94.6%. ¹H NMR (DMSOd₆) [symbol] : 13.03 and 12.96 (m, 1H), 11.92 and 11.05 (s, 1H), 8.63 and 8.59 (s, 1H), 8.54 and 8.48 (s, 1H), 8.05 and 7.99 (s, 1H) ppm.

Step 3: A suspension of C19 in DCM was treated drop-wise with thionyl chloride (10.38 g, 87 mmol, 10 eq.) and stirred at 25° C. After 5 hr. an additional 2 mL of SOCl₂ was added, and the reaction mixture was stirred overnight at 25° C. The reaction mixture was then heated at 45° C. for 1 hr., cooled to 25° C., and concentrated. The resultant residue was treated with ethyl acetate, water and saturated sodium bicarbonate. The precipitate that formed in the separatory funnel was filtered. The filtrate was then extracted with ethyl acetate, and the combined organics were washed with brine, dried over Na₂SO₄, filtered, and concentrated to provide 0.5 g of 4-chloro-7H-pyrrolo[2,3-d]pyrimidine-5-carbonitrile (C20). The isolated precipitate (1.02 g) was stirred with aqueous ammonium chloride and ethyl acetate, the organic phase was collected, and the aqueous layer was extracted with ethyl acetate. The combined organics were concentrated to provide an additional 0.89 g of C20. Yield (total): 1.39 g, 89.4%). ¹H NMR (DMSOd₆) [symbol] : 13.70 (br s, 1H), 8.78 (s, 1H), 8.70 (s, 1H) ppm.

Step 4: A mixture of C20 (1.739 g, 9.7 mmol), C14 (2.106 g, 9.7 mmol) and DIIPEA (3.56 mL, 20.5 mmol) was heated for five hr. at 80° C. The DMF was removed under reduced pressure, and the resultant residue was treated with water and ethyl acetate. The mixture was then extracted with ethyl acetate (7×25 mL), and the combined organic phases were washed with saturated brine, dried over Na₂SO₄, filtered, and concentrated under reduced pressure. The resultant residue (3.36) was treated with DCM/MeOH, preabsorbed on 6.6 g silica gel, and chromatographed on Biotage Flash 40 L column, eluting with 6% MeOH/0.6% conc. NH₄OH/DCM. The fractions containing product were combined and concentrated to provide [1-(5-cyano-7H-pyrrolo[2,3-d]pyrimidin-4-yl)-3-hydroxymethyl-pyrrolidin-3-yl]-carbamic acid tert-butyl ester (C21) as an off-white solid. Yield: 2.092 g, 60%. TLC (8% MeOH/DCM) R_{f}: 0.29. HPLC t_{R}=4.121 min.

Step 5: A solution of C21 (2.092 g, 5.84 mmol) and TEA (2.44 mL, 17.5 mmol) in DMSO (30 mL) was cooled to 0° C. A slurry of an SO₃-pyridine complex in DMSO (10 mL) was added to the solution. The reaction mixture was allowed to warm to 25° C., and the mixture was stirred for 20 min. at 25° C. The reaction mixture was treated with ethyl acetate (50 mL) and cooled to 0° C. A saturated aqueous solution of copper sulfate was slowly added to the chilled mixture. The resultant slurry was filtered, and the precipitate was washed with ethyl acetate. The combined filtrates were then extracted with ethyl acetate (3×). The combined extracts were washed with brine, dried over Na₂SO₄, filtered, and concentrated under reduced pressure to provide [1-(5-cyano-7H-pyrrolo[2,3-d]pyrimidin-4-yl)-3-formyl-pyrrolidin-3-yl]-carbamic acid tert-butyl ester (C22) as a dark colored solid in quantitative yield. TLC (7.5% MeOH/DCM) R_{f}: 0.35. HPLC t_{R}=4.779 min.

Step 6: [3-[(4-chloro-phenylamino)-methyl]-1-(5-cyano-7H-pyrrolo[2,3-d]pyrimidin-4-yl)-pyrrolidin-3-yl]-carbamic acid tert-butyl ester (C23) was prepared in a manner similar to that described for making C17 in Step 3 of Example 3 by reacting C22 (50 mg, 0.14 mmol) with 4-chloro-phenylamine to provide C23. The compound was used without further purification in Step 7 of this Example.

Step 7: A solution of C23 in DCM (2 mL) was treated with TFA (1 mL), shaken for 4 hr., and concentrated under reduced pressure. The resultant residue was treated with a sufficient amount of DMSO to provide 2 mL of a solution. The DMSO solution was then purified by preparative HPLC (TFA mobile phase) to provide 4 as mono TFA salt. Yield: 45.1 mg, 67%.

### Example 5

### Preparation of 2-{[3-Amino-1-(3-methyl-1H-pyrazolo[3,4-d]pyrimidin-4-yl)-pyrrolidin-3-ylmethyl]-amino}-benzoic acid methyl ester (5)

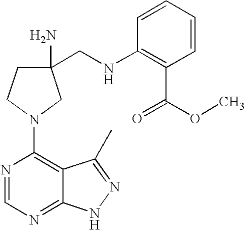

Step 1: A solution of 1-(4,6-dichloropyrimidin-5-yl)ethanone (3.81 g, 19.9 mmol) (see Clark et al., J. C. S. Perkin 1, 1976, 1004) in dioxane (90 mL) was cooled to 0° C., and the chilled solution was treated with TEA (2.78 mL, 19.9 mmol) and hydrazine hydrate (1.16 mL, 23.9 mmol). The reaction mixture was then stirred for 18 hr. at 25° C. The mixture was filtered and the precipitate washed with dioxane. The combined filtrates were concentrated, and the resultant residue was chromatographed on silica gel, eluting with 30-35% ethyl acetate/hexanes to provide 4-chloro-3-methyl-1H-pyrazolo[3,4-d]pyrimidine (C24). Yield: 2.98 g, 89%. ¹H NMR (500 MHz, DMSO-d₆) δ: 8.71 (1H), 2.60 (3H) ppm.

Step 2: A mixture of C24 (0.780 g, 4.62 mmol), C14 (1.003 g, 4.62 mmol) and DIIPEA (2.01 mL, 11.6 mmol) in DMF (9.5 mL) was heated at 70° C. for 2 hr. The mixture was concentrated, and the resultant residue was treated with water and ethyl acetate. The mixture was then extracted with ethyl acetate (6×25 mL), and the combined organic phases were washed with brine, dried over Na₂SO₄, filtered, and concentrated. The resultant residue was triturated with ethyl acetate to provide [3-hydroxymethyl-1-(3-methyl-1H-pyrazolo[3,4-d]pyrimidin-4-yl)-pyrrolidin-3-yl]-carbamic acid tert-butyl ester (C25). Yield: 0.901 g, 56%. TLC (6% MeOH/DCM) R_{f}: 0.13. HPLC t_{R}=3.778 min.

Step 3: A solution of C25 (0.891 g, 2.56 mmol) in DMSO (20 mL) was treated with triethylamine (TEA) followed by treatment with a solution of a SO₃-Pyridine complex. The mixture was treated with dichloromethane (DCM) and washed in this order with brine (100 mL), H₂O (100 mL), 5% aq. Na₂HCO₃ (100 mL) and saturated aq. CuSO₄. The organic phase was collected, dried over Na₂SO₄, filtered, and concentrated to provide 3-formyl-1-(3-methyl-1H-pyrazolo[3,4-d]pyrimidin-4-yl)-pyrrolidin-3-yl]-carbamic acid tert-butyl ester (C26). Yield: 0.664 g, 75%.

Step 4: Compound 5 was prepared in a manner similar to that described in Steps 6 and 7 of Example 4, except that C26 (30 mg, 0.09 mmol) and 4-amino-3-methylbenzoic acid were used instead of C22 and 4-chloro-phenylamine, respectively. Yield: 14.5 mg, 27%.

### Example 6

### Preparation of 1-(5-Chloro-7H-pyrrolo[2,3-d]pyrimidin-4-yl)-3-[(3-phenoxy-phenylamino)-methyl]-pyrrolidin-3-ylamine (6)

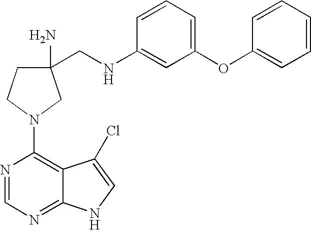

Step 1: A mixture of 4,5-dichloro-7H-pyrrolo[2,3-d]pyrimidine (C16) (1.635 g, 8.71 mmol) (see Townsend, J. Med. Chem. 1988, 31, 2086), C14 (1.883 g, 8.71 mmol), DIIPEA (3.34 mL, 19.2 mmol) and DMF and was heated for seven hr. at 60° C. The mixture was concentrated under reduced pressure, and the resultant residue was treated with water and ethyl acetate. The mixture was extracted with ethyl acetate (7×25 mL), and the combined extracts were washed with brine, dried over Na₂SO₄, filtered, and concentrated under reduced pressure. The resultant reside (2.73 g crude) was treated with DCM/MeOH, pre-absorbed on 5.8 g silica gel and chromatographed with 5% methanol/0.25% conc. NH₄OH_{(aq)}/DCM to provide [1-(5-chloro-7H-pyrrolo[2,3-d]pyrimidin-4-yl)-3-hydroxymethyl-pyrrolidin-3-yl]-carbamic acid tert-butyl ester (C27) as an off-white solid. Yield: 1.755 g, 55%. TLC (7% MeOH/DCM) R_{f}: 0.18. HPLC t_{R}=4.678 min.

Step 2: [1-(5-chloro-7H-pyrrolo[2,3-d]pyrimidin-4-yl)-3-formyl-pyrrolidin-3-yl]-carbamic acid tert-butyl ester (C28) was prepared in a manner similar to that described in Step 3 of Example 5, except that C27 (1.752 g, 4.76 mmol) was used instead of C25. Yield: 2.083 g, 100%. TLC (7% MeOH/DCM) R_{f}: 0.30. HPLC t_{R}=5.379 min.

Step 3. Compound 6 was prepared in a manner similar to that described in Steps 6 and 7 of Example 4, except that C28 (40 mg, 0.11 mmol) and 3-phenoxy-phenylamine were used instead of C22 and 4-chloro-phenylamine, respectively. Yield: 15.9 mg, 34%.

### Example 7

### Preparation of 4-(3-amino-3-((3-chloro-2-fluorophenylamino)methyl)pyrrolidin-1-yl)-1H-pyrazolo[3,4-d]pyrimidine-3-carbonitrile (7)

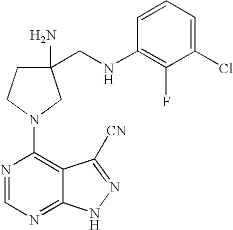

Step 1: 4-Hydroxy-1H-pyrazolo[3,4-d]pyrimidine-3-carbonitrile (3.54 g, 22 mmol) (see Taylor et al., J. Org. Chem. 1965, 31, 342) was added to phosphorus oxychloride (30 mL, 329 mmol) with stirring, and the resultant suspension was added to N,N-dimethylaniline (2.78 mL (22 mmol). The suspension was maintained at 100° C. for 45 min. and at 25° C. for 1 hr. The suspension was concentrated at 60° C. under reduced pressure, and the resultant residue was poured into ice water. The mixture was extracted with 4:1 ethyl acetate/hexanes (1×) and ether (2×). The combined organic extracts were washed with brine, dried over Na₂SO₄, filtered, and concentrated under reduced pressure. The resultant red solid (2.96 g) was treated with ethyl acetate (250 mL), preabsorbed onto 5.4 g of silica gel, and chromatographed on a Biotage Flash 40 L cartridge (120 g silica), eluting with 50% ethyl acetate/hexanes. Fractions containing product were combined and concentrated under reduced pressure to provide 4-chloro-1H-pyrazolo[3,4-d]pyrimidine-3-carbonitrile (C29) as a red solid. Yield: 2.22 g, 56%. ¹H NMR (DMSO-d₆) δ: 8.975 (s, 1H). HPLC 3.892 min.

Step 2: A mixture of C29, (1.385 g, 7.7 mmol), C14 (1.668 g (7.7 mmol) and DIIPEA (3.36 mL, 19.3 mmol) in DMF was heated for 2 hrs at 55° C. The mixture was then concentrated under reduced pressure. The resultant residue was treated with water/ethyl acetate and extracted with ethyl acetate (5×35 mL). The aqueous phase was then treated with brine (20 mL) and extracted with ethyl acetate (2×30 mL). The combined organic extracts were washed with brine, dried over Na₂SO₄, filtered, and concentrated under reduced pressure. The resultant reside (2.37 g) was treated with DCM/MeOH, preabsorbed onto 4.1 g silica gel, and chromatographed on Biotage Flash 40M column, eluting with 6.3% MeOH/DCM. The fractions containing product were combined and concentrated to provide [1-(3-Cyano-1H-pyrazolo[3,4-d]pyrimidin-4-yl)-3-hydroxymethyl-pyrrolidin-3-yl]-carbamic acid tert-butyl ester (C30) as an off-white solid. Yield: 1.308 g, 47%. TLC (7% MeOH/DCM) R_{f}: 0.15. HPLC t_{R}=4.261 min.

Step 4: [1-(3-Cyano-1H-pyrazolo[3,4-d]pyrimidin-4-yl)-3-formyl-pyrrolidin-3-yl]-carbamic acid tert-butyl ester (C31) was prepared in a manner similar to that described in Step 3 of Example 5 except that C30 (1.303 g, 3.63 mmol) was used instead of C25 to provide C31 as a dark foam. Yield: 1.265 g, 98%. TLC (7% MeOH/DCM) R_{f}: 0.27. HPLC t_{R}=4.961 min.

Step 5. Compound 7 was prepared in a manner similar to that described in Steps 6 and 7 of Example 4, except that C31 (8.5 mg, 20%) and 3-chloro-2-fluoroaniline were used instead of C22 and 4-chloro-phenylamine, respectively, and the basic mobile phase was used for purification. Yield: 19.1 mg, 47%.

### Example 8

### Preparation of 4-{3-Amino-3-[(3-chloro-phenylamino)-methyl]-pyrrolidin-1-yl}-1H-pyrazolo[3,4-d]pyrimidine-3-carbonitrile (8)

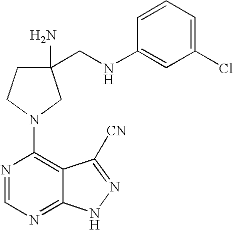

Compound 8 was prepared in a manner similar to that described for compound 7 in Example 7 except that 3-chloroaniline was used instead of 3-chloro-2-fluoroaniline in Step 5. Yield: 19.1 mg, 47%.

### Example 9

### Preparation of 1-(5-ethyl-7H-pyrrolo[2,3-d]pyrimidin-4-yl-3-({[3-(trifluoromethyl)phenyl]amino}-methyl)pyrrolidin-3-amine (9)

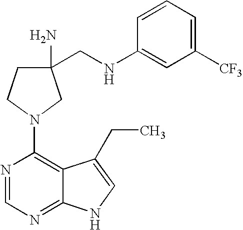

Step 1: A solution of C7 (0.161 g, 0.886 mmol), C14 (0.192 g, 0.886 mmol) and DIIPEA (0.324 mL, 1.862 mmol) in isopropyl alcohol was heated for 16 hr. at 80° C. The mixture was then concentrated under reduced pressure. The resultant residue was treated with water and ethyl acetate, and the mixture was extracted with ethyl acetate (3×25 mL). The combined organic extracts were dried over Na₂SO₄, filtered, and concentrated under reduced pressure. The resultant reside (0.30 g) was treated with DCM/MeOH, preabsorbed onto 4.5 g silica gel, and chromatographed on Biotage Flash 40S column, eluting with 9% MeOH/DCM. The fractions containing product were combined and concentrated under reduced pressure to provide tert-butyl 1-(5-ethyl-7H-pyrrolo[2,3-d]pyrimidin-4-yl-3-(hydroxymethyl)pyrrolidin-3-ylcarbamate (C32) as a white solid. Yield: 0.180 g, 56%. TLC (9% MeOH/DCM) R_{f}: 0.29. HPLC t_{R}=4.642 min. MS ES+: 362.3, t_{R} (LC-MS STD)=1.2 min.

Step 2: A solution of C32 in DMSO (2.2 mL) was treated with TEA (0.184 mL, 1.32 mmol) and cooled to 0° C. The chilled solution was treated with a slurry of a SO₃-pyridine complex (0.21 g, 1.32 mmol) in DMSO (0.66 mL), and the reaction mixture was allowed to warm to 25° C. After 1 hr. the reaction mixture was treated with ethyl acetate (10 mL), cooled to 0° C., and treated with a saturated solution of copper sulftate (20 mL). The resultant slurry was filtered, and the precipitate was washed with ethyl acetate. The combined filtrates were then extracted with ethyl acetate (3×). The combined extracts were washed with brine, dried over Na₂SO₄, filtered, and concentrated under reduced pressure to provide tert-butyl 1-(5-ethyl-7H-pyrrolo[2,3-d]pyrimidin-4-yl-3-formylpyrrolidin-3-ylcarbamate (C33) as a dark colored solid. Yield: 0.13 g, 82%. TLC (9% MeOH/DCM) R_{f}: 0.38. HPLC t_{R}=5.496 min. MS ES+: 360.3, t_{R}(LC-MS STD)=1.4 min.

Step 3: tert-butyl 1-(5-ethyl-7H-pyrrolo[2,3-d]pyrimidin-4-yl)-3-((3-(trifluoromethyl)-phenylamino)-methyl)pyrrolidin-3-ylcarbamate (C34) was prepared in a manner similar to that described in Step 3 of Example 3, except that C33 (45 mg, 0.126 mmol) and 3-trifluoromethylaniline were used instead of C16 and 2-fluoro-3-(trifluoromethyl)aniline, respectively.

Step 4: A solution of C34 and TFA (1 mL) in DCM (1 mL) was shaken for 4 hr. and concentrated under reduced pressure. The resultant residue was dissolved in a sufficient amount of DMSO to provide 2 mL of solution and purified by prep HPLC (TFA mobile phase) to provide 9. Yield: 11.8 mg, 23%.

### Example 10

### Preparation of 3-{[(2,3-dichlorophenyl)amino]methyl}-1-(5-methylpyrrolo[2,1-f][1,2,4]triazin-4-yl)pyrrolidin-3-amine (10)

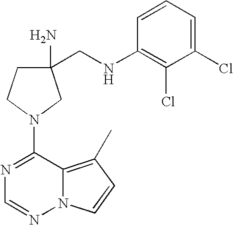

Step 1: A mixture of 4-Chloro-5-methylpyrrolo[2,1-f][1,2,4]triazine (0.150 g, 0.895 mmol) (see WO 2003042172), C14 (0.194 g, 0.895 mmol), and DIEA (0.327 mL, 1.879 mmol) in DMF (1.79 mL) was heated at 80° C. for 3 hr. The mixture was then concentrated under reduced pressure, and the resultant residue was treated with water and ethyl acetate. The resultant biphasic mixture was extracted with ethyl acetate (3×), and the combined organics were washed with brine, dried over Na₂SO₄, filtered, concentrated under reduced pressure. The resultant residue (0.31 g) was treated with ethyl acetate, preabsorbed onto 4.5 g silica gel, and chromatographed on Biotage Flash 40S column, eluting with 50% ethyl acetate/hexane. The fractions containing product were combined and concentrated to provide tert-butyl 3-(hydroxymethyl)-1-(5-methylpyrrolo[2,1-f][1,2,4]triazin-4-yl)pyrrolidin-3-ylcarbamate (C35) as a white solid. Yield: 0.234 g, 75%. TLC (50% EtOAc/Hexane) R_{f}: 0.22. HPLC t_{R}=5.207 min. MS ES+: 348.3, t_{R}(LC-MS STD)=1.7 min

Step 2: A solution of C35 (1.387 g, 3.993 mmol) in DMSO (20 mL) was treated with TEA (2.226 mL, 16.0 mmol), cooled to 0° C., and treated with a slurry of a SO₃-pyridine complex (2.542 g, 16.0 mmole) in DMSO (8 mL). The mixture was allowed to warm to 25° C. and stirred for an additional 1 hr. The mixture was then treated with 50 mL ethyl acetate, cooled to 0° C., and slowly treated with a saturated solution of copper sulftate (200 mL). The resultant slurry was filtered and the precipitate was washed with ethyl acetate. The combined filtrates were extracted with ethyl acetate (3×), and the combined extracts were washed with brine, dried over Na₂SO₄, filtered, and concentrated under reduced pressure to provide tert-butyl 3-formyl-1-(5-methylpyrrolo[2,1-f][1,2,4]triazin-4-yl)pyrrolidin-3-ylcarbamate (C36) as a solid. Yield: 1.5 g, 100%. TLC (9% MeOH/DCM) R_{f}: 0.47. MS ES+: 346.3, t_{R}(LC-MS STD)=1.9 min

Step 3: tert-butyl 3-{[(2,3-dichlorophenyl)amino]methyl}-1-(5-methylpyrrolo[2,1-f][1,2,4]triazin-4-yl)pyrrolidin-3-ylcarbamate (C37) was prepared in a manner similar to that described in Step 3 of Example 3, except that C36 (45 mg, 0.126 mmol) and 2,3-dichlorophenylamine were used instead of C16 and 2-fluoro-3-(trifluoromethyl)aniline, respectively.

Step 4: A solution of C37 and TFA (1 mL) in DCM (1 mL) shaken for 4 hr. and concentrated under reduced pressure. The resultant mixture was dissolved in a sufficient amount of DMSO to provide 2 mL of solution and purified by preparative HPLC (TFA mobile phase) to provide 10. Yield: 5.0 mg, 10%.

### Example 11

### Preparation of 4-(3-amino-3-{[(2-phenoxyphenyl)amino]methyl}pyrrolidin-1-yl)-1H-pyrrolo[2,3-b]pyridine-3-carbonitrile (11)

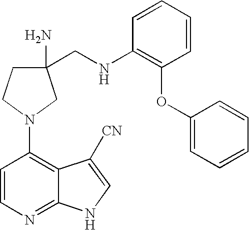

Step 1: A solution of C11 (9 g, 59.016 mM) in acetone (60 mL) was slowly added to a solution of N—N-bromosuccinimide (NBS) (10.44 g, 59.01 mM) in acetone (100 mL) at 25° C., and the reaction mixture was stirred for 1 hr. at 25° C. The solids were collected by filtration, washed with chilled acetone (50 mL), and dried under reduced pressure to provide 3-bromo-4-chloro-1H-pyrrolo[2,3-b]pyridine (C38) as pale yellow solid. Yield: 7.5 g, 58%. ¹HNMR (CDCl₃) δ: 11.6-11.7 (b, 1H), 8.1-8.2 (d, 1H), 7.2-7.4 (s, 1H), 7.0-7.01 (d, 1H). Mass: (M+1) 231 calculated for mol. form. C₇H₄BrClN₂.

Step 2: A solution of C38 (8 g, 34.632 mM) in THF (160 mL) was cooled to −78° C., treated with n-BuLi (1.6M, 50 mL, 79.63 mM), and stirred for 30 min. at −78° C. The cold solution was then slowly treated with DMF (5.056 g, 69.264 mM). The reaction mixture was allowed to warm to 25° C., stirred for 2 hr., and treated with water (2 mL) to quench the reaction. The reaction mixture was concentrated under reduced pressure and treated with saturated aq. NH₄Cl (28 mL), and the solids were collected and dried under reduced pressure. The resultant pale yellow solid (3.5 g, 52%) was then purified using silica gel column to provide 4-chloro-1H-pyrrolo[2,3-b]pyridine-3-carbaldehyde (C39). ¹HNMR (CDCl₃) δ: 12.6-12.8 (b, 1H), 10.4-10.6 (s, 1H), 8.26-8.3 (d, 1H), 8.12-8.18 (s, 1H), 7.26-7.3 (d, 1H). Mass: (M+1) 181 calculated for mol. form. C₈H₅ClN₂O.

Step 3: A solution of C39 (3.5 g, 19.4 mM) in EtOH (35 mL) was treated at 25° C. with hydroxylamine-HCl (2.70 g, 38.88 mM) followed by aqueous NaOH (1.55 g, 38.88 mM) and stirred for 1 hr. The reaction mixture was concentrated under reduced pressure, treated with water (50 mL), and stirred for 10 min. The resultant solids were collected by filtration and dried under reduced pressure for 2 hr. to provide (E)-4-chloro-1H-pyrrolo[2,3-b]pyridine-3-carbaldehyde oxime (C40) as pale yellow solid. Yield: 3.5 g, 85%. ¹HNMR (CDCl₃) (Major isomer) δ: 11.6-11.8 (b, 1H), 10.3-10.4 (s, 1H), 8.7-8.8 (s, 1H), 8.1-8.2 (d, 1H), 7.8 (s, 1H), 7.0-7.2 (d, 1H). Mass: (M+1) 196.2 calculated for mol. form. C₈H₆ClN₃O.

Step 4: A suspension of C40 (3.2 g, 16.4 mM) in DCM (50 mL) was treated with SOCl₂ (1.95 g, 16.4 mM) at 25° C., heated to reflux, and stirred for 4 hr. The reaction mixture was cooled to 25° C. and filtered. The solids was then suspended in saturated aq. NaHCO₃ (10 mL) and stirred for 15 min. The solids were collected by filtration, washed with water (50 mL), dried under vacuum for 2 hr. The resultant residue was suspended in acetonitrile (20 mL), and the mixture was refluxed for 1 hr., cooled to 25° C., filtered and dried under reduced pressure to provide 4-chloro-3-cyano-1H-Pyrrolo[2,3-b]pyridine (C41) as a pale brown solid. Yield: 2 g, 70%. IR (KBR): 3136, 2857, 2228, 1609, 1573, 1511, 1455, 1398, 1336, and 1312. ¹HNMR (CDCl₃) δ: 13.1-13.3 (b, 1H), 8.6 (s, 1H), 8.3-8.4 (d, 1H), 7.4-7.5 (d, 1H). Mass (M+H) 178.2 calculated for C₈H₄ClN₃.

Step 5: A mixture of C41 (2.039 g, 11.48 mmol), C14 (2.483 g (11.48 mmol) and DIIPEA (4.2 mL, 24.10 mmol) was heated for 15 hr. at 120° C. The mixture was allowed to cool to 25° C. and treated with water and ethyl acetate. The resultant biphasic mixture was then extracted with ethyl acetate (3×250 mL), and the combined organic extracts were dried over Na₂SO₄, filtered, and concentrated under reduced pressure. The resultant residue (2.0 g) was treated with DCM/methanol, preabsorbed onto 5.0 g silica gel, and chromatographed on Biotage Flash 40M column, eluting with 9% methanol/DCM. The fractions containing product were combined and concentrated to provide tert-butyl 1-(3-cyano-1H-pyrrolo[2,3-b]pyridin-4-yl)-3-(hydroxymethyl)pyrrolidin-3-ylcarbamate (C42) as a light brown solid. Yield: 1.20 g, 30% yield. TLC (7% MeOH/DCM) R_{f}: 0.28. HPLC t_{R}=4.182 min. MS ES+: 358.3 t_{R} (LC-MS STD)=1.1 min.

Step 6: A solution of C42 (1.2 g, 3.36 mmol) in DMSO (17 mL) was treated with TEA (1.883 mL, 13.5 mmol) and cooled to 0° C. The chilled solution was treated with a slurry of a SO₃-pyridine complex (2.150 g, 13.5 mmol) in DMSO (6.77 mL). After addition, the reaction mixture was allowed to warm to 25° C. and stirred for 1 hr. The reaction mixture was then treated with ethyl acetate (50 mL), cooled to 0° C., and slowly treated with a saturated solution of copper sulfate (200 mL). The resultant slurry was filtered and the precipitate washed with ethyl acetate. The combined filtrates were extracted with ethyl acetate (3×), and the combined organic extracts were washed with brine, dried over Na₂SO₄, filtered, and concentrated under reduced pressure to provide tert-butyl 1-(3-cyano-1H-pyrrolo[2,3-b]pyridin-4-yl)-3-formylpyrrolidin-3-ylcarbamate (C43) as a gum. Yield: 1.0 g, 84%. TLC (9% MeOH/DCM) R_{f}: 0.27. MS ES−: 345.2, t_{R} (LC-MS STD)=1.3 min.

Step 7: tert-butyl 1-(3-cyano-1H-pyrrolo[2,3-b]pyridin-4-yl)-3-((2-phenoxyphenylamino)-methyl) pyrrolidin-3-ylcarbamate (C44) was prepared in a manner similar to that described in Step 3 of Example 3, except that C43 (45 mg, 0.126 mmol) and (2-phenoxyphenyl)amine were used instead of C16 and 2-fluoro-3-(trifluoromethyl)aniline, respectively. The product was used in Step 10 without further purification.

Step 8: A solution of C44 and TFA (1 mL) in DCM (1 mL) was shaken for 4 hr. and concentrated under reduced pressure. The resultant residue was dissolved in a sufficient amount of DMSO to provide 2 mL of solution and purified by preparative HPLC (TFA mobile phase) to provide 11. Yield: 4.0 mg, 7%.

### Examples 12-59

Examples 12 to 59 (Table 3) were prepared according to the procedures described in Examples 1 to 11 above.

Table 3 also contains the Akt kinase activity for compounds I-59.

### Example 60

### Preparation of 1-(5-Methyl-7H-pyrrolo[2,3-d]pyrimidin-4-yl)-3-(3-trifluoromethyl-phenoxymethyl) pyrrolidin-3-ylamine (60)

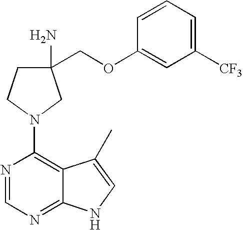

Step 1: A solution of 3-trifluoromethyl-phenol (2.1 mL, 17.3 mmol) in dry acetone (250 mL) was treated with ethyl bromomethyl acrylate (5.0 g, 25.9 mmol) and anhydrous potassium carbonate (8.36 g, 60.5 mmol). The mixture was then heated at reflux for 1.5 hr. The reaction mixture was cooled and filtered, and the solids were washed with DCM. The filtrate was evaporated and the crude material was purified by column chromatography, eluting with 7% ethyl acetate/hexanes. The resulting oil was re-dissolved in ethyl acetate and TEA (5 mL) was added. The solution was stirred for 10 min. and the resulting precipitate was filtered through Celite. The filtrate was washed with 1N HCl and the organic layer was washed with brine, dried over MgSO₄, filtered, and concentrated to provide 2-(3-Trifluoromethyl-phenoxy)-acrylic acid ethyl ester (C45) as a colorless oil. Yield: 4.56 g, 96% yield. GC/MS: ret. time: 2.54; mass 274.

Step 2: A solution of C45 (3.0 g, 10.9 mmol)) and N-Benzyl-N-(methoxymethyl)-N-((trimethylsilane)methyl)amine (5.58 mL, 21.8 mmol) in DCM (60 mL) was cooled to 0° C. followed by drop-wise addition of TFA (168 μL). The reaction mixture was stirred at 0° C. for 30 min., warmed to 25° C., stirred for an additional 2 hr., and concentrated under reduced pressure. The resultant crude residue was chromatographed on a silica gel column, eluting with 25% ethyl acetate/hexanes with several drops of TEA to basify the column to provide 1-benzyl-3-(3-trifluoromethyl-phenoxymethyl)-pyrrolidine-3-carboxylic acid ethyl ester (C46) as a colorless oil. Yield: 5.40 g. GC/MS: ret. time −5.47 min.; mass 407. The product was used directly in the next reaction step without further purification.

Step 3. A solution of C46 (3.70 g, 9.08 mmol) and ammonium formate (2.86 g, 45.4 mmol) in ethanol (40 mL) was carefully treated with 10% palladium on carbon (2.89 g) under a stream of nitrogen. The reaction mixture was heated to 80° C., mixed for 3 hr., cooled to 25° C., and filtered through Celite. The filtrate was then concentrated to provide 3-(3-trifluoromethyl-phenoxymethyl) -pyrrolidine-3-carboxylic acid ethyl ester (C47) as a clear oil. Yield: 2.29 g, 80% yield over two steps. LCMS: 318.4H⁺. ¹H NMR (400 MHz, DMSO-d₆) δ: 7.47 (t, 1H), 7.23 (m, 3H), 4.10 (m, 4H), 2.94 (d, 1H), 2.84 (m, 2H), 2.74 (m, 1H), 2.06 (m, 1H), 1.73 (m, 1H), 1.09 (t, 3H).

Step 4: A mixture of C13 (0.352 g, 2.1 mmol), C47 (0.80 g, 2.52 mmol), and DIIPEA (0.801 mL, 4.6 mmol) in DMF (3 mL) was heated at 80° C. overnight followed by drop-wise addition to stirred water. The reaction mixture was then extracted with ethyl acetate (3×), and the combined organic layers were washed with brine, dried over MgSO₄, filtered, and concentrated to provide 1-(5-Methyl-7H-pyrrolo[2,3-d]pyrimidin-4-yl)-3-(3-trifluoromethyl-phenoxymethyl)-pyrrolidine-3-carboxylic acid ethyl ester (C48) as a light yellow solid. Yield: 1.05 g. LCMS: 449.3H⁺. ¹H NMR (400 MHz, DMSO-d₆) δ: 11.19 (s, 1H), 8.06 (s, 1H), 7.49 (t, 1H), 7.24 (m, 3H), 6.97 (s, 1H), 4.35 (q, 2H), 4.10 (m, 3H), 3.80 (m, 3H), 2.33 (m, 5H), 1.07 (t, 3H).

Step 5: A solution of C48 (1.05 g) in THF (15 mL) was treated with LiOH (3.2 mL of 2M solution, 3 eq) at 25° C., and the reaction mixture was stirred at 25° C. overnight. The THF was removed by evaporation, and the resultant aqueous residue was washed with ether followed by treatment with 0.5M HCl and 0.1N HCl until a ˜6 pH was achieved. A precipitate formed which was collected by filtration to provide 1-(5-Methyl-7H-pyrrolo[2,3-d]pyrimidin-4-yl) -3-(3-trifluoromethyl-phenoxymethyl)-pyrrolidine-3-carboxylic acid (C49) as a white solid. Yield: 707 mg (80% over two steps). ¹H NMR (400 MHz, DMSO-d₆) δ: 12.9 (br, 1H), 11.39 (s, 1H), 8.05 (s, 1H), 7.48 (t, 1H), 7.25 (m, 3H), 7.96 (s, 1H), 4.31 (dd, 2H), 4.10 (d, 1H), 3.72 (m, 3H), 2.2 (m, 5H).

Step 6: A mixture of C49 (300 mg) and DCM (4 mL) was treated with thionyl chloride (216 μL, 2.96 mmol) and one drop of DMF and heated at reflux overnight. The mixture was concentrated under reduced pressure, and the resultant residue was dissolved in acetone (3 mL), cooled to 0° C., and treated with sodium azide (226 mg, 3.48 mmol) in 1 mL of H₂O. The temperature was monitored as the sodium azide solution was added so it did not exceed 15° C. The reaction mixture was then stirred at 25° C. for 3 hr. The reaction mixture was added to water (10 mL) and stirred. The solids were collected by filtration, dissolved into acetic acid (8 mL) and water (0.8 mL), and refluxed for 5 hr. The mixture was then concentrated under reduced pressure and the resultant solids treated with ethyl acetate (2×). The combined organic extracts were concentrated. The resultant dark red crude residue was purified by column chromatography eluting with 4% MeOH/0.4% NH₄OH in CHCl₃ to provide 60 as a white solid. Yield: 85 mg, 30%.

### Examples 61 to 65

Examples 61 to 65 (Table 4) were prepared according to the procedures described in Example 60 above.

Table 4 also contains the Akt kinase activity for compounds 60-65.

### Example 66

### 1-(5-methyl-7H-pyrrolo[2,3-d]pyrimidin-4-yl)-3-((3-(trifluoromethyl)phenylthio)methyl) -pyrrolidin-3-amine (66)

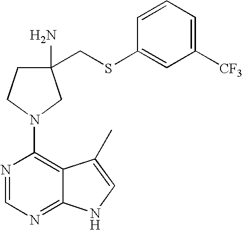

Step 1: In a similar manner as described in J. Org. Chem. 67(15); 2002; 5164-5169, a solution of thionyl chloride (2.14 mL, 29.3 mmol) in dry acetonitrile (15 mL) under N₂ atmosphere was cooled to −40° C. The chilled solution was then treated drop-wise with a solution of C15 (3.70 g, 11.7 mmol) in acetonitrile (20 mL) and stirred for 5 min. Pyridine (4.73 mL, 58.5 mmol) was then added, and the reaction mixture was stirred for 2 hr. while slowly warming to 25° C. The solvent volume was reduced by half, ethyl acetate was added, and the resulting mixture was filtered. The filtrate was concentrated, and the resultant residue was chromatographed on silica gel eluting with 25% EtOAc/Hexane with 2 mL of TEA. The fractions containing the product were combined and concentrated to provide pyrrolidino-spiro-sulfinyl-oxylactam (C50) as a thick yellow oil. Yield: 3.33 g, 79%. TLC Rf=0.6 in 50% EtOAc/Hex. ¹H NMR (400 MHz, DMSO-d₆) δ: 4.85 (m, 2H), 3.83 (d, 0.5H), 3.55-3.19 (m, 3.5H), 2.71 (m, 0.5H), 2.25 (m, 0.5H), 1.95 (m, 1H), 1.43 (m, 18H).

Step 2: A solution of C50 (5.5 g, 15.2 mmol) in acetonitrile (40 mL) was cooled to 0° C. and treated in this order with ruthenium(III) chloride (˜5 mg), sodium periodate (3.25 g, 15.2 mmol), and H₂O (25 mL). The reaction mixture was then stirred for 3 days at 25° C. The mixture was treated with equal volumes H₂O and ether, the organic layer was collected, and the aqueous layer was extracted with ether (3×). The combined organic layers were dried over MgSO₄, filtered, and concentrated to provide pyrrolidino-spiro-sulfonyl-oxylactam (C51) as a light yellow syrup. Yield: 5.35 g, 93%. ¹H NMR (400 MHz, DMSO-d₆) δ: 4.73 (m, 2H), 3.66 (m, 1H), 3.49 (m, 2H), 3.34 (m, 1H), 2.46 (m, 1H), 2.30 (m, 1H), 1.44 (s, 9H), 1.37 (s, 9H).

Step 3: A well-stirred solution of C51 (1.5 g, 3.96 mmol) and m-trifluoromethyl thiophenol (1.41 g, 7.92 mmol) in DMF (20 mL) was treated with cesium carbonate (2.58 g, 7.92 mmol) and stirred at 25° C. for 18 hr. The DMF was removed by evaporation and the remaining residue was treated with DCM and H₂O. The water layer was acidified to pH 5 with 0.5M HCl, and the biphasic mixture was vigorously stirred at 25° C. for 18 hr. The water layer was then neutralized with several drops of saturated NaHCO₃. The layers were separated, and the aqueous layer was extracted with DCM (3×). The combined organic layers were washed with brine, dried over MgSO₄, filtered, and concentrated. The resultant residue was purified by column chromatography over silica eluting with 20% EtOAc/Hexane with 1-2 mL of TEA to basify the column. The fractions containing product were combined and concentrated to provide tert-butyl 3-(tert-butoxycarbonyl)-3-((3-(trifluoromethyl)phenylthio)methyl) pyrrolidine-1-carboxylate (C52) as a yellow oil. Yield: 1.85 g, 97%. HPLC: 8.802. ¹H NMR (400 MHz, DMSO-d₆) δ: 7.64 (s, 2H), 7.50 (m, 2H), 7.20 (s, 1H), 3.67-3.53 (m, 2H), 3.45 (d, 1H), 3.25-3.13 (m, 3H), 2.12 (m, 1H), 1.87-1.84 (m, 1H), 1.3 (m, 18H).

Step 4: A solution of C52 (1.6 g) in DCM (4 mL) was cooled to 0° C., treated with TFA (6 mL), warmed to 25° C. over 1.5 hr., and concentrated under reduced pressure. The resultant residue was azotroped with ethyl acetate (2×), and the combined organic extracts were concentrated to provide the bis trifluoro acetate salt of 3-((3-(trifluoromethyl)phenylthio)methyl)pyrrolidin-3-amine (C53) as a dark-colored syrup. Yield: 1.82 g, 88%. yield. LC/MS: ret. time −0.3; 277.1 (+H). ¹H NMR (400 MHz, DMSO (˜0.1 mL D₂O added)) δ: 7.7 (m, 2H), 7.58 (m, 2H), 3.49 (s, 2H), 3.35 (m, 4H), 2.14 (m, 2H).

Step 5. A solution of C13 (168 mg, 1.00 mmol), C53 (680 mg, 1.1 mmol), and DIIPEA (766 μL, 4.4 mmol) in DMF (1.5 mL) was heated at 80° C. for 18 hr. The solution was cooled to 25° C., and it was added drop-wise to water. The reaction mixture was extracted with ethyl acetate (3×), and the combined organic layers were dried over MgSO₄, filtered, and concentrated. The resultant residue was purified by column chromatography over silica eluting with 5% MeOH/CHCl₃. The fractions containing product were combined and concentrated to provide 66 as a tan solid. Yield: 150 mg, 37%.

### Example 67

### Preparation of 1-(5-methyl-7H-pyrrolo[2,3-d]pyrimidin-4-yl)-3-{2-[3-(trifluoromethyl) -phenyl]ethyl}pyrrolidin-3-amine (67)

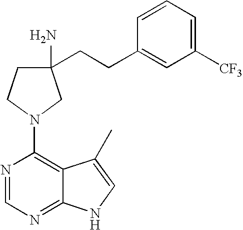

Step 1: Sodium hydride (38 mg, 0.995 mmol) and dry DMSO (1.50 mL) were charged to a round-bottom flash with nitrogen purging, and the contents of the flask were heated at 75° C. under nitrogen atmosphere for 45 min. The contents of the flask were cooled to 0° C., and triphenyl-(3-trifluoromethyl-benzyl)-phosphanyl bromide (500 mg, 0.995 mmol) (JACS, 1986 (108), 7664) was added at such a rate to ensure that no freezing occurred. The resultant orange-red solution was stirred under nitrogen atmosphere for 1 hr., treated with C16 (23 mg, 0.646 mmol), stirred at 25° C. for 1 hr., cooled to 0° C., and treated with H₂O (1.00 mL). Diethyl ether was added, and the phases were separated. The organic phase was washed with 1.0 M sodium hydroxide, dried over MgSO₄, filtered, and concentrated under reduced pressure. The resultant oil was purified over silica (40% ethyl acetate in hexanes) to provide the Boc-protected analog of 1-(5-Methyl-7H-pyrrolo[2,3-d]pyrimidin-4-yl)-3-[2-(3-trifluoromethyl-phenyl)-vinyl]-pyrrolidin-3-ylamine. The compound was treated with a solution of TFA/DCM at 0° C., and the reaction mixture was concentrated under reduced pressure. The resultant residue was triturated with toluene (3×) to provide the trifluoroacetic acid salt of 1-(5-Methyl-7H-pyrrolo[2,3-d]pyrimidin-4-yl)-3-[2-(3-trifluoromethyl-phenyl)-vinyl]-pyrrolidin-3-ylamine. The salt was extracted with ethyl acetate, and the combined extracts were washed with saturated sodium bicarbonate, dried over anhydrous MgSO₄, filtered, and concentrated to provide 1-(5-Methyl-7H-pyrrolo[2,3-d]pyrimidin-4-yl)-3-[2-(3-trifluoromethyl-phenyl)-vinyl]-pyrrolidin-3-ylamine (C54) as a ˜1:1 mixture of cis- and trans-isomers. Yield: 110 mg. The compound was used in Step 2 without further purification.

Step 2: A solution of C54 in ethanol (50.0 mL) in a Parr reactor was carefully treated with palladium on carbon (110 mg, 0.103 mmol. The reactor was then pressurized with 43 psi H₂. After 2 hr. the reactor was vented, and the reaction mixture was carefully filtered through diatomaceous earth. The filtrate was concentrated, and the resultant black solid was purified over silica (95:5:0.5 CHCl₃:CH₃OH:NH₄OH) and concentrated to provide 67 as a white foam. Yield: 13 mg, 0.0334 mmol, 12% yield.

### Example 68

### Preparation of (E)-3-(3-trifluoromethyl)styryl)-1-(5-methyl-7H-pyrrolo[2,3-d]pyrimidin-4-yl)-3-amine (68)

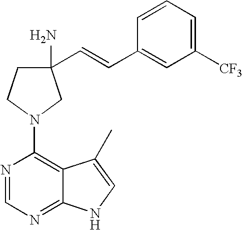

A solution of the E isomer of the Boc-protected analog of C54 (see Step 1 of Example 67) (10.5 mg, 0.025 mmol) in 400 μL of DCM was cooled to 0° C. The solution was treated with TFA (600 μL) and stirred for 2 hr. while warming to 25° C. The mixture was then concentrated under reduced pressure, and the resultant residue was azotroped with ethyl acetate (2×). The product was precipitated with ethyl acetate and hexanes to provide the TFA salt of 68 as a white solid. Yield: 8.4 mg, 78%.

### Example 69

### Preparation of N-((3-amino-1-(5-methyl-7H-pyrrolo[2,3-d]pyrimidin-4-yl)pyrrolidin-3-yl)methyl)benzamide (69)

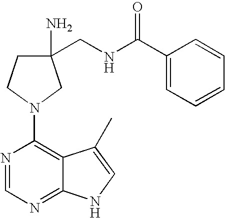

Step 1: A solution of C13 (9.87 g; 57.5 mmol) in 2-propanol (100 mL) was treated with DIIPEA (16 mL; 86.25 mmol) followed by 3-pyrrolidinol (5.26 g; 60.38 mmol). The reaction mixture was stirred at 80° C. overnight. The reaction mixture was concentrated and the resultant slurry mixture was treated with ethyl acetate (40 mL). The resultant precipitate was collected by filtration, rinsed with ethyl acetate (2×75 mL) and dried to provide 1-(5-methyl-7H-pyrrolo[2,3-d]pyrimidin-4-yl)pyrrolidin-3-ol (C55). Yield: 10.58 g, 84%. LRMS (M+): 219.1; t_{R} (LCMS polar): 0.5 min.

Step 2: A solution of C55 (10.58 g; 48.5 mmol) in DMSO (22.2 mL) was cooled to 0° C. and treated with TEA (22.2 mL; 124.16 mmol). After stirring at 0° C. for 10 min., the reaction mixture was treated with a SO₃-Pyridine complex (10.62 g; 67.9 mmol) and stirred at 25° C. overnight. The mixture was treated with chloroform (40 mL), and the resultant precipitate was collected by filtration and rinsed with chloroform to provide 1-(5-methyl-7H-pyrrolo[2,3-d]pyrimidin-4-yl)pyrrolidin-3-one (C56) Yield: (7.0 g, 67%). LRMS (M+): 217.1; t_{R} (LCMS standard): 0.96 min.

Step 3: In a manner similar to that described in Synthetic Communications 14 (14), 1299-1304 (1984), a mixture of C56 (5.68 g; 26.25 mmol), trimethylsilyl cyanide (1.65 mL; 30.78 mmol) and ZnCl₂ (473 mg; 3.47 mmol) was stirred at 25° c. for 15 min. The mixture was then treated with a solution of 2,4-dimethoxybenzylamine (4.42 mL; 28.89 mmol) in MeOH (130 mL) and stirred at 80° C. for 1 day. The resultant precipitate was collected by filtration, rinsed with ethyl acetate (2×100 mL), and dried to provide 3-(2,4-dimethoxybenzylamino)-1-(5-methyl-7H-pyrrolo[2,3-d]pyrimidin-4-yl)pyrrolidine-3-carbonitrile (C57). Yield: 7.5 g, 73%. LRMS (M+): 393.3; t_{R} (LCMS standard): 1.74 min.

Step 4: Lithium aluminum hydride (1.0 M solution in THF) (9.0 mL; 9.0 mmol) was added to a solution of C57 (3.0 g; 7.64 mmol) in DCM (40 mL), and the resultant mixture was stirred at 25° C. overnight. The mixture was then was treated with 1 N aq NaOH (50 mL), and the resultant organic phase was collected. The aqueous phase was extracted with ethyl acetate (3×150 mL), and the combined organic phases were washed with brine (75 mL), dried over Na₂SO₄, filtered, and concentrated to provide N-(2,4-dimethoxybenzyl)-3-(aminomethyl)-1-(5-methyl-7H-pyrrolo[2,3-d]pyrimidin-4-yl)pyrrolidin-3-amine (C58). Yield: 3.0 g, 99%. LRMS (M+): 397.3; t_{R} (LCMS standard): 0.36 min.

Step 5: A solution of C58 (50 mg; 0.126 mmol) in DMF (1.2 mL) was treated with 1-hydroxybenzotriazole (HOBt) (26 mg; 0.189 mmol), benzoic acid (16 mg; 0.126 mmol) and PS-carbodiimide (160 mg; 0.252 mmol). The resultant reaction mixture was stirred at 25° for 6 hr., treated with MP-carbonate (160 mg; 0.504 mmol), and stirred overnight. The mixture was filtered and the solids rinsed with MeOH. The combined filtrates were evaporated, and the resultant residue was treated with TFA (0.5 mL) and heated at 80° C. for 3 hr. TFA was evaporated from the reaction mixture, and the resultant residue was purified by preparative HPLC (TFA/acetonitrile/water mobile) to provide 69. Yield: 11.6 mg, 26%.

### Example 70

### Preparation of N—(((S)-3-amino-1-(5-chloro-7H-pyrrolo[2,3-d]pyrimidine chlorobenzamide (70)-4-yl)pyrrolidin-3-yl)methyl)-4

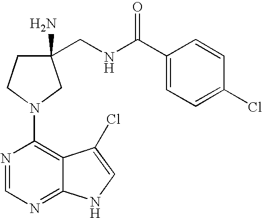

Step 1: A solution of C13 (1.51 g; 9.25 mmol) in 2-propanol (16 mL) was treated with DIIPEA (5 mL; 27.25 mmol) followed by C14 (2.00 g; 9.25 mmol). The reaction mixture was then stirred at 80° C. overnight and concentrated under reduced pressure. The resultant slurry was treated with ethyl acetate (100 mL), and the resultant precipitate was collected by filtration and rinsed with ethyl acetate (2×75 mL) to provide tert-butyl [3-(hydroxymethyl)-1-(5-methyl-7H-pyrrolo[2,3-d]pyrimidin-4-yl)pyrrolidin-3-yl]carbamate (C59). Yield: 2.31 g, 72%. LRMS (M+): 348.1; t_{R} (LCMS standard): 1.11 min.

Step 2: A solution of C59 (1.40 g; 4.0 mmol) in DMSO (19 mL) was cooled to 0° C., treated with TEA (1.65 mL; 12.04 mmol), and stirred at 0° C. for 10 min. The reaction mixture was then treated with a solution of a SO₃-Pyridine complex (1.90 g; 12.04 mmol) in DMSO (6 mL) and stirred at 25° C. for 2 hr. The reaction mixture was then treated with ethyl acetate (300 mL), and the resultant organic phase was collected and washed in this order with brine (75 mL), water (75 mL), 5% aq Na₂HCO₃ (75 mL) and saturated aq. CuSO₄. The organic phase was then dried over Na₂SO₄, filtered, and concentrated to provide tert-butyl 3-formyl-1-(5-methyl-7H-pyrrolo[2,3-d]pyrimidin-4-yl)pyrrolidin-3-ylcarbamate (C60). Yield: 1.40 g, 93%. LRMS (M+): 346.1; t_{R} (LCMS standard): 1.09 min.

Step 3: 2,4-dimethoxy benzyl amime (2.4 mL; 15.92 mmol) and 4 Å molecular sieves (1.0 mg) were added to a solution of C60 (5.63 g; 14.47 mmol) and acetic acid (3 mL; 5% in V) in MeOH (57 mL). The resulting reaction mixture was stirred at 25° C. for 4 hr., treated with MP-cyanoborohydride (16.3 g, 2.5 mmol/g, 40.75 mmol), and stirred at 25° overnight. The mixture was filtered and the solids rinsed with MeOH. The combined filtrates were concentrated, and the resultant residue purified by chromatography on silica gel (eluting with aq 30%-40% NH₄OH/DCM/MeOH gradient) to provide tert-butyl (S)-3-((2,4-dimethoxybenzylamino)methyl)-1-(5-chloro-4-a,7-dihydro-4H-pyrrolo[2,3-d]pyrimidin-4-yl)pyrrolidin-3-ylcarbamate (C61). Yield: 1.40 g, 93%. MS (M+): 517.5; t_{R} (LCMS polar): 2.49 min.

Step 4: A solution of C61 (4.6 g; 8.88 mmol) in TFA (45 mL) was stirred at 70° C. for 3 hr. and concentrated under reduced pressure. The resultant residue was purified by chromatography on silica (aq 30%-40% NH₄OH/DCM/MeOH) to provide (S)-3-(aminomethyl)-1-(5-chloro-4-a,7-dihydro-4H-pyrrolo[2,3-d]pyrimidin-4-yl)pyrrolidin-3-amine (C62). Yield: 1.7 g; 72%. LCMS (M+): 268.3; t_{R} (LCMS polar): 0.34 min.

Step 5: A solution of C62 (250 mg; 0.94 mmol) in DMF (3 mL) was treated with HOBt (133 mg; 1.43 mmol), 4-chloro-benzoic acid (147 mg; 0.94 mmol) and PS-carbodiimide (505 mg; 2.35 mmol). The resulting reaction mixture was stirred at 25° C. for 2.5 hr., treated with MP-carbonate (738 mg; 1.87 mmol), and stirred at 25° C. overnight. The mixture was filtered and the solids rinsed with MeOH. The combined filtrates were concentrated, and the resultant residue was purified by chromatography on silica (eluting with aq 30%-40% NH₄OH/DCM/MeOH gradient) to provide 70. Yield: 254 mg, 64%.

### Example 71

### Preparation of N—(((S)-3-amino-1-(5-methyl-7H-pyrrolo[2,3-d]pyrimidin-4-yl)pyrrolidin-3-yl)methyl)-2-chlorobenzamide (71)

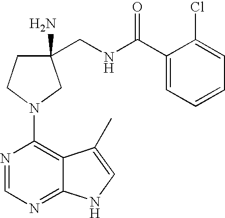

Step 1. A solution of the racemate of C2 (300 g, 979 mmol) in ethanol (700 mL) was slowly treated with a solution of (−)-tartaric acid (147 g, 979 mmol) in ethanol (300 mL), and the resultant yellow solution was stirred at 25° C. for 20 min. under N₂ atmosphere of nitrogen for twenty minutes. The stirring was stopped, and mixture was allowed to stand without agitation for 16 hr. The resultant solids were collected by filtration to provide 180 g of the tartrate salt of (R)-tert-butyl 1-benzyl-3-(hydroxymethyl)pyrrolidin-3-ylcarbamate (C63) as crystalline solids. These solids were recrystallized from ethanol (500 mL) to provide the tartrate salt of C63. Yield: 153 g, 335 mmol, 68%. C21H32N2O9. HPLC retention time (on 4.6 mm×15 cm Chiralpak AD-H column using an 85:15 heptane/ethanol with 0.2% diethylamine mobile phase flowing at 1.5 mL/min flow rate) 2.23 min. ¹H NMR (DMSO-d₆) δ: 7.4-7.3 (m, 5H), 6.78 (bs, 1H), 4.19 (s, 2H), 3.8 (bs, 2H), 3.44 (q, 2H), 2.9-2.78 (m, 4H), 1.94 (m, 2H), 1.4 (s, 9H) ppm.

Step 2: A solution of the C63 (62.0 g, 136 mmol) in MeOH (250 mL) and THF (750 mL) was treated with DIIPEA (71.1 mL, 408 mmol), BOC anhydride (29.7 g, 136 mmol), and 10 g of palladium hydroxide. The resultant mixture was then hydrogenated in a Parr vessel at 40 psi of H₂ for 5 hr. The reaction mixture was filtered through Celite and the solids washed with MeOH. The combined filtrates were then concentrated. The resultant reside was dissolved in ethyl acetate, treated with H₂O, and the resultant organic phase was collected and washed with 1N HCl, saturated NaHCO₃, and brine. The organic phase was then dried over MgSO₄, filtered, and concentrated to provide (R)-tert-butyl 3-(tert-butoxycarbonyl)-3-(hydroxymethyl)pyrrolidine-1-carboxylate (C64) as a white solid. Yield: 43.11 g, 99%. GC ret. time: 4.55. ¹H NMR (400 MHz, DMSO-d₆) δ: 6.79 (s, 1H), 4.88 (t, 1H, J=5.07 Hz), 3.48-3.46 (m, 3H), 3.25-3.20 (m, 3H), 2.10-1.80 (m, 2H), 1.38 (s, 18H).

Step 3: A solution of a SO₃-pyridine complex (63.0 g, 396 mmol) in 340 mL DMSO under a N₂ atmosphere was cooled in a salt-packed ice bath and treated with TEA (57 mL, 409 mmol). The resultant slurry was then treated drop-wise with a solution of C64 (42.0 g, 132 mmol) in DMSO (170 mL) while maintaining an internal temperature below 25° C. The reaction mixture was slowly warmed to 25° C. and stirred for 3 hr. The reaction mixture was cooled to 0° C. and treated with pre-chilled ethyl acetate (1.2 L). The mixture was treated with brine (420 mL), and the resultant organic phase was collected and washed with brine (420 mL), water (420 mL), sat. NaHCO₃ (2× the 420 mL), and saturated aq. CuSO₄ (2×175 mL). The organic phase was dried over MgSO₄, filtered, and concentrated to provide (R)-tert-butyl 3-(tert-butoxycarbonyl)-3-formylpyrrolidine-1-carboxylate (C65) as a white solid. Yield: 39.4 g, 95%. GC ret. time: 4.20. ¹H NMR (400 MHz, DMSO-d₆) δ: 9.41 (s, 1H), 7.90 (m, 1H), 3.63 (m, 1H), 3.40-3.20 (5H), 1.83-2.08 (m, 2H), 1.39 (s, 18H).

Step 4: A mixture of C65 (38.0 g, 121 mmol), benzyl amine (13.2 mL, 121 mmol) and molecular sieves (16 g) in anhydrous MeOH (500 mL) was stirred at 25° C. under N₂ atmosphere for 18 hr. Sodium borohydride (6.89 g, 182 mmol) was added portion-wise, and the reaction mixture was stirred for 3 hr. The mixture was concentrated, and the resultant residue was treated with ethyl acetate. The resultant organic phase was washed with 1N NaOH (3×), and the combined organic phases were dried over NaSO₄, filtered, and concentrated. The resultant residue was passed through a plug of silica eluting with 100% EtOAc to provide (S)-tert-butyl 3-((benzylamino)methyl)-3-(tert-butoxycarbonyl)pyrrolidine-1-carboxylate (C66) as a colorless residue. Yield: 48.6 g, 99%. GC ret. time: 4.43. ¹H NMR (400 MHz, methanol-d₄) δ: 7.30-7.28 (m, 5H), 4.86 (s, 2H), 3.77-3.53 (m, 2H), 3.38-3.28 (m, 2H), 2.8 (dd, 2H), 2.2-1.8 (m, 2H), 1.43 (m, 18H).

Step 5: A solution of C66 (48.0 g, 118 mmol) in THF (140 mL) and MeOH (570 mL) was treated with 10% palladium on carbon (15 g, 50% wet). The reaction mixture was then hydrogenated for 18 h at 40 psi of H₂. The mixture was filtered through Celite and the solids washed with ethyl acetate. The combined filtrates were concentrated to provide (S)-tert-butyl 3-(aminomethyl)-3-(tert-butoxycarbonyl)pyrrolidine-1-carboxylate (C67) as a white solid. Yield: 35.5 g, 95% yield. LC/MS: 316.5 (⁺H). ¹H NMR (400 MHz, methanol-d₄) δ: 3.7-3.5 (m, 2H), 3.4-3.2 (m, 3H), 3.0-2.7 (m, 1H), 2.2-1.85 (m, 2H), 1.45 (m, 18H).

Step 6: A solution of C67 in THF (14 mL) was treated with HOBt (193 mg; 1.26 mmol), 2-chloro-benzoic acid (197 mg; 1.26 mmol) and PS-carbodiimide (738 mg; 2.71 mmol) was stirred at 25° C. overnight. The mixture was treated with MP-carbonate (738 mg; 2.32 mmol) and stirred for 5 hr. The mixture was then filtered and the solids rinsed with MeOH. The combined filtrates were concentrated, and the resultant residue was treated with DCM (30 mL) and TFA (30 mL), stirred for 3 hr., and concentrated. The resultant residue was purified by chromatography on silica (eluting with aq 30%-40% NH₄OH/DCM/MeOH gradient) to provide N—(((R)-3-aminopyrrolidin-3-yl)methyl)-2-chlorobenzamide (C68). Yield: 288 mg; 90%. LCMS (M+): 254.4; t_{R} (LCMS polar): 0.19 min.

Step 7: A solution of C13 (95.2 mg; 0.57 mmol) in 2-propanol (0.35 mL) and isopropyl alcohol (IPA) (0.35 mL) was treated with DIIPEA (300 uL; 2.0 mmol) followed by C68 (144 mg; 0.57 mmol). The reaction mixture was stirred at 80° C. overnight. The reaction mixture was then concentrated under reduced pressure, and the resultant residue was purified by chromatography on silica (eluting with aq 30%-40% NH₄OH/DCM/MeOH gradient) to provide 71. Yield: 170 mg, 78%.

### Example 72

### Preparation of N-{[(3S)-3-amino-1-(5-ethyl-7H-pyrrolo[2,3-d]pyrimidin-4-yl)pyrrolidin-3-yl]methyl}-2-methylpropanamide (72)

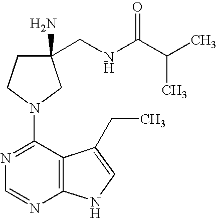

Step 1: (S)-1-(tert-butoxycarbonyl)-3-(aminomethyl)pyrrolidin-3-ylcarbamate (C69) was prepared in a manner similar to that described for C5 in Step 4 of Example 1 except that methylamine was used instead of 5-methylisoxazol-3-amine to provide C69.

Step 2. A stirred solution of C69 (2.2 g; 6.97 mmol) in THF (23 mL) and DMF (100 ul) under N₂ atmosphere was cooled to 0° C. and treated drop-wise with benzyl chloroformate (1.04 ml; 6.97 mmol). The mixture was stirred for an additional 2 min., and the resulting mixture was maintained at 0° C. and treated with TEA (1.27 ml; 9.06 mmol). The mixture was then stirred at 0° C. overnight. The mixture was treated with aq 0.5 N HCl (50 ml) for 5 min. and extracted with ethyl acetate (2×40 ml). The combined organic phases were washed with saturated aq Na₂HCO₃ (40 ml) and brine (40 ml). The separated organic phases were then dried over Na₂SO₄, filtered, and concentrated to provide (S)-3-(benzyloxycarbonylamino-methyl)-3-tert-butoxycarbonylamino-pyrrolidine-1-carboxylic acid tert-butyl ester (C70). Yield: 3.1 g, 99%. LRMS (M+): 450.5; t_{R} (LCMS standard): 2.65 min.

Step 3: A mixture of C70 (4.92 g; 11 mmol) and TFA (22 ml) in DCM (12 ml) was stirred at 40° C. for 3 hr. The mixture was concentrated, and the resultant residue was purified by chromatography on silica (eluting with aq 30%-40% NH₄OH/DCM/MeOH gradient) to provide 1.5 eq of the TFA salt of benzyl {[(3R)-3-aminopyrrolidin-3-yl]methyl}carbamate (C71). Yield: 4.49 g, 85%. LCMS (M+): 250.4; t_{R} (LCMS polar): 0.29 min.

Step 4: A solution of C7 (0.89 g; 4.91 mmol) in ethyl acetate (11 mL) was treated with DIIPEA (2.02 g; 15.6 mmol) followed by C71 (2.13 g; 5.06 mmol) and stirred at 80° C. overnight. The reaction mixture was then concentrated under reduced pressure, and the resultant residue was purified by chromatography on silica (eluting with aq 30%-40% NH₄OH/DCM/MeOH gradient) to provide benzyl ((S)-3-amino-1-(5-ethyl-7H-pyrrolo[2,3-d]pyrimidin-4-yl)pyrrolidin-3-yl)methylcarbamate (C72). Yield: 1.76 g, 85%. LCMS (M+): 395.2; t_{R} (LCMS polar): 2.18 min.

Step 5: A mixture of C72 (1.76 g; 4.46 mmol) and TFA (30 ml) was stirred at 70° C. for 3 hr. The mixture was then concentrated under reduced pressure, and the resultant residue was purified by chromatography on silica (eluting with aq 30%-40% NH₄OH/DCM/MeOH gradient) to provide (S)-3-(aminomethyl)-1-(5-ethyl-7H-pyrrolo[2,3-d]pyrimidin-4-yl)pyrrolidin-3-amine (C73). Yield: 0.63 g, 54%. LCMS (M+): 261.2; t_{R} (LCMS polar): 0.25 min.

Step 6: A solution C73 (27 mg; 0.103 mmol) in DMF (1 mL) was treated with HOBt (22 mg; 0.154 mmol), isobutyric acid (8.81 mg; 0.1 mmol) and PS-carbodiimide (131 mg; 0.206 mmol). The resulting reaction mixture was stirred at 25° C. for 3 hr., treated with MP-carbonate (160 mg; 0.503 mmol), and stirred at 25° C. overnight. The mixture was filtered and the precipitate rinsed with MeOH. The combined filtrates were evaporated, and the resultant residue was purified by preparative HPLC (NH₄OH/CAN/water mobile) to provide 72. Yield: 12 mg; 36%.

### Example 73

### Preparation of N-{[(3S)-3-amino-1-(5-ethyl-7H-pyrrolo[2,3-d]pyrimidin-4-yl)pyrrolidin-3-yl]methyl}-2,4-difluorobenzamide (73)

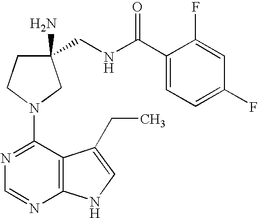

Step 1: A solution of C67 (36.06 g, 114 mmol) in THF (384 mL) and DMF (1 mL) was cooled to 0° C. and treated with 2,4-difluorobenzoyl chloride. The resulting mixture was stirred at 0° C. for 10 min., and N,N-diisopropylethylamine (23.87 mL, 137 mmol) was added over 10 min. The reaction mixture was then stirred at 20° C. overnight. The mixture was treated with water (250 mL) and extracted with ethyl acetate (3×200 mL). The combined organic extracts were washed with water (1×100 mL) and brine (1×100 mL), dried over Na₂SO₄, filtered, and concentrated under reduced pressure. The resultant white foam was purified by silica gel chromatography (300 g, 0-100% ethyl acetate in hexanes) to provide (S)-tert-butyl 3-(tert-butoxycarbonyl)-3-((2,4-difluorobenzamido)methyl)pyrrolidine-1-carboxylate (C74) as a white solid. Yield: 48.8 g, 93%. TLC R_{f}=0.25 (1:1 ethyl acetate/hexanes). APCI MS (M+1) 490.1.

Step 2: Acetyl chloride (76 mL, 1071 mmol) was added to anhydrous methanol (300 mL) with stirring at 0° C. After 30 min., a solution of C74 (48.8 g, 107 mmol) in methanol (170 mL) was added drop-wise, and the reaction mixture was warmed to 20° C. and stirred for 2 hr. The mixture was then concentrated under reduced pressure to provide (R)—N-((3-aminopyrrolidin-3-yl)methyl)-2,4-difluorobenzamide (C75) as a white solid. Yield: 35 g, 99%. HPLC r.t.=1.29 min. TLC Rf=0.1 (1:1 ethyl acetate/hexanes).

Step 3: A mixture of C75 (16 g, 49 mmol), C13 (8.85 g, 49 mmol) and sodium bicarbonate (20.5 g, 244 mmol) in ethanol (150 mL) was refluxed for 10 hr. The mixture was then filtered hot through Celite, and the filtrate was concentrated under reduced pressure. The resultant residue was partitioned between ethyl acetate (100 mL) and water (200 ml), and the organic phase was collected. The aqueous phase was extracted with ethyl acetate (2×100 mL), and the combined organic extracts were washed with water (2×100 mL), brine (80 mL), dried over Na₂SO₄, filtered, and concentrated under reduced pressure to provide 73 as a yellow solid. Yield: 17.4 g, 89.3%. TLC Rf=0.42 (10% methanol/DCM).

Preparation of a hemisolvate salt of 73: A solution of 73 (450 mg, 1.124 mmol) in acetonitrile (3.00 mL) was treated with sulfuric acid (120 mL, 2.25 mmol). The resultant mixture was then treated with ethanol (1.00 mL), and the resultant solution was heated in a sealed vial on shaker plate at 80° C. for sixteen hours. The reaction mixture was allowed to cool to 25° C., and the volatiles were allowed to evaporate with no perturbation. During this time large crystals began to form. The solids were collected via filtration and dried under reduced pressure. An additional amount product was subsequently collected from the filtrate. Analysis indicated that the product contained 1.5 equivalents of sulfate and 0.5 equivalents of ethanol per equivalent of 73.

### Example 74

### Preparation of (S)—N-((3-amino-1-(5-methyl-7H-pyrrolo[2,3-d]pyrimidin-4-yl)pyrrolidin-3-yl)methyl)-4-chlorobenzamide (74)

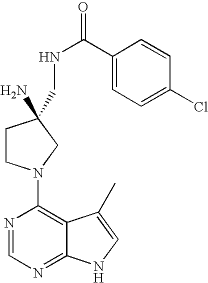

Step 1: A solution of chloro benzylformate (1.98 mL, 13.9 mmol) in DCM (3 mL) was treated drop-wise with a chilled solution (−60° C.) solution of tert-butyl 3-(hydroxymethyl)pyrrolidin-3-ylcarbamate (3.0 g, 13.9, mmol) (see Tomita et al., J. Med. Chem. 2002, 45, 5564) and TEA (5.81 mL, 41.7 mmol) in DCM (5 mL). The reaction mixture was then allowed to warm to 25° C. overnight. The reaction mixture was diluted with DCM and washed with 0.1N HCl (2×) and sat. NaHCO₃. The organic layer was dried over MgSO₄, filtered, and concentrated. The resultant residue was dissolved in ethyl acetate and the solution cooled to 0° C. The precipitates that formed upon cooling were collected by filtration to provide benzyl 3-(tert-butoxycarbonyl)-3-(hydroxymethyl)pyrrolidine-1-carboxylate (C76) as a white solid. Yield: 2.08 g. ¹H NMR (400 MHz, DMSO-d₆) δ: 7.35-7.25 (m, 5H), 6.84 (s, 1H), 5.02 (s, 2H), 4.88 (br s, 1H), 3.61-3.26 (m, 6H), 2.01-1.85 (m, 2H), 1.34 (s, 9H).

Step 2: A solution of thionyl chloride (0.52 mL, 7.13 mmol) in acetonitrile (9 mL) was chilled to −40° C. and treated with solid C76 (1.0 g, 2.85 mmol). Pyridine (1.13 g, 14.3 mmol) was added drop-wise over 5 min., and the resultant solution was stirred at 25° C. for 30 min. The solution was then concentrated, treated with ethyl acetate (20 mL), and filtered. The filtrate was concentrated, and the resultant residue was dissolved in DCM, filtered through a plug of silica gel, and washed with 1:1 ethyl acetate/hexane to provide the corresponding spirocyclic sulfinyl urethane (C77). Yield: 1.0 g, 94%. TLC Rf=0.45 (10% MeOH/DCM).

Step 3: Water (31 mL, 1750 mmol) was added drop-wise to a chilled (0° C.) suspension of C77 (3.0 g, 7.6 mmol), sodium periodate (1.79 g, 8.4 mmol) and ruthenium trichloride hydrate (0.16 g, 0.76 mmol) in acetonitrile (47 mL), and the mixture was stirred at 20° C. overnight. The mixture was concentrated, treated with saturated NaHCO₃ (20 mL), and extracted with ethyl acetate (3×12 mL). The combined extracts were dried over Na₂SO₄, filtered, and concentrated to provide the corresponding spirocyclic sulfonyl urethane (C78). Yield: 3.0 g, 95%. TLC Rf=0.95 (10% MeOH/DCM).

Step 4: Sodium azide was added to a solution of C78 (15.8 g, 38.33 mmol) in DMF (50 mL), and the mixture was stirred vigorously at 25° C. for 40 hr. The mixture was then partitioned between DCM (250 mL) and water (250 mL), 0.05N HCl was added until pH=5.0 was achieved, and the mixture was stirred at 25° C. for 2 hr. The organic phase was collected, and the aqueous phase was extracted with DCM (2×100 mL). The combined organic phases were dried over Na₂SO₄, filtered, and concentrated. The resultant residue was dissolved in 4:1 THF/water (170 mL), combined with triphenylphosphine (17.6 g, 67 mmol), and stirred at 20° C. for 20 hr. The mixture was then diluted with water (250 mL) and ethyl acetate (250 mL). The organic layer was removed, and the aqueous layer was acidified with 10% aqueous citric acid until pH=3 was achieved and washed with ethyl acetate (3×50 mL). The aqueous layer was then saponified to pH 9 with 1N sodium hydroxide and extracted with diethyl ether (3×250 mL). The combined diethyl ether extracts were washed with water (2×100 mL), dried over MgSO₄, filtered, and concentrated under reduced pressure to provide (S)-benzyl 3-(aminomethyl)-3-(tert-butoxycarbonyl)pyrrolidine-1-carboxylate (C79). Yield: 8.7 g, 65.1%. LCMS STD r.t.=1.6 min. MS (M+1)=350.5.

Step 5: A solution of C79 (0.95 g, 2.73 mmol) in THF (3.2 mL) and DMF (0.1 mL) was cooled to 0° C. and treated with p-chlorobenzoylchloride (0.45 mL, 3.5 mmol) and TEA (drop-wise) (0.49 mL, 3.5 mmol). The resultant white mixture was stirred at 25° C. for 3 hr. The mixture was then treated with ethyl acetate (10 mL) and water (20 mL). The organic phase was collected, and the aqueous phase was extracted with ethyl acetate (2×10 mL). The combined organic phase were washed with 50% brine (2×20 mL), dried over Na₂SO₄, filtered, and concentrated to provide (S)-benzyl 3-(tert-butoxycarbonyl)-3-((4-chlorobenzamido)methyl)pyrrolidine-1-carboxylate (C80). Yield: 1.1 g, 83%. LCMS_STD r.t. 2.9 min. MS (M+1)=488.5.

Step 6. A solution C80 (1 gram) in 10 mL of TFA was stirred at 70° C. for 4 hr. The reaction mixture was then concentrated under reduced pressure to provide the bis-trifluoroacetic acid salt of (R)—N-((3-aminopyrrolidin-3-yl)methyl)-4-chlorobenzamide (C81) as a viscous oil. The product was carried on to next step without further purification. MS (M+1) 254.4.

Step 7. A mixture of C81 (60 mg, 0.2 mmol), C13 (48 mg, 0.28 mmol), and N,N-diisopropylethylamine (0.25 mL, 1.4 mmol) were combined in pyridine (0.4 mL) and reacted under microwave heating at 120° C. for 22 min. The reaction mixture was treated with water (5 mL) and extracted with ethyl acetate (3×15 mL). The combined organic extracts were washed with 50% brine (15 mL), dried over MgSO₄, filtered, and concentrated. The resultant pale yellow oil was purified by column chromatography (0-20% MeOH/DCM) to provide 74 as a white solid. Yield: 65 mg, 70%. TLC R_{f}=0.32 (10% methanol in DCM).

### Example 75

### Preparation of 1-{[3-amino-1-(5-methyl-7H-pyrrolo[2,3-d]pyrimidin-4-yl)pyrrolidin-3-yl]methyl}-3-(2,3-dimethylphenyl)urea (75)

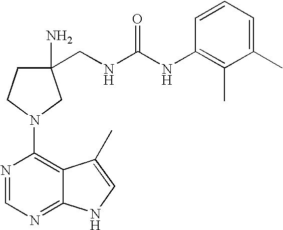

Step 1. [3-Aminomethyl-1-(5-methyl-7H-pyrrolo[2,3-d]pyrimidin-4-yl)-pyrrolidin-3-yl]-(2,4-dimethoxy-benzyl)-amine (C82) was prepared in a manner similar to that described for making 77 in Example 77 except that 2,3-dimethylformanilide was used instead of benzensulfonyl chloride.

Step 2. A solution of C82 (100 mg, 0.252 mmol) in dry DCM (1.00 mL) was treated with DIIPEA (103 μL, 0.592 mmol) and 2,3-dimethylphenyl isocyanate (48.3 mg, 0.328 mmol). The reaction mixture was then allowed to stir in a sealed vial for 16 hr. The resultant slurry was treated with 200 μL water and allowed to stir at 25° C. for 4 hr. The organic layer was collected and concentrated under reduced pressure. The resultant oily residue was purified over silica (97:3:0.3 CHCl₃:CH₃OH:NH₄OH) and the fractions containing product were concentrated. The resultant white foam was treated with TFA (2.00 mL) and heated at 80° C. for 2 hr. The mixture was then concentrated under reduced pressure, and the resultant violet foam was purified over silica (97:3:0.3 CHCl₃:CH₃OH:NH₄OH) to provide 75 as a light yellow solid. Yield: 17 mg, 0.0432 mmol, 13%.

### Example 76

### Preparation of 1-{[3-amino-1-(5-methyl-7H-pyrrolo[2,3-d]pyrimidin-4-yl)pyrrolidin-3-yl]methyl}-1-(2-methoxyethyl)-3-phenylurea (76)

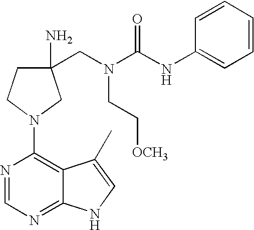

Step 1: A solution of C13 (1.51 g; 9.25 mmol) in 2-propanol (16 mL) was treated with DIIPEA (5 mL; 27.25 mmol) followed by C14 (2.00 g; 9.25 mmol). The reaction mixture was stirred at 80° C. overnight. The reaction mixture was then concentrated under reduced pressure, and the resultant slurry was treated with ethyl acetate (100 mL). The resultant precipitate was collected by filtration, rinsed with ethyl acetate (2×75 mL), and dried to provide tert-butyl [3-(hydroxymethyl)-1-(5-methyl-7H-pyrrolo[2,3-d]pyrimidin-4-yl)pyrrolidin-3-yl] (C83). Yield: 2.31 g, 72%. LRMS (M+): 348.1; t_{R} (LCMS standard): 1.11 min.

Step 2: A solution of C83 (1.40 g; 4.0 mmol) in DMSO (19 mL) was cooled to 0° C., treated with TEA (1.65 mL; 12.04 mmol), and stirred at 0° C. for 10 min. The reaction mixture was treated with a solution of a SO₃-Pyridine complex (1.90 g; 12.04 mmol) in DMSO (6 mL) and stirred at 25° C. for 2 hr. The mixture was then treated with ethyl acetate (300 mL), and the organic phase was washed in this order with brine (75 mL), H₂O (75 mL), 5% aq. Na₂HCO₃ (75 mL) and saturated aq CuSO₄. The organic phase was dried over Na₂SO₄, filtered, and concentrated to provide tert-butyl 3-formyl-1-(5-methyl-7H-pyrrolo[2,3-d]pyrimidin-4-yl)pyrrolidin-3-ylcarbamate (C84). Yield: 1.40 g, 93%. LRMS (M+): 346.1; t_{R} (LCMS standard): 1.09 min.

Step 3: A solution of C84 (0.5 g; 1.45 mmol) and acetic acid (2 mL; 16% in V) in MeOH (10 mL) was treated with 2-methoxyethanamine (150 uL; 1.74 mmol) and 3 Å molecular sieves (250 mg), and the resultant mixture was stirred at 50° C. overnight. The mixture was then treated with MP-Cyanoborohydride (1.45 g, 2.5 mmol/g; 3.65 mmol) and stirred at 50° C. for 5 hr. The mixture was filtered and the solids rinsed with MeOH. The combined filtrates were concentrated, and the resultant residue was purified by chromatography on silica gel (eluting with aq. 30%-40% NH₄OH/DCM/MeOH gradient) to provide tert-butyl 3-((2-methoxyethylamino)methyl)-1-(5-methyl-7H-pyrrolo[2,3-d]pyrimidin-4-yl)pyrrolidin-3-ylcarbamate (C85). Yield: 440 mg, 74%. MS (M+): 405.4; t_{R} (LCMS polar): 0.78 min.

Step 4: A solution of C85 (51 mg; 0.126 mmol) in DMF (1.0 mL) was treated with 1-isocyanatobenzene (16 uL; 0.126 mmol), and the resultant mixture was stirred at 25° C. for 3 hr. The mixture was then concentrated under reduced pressure. The resultant residue was treated with DCM (0.5 mL) and TFA (0.5 mL) and stirred for 3 hr. The mixture was then concentrated, and the resultant residue was purified by preparative HPLC (NH₄OH/acetonitrile/water mobile) to provide 76. Yield: 11.4 mg; 22%.

### Example 77

### Preparation of N-{[3-amino-1-(5-methyl-7H-pyrrolo[2,3-d]pyrimidin-4-yl)pyrrolidin-3-yl]methyl}benzenesulfonamide (77)

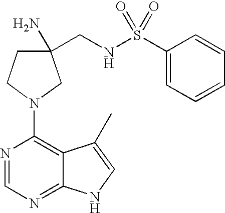

A solution of C58 (50 mg; 0.126 mmol) in DMF (0.5 mL) was treated with DIIPEA (22 uL; 0.127 mmol) followed by benzenesulfonyl chloride (15 uL; 0.127 mmol). The resulting mixture was stirred at 25° C. for 12 hr. and concentrated under reduced pressure. The resultant residue was treated with TFA (0.5 mL), stirred at 80° C. for 3 hr., and concentrated. The resultant residue was purified by preparative HPLC (TFA/acetonitrile/water mobile) to provide 77. Yield: 12 mg; 26%.

### Examples 78 to 198

Examples 78 to 198 (Table 5) were prepared according to the methods described in Examples 69-73 and 75-77.

Table 1 also contains the Akt kinase activity for compounds 66-198.

### Example 199

### Preparation of 3-((phenylamino)methyl)-1-(5-methyl-7H-pyrrolo[2,3-d]pyrimidin-4-yl)pyrrolidin-3-amine (199)

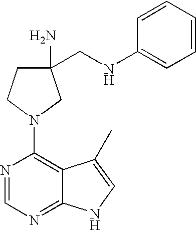

Compound 199 was prepared in a manner similar to that described for compound 3 in Example 3 except that aniline was used instead of 2-fluoro-3-(trifluoromethyl)aniline. ¹H NMR (500 MHz, methanol-d₄) δ: 2.03 (1H, m), 2.21 (1H, m), 2.38 (3H, s), 3.37 (2H, m), 3.68 (1H, d), 3.89 (1H, d), 3.91 (1H, m), 4.05 (1H, q), 6.60 (1H, t), 6.69 (2H, d), 6.92 (1H, s), 7.08 (2H, m), 8.08 (1H, s) ppm.

### Example 200

### Preparation of 3-(4-chloro(phenylamino)methyl)-1-(5-methyl-7H-pyrrolo[2,3-d]pyrimidin-4-yl)pyrrolidin-3-amine (200)

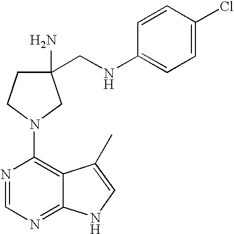

Compound 200 was prepared in a manner similar to that described for compound 3 in Example 3 except that 4-chloroaniline was used instead of 2-fluoro-3-(trifluoromethyl)aniline. ¹H NMR (500 MHz, methanol-d₄) δ: 1.88-2.01 (4H, m), 2.38 (3H, s), 2.87 (2H, s), 3.42-3.49 (2H, m), 3.71-3.77 (2H, m), 3.84 (2H, s), 7.00 (1H, s), 7.32-7.33 (2H, d), 7.34-7.40 (2H, d), 8.19 (1H, s), 8.21 (1H, s) ppm.

### Example 201

Piperidinyl analogs of compounds 199 and 200, which are depicted below as compounds AA and BB, respectively.

| Piperidinyl Derivative | Structure |
|---|---|
| (4-((phenylamino)methyl)- 1-(5-methyl-7H-pyrrolo[2, 3-d]pyrimidin-4- yl)piperidin-4-amine (AA)) | 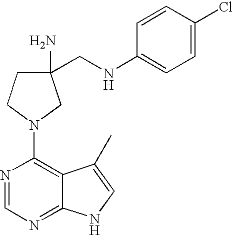 |
| (4-(4-chloro(phenylamino) methyl)-1-(5-methyl-7H- pyrrolo[2,3-d]pyrimidin- 4-yl)piperidin-4-amine (BB)) | 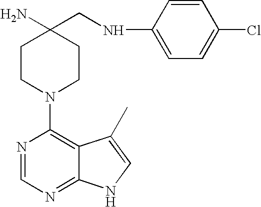 |

The compounds AA and BB were prepared in a manner similar to that described above for compounds 199 and 200, respectively, by using 4-(hydroxymethyl)piperidin-4-ylcarbamate instead of the corresponding pyrrolidine reagent.

Selectivity Study:

A selectivity study was carried out on compounds AA and BB and exemplary compounds 199 and 200. The Akt and PKA kinase activity of each of the compounds were determined by the procedures described above in the Detailed Description of the Invention. The PKA/Akt selectivity ratio for each of the compounds was calculated by dividing the PKA kinase activity by the Akt kinase activity, using the experimentally determined IC50 values. The results of the study are shown below in Table 6.

TABLE 6
Comparison of the selectivity (PKA/Akt) of compounds of formula I
and piperidinyl analogs.

| | Akt Kinase | PKA Kinase | Selectivity Ratio |
|---|---|---|---|
| Compound | Activity (μM) | Activity (μM) | (PKA/Akt) |
| 199 | 0.162 | 2.580 | 15.93 |
| AA | 0.145 | 0.081 | 0.56 |
| 200 | 0.118 | 0.279 | 2.36 |
| BB | 0.0459 | 0.0263 | 0.57 |

The results of the selectivity study (Table 6) show that the exemplary compounds of formula I (which contain a pyrrolidinyl moiety) are more selective for Akt kinase than are the corresponding piperidinyl analogs. The results of the study suggest that compounds having a 5-membered cyclic amine moiety (e.g., the compounds of formula I) are more selective for Akt kinase than are analogous compounds having a 6-membered cyclic amine moiety (e.g., piperidinyl derivatives).

TABLE 3
Examples 1 to 59.

| | | | | | | | | | Avg Akt |
|---|---|---|---|---|---|---|---|---|---|
| | | | LCMS/ | LCMS | HPLC | MS | APCI | | kinase, |
| | | | HPLC | RT, | RT, | Peak 1 | Acid | | μM (50% |
| Ex. | Name | Prep. | Method | min | min | (Peak 2) | Basic | ¹H NMR δ, ppm | inhibition) |
| 1 | N-(((S)-3-amino-1-(5-ethyl-7H- | Ex. 1 | LCMS | 0.47 | | 342.5 | | (400 MHz, methanol-d₄) 8.05 (s, | 0.01542 |
| | pyrrolo[2,3-d]pyrimidin-4- | | Polar | | | | | 1H), 6.90 (s, 1H), 5.63 (s, 1H), | |
| | yl)pyrrolidin-3-yl)methyl)-5- | | | | | | | 3.96-4.01 (m, 1H), 3.83-3.87, (m, | |
| | methylisoxazol-3-amine (1) | | | | | | | 3H), 3.60 (d, 1H), 3.37 (d, 1H), 2.75- | |
| | | | | | | | | 2.80 (m, 2H), 2.07-2.13 (m, 1H), | |
| | | | | | | | | 1.88-1.93 (m, 1H), 2.24 (s, 3H) | |
| | | | | | | | | 1.25 (t, 3H). | |
| 2 | N-(((S)-3-amino-1-(3-chloro- | Ex. 2 | LCMS | 0.15 | | 357.4 | | (400 MHz, methanol-d₄) 8.18 (s, | |
| | 1H-pyrrolo[2,3-b]pyridin-4- | | Polar | | | | | 1H), 7.96-8.00 (m, 1H), 7.90 (d, | |
| | yl)pyrrolidin-3-yl)methyl)-2- | | | | | | | 1H), 7.69-7.72 (m, 1H), 7.36 (d, | |
| | methylpyridin-3-amine (2) | | | | | | | 1H) 7.06 (d, 1H), 4.54-4.60 | |
| | | | | | | | | (m, 2H), 4.44-4.51 (m, 2H), 3.92 (d, | |
| | | | | | | | | 2H), 2.68 (s, 3H), 2.48-2.53 (m, | |
| | | | | | | | | 1H) 2.41-2.46 (m, 1H). | |
| 3 | 3-(2-fluoro-3- | Ex. 3 | LCMS | 1.2 | | 409.3 | | (500 MHz, methanol-d₄) 1.91-1.98 | 0.0012 |
| | (trifluoromethyl)phenylamino)- | | STD | | | | | (1H, m), 2.02-2.14 (1H, m), 2.30 | |
| | methyl)-1-(5-methyl-7H- | | | | | | | (3H, s), 3.28-3.33 (2H, m), 3.59 | |
| | pyrrolo[2,3-d]pyrimidin-4- | | | | | | | (1H, d), 3.78 (1H, d), 3.80-3.85 | |
| | yl)pyrrolidin-3-amine (3) | | | | | | | (1H, m), 3.95-3.99 (1H, m), 6.74- | |
| | | | | | | | | 6.78 (1H, m), 6.84 (1H, s), 6.95- | |
| | | | | | | | | 7.00 (2H, m), 8.03 (1H, s) | |
| 4 | 4-{3-amino-3-[(4-chloro- | Ex. 4 | B | | 4.210 | 368.4 | | (500 MHz, DMSO-d₆) 2.20-2.31 | 0.041 |
| | phenylamino)-methyl]- | | | | | | | (m, 1H) 2.33-2.45 (m, 1H) 3.48 | |
| | pyrrolidin-1-yl}-7H-pyrrolo[2,3- | | | | | | | (s, 2H) 3.87 (d, J = 12.96Hz, 1H) | |
| | d]pyrimidine-5-carbonitrile (4) | | | | | | | 3.99-4.10 (m, 2H) 4.10-4.25 (m, | |
| | | | | | | | | 1H) 6.14 (br. s., 1H) 6.73 (d, | |
| | | | | | | | | J = 8.81Hz, 2H) 7.14 (d, J = 8.81 | |
| | | | | | | | | Hz, 2H) 7.93-8.67 (m, 4H) 12.91 | |
| | | | | | | | | (br. s., 1H). | |
| 5 | 2-{[3-Amino-1-(3-methyl-1H- | Ex. 5 | LCMS | 1.12 | | 382.2 | | | 0.394 |
| | pyrazolo[3,4-d]pyrimidin-4-yl)- | | STD | | | | | | |
| | pyrrolidin-3-ylmethyl]-amino}- | | | | | | | | |
| | benzoic acid methyl ester (5) | | | | | | | | |
| 6 | 1-(5-Chloro-7H-pyrrolo[2,3- | Ex. 6 | LCMS | 2.68 | | 435.3 | | | 0.0134 |
| | d]pyrimidin-4-yl)-3-[(3-phenoxy- | | Polar | | | | | | |
| | phenylamino)-methyl]- | | | | | | | | |
| | pyrrolidin-3-ylamine (6) | | | | | | | | |
| 7 | 4-(3-amino-3-((3-chloro-2- | Ex. 7 | LCMS | 2.15 | | 369.2 | | | 0.0523 |
| | fluorophenylamino)methyl)- | | Polar | | | | | | |
| | pyrrolidin-1-yl)-1H-pyrazolo[3,4- | | | | | | | | |
| | d]pyrimidine-3-carbonitrile (7) | | | | | | | | |
| 8 | 4-{3-Amino-3-[(3-chloro- | Ex. 8 | LCMS | 2.15 | | 369.2 | | | 0.249 |
| | phenylamino)-methyl]- | | Polar | | | | | | |
| | pyrrolidin-1-yl}-1H-pyrazolo[3,4- | | | | | | | | |
| | d]pyrimidine-3-carbonitrile (8) | | | | | | | | |
| 9 | 1-(5-ethyl-7H-pyrrolo[2,3- | Ex. 9 | LCMS | 1.5 | | 405.3 | | | 0.002 |
| | d]pyrimidin-4-yl-3-({[3- | | STD | | | | | | |
| | (trifluoromethyl)phenyl]amino}- | | | | | | | | |
| | methyl)pyrrolidin-3-amine (9) | | | | | | | | |
| 10 | 3-{[(2,3- | Ex. 10 | LCMS | 1.8 | | 391.3 | | | 0.003 |
| | dichlorophenyl)amino]methyl}- | | STD | | | | | | |
| | 1-(5-methylpyrrolo[2,1- | | | | | | | | |
| | f][1,2,4]triazin-4-yl)pyrrolidin-3- | | | | | | | | |
| | amine (10) | | | | | | | | |
| 11 | 4-(3-amino-3-{[(2- | E. 11 | LCMS | 1.5 | | 425.4 | | | |
| | phenoxyphenyl)amino]methyl}- | | STD | | | | | | |
| | pyrrolidin-1-yl)-1H-pyrrolo[2,3- | | | | | | | | |
| | b]pyridine-3-carbonitrile (11) | | | | | | | | |
| 12 | 4-({[3-amino-1-(5-methyl-7H- | Ex. 3 | LCMS | 1.4 | | 491.2 | | | 0.0105 |
| | pyrrolo[2,3-d]pyrimidin-4- | | Polar | | | | | | |
| | yl)pyrrolidin-3-yl]methyl}amino)- | | | | | | | | |
| | N-benzylbenzenesulfonamide | | | | | | | | |
| | (12) | | | | | | | | |
| 13 | (3S)-1-(5-methyl-7H- | Ex. 3 | LCMS | 2.48 | | 391.3 | | (400 MHz, DMSO-d₆) 1.24 (br. s., | 0.00226 |
| | pyrrolo[2,3-d]pyrimidin-4-yl)-3- | | Polar | | | | | 1H) 2.38 (br. s., 5H) 3.44 (br. s., | |
| | ({[3- | | | | | | | 2H) 3.69 (br. s., 1H) 3.90 (br. s., | |
| | (trifluoromethyl)phenyl]amino}- | | | | | | | 3H) 4.18 (br. s., 2H) 5.08 (br. s., | |
| | methyl)pyrrolidin-3-amine (13) | | | | | | | 1H) 6.66 (s, 1H) 6.84 (br. s., 1H) | |
| | | | | | | | | 7.23 (br. s., 5H) 7.47 (br. s., 2H) | |
| | | | | | | | | 7.70 (br. s., 1H) 8.97 (br. s., 2H) | |
| | | | | | | | | 12.67 (br. s., 1H | |
| 14 | 1-(9H-purin-6-yl)-3-({[3- | Ex. 3 | | | | 378 | | (400 MHz, DMSO-d₆) 12.7 (s, 1H) | 0.108 |
| | (trifluoromethyl)phenyl]amino}- | | | | | | | 9.00 (s, 3H), 8.34 (s, 1H), 7.30 (s, | |
| | methyl)pyrrolidin-3-amine (14) | | | | | | | 1H), 7.26 (t, 1H), 7.02 (d, 1H) 6.99 | |
| | | | | | | | | (s, 1H), 6.83 (d, 1H), 4.26 (d, 1H), | |
| | | | | | | | | 4.14-4.21 (m, 2H), 3.98 (d, 1H), | |
| | | | | | | | | 3.89-3.97 (m, 1H), 3.64-3.73 | |
| | | | | | | | | (m, 2H), 2.36-2.44 (m, 4H), 2.30 (s, | |
| | | | | | | | | 3H) | |
| 15 | 3-{[(2-{[(3R)-3-fluoropyrrolidin- | Ex. 3 | LCMS | 1.37 | | 473.2 | | (DMSO-d₆) 12.8-13.4 (m, 1H), | 0.0154 |
| | 1- | | Polar | | | | | 8.1-8.4 (m, 3H), 7.2-7.4 (m, 1H), | |
| | yl]sulfonyl}phenyl)amino]methyl}- | | | | | | | 6.82-7.05 (m, 2H), 6.28-6.6 (s, | |
| | 1-(5-methyl-7H-pyrrolo[2,3- | | | | | | | 1H), 3.7-4.42 (m, 5H), 3.45-3.65 | |
| | d]pyrimidin-4-yl)pyrrolidin-3- | | | | | | | (m, 2H), 2.04-2.4 (m, 2H). | |
| | amine (15) | | | | | | | | |
| 16 | 2-[4-({[3-amino-1-(5-methyl-7H- | Ex. 3 | LCMS | 1.87 | | 379.2 | | | 0.0143 |
| | pyrrolo[2,3-d]pyrimidin-4- | | Polar | | | | | | |
| | yl)pyrrolidin-3- | | | | | | | | |
| | yl]methyl}amino)phenyl]- | | | | | | | | |
| | acetamide (16) | | | | | | | | |
| 17 | 3-{[(4- | Ex. 3 | LCMS | 1.4 | | 378.1 | | | 0.331 |
| | chlorophenyl)amino]methyl}-1- | | Polar | | | | | | |
| | (1H-pyrrolo[2,3-b]pyridin-4- | | | | | | | | |
| | yl)pyrrolidin-3-amine (17) | | | | | | | | |
| 18 | (3S)-3-{[(4- | Ex. 3 | LCMS | 1.02 | | 357.1 | | | 0.002 |
| | chlorophenyl)amino]methyl}-1- | | STD | | | | | | |
| | (5-methyl-7H-pyrrolo[2,3- | | | | | | | | |
| | d]pyrimidin-4-yl)pyrrolidin-3- | | | | | | | | |
| | amine (18) | | | | | | | | |
| 19 | methyl 2-({[3-amino-1-(5- | Ex. 3 | LCMS | 1.26 | | 381.3 | | (500 MHz, methanol-d₄) δ 2.03 | 0.01 |
| | methyl-7H-pyrrolo[2,3- | | STD | | | | | (1H, m), 2.21 (1H, m), 2.38 (3H, | |
| | d]pyrimidin-4-yl)pyrrolidin-3- | | | | | | | s), 3.32 (2H, m), 3.67 (1H, d), 3.87 | |
| | yl[methyl}amino)benzoate (19) | | | | | | | (1H, d), 3.92 (1H, m), 4.03 (1H, q), | |
| | | | | | | | | 6.65 (2H, d), 6.93 (1H, s), 7.03 | |
| | | | | | | | | (2H, d), 8.08 (1H, s). | |
| 20 | 1-(5-methyl-7H-pyrrolo[2,3- | Ex. 3 | LCMS | 1.78 | | 415.3 | | | 0.0155 |
| | d]pyrimidin-4-yl)-3-{[(2- | | STD | | | | | | |
| | phenoxyphenyl)amino]methyl}- | | | | | | | | |
| | pyrrolidin-3-amine (20) | | | | | | | | |
| 21 | 3-{[(3-chloro-2- | Ex. 3 | LCMS | 1.35 | | 375.2 | | | 0.00553 |
| | fluorophenyl)amino]methyl}-1- | | STD | | | | | | |
| | (5-methyl-7H-pyrrolo[2,3- | | | | | | | | |
| | d]pyrimidin-4-yl)pyrrolidin-3- | | | | | | | | |
| | amine (21) | | | | | | | | |
| 22 | 4-({[3-amino-1-(5-methyl-7H- | Ex. 3 | LCMS | 1.22 | | 382.2 | | | 0.0161 |
| | pyrrolo[2,3-d]pyrimidin-4- | | STD | | | | | | |
| | yl)pyrrolidin-3-yl]methyl}amino)- | | | | | | | | |
| | 2-chlorobenzonitrile (22) | | | | | | | | |
| 23 | 1-(5-methyl-7H-pyrrolo[2,3- | Ex. 3 | LCMS | 1.52 | | 391.2 | | | 0.00669 |
| | d]pyrimidin-4-yl)-3-({[3- | | STD | | | | | | |
| | (trifluoromethyl)phenyl]amino}- | | | | | | | | |
| | methyl)pyrrolidin-3-amine (23) | | | | | | | | |
| 24 | 1-(5-methyl-7H-pyrrolo[2,3- | Ex. 3 | LCMS | 1.74 | | 406.3 | | (500 MHz, methanol-d₄) 2.28-2.33 | 0.0108 |
| | d]pyrimidin-4-yl)-3-{[(2- | | STD | | | | | (1H, m), 2.40 (3H, s), 2.43-2.49 | |
| | piperidin-1- | | | | | | | (1H, m), 3.57-3.66 (2H, q), 3.97- | |
| | ylphenyl)amino]methyl}pyrrol- | | | | | | | 4.14 (4H, m), 6.83 (1H, d), 6.89 | |
| | idin-3-amine (24) | | | | | | | (1H, d), 6.92 (2H, d), 7.21 (1H, t), | |
| | | | | | | | | 8.07 (1H, s) | |
| 25 | 1-(5-methyl-7H-pyrrolo[2,3- | Ex. 3 | LCMS | 1.7 | | 407.2 | | | 0.0145 |
| | d]pyrimidin-4-yl)-3-({[3- | | STD | | | | | | |
| | (trifluoromethoxy)phenyl]amino} | | | | | | | | |
| | methyl)pyrrolidin-3-amine (25) | | | | | | | | |
| 26 | 3-{[(2,3- | Ex. 3 | LCMS | 1.25 | | 391.1 | | | 0.002 |
| | dichlorophenyl)amino]methyl}- | | STD | | | | | | |
| | 1-(5-methyl-7H-pyrrolo[2,3- | | | | | | | | |
| | d]pyrimidin-4-yl)pyrrolidin-3- | | | | | | | | |
| | amine (26) | | | | | | | | |
| 27 | 2-({[3-amino-1-(5-methyl-7H- | Ex. 3 | LCMS | 1.26 | | 456.3 | | (500 MHz, methanol-d₄) 2.03-2.10 | 5.29 |
| | pyrrolo[2,3-d]pyrimidin-4- | | polar | | | | | (1H, m), 2.18-2.23 (1H, m), 2.33 | |
| | yl)pyrrolidin-3-yl]methyl}amimo)- | | | | | | | (3H, s), 3.29 (2H, t), 3.69 (1H, d), | |
| | N-benzylbenzamide (27) | | | | | | | 3.88 (1H, d), 3.89-3.93 (1H, m), | |
| | | | | | | | | 3.96-4.03 (1H, m), 6.69-6.74 (2H, | |
| | | | | | | | | m), 6.89 (1H, s), 6.95-6.70 (1H, t), | |
| | | | | | | | | 8.07 (1H, s) | |
| 28 | 3-{[(3-fluoro-2- | Ex. 3 | LCMS | 1.26 | | 355.3 | | (500 MHz, methanol-d₄) 8.29 (s, | 0.00952 |
| | methylphenyl)amino]methyl}-1- | | STD | | | | | 1H), 7.59 (d, 1H), 7.31-7.36 (m, | |
| | (5-methyl-7H-pyrrolo[2,3- | | | | | | | 5H), 7.25 (t, 1H), 7.19 (s, 1H), 6.96 | |
| | d]pyrimidin-4-yl)pyrrolidin-3- | | | | | | | (d, 1H), 6.74 (t, 1H), 4.55 (s, 2H) | |
| | amine (28) | | | | | | | 4.26 (d, 2H), 4.14-4.22 (m, 2H), | |
| | | | | | | | | 4.17 (d, 2H), 2.55-2.62 (m, 1H), | |
| | | | | | | | | 2.45-2.52 (m, 1H) 2.43 (s, 3H) | |
| 29 | 1-(5-methyl-7H-pyrrolo[2,3- | Ex. 3 | LCMS | 1.01 | | 408.3 | | | 0.0101 |
| | d]pyrimidin-4-yl)-3-{[(2- | | STD | | | | | | |
| | morpholin-4- | | | | | | | | |
| | ylphenyl)amino]methyl}pyrrol- | | | | | | | | |
| | idin-3-amine (29) | | | | | | | | |
| 30 | 3-({[3-methoxy-5- | Ex. 3 | LCMS | 1.21 | | 421.2 | | | 0.0135 |
| | (trifluoromethyl)phenyl]amino}- | | STD | | | | | | |
| | methyl)-1-(5-methyl-7H- | | | | | | | | |
| | pyrrolo[2,3-d]pyrimidin-4- | | | | | | | | |
| | yl)pyrrolidin-3-amine (30) | | | | | | | | |
| 31 | 3-{[(4-chloro-2- | Ex. 3 | LCMS | 1.26 | | 375.3 | | | 0.00514 |
| | fluorophenyl)amino]methyl}-1- | | STD | | | | | | |
| | (5-methyl-7H-pyrrolo[2,3- | | | | | | | | |
| | d]pyrimidin-4-yl)pyrrolidin-3- | | | | | | | | |
| | amine (31) | | | | | | | | |
| 32 | 3-{[(2,3- | Ex. 3 | LCMS | 1.09 | | 359.3 | | | 0.0105 |
| | difluorophenyl)amino]methyl}-1- | | STD | | | | | | |
| | (5-methyl-7H-pyrrolo[2,3- | | | | | | | | |
| | d]pyrimidin-4-yl)pyrrolidin-3- | | | | | | | | |
| | amine (32) | | | | | | | | |
| 33 | 3-({[2-(4-chlorophenoxy)-5- | Ex. 3 | LCMS | 2.21 | | 517.3 | | | 0.00859 |
| | (trifluoromethyl)phenyl]amino}- | | STD | | | | | | |
| | methyl)-1-(5-methyl-7H- | | | | | | | | |
| | pyrrolo[2,3-d]pyrimidin-4- | | | | | | | | |
| | yl)pyrrolidin-3-amine (33) | | | | | | | | |
| 34 | 3-{[(2,4- | Ex. 3 | LCMS | 1.09 | | 359.3 | | | 0.00771 |
| | difluorophenyl)amino]methyl}-1- | | STD | | | | | | |
| | (5-methyl-7H-pyrrolo[2,3- | | | | | | | | |
| | d]pyrimidin-4-yl)pyrrolidin-3- | | | | | | | | |
| | amine (34) | | | | | | | | |
| 35 | 1-(5-methyl-7H-pyrrolo[2,3- | Ex. 3 | LCMS | 1.48 | | 377.4 | | | 0.00797 |
| | d]pyrimidin-4-yl)-3-[(5,6,7,8- | | STD | | | | | | |
| | tetrahydronaphthalen-1- | | | | | | | | |
| | ylamino)methyl]pyrrolidin-3- | | | | | | | | |
| | amine (35) | | | | | | | | |
| 36 | (3S)-3-({[2-fluoro-3- | Ex. 3 | LCMS | 2.37 | | 409.2 | | | 0.002 |
| | (trifluoromethyl)phenyl]amino}- | | Polar | | | | | | |
| | methyl)-1-(5-methyl-7H- | | | | | | | | |
| | pyrrolo[2,3-d]pyrimidin-4- | | | | | | | | |
| | yl)pyrrolidin-3-amine (36) | | | | | | | | |
| 37 | (3S)-3-{[(3- | Ex. 3 | LCMS | 0.91 | | 341.1 | | (500 MHz, methanol-d₄) 1.96- | 0.00329 |
| | fluorophenyl)amino]methyl}-1- | | STD | | | | | 2.01 (1H, m), 2.12-2.17 (1H, m), | |
| | (5-methyl-7H-pyrrolo[2,3- | | | | | | | 2.35 (3H, s), 3.34-3.42 (2H, q), | |
| | d]pyrimidin-4-yl)pyrrolidin-3- | | | | | | | 3.62 (1H, d), 3.83 (1H, d), 3.88- | |
| | amine (37) | | | | | | | 3.92 (1H, m), 3.97-4.02 (1H, m), | |
| | | | | | | | | 6.76-6.79 (1H, t), 6.88 (1H, s), | |
| | | | | | | | | 6.97-7.03 (2H, q), 8.03 (1H, s) | |
| 38 | 4-{3-amino-3-[(5,6,7,8- | Ex. 4 | LCMS | 1.86 | | 388.2 | | (500 MHz, methanol-d₄) δ 8.32 (s, | 0.00371 |
| | tetrahydronaphthalen-1- | | STD | | | (386.2) | | 1H), 7.21 (s, 1H), 7.24 (s, 1H), | |
| | ylamino)methyl]pyrrolidin-1-yl}- | | | | | | | 7.08-7.13 (m, 1H), 6.57 (dd, 1H), | |
| | 7H-pyrrolo[2,3-d]pyrimidine-5- | | | | | | | 6.49 (dt, 1H), 6.39 (t, 1H), 4.14- | |
| | carbonitrile (38) | | | | | | | 4.25 (m, 4H) 3.66 (d, 2H), 4.17- | |
| | | | | | | | | 4.24 (m, 2H), 4.15 (d, 1H), 3.75- | |
| | | | | | | | | 3.83 (d, 2H), 2.56-2.62 (m, 1H), | |
| | | | | | | | | 2.45-2.51 (m, 1H) 2.44 (s, 3H) | |
| 39 | 4-{3-amino-3-[(1,2,3,4- | Ex. 4 | LCMS | 1.05 | | 389.3 | | | 4.1 |
| | tetrahydroisoquinolin-7- | | Polar | | | | | | |
| | ylamino)methyl]pyrrolidin-1-yl}- | | | | | | | | |
| | 7H-pyrrolo[2,3-d]pyrimidine-5- | | | | | | | | |
| | carbonitrile (39) | | | | | | | | |
| 40 | 4-[3-amino-3-({[2-fluoro-3- | Ex. 4 | LCMS | 1.79 | | 420.3 | | (500 MHz, DMSO-d₆) 2.21-2.31 | 0.00807 |
| | (trifluoromethyl)phenyl]amino}- | | STD | | | (418.3) | | (m, 1H) 2.33-2.44 (m, 1H) 2.83 | |
| | methyl)pyrrolidin-1-yl]-7H- | | | | | | | (t, J = 5.96Hz, 2H) 3.29-3.37 (m, | |
| | pyrrolo[2,3-d]pyrimidine-5- | | | | | | | 2H) 3.42-3.52 (m, 2H) 3.88 (d, 2 | |
| | carbonitrile (40) | | | | | | | H) 4.01-4.09 (m, 2H) 4.13 (s, 3 | |
| | | | | | | | | H) 5.98 (br. s., 1H) 6.48 (d, J = 2.07 | |
| | | | | | | | | Hz, 1H) 6.65 (dd, J = 8.29, 2.07Hz, | |
| | | | | | | | | 1H) 6.98 (d, 1H) 8.32 (d, J = 5.18 | |
| | | | | | | | | Hz, 2H) 8.92 (br. s., 2H) 12.91 (s, | |
| | | | | | | | | 1H) | |
| 41 | 3-{[(3-chloro-2- | Ex. 5 | LCMS | 1.1 | | 376.3 | | (500 MHz, methanol-d₄) 2.05- | 0.0134 |
| | fluorophenyl)amino]methyl}-1- | | STD | | | | | 2.19 (m, 1H) 2.19-2.38 (m, 1H) | |
| | (3-methyl-1H-pyrazolo[3,4- | | | | | | | 2.63 (t, J = 7.00Hz, 1H) 2.80 (t, | |
| | d]pyrimidin-4-yl)pyrrolidin-3- | | | | | | | J = 7.00Hz, 1H) 3.85 (d, J = 11.92 | |
| | amine (41) | | | | | | | Hz, 1H) 3.98 (d, J = 11.40Hz, 1H) | |
| | | | | | | | | 4.07-4.13 (m, 1H) 4.14-4.26 (m, | |
| | | | | | | | | 1H) 6.82 (t, J = 6.74Hz, 1H) 6.96- | |
| | | | | | | | | 7.18 (m, 2H) 7.97 (s, 1H) 8.21 (s, | |
| | | | | | | | | 1H) | |
| 42 | 1-(1H-pyrrolo[2,3-b]pyridin-4- | Ex. 5 | LCMS | 1.1 | | | | | 0.0354 |
| | yl)-3-({[3- | | Std | | | | | | |
| | (trifluoromethyl)phenyl]amino}- | | | | | | | | |
| | methyl)pyrrolidin-3-amine (42) | | | | | | | | |
| 43 | 3-{[(3-chloro-2- | Ex. 6 | LCMS | 2.59 | | 391.2 | | (500 MHz, methanol-d₄) 7.77 (d, | 0.002 |
| | methylphenyl)amino]methyl}-1- | | Polar | | | | | J = 5.70Hz, 1H), 7.23 (t, J = 7.78 | |
| | (5-chloro-7H-pyrrolo[2,3- | | | | | | | Hz, 1H), 7.03 (d, J = 3.11Hz, 1H), | |
| | d]pyrimidin-4-yl)pyrrolidin-3- | | | | | | | 6.94 (s, 1H), 6.91 (d, J = 8.29Hz, 1 | |
| | amine (43) | | | | | | | H), 6.84 (d, J = 7.78Hz, 1H), 6.67 | |
| | | | | | | | | (br. s., 1H), 6.08 (d, J = 5.18Hz, 1 | |
| | | | | | | | | H), 3.94 (m, 1H), 3.83 (m, 2H), | |
| | | | | | | | | 3.60 (d, J = 9.33Hz, 1H), 3.35 (s, 2 | |
| | | | | | | | | H), 2.18 (m, 1H), 2.00 (br. s., 1H) | |
| 44 | 1-(5-methyl-7H-pyrrolo[2,3- | Ex. 3 | LCMS | 0.74 | | 390.2 | | | 0.837 |
| | d]pyrimidin-4-yl)-3-({[3-(1,3- | | STD | | | | | | |
| | oxazol-5- | | | | | | | | |
| | yl)phenyl]amino}methyl)pyrrol- | | | | | | | | |
| | idin-3-amine (44) | | | | | | | | |
| 45 | 3-{[(4-chlorobenzyl)(methyl)- | Ex. 3 | LCMS | 1.19 | | 385 | | | 0.144 |
| | amino]methyl}-1-(5-methyl-7H- | | STD | | | | | | |
| | pyrrolo[2,3-d]pyrimidin-4- | | | | | | | | |
| | yl)pyrrolidin-3-amine (45) | | | | | | | | |
| 46 | N′-{[3-amino-1-(5-methyl-7H- | Ex. 3 | LCMS | 0.24 | | 318.1 | | | 0.125 |
| | pyrrolo[2,3-d]pyrimidin-4- | | STD | | | | | | |
| | yl)pyrrolidin-3-yl]methyl}-N,N- | | | | | | | | |
| | dimethylethane-1,2-diamine | | | | | | | | |
| | (46) | | | | | | | | |
| 47 | N-{[3-amino-1-(5-methyl-7H- | Ex. 3 | LCMS | 0.48 | | 408.1 | | | 0.172 |
| | pyrrolo[2,3-d]pyrimidin-4- | | STD | | | | | | |
| | yl)pyrrolidin-3-yl]methyl}-N- | | | | | | | | |
| | benzyl-N′,N′-dimethylethane- | | | | | | | | |
| | 1,2-diamine (47) | | | | | | | | |
| 48 | 3- | Ex. 3 | LCMS | 1.09 | | 337.3 | | | 0.12 |
| | {[methyl(phenyl)amino]methyl}- | | STD | | | | | | |
| | 1-(5-methyl-7H-pyrrolo[2,3- | | | | | | | | |
| | d]pyrimidin-4-yl)pyrrolidin-3- | | | | | | | | |
| | amine (48) | | | | | | | | |
| 49 | 3-(3,4-dihydroquinolin-1(2H)- | Ex. 3 | LCMS | 1.39 | | 363.3 | | | 0.131 |
| | ylmethyl)-1-(5-methyl-7H- | | STD | | | | | | |
| | pyrrolo[2,3-d]pyrimidin-4- | | | | | | | | |
| | yl)pyrrolidin-3-amine (49) | | | | | | | | |
| 50 | 3-{[ethyl(phenyl)amino]methyl}- | Ex. 3 | LCMS | 1.3 | | 351.3 | | | 0.116 |
| | 1-(5-methyl-7H-pyrrolo[2,3- | | STD | | | | | | |
| | d]pyrimidin-4-yl)pyrrolidin-3- | | | | | | | | |
| | amine (50) | | | | | | | | |
| 51 | 3-(2,3-dihydro-1H-indol-1- | Ex. 3 | LCMS | 1.29 | | 349.3 | | | 0.0362 |
| | ylmethyl)-1-(5-methyl-7H- | | STD | | | | | | |
| | pyrrolo[2,3-d]pyrimidin-4- | | | | | | | | |
| | yl)pyrrolidin-3-amine (51) | | | | | | | | |
| 52 | N-{[3-amino-1-(5-methyl-7H- | Ex. 1 | LCMS | 0.39 | | 359.3 | | | 0.082 |
| | pyrrolo[2,3-d]pyrimidin-4- | | STD | | | | | | |
| | yl)pyrrolidin-3-yl]methyl}-6- | | | | | | | | |
| | chloropyridazin-3-amine (52) | | | | | | | | |
| 53 | (3S)-3-({[(5-methylisoxazol-3- | Ex. 3 | LCMS | 0.39 | | 342.1 | | | 0.0293 |
| | yl)methyl]amino}methyl)-1-(5- | | STD | | | | | | |
| | methyl-7H-pyrrolo[2,3- | | | | | | | | |
| | d]pyrimidin-4-yl)pyrrolidin-3- | | | | | | | | |
| | amine (53) | | | | | | | | |
| 54 | 3-{[(cyclopropylmethyl) | Ex. 4 | LCMS | 0.1 | | 301.5 | 301.5 | (400 MHz, methanol-d₄) δ 8.35 (s, | 0.739 |
| | amino]methyl}-1-(5-methyl-7H- | | STD | | | | | 1H), 7.26 (s, 1H), 6.23 (s, 1H), | |
| | pyrrolo[2,3-d]pyrimidin-4- | | | | | | | 7.69-7.72 (m, 1H), 7.36 (d, 1H) | |
| | yl)pyrrolidin-3-amine (54) | | | | | | | 7.06 (d, 1H), 4.20 (d, 2H), 4.18- | |
| | | | | | | | | 4.23 (m, 1H), 4.11-4.16(m, 1H), | |
| | | | | | | | | 4.09 (s, 2H), 3.26 (s, 2H), 2.51 (s, | |
| | | | | | | | | 3H), 2.47-2.50 (m, 2H) 2.43 (s, 3H) | |
| 55 | 3-{[(4-chlorophenyl)(methyl)- | Ex. 3 | LCMS | 2.61 | | 391.3 | | (500 MHz, methanol-d₄) 8.07 (s, | 0.012 |
| | amino]methyl}-1-(5-chloro-7H- | | Polar | | | | | 1H), 6.95 (s, 1H), 4.04 (m, 1H), | |
| | pyrrolo[2,3-d]pyrimidin-4- | | | | | | | 3.88 (td, J = 9.59, 4.15Hz, 1H), | |
| | yl)pyrrolidin-3-amine (55) | | | | | | | 3.80 (m, 1H), 3.70 (d, J = 11.40Hz, | |
| | | | | | | | | 1H), 3.49 (q, J = 6.91Hz, 1H), | |
| | | | | | | | | 2.99 (s, 2H), 2.71 (d, J = 7.26Hz, | |
| | | | | | | | | 2H), 2.45 (s, 3H), 2.12 (ddd, | |
| | | | | | | | | J = 12.57, 8.55, 8.42Hz, 1H), 2.03 | |
| | | | | | | | | (m, 1H), 1.18 (t, J = 7.00Hz, 1H), | |
| | | | | | | | | 1.06 (m, J = 8.03, 8.03, 4.92, 3.89 | |
| | | | | | | | | Hz, 1H), 0.60 (q, J = 5.01Hz, 2H), | |
| | | | | | | | | 0.28 (q, J = 4.84Hz, 2H) | |
| 56 | 6-({[(3S)-3-amino-1-(5-methyl- | Ex. 1 | LCMS | 0.24 | | 340.2 | | | 0.014 |
| | 7H-pyrrolo[2,3-d]pyrimidin-4- | | STD | | | | | | |
| | yl)pyrrolidin-3- | | | | | | | | |
| | yl]methyl}amino)pyridin-2(3H)- | | | | | | | | |
| | one (56) | | | | | | | | |
| 57 | N-{[3-amino-1-(5-methyl-7H- | Ex. 2 | H | | 5.83 | | | (400 MHz, methanol-d₄) δ 8.34 (s, | 0.0287 |
| | pyrrolo[2,3-d]pyrimidin-4- | | | | | | | 1H), 7.44 (t, 1H), 7.24 (s, 1H), 6.11 | |
| | yl)pyrrolidin-3-yl]methyl}-4- | | | | | | | (d, 1H), 5.97 (d, 1H) 4.30 (d, 1H), | |
| | (trifluoromethyl)pyrimidin-2- | | | | | | | 4.17-4.24 (m, 2H), 4.15 (d, 1H), | |
| | amine (57) | | | | | | | 3.75-3.83(d, 2H), 2.55-2.61 (m, | |
| | | | | | | | | 1H), 2.48-2.52 (m, 1H) 2.46 (s, 3H) | |
| 58 | N-{[3-amino-1-(5-methyl-7H- | Ex. 2 | H | | 3.19 | | 382.3 | (DMSO-d₆) 11.32 (s, 1H), 8.52 | 0.123 |
| | pyrrolo[2,3-d]pyrimidin-4- | | | | | | (MH⁺) | (bs, 1H), 8.04 (s, 1H), 7.76-7.74 | |
| | yl)pyrrolidin-3- | | | | | | | (m, 2H), 6.93 (s, 1H), 3.70-3.32 | |
| | yl]methyl}pyrimidin-2-amine | | | | | | | (m, 6H), 2.26 (s, 3H), 2.05-1.97 | |
| | (58) | | | | | | | (m, 2H). | |
| 59 | N-{[3-amino-1-(1H-pyrrolo[2,3- | Ex. 2 | H | | 6.4 | | 394.3 | (DMSO-d₆) 11.30 (s, 1H), 8.49 | 0.0035 |
| | b]pyridin-4-yl)pyrrolidin-3- | | | | | | (MH⁺) | (bs, 1H), 8.02 (s, 1H), 7.76-7.74 | |
| | yl]methyl}-5-chloropyrimidin-2- | | | | | | | (m, 3H), 6.92 (s, 1H), 3.70-3.32 | |
| | amine (59) | | | | | | | (m, 8H), 2.26 (s, 3H), 2.05-1.97 | |
| | | | | | | | | (m, 4H). | |

TABLE 4
Examples 60 to 65.

| | | | | | | | | | Avg Akt |
|---|---|---|---|---|---|---|---|---|---|
| | | | LCMS/ | LCMS | HPLC | | APCl | | kinase, μM |
| | | | HPLC | RT, | RT, | MS | Acid | | (50% |
| Ex. | Name | Prep. | Method | min | min | Peak 1 | Basic | ¹H NMR δ, ppm | inhibition) |
| 60 | 41-(5-Methyl-7H-pyrrolo[2,3- | | | | | 392.4 | | (400 MHz, DMSO-d₆) 11.3 (s, 1H), | 0.0504 |
| | d]pyrimidin-4-yl)-3-(3- | | | | | | | 8.02 (s, 1H), 7.50 (t, 1H), 7.26 (m, | |
| | trifluoromethyl- | | | | | | | 3H), 6.92 (s, 1H), 4.04 (s, 2H), | |
| | phenoxymethyl)pyrrolidin-3- | | | | | | | 3.88 (m, 1H), 3.74 (m, 2H), 3.48 | |
| | ylamine (60) | | | | | | | (d, 1H), 2.30 (s, 3H), 2.05 (m, 1H), | |
| | | | | | | | | 1.84 (m, 3H). | |
| 61 | (3R)-1-(5-methyl-7H- | Ex. 60 | LCMS | 1.3 | | 392.4 | | (400 MHz, DMSO-d₆) 11.34 (s, | 0.0335 |
| | pyrrolo[2,3-d]pyrimidin-4-yl)-3- | | STD | | | | | 1H), 8.02 (s, 1H), 7.50 (t, 1H), 7.26 | |
| | {[3-(trifluoromethyl)- | | | | | | | (m, 3H), 6.92 (s, 1H), 4.04 (s, 2H), | |
| | phenoxy]methyl}pyrrolidin-3- | | | | | | | 3.88 (m, 1H), 3.74 (m, 2H), 3.48 | |
| | amine (61) | | | | | | | (d, 1H), 2.30 (s, 3H), 2.05 (m, 1H), | |
| | | | | | | | | 1.84 (m, 3H). | |
| 62 | (3S)-1-(5-methyl-7H- | Ex. 60 | LCMS | 1.3 | | 392.4 | | (400 MHz, DMSO-d₆) 11.34 (s, | 2.69 |
| | pyrrolo[2,3-d[pyrimidin-4-yl)-3- | | STD | | | | | 1H), 8.02 (s, 1H), 7.50 (t, 1H), 7.26 | |
| | {[3- | | | | | | | (m, 3H), 6.92 (s, 1H), 4.04 (s, 2H), | |
| | (trifluoromethyl)phenoxy]methyl} | | | | | | | 3.88 (m, 1H), 3.74 (m, 2H), 3.48 | |
| | pyrrolidin-3-amine (62) | | | | | | | (d, 1H), 2.30 (s, 3H), 2.05 (m, 1H), | |
| | | | | | | | | 1.84 (m, 3H). | |
| 63 | 4-(3-amino-3-{[3- | Ex. 60 | LCMS | 2.4 | | 403.4 | | (400 MHz, DMSO-d₆) 8.20 (d, 2H), | 0.0641 |
| | (trifluoromethyl)phenoxy]methyl} | | STD | | | | | 7.50 (t, 1H), 7.26 (m, 2H), 4.01 (m, | |
| | pyrrolidin-1-yl)-7H-pyrrolo[2,3- | | | | | | | 3H), 3.87 (m, 2H), 3.63 (d, 1H), | |
| | d]pyrimidine-5-carbonitrile (63) | | | | | | | 2.11 (m, 1H), 1.90 (m, 1H). | |
| 64 | 1-(5-ethyl-7H-pyrrolo[2,3- | Ex. 60 | LCMS | 2.5 | | 406.4 | | (400 MHz, DMSO-d₆) 11.40 (s, | 0.0215 |
| | d]pyrimidin-4-yl)-3-{]3- | | STD | | | | | 1H), 8.06 (s, 1H), 7.53 (t, 1H), 7.3 | |
| | (trifluoromethyl)phenoxy]methyl} | | | | | | | (m, 3H), 6.93 (s, 1H), 3.95 (dd, | |
| | pyrrolidin-3-amine (64) | | | | | | | 1H), 3.80 (d, 1H), 3.74 (m, 1H), | |
| | | | | | | | | 3.49 (d, 1H), 2.74 (q, 2H), 2.05 (m, | |
| | | | | | | | | 1H), 1.83 (m, 1H), 1.20 (t, 3H). | |
| 65 | 1-(5-ethyl-7H-pyrrolo[2,3- | Ex. 60 | LCMS | 2.4 | | 352.4 | | (400 MHz, DMSO-d₆) 11.38 (s, | 0.0948 |
| | d]pyrimidin-4-yl)-3-[(3- | | STD | | | | | 1H), 8.02 (s, 1H), 7.12 (t, 1H), 6.89 | |
| | methylphenoxy)methyl]pyrrol- | | | | | | | (s, 1H), 6.72 (t, 3H), 3.94-3.67 (m, | |
| | idin-3-amine (65) | | | | | | | 6H), 3.43 (d, 1H), 2.70 (q, 2H), | |
| | | | | | | | | 2.23 (s, 3H), 2.02 (m, 1H), 1.77 | |
| | | | | | | | | (m, 1H), 1.17 (t, 3H). | |

TABLE 5
Examples 66 to 198

| | | | | | | | | | Avg Akt kinase, μM |
|---|---|---|---|---|---|---|---|---|---|
| | | | LCMS/HPLC | | HPLC RT, | | | | (50% |
| Ex. | Name | Prep. | Method | LCMS RT, min | min | MS Peak 1 | APCI Acid Basic | ¹H NMR δ, ppm | inhibition) |
| 66 | 1-(5-methyl-7H-pyrrolo[2,3- | | LCMS | 1.2 | | 408.1 | | (400 MHz, DMSO-d₆) 11.28 (s, | 0.701 |
| | d]pyrimidin-4-yl)-3-((3- | | STD | | | | | 1H), 8.00 (s, 1H), 7.66 (s, 2H), | |
| | (trifluoromethyl)phenylthio)- | | | | | | | 7.47 (m, 2H), 6.90 (s, 1H), | |
| | methyl)-pyrrolidin-3-amine (66) | | | | | | | 3.83 (m, 1H), 3.6 (m, 2H), 3.42 (d, 1H), | |
| | | | | | | | | 3.34 (m, 2H), 2.17 (s, 3H), | |
| | | | | | | | | 1.95-1.87 (m, 4). | |
| 67 | 1-(5-methyl-7H-pyrrolo[2,3- | | LCMS | 1.4 | | 390.3 (236.8) | | (DMSO-d₆) 11.32 (s, 1H), 8.01 (s, | 0.126 |
| | d]pyrimidin-4-yl)-3-{2-[3- | | STD | | | | | 1H), 7.58-7.46 (m, 4H), 6.91 (s, 1H), | |
| | (trifluoromethyl)- | | | | | | | 3.87 (dd, J = 10.4, 7.0 Hz., 1H), | |
| | phenyl]ethyl}pyrrolidin-3-amine | | | | | | | 3.65 (dd, J = 10.8, 7.0 Hz., 1H), | |
| | (67) | | | | | | | 3.53 (d, J = 10.4 Hz., 1H), | |
| | | | | | | | | 3.43 (d, J = 10.8 Hz., 1H), | |
| | | | | | | | | 2.87-2.75 (m, 4H), 2.28 (s, 3H), | |
| | | | | | | | | 1.87-1.77 (m, 4H). ¹⁹F NMR (DMSO- | |
| | | | | | | | | d₆) δ, ppm: −61.31. | |
| 68 | (E)-3-(3-trifluoromethyl)styryl)- | | LCMS | 2.2 | | 320.3 | | (400 MHz, DMSO-d₆) 11.9 (s, 1H), | 1.14 |
| | 1-(5-methyl-7H-pyrrolo[2,3- | | STD | | | | | 8.55 (m, 2H), 8.23 (s, 1H), 7.49 (d, | |
| | d]pyrimidin-4-yl)-3-amine (68) | | | | | | | 2H), 7.39-7.15 (m, 4H), 6.80 (d, | |
| | | | | | | | | 1H), 6.54 (d, 1H), 4.10-3.82 (m, | |
| | | | | | | | | 6H), 2.37 (s, 3H). | |
| 69 | N-((3-amino-1-(5-methyl-7H- | | LCMS | 0.65 | | 351.3 | | | 0.0623 |
| | pyrrolo[2,3-d]pyrimidin-4- | | STD | | | | | | |
| | yl)pyrrolidin-3- | | | | | | | | |
| | yl)methyl)benzamide (69) | | | | | | | | |
| 70 | N-(((S)-3-amino-1-(5-chloro- | | LCMS | 2.21 | | 405.3 | | (500 MHz, methanol-d₄) | 0.00544 |
| | 7H-pyrrolo[2,3-d]pyrimidin-4- | | Polar | | | | | 1.97-1.99 (1H, m), 2.15-2.18 (1H, m), | |
| | yl)pyrrolidin-3-yl)methyl)-4- | | | | | | | 3.64 (2H, d), 3.76 (1H, d), 4.01 (1H, d), | |
| | chlorobenzamide (70) | | | | | | | 4.04-4.09 (2H, m), 7.18 (1H, s), | |
| | | | | | | | | 7.48 (1H, s), 7.49 (1H, s), | |
| | | | | | | | | 7.82 (1H, s), 7.84 (1H, s), 8.10 (1H, s) | |
| 71 | N-(((S)-3-amino-1-(5-methyl- | | LCMS | 1.85 | | 385.4 | | (500 MHz, methanol-d₄) | 0.00797 |
| | 7H-pyrrolo[2,3-d]pyrimidin-4- | | Polar | | | | | 1.95-2.01 (1H, m), 2.17-2.23 (1H, m), | |
| | yl)pyrrolidin-3-yl)methyl)-2- | | | | | | | 2.46 (3H, s), 3.58-3.67 ((3H, m), | |
| | chlorobenzamide (71) | | | | | | | 3.91-3.94 (2H, m), 4.02-4.05 (1H, m), | |
| | | | | | | | | 6.94 (1H, s), 7.39-7.41 (1H, m), | |
| | | | | | | | | 7.45-7.50 (2H, m), 8.07 (1H, s). | |
| 72 | N-{[(3S)-3-amino-1-(5-ethyl-7H- | | LCMS | 1.2 | | 331.2 | | | |
| | pyrrolo[2,3-d]pyrimidin-4- | | Basic | | | | | | |
| | yl)pyrrolidin-3-yl]methyl}-2- | | | | | | | | |
| | methylpropanamide (72) | | | | | | | | |
| 73 | N-{[(3S)-3-amino-1-(5-ethyl-7H- | | | | | 402.1 | | (500 MHz, methanol-d₄) 8.07 (s, 1H), | 0.000269 |
| | pyrrolo[2,3-d]pyrimidin-4- | | | | | | | 7.80 (q, J = 8.64 Hz, 1H), | |
| | yl)pyrrolidin-3-yl]methyl}-2,4- | | | | | | | 7.10 (q, J = 8.12 Hz, 2H), 6.94 (s, 1H), | |
| | difluorobenzamide (73 | | | | | | | 3.98 (m, 3H), 3.65 (m, 3H), | |
| | | | | | | | | 2.87 (q, J = 7.08 Hz, 2H), 2.17 (m, 1H), | |
| | | | | | | | | 1.97 (m, 1H), 1.30 (t, J = 7.26 Hz, 3H). | |
| 74 | (S)-N-((3-amino-1-(5-methyl- | | | | | 385.4 | | (500 MHz, methanol-d₄) 8.06 (s, 1H), | 0.0114 |
| | 7H-pyrrolo[2,3-d]pyrimidin-4- | | | | | | | 7.83 (d, 2H), 7.49 (d, J = 8.81 Hz, | |
| | yl)pyrrolidin-3-yl)methyl)-4- | | | | | | | 2H), 6.93 (s, 1H), | |
| | chlorobenzamide (74) | | | | | | | 3.86-4.05 (m, 3H), 3.60-3.70 (m, 3H), | |
| | | | | | | | | 2.45 (s, 3H), 2.13-2.21 (m, 1H), | |
| | | | | | | | | 1.96-2.03 (m, 1H). | |
| 75 | 1-{[3-amino-1-(5-methyl-7H- | | LCMS | 5.95 | | 394.6 | | (DMSO-d₆) 11.23 (s, 1H), 8.02 (s, | 0.185 |
| | pyrrolo[2,3-d]pyrimidin-4- | | Polar | | | | | 1H), 7.75 (bs, 1H), 7.48 (d, J = 7.9 Hz., | |
| | yl)pyrrolidin-3-yl]methyl}-3-(2,3- | | | | | | | 1H), 6.95-6.92 (m, 2H), | |
| | dimethylphenyl)urea (75) | | | | | | | 6.78 (d, J = 7.4 Hz., 1H), 6.67 (bs, | |
| | | | | | | | | 1H), 3.81-3.70 (m, 4H), 3.59 (d, J = 10.8 Hz., | |
| | | | | | | | | 1H), 3.32-3.25 (m, | |
| | | | | | | | | 3H), 3.19 (d, J = 5.8 Hz., 1H), | |
| | | | | | | | | 2.30 (s, 3H), 2.19 (s, 3H), 2.04 (s, | |
| | | | | | | | | 3H), 1.90-1.71 (m, 4H). | |
| 76 | 1-{[3-amino-1-(5-methyl-7H- | | LCMS | 1.54 | | 423.52 | | (500 MHz, methanol-d₄) | 0.523 |
| | pyrrolo[2,3-d]pyrimidin-4- | | Polar | | | | | 1.91-1.98 (1H, m), 2.02-2.14 (1H, m), | |
| | yl)pyrrolidin-3-yl]methyl}-1-(2- | | | | | | | 2.30 (3H, s), 3.38 (3H, s), | |
| | methoxyethyl)-3-phenylurea | | | | | | | 3.77-3.85 (7H, m), 3.88-3.92 (2H, m), | |
| | (76) | | | | | | | 3.96-4.01 (1H, m), 6.91 (1H, s), | |
| | | | | | | | | 6.96-7.00 (1H, m), 7.21-7.24 (2H, m), | |
| | | | | | | | | 7.26-7.29 (2H, m), 8.05 (1H, s). | |
| 77 | N-{[3-amino-1-(5-methyl-7H- | | LCMS | 0.70 | | 387.3 | | (500 MHz, methanol-d₄) | 0.177 |
| | pyrrolo[2,3-d]pyrimidin-4- | | STD | | | | | 2.39-2.64 (5H, m), 3.29 (2H, m), | |
| | yl)pyrrolidin-3- | | | | | | | 4.13-4.22 (4H, m), 7.22 (1H, s), | |
| | yl]methyl}benzenesulfonamide | | | | | | | 7.56-7.61 (2H, m), 7.63-7.65 (1H, m), | |
| | (77) | | | | | | | 7.88-7.90 (2H, m), 8.32 (1H, s). | |
| 78 | N-{[(3S)-3-amino-1-(5-chloro- | Ex. 69 | LCMS | 2.31 | | 389.1 | | 1H NMR (500 MHz, methanol-d₄) | 0.00267 |
| | 7H-pyrrolo[2,3-d]pyrimidin-4- | | Polar | | | | | 1.97-2.02 (1H, m), 2.14-2.20 (1H, | |
| | yl)pyrrolidin-3-yl]methyl}-4- | | | | | | | m), 3.64 (2H, d), 3.77 (1H, d), | |
| | fluorobenzamide (78) | | | | | | | 4.01 (1H, d), 4.03-4.11 (2H, m), | |
| | | | | | | | | 7.18-7.22 (3H, m), 7.89-7.92 (2H, q), | |
| | | | | | | | | 8.10 (1H, s) | |
| 79 | N-{[(3R)-3-amino-1-(5-chloro- | Ex. 69 | LCMS | 1.94 | | 376.5 | | (500 MHz, methanol-d₄) | 0.00377 |
| | 7H-pyrrolo[2,3-d]pyrimidin-4- | | Polar | | | | | 1.97-2.02 (1H, m), 2.14-2.20 (1H, m), | |
| | yl)pyrrolidin-3-yl]methyl}-5- | | | | | | | 3.64 (2H, d), 3.77 (1H, d), 4.01 (1H, d), | |
| | methylisoxazole-3-carboxamide | | | | | | | 4.03-4.11 (2H, m), 7.18-7.22 (3H, | |
| | (79) | | | | | | | m), 7.89-7.92 (2H, q), 8.10 (1H, s) | |
| 80 | N-{[(3S)-3-amino-1-(5-chloro- | Ex. 70 | LCMS | 1.97 | | 389.4 | | (500 MHz, methanol-d₄) | 0.00424 |
| | 7H-pyrrolo[2,3-d]pyrimidin-4- | | Polar | | | | | 1.96-1.99 (1H, m), 2.17-2.19 (1H, m), | |
| | yl)pyrrolidin-3-yl]methyl}-2- | | | | | | | 3.66 (2H, d), 3.78 (1H, d), 4.02 (1H, d), | |
| | fluorobenzamide (80) | | | | | | | 4.04-4.10 (2H, m), 7.19 (1H, s), | |
| | | | | | | | | 7.21-7.23 (1H, t), 7.25-7.29 (1H, t), | |
| | | | | | | | | 7.31 (1H, m), 7.72 (1H, t), | |
| | | | | | | | | 8.12 (1H, s) | |
| 81 | N-{[(3S)-3-amino-1-(5-chloro- | Ex. 70 | LCMS | 2.08 | | 407.3 | | (500 MHz, methanol-d₄) | 0.003464 |
| | 7H-pyrrolo[2,3-d]pyrimidin-4- | | Polar | | | | | 1.92-1.98 (1H, m), 2.10-2.16 (1H, m), | |
| | yl)pyrrolidin-3-yl]methyl}-2,3- | | | | | | | 3.62 (2H, d), 3.73 (1H, d), 3.96 (1H, d), | |
| | difluorobenzamide (81) | | | | | | | 3.97-4.10 (2H, m), 7.14 (1H, s), | |
| | | | | | | | | 7.20-7.25 (1H, m), 7.35-7.45 (2H, | |
| | | | | | | | | m), 8.07 (1H, s) | |
| 82 | N-{[(3S)-3-amino-1-(5-chloro- | Ex. 70 | LCMS | 2.01 | | 407.3 | | (500 MHz, methanol-d₄) | 0.00163 |
| | 7H-pyrrolo[2,3-d]pyrimidin-4- | | Polar | | | | | 1.92-1.99 (1H, m), 2.10-2.19 (1H, m), | |
| | yl)pyrrolidin-3-yl]methyl}-2,4- | | | | | | | 3.61 (2H, s), 3.73 (1H, d), 3.98 (1H, d), | |
| | difluorobenzamide (82) | | | | | | | 4.03-4.06 (2H, m), 7.05-7.08 (2H, | |
| | | | | | | | | m), 7.16 (1H, s), 7.75-7.76 (1H, | |
| | | | | | | | | m), 8.08 (1H, s) | |
| 83 | N-{[(3S)-3-amino-1-(5-chloro- | Ex. 70 | LCMS | 1.5 | | 423.3 | | (500 MHz, methanol-d₄) | 0.00915 |
| | 7H-pyrrolo[2,3-d]pyrimidin-4- | | STD | | | | | 1.97-2.02 (1H, m), 2.13-2.19 (1H, m), | |
| | yl)pyrrolidin-3-yl]methyl}-4- | | | | | | | 3.63 (2H, s), 3.76 (1H, d), 4.01 (1H, d), | |
| | chloro-3-fluorobenzamide (83) | | | | | | | 4.03-4.11 (2H, m), 7.19 (1H, s), | |
| | | | | | | | | 7.58-7.62 (1H, t), 7.66-7.68 (1H, | |
| | | | | | | | | dd), 7.72-7.75 (1H, dd), 8.10 (1H, | |
| | | | | | | | | s) | |
| 84 | N-{[(3S)-3-amino-1-(5-chloro- | Ex. 70 | LCMS | 1.74 | | 423.1 | | (500 MHz, methanol-d₄) | 0.01109 |
| | 7H-pyrrolo[2,3-d]pyrimidin-4- | | Polar | | | | | 1.96-2.01 (1H, m), 2.13-2.19 (1H, m), | |
| | yl)pyrrolidin-3-yl]methyl}-4- | | | | | | | 3.65 (2H, s), 3.77 (1H, d), 4.01 (1H, d), | |
| | chloro-2-fluorobenzamide (84) | | | | | | | 4.03-4.13 (2H, m), 7.19 (1H, s), | |
| | | | | | | | | 7.33-7.38 (2H, m), 7.70-7.73 (1H, | |
| | | | | | | | | t), 8.11 (1H, s) | |
| 85 | N-{[(3S)-3-amino-1-(5-chloro- | Ex. 70 | LCMS | 1.18 | | 406.3 | | (400 MHz, methanol-d₄) | 0.006435 |
| | 7H-pyrrolo[2,3-d]pyrimidin-4- | | STD | | | | | 1.93-1.99 (1H, m), 2.14-2.20 (1H, m), | |
| | yl)pyrrolidin-3-yl]methyl}-2- | | | | | | | 3.59 (2H, s), 3.76 (1H, d), | |
| | chlorobenzamide (85) | | | | | | | 3.98-4.12 (3H, m), 7.17 (1H, s), | |
| | | | | | | | | 7.34-7.48 (3H, m), 8.09 (1H, s) | |
| 86 | N-{[(3S)-3-amino-1-(5-cyano- | Ex. 69 | | | | | 396.4 (M + 1) | (500 MHz, methanol-d₄) 8.21 (s, 1H), | 0.0175 |
| | 7H-pyrrolo[2,3-d]pyrimidin-4- | | | | | | | 7.98 (s, 1H), 7.86 (d, J = 8.29 Hz, | |
| | yl)pyrrolidin-3-yl]methyl}-4- | | | | | | | 2H), 7.49 (d, J = 6.74 Hz, 2H), | |
| | chlorobenzamide (86) | | | | | | | 4.14 (m, 2H), 4.03 (d, J = 11.40 Hz, | |
| | | | | | | | | 1H), 3.86 (d, J = 11.92 Hz, 1H), | |
| | | | | | | | | 3.70 (s, 2H), 2.29 (m, 1H), | |
| | | | | | | | | 2.13 (m, 1H) | |
| 87 | N-{[(3S)-3-amino-1-(5-methyl- | Ex. 71 | H | | 5.07 | | 387.2 (M + 1) | (DMSO-d₆) 11.31 (bs, 1H), | 0.007575 |
| | 7H-pyrrolo[2,3-d]pyrimidin-4- | | | | | | | 8.60 (t, J = 5.7 Hz., 1H), 8.05 (s, 1H), | |
| | yl)pyrrolidin-3-yl]methyl}-2,3- | | | | | | | 7.59-7.53 (m, 1H), 7.425-7.39 (m, | |
| | difluorobenzamide (87) | | | | | | | 1H), 7.31-7.27 (m, 1H), 6.95 (s, | |
| | | | | | | | | 1H), 3.89-3.84 (m, 1H), | |
| | | | | | | | | 3.75-3.71 (m, 1H), 3.68 (d, J = 10.9 Hz., 1H), | |
| | | | | | | | | 3.46-3.43 (m, 3H), 2.33 (s, 3H), | |
| | | | | | | | | 1.98-1.74 (m, 4H). ¹⁹F | |
| | | | | | | | | NMR (DMSO-d₆) δ, ppm: −138.9, −141.1 | |
| 88 | N-{[(3S)-3-amino-1-(5-ethyl-7H- | Ex. 69 | | | | | 399.4 (M + 1) | (500 MHz, methanol-d₄) 8.05 (s, 1H), | 0.0018 |
| | pyrrolo[2,3-d]pyrimidin-4- | | | | | | | 7.83 (d, J = 8.29 Hz, 2H), | |
| | yl)pyrrolidin-3-yl]methyl}-4- | | | | | | | 7.49 (d, J = 8.29 Hz, 2H), 6.93 (s, 1H), | |
| | chlorobenzamide (88) | | | | | | | 3.85-4.05 (m, 3H), | |
| | | | | | | | | 3.57-3.67 (m, 3H), 2.79-2.91 (m, 2H), | |
| | | | | | | | | 2.10-2.20 (m, 1H), 1.90-1.98 (m, 1H), | |
| | | | | | | | | 1.28 (t, J = 7.26 Hz, 3H) | |
| 89 | N-{[(3S)-3-amino-1-(5-ethyl-7H- | Ex. 71 | H | | 5.6 | | 401.2 (M + 1) | (DMSO-d₆) 11.35 (bs, 1H), | 0.002775 |
| | pyrrolo[2,3-d]pyrimidin-4- | | | | | | | 8.60 (t, J = 5.7 Hz., 1H), 8.05 (s, 1H), | |
| | yl)pyrrolidin-3-yl]methyl}-2,3- | | | | | | | 7.59-7.547 (m, 1H), 7.42-7.39 (m, | |
| | difluorobenzamide (89) | | | | | | | 1H), 7.31-2.27 (m, 1H), 6.91 (s, | |
| | | | | | | | | 1H), 3.93-3.87 (m, 1H), | |
| | | | | | | | | 3.74-3.69 (m, 1H), 3.70 (d, J = 10.9 Hz., 1H), | |
| | | | | | | | | 3.46-3.38 (m, 3H), 2.76 (q, J = 7.3 Hz., | |
| | | | | | | | | 2H), 1.96-1.73 (m, 4H), | |
| | | | | | | | | 1.18 (t, J = 7.3 Hz., 3H). ¹⁹F | |
| | | | | | | | | NMR (DMSO-d₆) δ, ppm: −139.0, | |
| | | | | | | | | −141.1. | |
| 90 | N-{[(3S)-3-amino-1-(5-cyano- | Ex. 71 | | | | | 398.0 (M + 1) | (DMSO-d₆) 8.59 (t, J = 5.5 Hz., | 0.03945 |
| | 7H-pyrrolo[2,3-d]pyrimidin-4- | | | | | | | 1H), 8.24 (s, 1H), 8.21 (s, 1H), | |
| | yl)pyrrolidin-3-yl]methyl}-2,3- | | | | | | | 7.59-7.53 (m, 1H), 7.44-7.41 (m, | |
| | difluorobenzamide (90) | | | | | | | 1H), 7.31-7.27 (m, 1H), | |
| | | | | | | | | 4.02-3.99 (m, 1H), 3.72 (d, J = 10.9 Hz., 1H), | |
| | | | | | | | | 3.48-3.46 (m, 2H), 2.07-1.85 (m, | |
| | | | | | | | | 4H). ¹⁹F NMR (DMSO-d₆) δ, | |
| | | | | | | | | ppm: −138.9, −141.0. | |
| 91 | N-{[(3R)-3-amino-1-(5-ethyl-7H- | Ex. 71 | LCMS | 2.0 | | 370.4 | | (400 MHz, methanol-d₄) | 0.003915 |
| | pyrrolo[2,3-d]pyrimidin-4- | | Polar | | | | | 1.21-1.25 (3H, t), 1.85-1.92 (1H, m), | |
| | yl)pyrrolidin-3-yl]methyl}-5- | | | | | | | 2.04-2.11 (1H, m), 2.43 (3H, s), | |
| | methylisoxazole-3-carboxamide | | | | | | | 2.78-2.81 (2H, q), 3.51-3.60 (3H, m), | |
| | (91) | | | | | | | 3.85 (1H, d), 3.86-3.97 (2H, m), | |
| | | | | | | | | 6.40 (1H, s), 6.87 (1H, s), | |
| | | | | | | | | 8.01 (1H, s) | |
| 92 | N-{[(3S)-3-amino-1-(5-methyl- | Ex. 71 | B | | 2.72 | | 386.2 (M + 1) | (DMSO-d₆) 11.3 (bs, 1H), 8.56 (t, | 0.00303 |
| | 7H-pyrrolo[2,3-d]pyrimidin-4- | | | | | | | J = 5.7 Hz., 1H), 8.04 (s, 1H), | |
| | yl)pyrrolidin-3-yl]methyl}-4- | | | | | | | 7.94 (dd, J = 8.8, 5.7 Hz., 2H), 7.30 (dd, | |
| | fluorobenzamide (92) | | | | | | | J = 8.8, 8.8 Hz., 2H), 6.93 (s, 1H), | |
| | | | | | | | | 3.88-3.82 (m, 1H), 3.75-3.71 (m, | |
| | | | | | | | | 1H), 3.68 (d, J = 10.9 Hz., 1H), | |
| | | | | | | | | 3.47 (m, 3H), 2.31 (s, 3H), | |
| | | | | | | | | 1.95-1.73 (m, 4H). ¹⁹F NMR (DMSO- | |
| | | | | | | | | d₆) δ, ppm: −110.0. | |
| 93 | N-{[(3S)-3-amino-1-(5-ethyl-7H- | Ex. 71 | B | | 1.51 | | 383.3 (M + 1) | (DMSO-d₆) 11.3 (bs, 1H), 8.57 (t, | 0.001905 |
| | pyrrolo[2,3-d]pyrimidin-4- | | | | | | | J = 5.9 Hz., 1H), 8.04 (s, 1H), | |
| | yl)pyrrolidin-3-yl]methyl}-4- | | | | | | | 7.95 (dd, J = 8.8, 5.7 Hz., 2H), 7.31 (dd, | |
| | fluorobenzamide (93) | | | | | | | J = 8.8, 8.8 Hz., 2H), 6.90 (s, 1H), | |
| | | | | | | | | 3.91-3.86 (m, 1H), 3.73-3.71 (m, | |
| | | | | | | | | 1H), 3.70 (d, J = 10.3 Hz., 1H), | |
| | | | | | | | | 3.46-3.34 (m, 3H), 2.75 (q, J = 7.3 Hz., | |
| | | | | | | | | 2H), 1.97-1.71 (m, 4H), | |
| | | | | | | | | 1.18 (t, J = 7.3 Hz., 3H). ¹⁹F | |
| | | | | | | | | NMR (DMSO-d₆) δ, ppm: −110.0. | |
| 94 | N-{[(3S)-3-amino-1-(5-cyano- | Ex. 71 | B | | 1.53 | | 380.2 (M + 1) | (DMSO-d₆) 8.53 (t, J = 5.7 Hz., | 0.009225 |
| | 7H-pyrrolo[2,3-d]pyrimidin-4- | | | | | | | 1H), 8.22 (s, 1H), 8.19 (s, 1H), | |
| | yl)pyrrolidin-3-yl]methyl}-4- | | | | | | | 7.95 (dd, J = 8.8, 5.7 Hz., 2H), | |
| | fluorobenzamide (94) | | | | | | | 7.30 (dd, J = 8.8, 8.8 Hz., 2H), | |
| | | | | | | | | 3.93-3.90 (m, 1H), 3.71 (d, J = 11.4 Hz., | |
| | | | | | | | | 1H), 3.57 (d, J = 11.4 Hz., | |
| | | | | | | | | 1H), 3.47 (d, J = 5.7 Hz., 2H), | |
| | | | | | | | | 3.48-3.46 (m, 1H), 2.05-1.83 (m, | |
| | | | | | | | | 4H). ¹⁹F NMR (DMSO-d₆) δ, ppm: | |
| | | | | | | | | −110.0. | |
| 95 | N-{[(3R)-3-amino-1-(5-propyl- | | LCMS | 1.2 | | 384.4 | | (400 MHz, methanol-d₄) | 0.00433 |
| | 7H-pyrrolo[2,3-d]pyrimidin-4- | | Polar | | | | | 0.88-0.93 (3H, t), 1.58-1.67 (2H, q), | |
| | yl)pyrrolidin-3-yl]methyl}-5- | | | | | | | 1.86-1.93 (1H, m), 2.05-2.12 (1H, m), | |
| | methylisoxazole-3-carboxamide | | | | | | | 2.45 (3H, s), 2.70-2.79 (2H, m), | |
| | (95) | | | | | | | 3.50-3.61 (3H, m), 3.83 (1H, d), | |
| | | | | | | | | 3.85-3.97 (2H, m), 6.41 (1H, s), | |
| | | | | | | | | 6.88 (1H, s), 8.01 (1H, s) | |
| 96 | N-{[(3S)-3-amino-1-(5-cyano- | Ex. 69 | LCMS | 1.01 | | 398.4 | | (500 MHz, methanol-d₄) 8.22 (s, 1H), | 0.00771 |
| | 7H-pyrrolo[2,3-d]pyrimidin-4- | | STD | | | | | 7.99 (s, 1H), 7.83 (m, 1H), | |
| | yl)pyrrolidin-3-yl]methyl}-2,4- | | | | | | | 7.10 (m, 2H), 4.15 (m, 2H), | |
| | difluorobenzamide (96) | | | | | | | 4.02 (d, J = 11.92 Hz, 1H), 3.85 (d, | |
| | | | | | | | | J = 11.40 Hz, 1H), 3.70 (q, J = 13.82 Hz, | |
| | | | | | | | | 2H), 2.28 (m, 1H), 2.12 (m, 1H) | |
| 97 | N-{[(3S)-3-amino-1-(5-methyl- | Ex. 69 | LCMS | 0.5 | | 387.4 | | (500 MHz, methanol-d₄) 8.07 (s, 1H), | 0.000979 |
| | 7H-pyrrolo[2,3-d]pyrimidin-4- | | STD | | | | | 7.79 (m, 1H), 7.11 (m, 2H), | |
| | yl)pyrrolidin-3-yl]methyl}-2,4- | | | | | | | 6.94 (s, 1H), 4.02 (t, J = 9.07 Hz, 1H), | |
| | difluorobenzamide (97) | | | | | | | 3.93 (m, 2H), 3.66 (m, 3H), | |
| | | | | | | | | 2.44 (s, 3H), 2.18 (m, 1H), | |
| | | | | | | | | 1.99 (m, 1H) | |
| 98 | N-{[(3S)-3-amino-1-(5-ethyl-7H- | | LCMS | 0.49 | | 383.4 | | (400 MHz, methanol-d₄) | 0.000683 |
| | pyrrolo[2,3-d]pyrimidin-4- | | STD | | | | | 1.24-1.28 (3H, t), 1.90-1.95 (1H, m), | |
| | yl)pyrrolidin-3-yl]methyl}-2- | | | | | | | 2.09-2.16 (1H, m), 2.81-2.87 (2H, q), | |
| | fluorobenzamide (98) | | | | | | | 3.56-3.65 (3H, m), 3.88-4.02 (3H, | |
| | | | | | | | | m), 6.91 (1H, s), 7.18-7.28 (2H, | |
| | | | | | | | | m), 7.49-7.53 (1H, m), | |
| | | | | | | | | 7.67-7.71 (1H, m), 8.03 (1H, s) | |
| 99 | N-{[(3S)-3-amino-1-(5-cyano- | Ex. 69 | LCMS | 1.11 | | 414.4 | | (400 MHz, methanol-d₄) δ: 8.15 (s, | 0.0186 |
| | 7H-pyrrolo[2,3-d]pyrimidin-4- | | STD | | | | | 1H), 7.91 (s, 1H), 7.71 (t, J = 8.31 Hz, | |
| | yl)pyrrolidin-3-yl]methyl}-4- | | | | | | | 1H), 7.30 (m, 2H), 4.06 (m, 2H), | |
| | chloro-2-fluorobenzamide (99) | | | | | | | 3.92 (d, J = 11.22 Hz, 1H), | |
| | | | | | | | | 3.75 (d, J = 11.63 Hz, 1H), 3.64 (q, | |
| | | | | | | | | J = 13.71 Hz, 2H), 2.19 (m, 1H), | |
| | | | | | | | | 2.02 (m, 1H) | |
| 100 | N-{[(3S)-3-amino-1-(5-methyl- | Ex. 69 | LCMS | 0.74 | | 402.9 | | (400 MHz, methanol-d₄) 8.04 (s, 1H), | 0.00972 |
| | 7H-pyrrolo[2,3-d]pyrimidin-4- | | STD | | | | | 7.53 (dd, J = 8.72, 5.82 Hz, 1H), | |
| | yl)pyrrolidin-3-yl]methyl}-2- | | | | | | | 7.30 (dd, 1H), 7.15 (dt, 1H), | |
| | chloro-4-fluorobenzamide (100) | | | | | | | 6.91 (s, 1H), 4.00 (m, 1H), | |
| | | | | | | | | 3.90 (m, 2H), 3.60 (m, 3H), 2.42 (s, 3H), | |
| | | | | | | | | 2.18 (m, 1H), 1.96 (m, 1H) | |
| 101 | N-{[(3S)-3-amino-1-(5-methyl- | Ex. 69 | LCMS | 0.39 | | 387.2 | | (400 MHz, methanol-d₄) 8.03 (s, 1H), | 0.015 |
| | 7H-pyrrolo[2,3-d]pyrimidin-4- | | STD | | | | | 7.43 (m, 1H), 7.25 (m, 2H), | |
| | yl)pyrrolidin-3-yl]methyl}-2,5- | | | | | | | 6.90 (s, 1H), 3.93 (m, 4H), | |
| | difluorobenzamide (101) | | | | | | | 3.62 (m, 4H), 2.40 (s, 3H), 2.15 (m, 1H), | |
| | | | | | | | | 1.96 (m, 1H) | |
| 102 | N-{[(3S)-3-amino-1-(5-methyl- | Ex. 69 | LCMS | 0.71 | | 403.4 | | (500 MHz, methanol-d₄) 8.07 (s, 1H), | 0.014 |
| | 7H-pyrrolo[2,3-d]pyrimidin-4- | | STD | | | | | 7.66-7.78 (m, 1H), | |
| | yl)pyrrolidin-3-yl]methyl}-4- | | | | | | | 7.31-7.41 (m, 2H), 6.94 (s, 1H), | |
| | chloro-2-fluorobenzamide (102) | | | | | | | 3.97-4.10 (m, 1H), 3.85-3.98 (m, 2H), | |
| | | | | | | | | 3.59-3.70 (m, 3H), 2.44 (s, 3H), | |
| | | | | | | | | 2.09-2.25 (m, 1H), | |
| | | | | | | | | 1.93-2.03 (m, 1H) | |
| 103 | N-{[(3S)-3-amino-1-(5-ethyl-7H- | Ex. 69 | LCMS | 0.86 | | 417.4 | | (500 MHz, methanol-d₄) 8.06 (s, 1H), | 0.00487 |
| | pyrrolo[2,3-d]pyrimidin-4- | | STD | | | | | 7.62-7.79 (m, 1H), | |
| | yl)pyrrolidin-3-yl]methyl}-4- | | | | | | | 7.21-7.44 (m, 2H), 6.94 (s, 1H), | |
| | chloro-2-fluorobenzamide (103) | | | | | | | 3.81-4.09 (m, 3H), 3.51-3.72 (m, 3H), | |
| | | | | | | | | 2.87 (q, J = 7.43 Hz, 2H), | |
| | | | | | | | | 2.08-2.21 (m, 1H), 1.88-2.03 (m, 1H), | |
| | | | | | | | | 1.30 (t, J = 7.26 Hz, 3H) | |
| 104 | N-{[(3S)-3-amino-1-(5-chloro- | Ex. 71 | LCMS | 2.2 | | 389.4 | | (400 MHz, methanol-d₄) | 0.00674 |
| | 7H-pyrrolo[2,3-d]pyrimidin-4- | | Polar | | | | | 1.92-1.98 (1H, m), 2.09-2.16 (1H, m), | |
| | yl)pyrrolidin-3-yl]methyl}-3- | | | | | | | 3.60 (2H, s), 3.72 (1H, d), | |
| | fluorobenzamide (104) | | | | | | | 3.91-4.07 (3H, m), 7.13 (1H, s), | |
| | | | | | | | | 7.23-7.28 (1H, m), 7.43-7.49 (1H, m), | |
| | | | | | | | | 7.55-7.57 (1H, m), 7.62-7.65 (1H, m), | |
| | | | | | | | | 8.05 (1H, s) | |
| 105 | N-{[(3S)-3-amino-1-(5-ethyl-7H- | Ex. 71 | LCMS | 0.52 | | 383.4 | | (400 MHz, methanol-d₄) | 0.0107 |
| | pyrrolo[2,3-d]pyrimidin-4- | | Polar | | | | | 1.22-1.26 (3H, t), 1.88-1.94 (1H, m), | |
| | yl)pyrrolidin-3-yl]methyl}-3- | | | | | | | 2.07-2.14 (1H, m), 2.78-2.84 (2H, q), | |
| | fluorobenzamide (105) | | | | | | | 3.54-3.63 (3H, m), 3.88-3.98 (3H, | |
| | | | | | | | | m), 6.89 (1H, s), 7.24-7.29 (1H, | |
| | | | | | | | | m), 7.43-7.49 (1H, m), | |
| | | | | | | | | 7.54-7.58 (1H, m), 7.63-7.66 (1H, m), | |
| | | | | | | | | 8.01 (1H, s) | |
| 106 | N-{[(3S)-3-amino-1-(5-chloro- | Ex. 71 | LCMS | 2.1 | | 407.4 | | (500 MHz, methanol-d₄) 8.12 (s, 1H), | 0.0025 |
| | 7H-pyrrolo[2,3-d]pyrimidin-4- | | Polar | | | | | 7.41-7.50 (m, 1H), | |
| | yl)pyrrolidin-3-yl]methyl}-2,5- | | | | | | | 7.22-7.33 (m, 2H), 7.19 (s, 1H), | |
| | difluorobenzamide (106) | | | | | | | 3.94-4.17 (m, 4H), 3.78 (d, J = 11.40 Hz, | |
| | | | | | | | | 1H), 3.65 (br. s., 2H), | |
| | | | | | | | | 2.12-2.22 (m, 1H), 1.94-2.04 (m, 1H) | |
| 107 | N-{[(3S)-3-amino-1-(5-chloro- | Ex. 71 | LCMS | 2.2 | | 423.4 | | (500 MHz, methanol-d₄) 8.12 (s, 1H), | 0.0011 |
| | 7H-pyrrolo[2,3-d]pyrimidin-4- | | polar | | | | | 7.56 (dd, J = 8.55, 5.96 Hz, 1H), | |
| | yl)pyrrolidin-3-yl]methyl}-2- | | | | | | | 7.33 (dd, J = 8.81, 2.59 Hz, 1H), | |
| | chloro-4-fluorobenzamide (107) | | | | | | | 7.10-7.24 (m, 2H), | |
| | | | | | | | | 3.98-4.20 (m, 3H), 3.79 (d, J = 11.92 Hz, | |
| | | | | | | | | 1H), 3.62 (s, 2H), 3.36 (s, 2H), | |
| | | | | | | | | 2.13-2.27 (m, 1H), | |
| | | | | | | | | 1.92-2.03 (m, 1H) | |
| 108 | N-{[(3S)-3-amino-1-(5-chloro- | Ex. 71 | LCMS | 2.1 | | 371.4 | | (500 MHz, methanol-d₄) 8.12 (s, 1H), | 0.00251 |
| | 7H-pyrrolo[2,3-d]pyrimidin-4- | | polar | | | | | 7.85 (d, J = 7.78 Hz, 2H), | |
| | yl)pyrrolidin-3- | | | | | | | 7.56 (t, J = 6.74 Hz, 1H), 7.43-7.52 (m, | |
| | yl]methyl}benzamide (108) | | | | | | | 2H), 7.19 (s, 1H), 3.96-4.19 (m, | |
| | | | | | | | | 3H), 3.80 (d, J = 11.92 Hz, 1H), | |
| | | | | | | | | 3.66 (s, 2H), 3.34 (s, 2H), | |
| | | | | | | | | 2.13-2.24 (m, 1H), 1.97-2.07 (m, 1H) | |
| 109 | N-{[(3S)-3-amino-1-(5-methyl- | Ex. 71 | LCMS | 2.05 | | 351.4 | | (400 MHz, methanol-d₄) 8.03 (s, 1H), | 0.00586 |
| | 7H-pyrrolo[2,3-d]pyrimidin-4- | | polar | | | | | 7.82 (d, J = 7.48 Hz, 2H), | |
| | yl)pyrrolidin-3- | | | | | | | 7.53 (t, J = 7.06 Hz, 1H), 7.40-7.49 (m, | |
| | yl]methyl}benzamide (109) | | | | | | | 2H), 6.90 (s, 1H), 3.85-4.05 (m, | |
| | | | | | | | | 3H), 3.54-3.68 (m, 3H), 3.31 (s, | |
| | | | | | | | | 2H), 2.39 (s, 3H), 2.08-2.19 (m, | |
| | | | | | | | | 1H), 1.92-2.01 (m, 1H) | |
| 110 | N-{[(3S)-3-amino-1-(5-ethyl-7H- | Ex. 71 | LCMS | 2.0 | | 365.5 | | (400 MHz, methanol-d₄) 8.02 (s, 1H), | 0.0073 |
| | pyrrolo[2,3-d]pyrimidin-4- | | polar | | | | | 7.82 (d, J = 7.06 Hz, 2H), | |
| | yl)pyrrolidin-3- | | | | | | | 7.51-7.56 (m, 1H), 7.45 (t, J = 7.48 Hz, 2H), | |
| | yl]methyl}benzamide (110) | | | | | | | 6.90 (s, 1H), 3.84-4.04 (m, 3H), | |
| | | | | | | | | 3.53-3.69 (m, 3H), 2.83 (q, | |
| | | | | | | | | J = 7.20 Hz, 2H), 2.09-2.16 (m, 1H), | |
| | | | | | | | | 1.88-1.92 (m, 1H), 1.25 (t, | |
| | | | | | | | | J = 7.48 Hz, 3H) | |
| 111 | N-{[(3S)-3-amino-1-(5-cyano- | Ex. 71 | LCMS | 2.04 | | 362.4 | | (400 MHz, methanol-d₄) δ 8.16 (s, | 0.0265 |
| | 7H-pyrrolo[2,3-d]pyrimidin-4- | | polar | | | | | 1H), 7.93 (s, 1H), 7.82 (d, J = 7.48 Hz, | |
| | yl)pyrrolidin-3- | | | | | | | 2H), 7.36-7.57 (m, 3H), | |
| | yl]methyl}benzamide (111) | | | | | | | 4.08-4.16 (m, 3H), 3.95 (d, 1H), | |
| | | | | | | | | 3.77 (d, J = 11.63 Hz, 1H), 3.65 (s, 2H), | |
| | | | | | | | | 2.16-2.22 (m, 1H), | |
| | | | | | | | | 2.03-2.06 (m, 1H) | |
| 112 | N-{[(3S)-3-amino-1-(5-chloro- | Ex. 71 | LCMS | 2.3 | | 405.4 | | (500 MHz, methanol-d₄) 8.11 (s, 1H), | 0.00376 |
| | 7H-pyrrolo[2,3-d]pyrimidin-4- | | polar | | | | | 7.87 (s, 1H), 7.77 (d, J = 7.78 Hz, | |
| | yl)pyrrolidin-3-yl]methyl}-3- | | | | | | | 1H), 7.57 (d, 1H), | |
| | chlorobenzamide (112) | | | | | | | 7.43-7.48 (m, 1H), 7.19 (s, 1H), | |
| | | | | | | | | 3.98-4.14 (m, 3H), 3.80 (d, J = 11.40 Hz, 1H), | |
| | | | | | | | | 3.65 (s, 2H), 2.18-2.19 (m, 1H), | |
| | | | | | | | | 1.99-2.03 (m, 1H) | |
| 113 | N-{[(3S)-3-amino-1-(5-chloro- | Ex. 71 | LCMS | 1.96 | | 423.4 | | (500 MHz, methanol-d₄) 8.12 (s, 1H), | 0.0332 |
| | 7H-pyrrolo[2,3-d]pyrimidin-4- | | polar | | | | | 7.44-7.50 (m, 1H), 7.34 (d, | |
| | yl)pyrrolidin-3-yl]methyl}-2- | | | | | | | J = 7.78 Hz, 1H), 7.16-7.24 (m, 2H), | |
| | chloro-6-fluorobenzamide (113) | | | | | | | 4.08-4.18 (m, 1H), 4.03 (d, | |
| | | | | | | | | J = 11.40 Hz, 2H), 3.80 (d, J = 11.40 Hz, | |
| | | | | | | | | 1H), 3.64 (dd, 2H), | |
| | | | | | | | | 2.18-2.22 (m, 1H), 1.95-1.99 (m, 1H) | |
| 114 | N-{[(3S)-3-amino-1-(5-methyl- | Ex. 71 | | | | | 387.2 | (500 MHz, methanol-d₄) 8.08 (s, 1H), | 0.0176 |
| | 7H-pyrrolo[2,3-d]pyrimidin-4- | | | | | | | 7.81 (m, 1H), 7.73 (m, 1H), | |
| | yl)pyrrolidin-3-yl]methyl}-3,4- | | | | | | | 7.39 (m, 1H), 6.96 (s, 1H), | |
| | difluorobenzamide (114) | | | | | | | 3.99 (m, 4H), 3.72 (m, 4H), 2.44 (s, 3H), | |
| | | | | | | | | 2.26 (m, 1H), 2.09 (m, 1H) | |
| 115 | N-{[(3S)-3-amino-1-(5-methyl- | Ex. 71 | | | | | 385.1 (M + 1) | (500 MHz, methanol-d₄) 8.07 (s, 1H), | 0.00998 |
| | 7H-pyrrolo[2,3-d]pyrimidin-4- | | | | | | | 7.88 (s, 1H), 7.78 (d, J = 7.78 Hz, | |
| | yl)pyrrolidin-3-yl]methyl}-3- | | | | | | | 1H), 7.57 (d, J = 8.29 Hz, 1H), | |
| | chlorobenzamide (115) | | | | | | | 7.47 (t, J = 7.78 Hz, 1H), 6.94 (s, 1H), | |
| | | | | | | | | 3.97 (m, 4H), 3.67 (m, 4H), | |
| | | | | | | | | 2.43 (s, 3H), 2.20 (m, 1H), | |
| | | | | | | | | 2.03 (m, 1H) | |
| 116 | N-{[(3S)-3-amino-1-(5-methyl- | Ex. 71 | | | | | 403.1 (M + 1) | (500 MHz, methanol-d₄) 8.07 (s, 1H), | 0.0681 |
| | 7H-pyrrolo[2,3-d]pyrimidin-4- | | | | | | | 7.46 (m, 1H), 7.34 (d, J = 8.29 Hz, | |
| | yl)pyrrolidin-3-yl]methyl}-2- | | | | | | | 1H), 7.21 (t, J = 8.55 Hz, 1H), | |
| | chloro-6-fluorobenzamide (116) | | | | | | | 6.93 (s, 1H), 4.04 (q, J = 7.95 Hz, 1H), | |
| | | | | | | | | 3.92 (m, 2H), 3.65 (m, 3H), | |
| | | | | | | | | 2.45 (s, 3H), 2.21 (m, 1H), | |
| | | | | | | | | 1.97 (m, 1H) | |
| 117 | N-{[(3S)-3-amino-1-(5-methyl- | Ex. 71 | H | | 5.98 | | 399.1 (M + 1) | (DMSO-d₆) 12.57 (bs, 1H), 8.93 (t, | 0.0135 |
| | 7H-pyrrolo[2,3-d]pyrimidin-4- | | | | | | 401.1 (M + 1) | J = 6.3 Hz., 1H), 8.84 (bs, 2H), | |
| | yl)pyrrolidin-3-yl]methyl}-2- | | | | | | | 8.31 (s, 1H), 7.60 (s, 1H), | |
| | chloro-3-methylbenzamide | | | | | | | 7.45-7.40 (m, 2H), 7.29-7.26 (m, 1H), | |
| | (117) | | | | | | | 4.16-3.95 (m, 4H), 3.81-3.76 (m, | |
| | | | | | | | | 1H), 3.71-3.66 (m, 1H), | |
| | | | | | | | | 2.47-2.32 (m, 2H), 2.46 (s, 3H), 2.31 (s, 3H). | |
| 118 | N-{[(3S)-3-amino-1-(5-ethyl-7H- | Ex. 71 | H | | 6.95 | | 413.1 (M + 1) | (DMSO-d₆) 12.52 (bs, 1H), 8.93 (t, | 0.0124 |
| | pyrrolo[2,3-d]pyrimidin-4- | | | | | | 415.1 (M + 1) | J = 6.2 Hz., 1H), 8.72 (bs, 2H), | |
| | yl)pyrrolidin-3-yl]methyl}-2- | | | | | | | 8.35 (s, 1H), 7.60 (s, 1H), 7.44 (d, | |
| | chloro-3-methylbenzamide | | | | | | | J = 7.8 Hz., 1H), 7.43 (d, J = 7.8 Hz., | |
| | (118) | | | | | | | 1H), 7.31 (t, J = 7.8 Hz., 1H), | |
| | | | | | | | | 4.14-3.97 (m, 4H), 3.81-3.76 (m, | |
| | | | | | | | | 1H), 3.71-3.66 (m, 1H), 2.82 (q, J = 7.3 Hz., | |
| | | | | | | | | 2H), 2.47-2.32 (m, 2H), | |
| | | | | | | | | 2.46 (s, 3H), 2.31 (s, 3H), 1.24 (t, J = 7.3 Hz., | |
| | | | | | | | | 3H). | |
| 119 | N-{[(3S)-3-amino-1-(5-chloro- | Ex. 71 | H | | 6.5 | | 419.1 (M + 1) | (DMSO-d₆) 12.64 (bs, 1H), 8.92 (t, | 0.00785 |
| | 7H-pyrrolo[2,3-d]pyrimidin-4- | | | | | | 421.1 (M + 1) | J = 6.3 Hz., 1H), 8.66 (bs, 2H), | |
| | yl)pyrrolidin-3-yl]methyl}-2- | | | | | | | 8.31 (s, 1H), 7.60 (s, 1H), 7.44 (d, | |
| | chloro-3-methylbenzamide | | | | | | | J = 7.8 Hz., 1H), 7.42 (d, J = Hz., | |
| | (119) | | | | | | | 1H), 7.31 (t, J = 7.8 Hz., 1H), | |
| | | | | | | | | 4.17-4.06 (m, 2H), 4.04-4.01 (m, 2H), | |
| | | | | | | | | 3.78-3.74 (m, 1H), 3.70-3.67 (m, | |
| | | | | | | | | 1H), 2.43-2.30 (m, 2H), 2.37 (s, | |
| | | | | | | | | 3H). | |
| 120 | N-{[(3S)-3-amino-1-(5-methyl- | Ex. 71 | H | | 0.37 | 405.1 | | (400 MHz, methanol-d₄) 8.04 (s, 1H), | 0.0255 |
| | 7H-pyrrolo[2,3-d]pyrimidin-4- | | | | | | | 7.40 (m, 1H), 7.07 (ddd, | |
| | yl)pyrrolidin-3-yl]methyl}-2,3,6- | | | | | | | J = 13.71, 6.23, 3.32 Hz, 1H), | |
| | trifluorobenzamide (120) | | | | | | | 6.91 (s, 1H), 4.01 (m, 1H), 3.88 (m, 2H), | |
| | | | | | | | | 3.63 (m, 3H), 2.41 (s, 3H), | |
| | | | | | | | | 2.15 (m, 1H), 1.94 (m, 1H) | |
| 121 | N-{[(3S)-3-amino-1-(5-methyl- | Ex. 71 | H | | 5.17 | | 383.1 (M + 1) | (DMSO-d₆) 12.55 (bs, 1H), 9.05 (t, | 0.0418 |
| | 7H-pyrrolo[2,3-d]pyrimidin-4- | | | | | | | J = 6.2 Hz., 1H), 8.90 (bs, 2H), | |
| | yl)pyrrolidin-3-yl]methyl}-2- | | | | | | | 8.37 (s, 1H), 7.37-7.31 (m, 2H), | |
| | fluoro-6-methylbenzamide | | | | | | | 7.11-7.06 (m, 2H), 4.18-4.03 (m, | |
| | (121) | | | | | | | 2H), 3.98-3.90 (m, 1H), | |
| | | | | | | | | 3.89-3.88 (m, 1H), 3.76-3.56 (m, 2H), | |
| | | | | | | | | 2.43-2.37 (m, 2H), 2.41 (s, 3H), 2.29 (s, | |
| | | | | | | | | 3H). | |
| 122 | N-{[(3S)-3-amino-1-(5-chloro- | Ex. 71 | H | | 5.84 | | 403. (M + 1) | (DMSO-d₆) 12.66 (bs, 1H), 9.00 (t, | 0.029 |
| | 7H-pyrrolo[2,3-d]pyrimidin-4- | | | | | | 405.1 (M + 1) | J = 6.2 Hz., 1H), 8.70 (bs, 2H), | |
| | yl)pyrrolidin-3-yl]methyl}-2- | | | | | | | 8.30 (s, 1H), 7.58 (s, 1H), | |
| | fluoro-6-methylbenzamide | | | | | | | 7.36-7.31 (m, 1H), 7.10-7.06 (m, 2H), | |
| | (122) | | | | | | | 4.16-3.99 (m, 4H), 3.85-3.81 (m, | |
| | | | | | | | | 1H), 3.72-3.68 (m, 1H), | |
| | | | | | | | | 2.37-2.30 (m, 2H), 2.28 (s, 3). | |
| 123 | N-{[(3S)-3-amino-1-(5-methyl- | Ex. 71 | LCMS | 1.90 | | 403.4 | | (500 MHz, methanol-d₄) | 0.00526 |
| | 7H-pyrrolo[2,3-d]pyrimidin-4- | | Polar | | | | | 1.96-2.01 (1H, m), 2.17-2.23 (1H, m), | |
| | yl)pyrrolidin-3-yl]methyl}-2- | | | | | | | 2.45 (3H, s), 3.58-3.67 (3H, m), | |
| | chloro-3-fluorobenzamide (123) | | | | | | | 3.91-3.95 (2H, m), 4.00-4.06 (1H, m), | |
| | | | | | | | | 6.94 (1H, s), 7.32-7.37 (2H, m), | |
| | | | | | | | | 7.40-7.44 (1H, m), 8.07 (1H, s) | |
| 124 | N-{[(3S)-3-amino-1-(5-chloro- | Ex. 71 | LCMS | 2.16 | | 423.3 | | (500 MHz, methanol-d₄) | 0.00691 |
| | 7H-pyrrolo[2,3-d]pyrimidin-4- | | Polar | | | | | 1.97-2.02 (1H, m), 2.16-2.22 (1H, m), | |
| | yl)pyrrolidin-3-yl]methyl}-2- | | | | | | | 3.62 (2H, s), 3.79 (1H, d), | |
| | chloro-3-fluorobenzamide (124) | | | | | | | 4.00-4.12 (3H, m), 7.18 (1H, s), | |
| | | | | | | | | 7.32-7.36 (2H, m), 7.39-7.43 (1H, m), | |
| | | | | | | | | 8.11 (1H, s) | |
| 125 | N-{[(3S)-3-amino-1-(5-ethyl-7H- | Ex. 71 | LCMS | 2.0 | | 417.4 | | (400 MHz, methanol-d₄) | 0.0139 |
| | pyrrolo[2,3-d]pyrimidin-4- | | Polar | | | | | 1.28-1.32 (3H, t), 1.93-1.98 (1H, m), | |
| | yl)pyrrolidin-3-yl]methyl}-2- | | | | | | | 2.14-2.18 (1H, m), 2.86-2.90 (2H, q), | |
| | chloro-3-fluorobenzamide (125) | | | | | | | 3.55-3.66 (3H, m), 3.92-4.04 (3H, | |
| | | | | | | | | m), 6.94 (1H, s), 7.31-7.37 (2H, | |
| | | | | | | | | m), 7.39-7.43 (1H, m), 8.07 (1H, s) | |
| 126 | N-{[(3S)-3-amino-1-(5-methyl- | Ex. 71 | LCMS | 0.34 | | 387.1 (M + 1) | | (400 MHz, methanol-d₄) 8.04 (s, 1H), | 0.0199 |
| | 7H-pyrrolo[2,3-d]pyrimidin-4- | | Polar | | | | | 7.48 (m, 1H), 7.06 (t, J = 8.10 Hz, | |
| | yl)pyrrolidin-3-yl]methyl}-2,6- | | | | | | | 1H), 6.91 (s, 1H), 4.01 (m, 1H), | |
| | difluorobenzamide (126) | | | | | | | 3.88 (m, 2H), 3.62 (m, 3H), | |
| | | | | | | | | 2.41 (s, 3H), 2.15 (m, 1H), | |
| | | | | | | | | 1.93 (m, 1H) | |
| 127 | N-{[(3S)-3-amino-1-(5-methyl- | Ex. 71 | LCMS | 0.49 | | 365.1 (M + 1) | | (400 MHz, methanol-d₄) 8.04 (s, 1H), | 0.0362 |
| | 7H-pyrrolo[2,3-d]pyrimidin-4- | | Polar | STD | | | | 7.37 (d, J = 7.89 Hz, 1H), | |
| | yl)pyrrolidin-3-yl]methyl}-2- | | | | | | | 7.32 (t, J = 6.85 Hz, 1H), 7.22 (m, 2H), | |
| | methylbenzamide (127) | | | | | | | 6.91 (s, 1H), 3.93 (m, 4H), | |
| | | | | | | | | 3.61 (m, 4H), 2.42 (s, 3H), 2.37 (s, 3H), | |
| | | | | | | | | 2.16 (m, 1H), 1.96 (m, 1H) | |
| 128 | N-{[(3S)-3-amino-1-(5-ethyl-7H- | Ex. 71 | LCMS | 2.0 | | 399.5 | | (500 MHz, methanol-d₄) | 0.0147 |
| | pyrrolo[2,3-d]pyrimidin-4- | | Polar | | | | | 1.30-1.33 (3H, t), 1.93-1.97 (1H, m), | |
| | yl)pyrrolidin-3-yl]methyl}-2- | | | | | | | 2.15-2.23 (1H, m), 2.87-2.92 (2H, q), | |
| | chlorobenzamide (128) | | | | | | | 3.56-3.66 (3H, m), 3.92-4.06 (3H, | |
| | | | | | | | | m), 6.94 (1H, s), 7.39-7.41 (1H, | |
| | | | | | | | | m), 7.43-7.49 (2H, m), 8.07 (1H, s) | |
| 129 | N-{[(3S)-3-amino-1-(5-chloro- | Ex. 71 | LCMS | 2.2 | | 407.3 | | (500 MHz, methanol-d₄) 8.10 (s, 1H), | 0.00334 |
| | 7H-pyrrolo[2,3-d]pyrimidin-4- | | polar | | | | | 7.79 (m, 1H), 7.71 (m, 1H), | |
| | yl)pyrrolidin-3-yl]methyl}-3,4- | | | | | | | 7.38 (m, 1H), 7.18 (s, 1H), | |
| | difluorobenzamide (129) | | | | | | | 4.06 (m, 2H), 4.01 (d, J = 11.40 Hz, 1H), | |
| | | | | | | | | 3.77 (d, J = 11.92 Hz, 1H), | |
| | | | | | | | | 3.63 (s, 2H), 2.16 (m, 1H), 2.00 (m, 1H) | |
| 130 | N-{[(3S)-3-amino-1-(5-chloro- | Ex. 71 | LCMS | 2.1 | | 407.5 | | (500 MHz, methanol-d₄) 8.13 (s, 1H), | 0.00906 |
| | 7H-pyrrolo[2,3-d]pyrimidin-4- | | polar | | | | | 7.42-7.59 (m, 1H), 7.20 (s, 1H), | |
| | yl)pyrrolidin-3-yl]methyl}-2,6- | | | | | | | 7.05-7.11 (m, 2H), | |
| | difluorobenzamide (130) | | | | | | | 3.96-4.18 (m, 3H), 3.81 (d, J = 11.92 Hz, | |
| | | | | | | | | 1H), 3.59-3.71 (m, 2H), | |
| | | | | | | | | 2.14-2.24 (m, 1H), 1.96-2.04 (m, 1H) | |
| 131 | N-{[(3S)-3-amino-1-(5-chloro- | Ex. 71 | | | | | 385.4 | (400 MHz, methanol-d₄) 8.10 (s, 1H), | 0.01 |
| | 7H-pyrrolo[2,3-d]pyrimidin-4- | | | | | | | 7.12-7.42 (m, 4H), | |
| | yl)pyrrolidin-3-yl]methyl}-2- | | | | | | | 3.94-4.15 (m, 3H), 3.78 (d, 1H), | |
| | methylbenzamide (131) | | | | | | | 3.60 (s, 2H), 3.33 (s, 1H), 2.38 (s, 2H), | |
| | | | | | | | | 2.14-2.22 (m, 1H), | |
| | | | | | | | | 1.94-2.03 (m, 1H) | |
| 132 | N-{[(3S)-3-amino-1-(5-ethyl-7H- | Ex. 71 | | | | | 419.1 (M + 1) | (400 MHz, methanol-d₄) 8.04 (s, 1H), | 0.0175 |
| | pyrrolo[2,3-d]pyrimidin-4- | | | | | | | 7.40 (m, J = 11.06, 11.06, 5.92, | |
| | yl)pyrrolidin-3-yl]methyl}-2,3,6- | | | | | | | 5.19 Hz, 1H), 7.07 (m, 1H), | |
| | trifluorobenzamide (132) | | | | | | | 6.91 (s, 1H), 4.01 (m, 1H), 3.89 (m, 2H), | |
| | | | | | | | | 3.61 (m, 3H), 2.85 (q, J = 7.20 Hz, | |
| | | | | | | | | 2H), 2.12 (m, 1H), 1.90 (m, 1H), | |
| | | | | | | | | 1.27 (t, J = 7.27 Hz, 3H) | |
| 133 | N-{[(3S)-3-amino-1-(5-ethyl-7H- | Ex. 71 | | | | | 399.1 (M + 1) | (400 MHz, methanol-d₄) 8.02 (s, 1H), | 0.00447 |
| | pyrrolo[2,3-d]pyrimidin-4- | | | | | | | 7.84 (m, 1H), 7.74 (d, J = 7.89Z Hz, | |
| | yl)pyrrolidin-3-yl]methyl}-3- | | | | | | | 1H), 7.54 (d, J = 7.89 Hz, 1H), | |
| | chlorobenzamide (133) | | | | | | | 7.44 (t, J = 7.89 Hz, 1H), 6.90 (s, 1H), | |
| | | | | | | | | 3.93 (m, 3H), 3.60 (m, 3H), | |
| | | | | | | | | 2.83 (q, J = 7.48 Hz, 2H), 2.13 (m, | |
| | | | | | | | | 1H), 1.94 (m, 1H), 1.25 (t, J = 7.48 Hz, | |
| | | | | | | | | 3H) | |
| 134 | N-{[(3S)-3-amino-1-(5-cyano- | Ex. 71 | | | | | 414.4 | (500 MHz, methanol-d₄) 8.19 (s, 1H), | |
| | 7H-pyrrolo[2,3-d]pyrimidin-4- | | | | | | | 7.94 (s, 1H), 7.60 (dd, J = 8.55, | |
| | yl)pyrrolidin-3-yl]methyl}-2- | | | | | | | 5.96 Hz, 1H), 7.31 (dd, J = 8.81, | |
| | chloro-4-fluorobenzamide (134) | | | | | | | 2.59 Hz, 1H), 7.12-7.20 (m, 1H), | |
| | | | | | | | | 4.02-4.19 (m, 2H), 3.96 (d, | |
| | | | | | | | | J = 11.40 Hz, 1H), 3.81 (d, J = 11.40 Hz, | |
| | | | | | | | | 4H), 3.65 (dd, 2H), 3.36 (s, 1H), | |
| | | | | | | | | 2.21-2.27 (m, 1H), | |
| | | | | | | | | 2.04-2.10 (m, 1H) | |
| 135 | N-{[(3S)-3-amino-1-(5-cyano- | Ex. 71 | | | | | 460.4 | (500 MHz, methanol-d₄) 8.20 (s, 1H), | |
| | 7H-pyrrolo[2,3-d]pyrimidin-4- | | | | | | | 7.97 (s, 1H), 7.46-7.51 (m, 1H), | |
| | yl)pyrrolidin-3-yl]methyl}-2,5- | | | | | | | 7.21-7.33 (m, 2H), | |
| | difluorobenzamide (135) | | | | | | | 4.06-4.19 (m, 2H), 3.99 (d, J = 11.40 Hz, | |
| | | | | | | | | 1H), 3.81 (d, J = 11.40 Hz, 1H), | |
| | | | | | | | | 3.67 (dd, 2H), 3.36 (s, 1H), | |
| | | | | | | | | 2.19-2.26 (m, 1H), 2.03-2.10 (m, 1H) | |
| 136 | N-{[(3S)-3-amino-1-(5-ethyl-7H- | Ex. 71 | | | | | 416.4 | (500 MHz, methanol-d₄) 8.07 (s, 1H), | |
| | pyrrolo[2,3-d]pyrimidin-4- | | | | | | | 7.55 (t, 1H), 7.33 (d, J = 8.81 Hz, | |
| | yl)pyrrolidin-3-yl]methyl}-2- | | | | | | | 1H), 7.18 (t, J = 8.55 Hz, 1H), | |
| | chloro-4-fluorobenzamide (136) | | | | | | | 6.95 (s, 1H), 3.89-4.06 (m, 3H), | |
| | | | | | | | | 3.55-3.68 (m, 3H), 2.89 (q, | |
| | | | | | | | | J = 7.26 Hz, 2H), 2.14-2.21 (m, 1H), | |
| | | | | | | | | 1.94-1.99 (m, 1H), 1.31 (t, | |
| | | | | | | | | J = 7.26 Hz, 3H) | |
| 137 | N-{[(3S)-3-amino-1-(5-ethyl-7H- | Ex. 71 | | | | | 401.3 | (500 MHz, methanol-d₄₄) 8.07 (s, | |
| | pyrrolo[2,3-d]pyrimidin-4- | | | | | | | 1H), 7.41-7.50 (m, 1H), | |
| | yl)pyrrolidin-3-yl]methyl}-2,5- | | | | | | | 7.23-7.33 (m, 2H), 6.94 (s, 1H), | |
| | difluorobenzamide (137) | | | | | | | 3.89-4.04 (m, 3H), 3.59-3.69 (m, 3H), | |
| | | | | | | | | 2.88 (q, 2H), 2.13-2.21 (m, 1H), | |
| | | | | | | | | 1.93-2.02 (m, 1H), 1.30 (t, 3H) | |
| 138 | N-{[(3S)-3-amino-1-(7H- | Ex. 71 | | | | | 389.5 | (400 MHz, methanol-d₄) 8.05 (s, 1H), | |
| | pyrrolo[2,3-d]pyrimidin-4- | | | | | | | 7.54 (dd, J = 8.52, 6.02 Hz, 1H), | |
| | yl)pyrrolidin-3-yl]methyl}-2- | | | | | | | 7.29 (d, 3H), 7.10-7.18 (m, 1H), | |
| | chloro-4-fluorobenzamide (138) | | | | | | | 7.05 (d, J = 3.74 Hz, 1H), | |
| | | | | | | | | 6.64 (d, 1H), 3.98 (br. s., 3H), | |
| | | | | | | | | 3.53-3.76 (m, 4H), 2.19 (d, J = 11.63 Hz, | |
| | | | | | | | | 1H), 1.98 (d, J = 4.57 Hz, 1H) | |
| 139 | N-{[(3S)-3-amino-1-(7H- | Ex. 71 | | | | | 373.4 | (400 MHz, methanol-d₄) | |
| | pyrrolo[2,3-d]pyrimidin-4- | | | | | | | 7.37-7.55 (m, 1H), 7.18-7.30 (m, 2H), | |
| | yl)pyrrolidin-3-yl]methyl}-2,5- | | | | | | | 7.05 (d, J = 3.74 Hz, 1H), 6.63 (d, | |
| | difluorobenzamide (139) | | | | | | | J = 3.74 Hz, 1H), 3.98 (br. s., 2H), | |
| | | | | | | | | 3.65-3.74 (m, 2H), 3.63 (d, | |
| | | | | | | | | J = 4.98 Hz, 2H), 3.27-3.31 (m, 2H), | |
| | | | | | | | | 2.09-2.23 (m, 1H), | |
| | | | | | | | | 1.93-2.03 (m, 1H). | |
| 140 | N-{[(3S)-3-amino-1-(5-ethyl-7H- | Ex. 71 | | | | | 417.2 (M + 1) | (500 MHz, methanol-d₄) 8.07 (s, 1H), | |
| | pyrrolo[2,3-d]pyrimidin-4- | | | | | | | 7.46 (m, 1H), 7.34 (d, J = 8.29 Hz, | |
| | yl)pyrrolidin-3-yl]methyl}-2- | | | | | | | 1H), 7.21 (t, J = 8.81 Hz, 1H), | |
| | chloro-6-fluorobenzamide (140) | | | | | | | 6.94 (s, 1H), 4.04 (m, 1H), | |
| | | | | | | | | 3.92 (m, 2H), 3.64 (m, 3H), 2.89 (q, | |
| | | | | | | | | J = 7.60 Hz, 2H), 2.19 (m, 1H), | |
| | | | | | | | | 1.94 (m, 1H), 1.31 (t, J = 7.52 Hz, 3H) | |
| 141 | N-{[(3S)-3-amino-1-(5-ethyl-7H- | Ex. 71 | | | | | 401.2 (M + 1) | (500 MHz, methanol-d₄) 8.07 (s, 1H), | |
| | pyrrolo[2,3-d]pyrimidin-4- | | | | | | | 7.51 (m, 1H), 7.09 (t, J = 8.03 Hz, | |
| | yl)pyrrolidin-3-yl]methyl}-2,6- | | | | | | | 1H), 6.94 (s, 1H), 4.04 (m, 1H), | |
| | difluorobenzamide (141) | | | | | | | 3.92 (m, 2H), 3.64 (m, 3H), | |
| | | | | | | | | 2.88 (q, J = 7.26 Hz, 2H), 2.17 (m, | |
| | | | | | | | | 1H), 1.94 (m, 1H), 1.31 (t, J = 7.52 Hz, | |
| | | | | | | | | 3H) | |
| 142 | N-{[(3S)-3-amino-1-(5-ethyl-7H- | Ex. 71 | | | | | 379.3 (M + 1) | (500 MHz, methanol-d₄) 8.08 (s, 1H), | |
| | pyrrolo[2,3-d]pyrimidin-4- | | | | | | | 7.39 (d, J = 7.78 Hz, 1H), | |
| | yl)pyrrolidin-3-yl]methyl}-2- | | | | | | | 7.35 (t, J = 7.00 Hz, 1H), 7.25 (m, 2H), | |
| | methylbenzamide (142) | | | | | | | 6.95 (s, 1H), 3.99 (m, 2H), | |
| | | | | | | | | 3.92 (d, J = 10.89 Hz, 1H), 3.61 (m, 3H), | |
| | | | | | | | | 2.89 (q, J = 7.43 Hz, 2H), | |
| | | | | | | | | 2.40 (s, 3H), 2.16 (m, 1H), 1.96 (m, 1H), | |
| | | | | | | | | 1.31 (t, J = 7.26 Hz, 3H) | |
| 143 | N-{[(3S)-3-amino-1-(5-methyl- | Ex. 71 | | | | | 383.2 (M + 1) | (500 MHz, methanol-d₄) 8.08 (s, 1H), | |
| | 7H-pyrrolo[2,3-d]pyrimidin-4- | | | | | | | 7.25 (m, 2H), 7.15 (t, J = 8.81 Hz, | |
| | yl)pyrrolidin-3-yl]methyl}-3- | | | | | | | 1H), 6.94 (s, 1H), 4.03 (m, 1H), | |
| | fluoro-2-methylbenzamide | | | | | | | 3.95 (m, 1H), 3.90 (d, J = 10.89 Hz, | |
| | (143) | | | | | | | 1H), 3.64 (m, 4H), 2.45 (s, 3H), | |
| | | | | | | | | 2.30 (s, 3H), 2.18 (m, 1H), | |
| | | | | | | | | 2.00 (m, 1H) | |
| 144 | N-{[(3S)-3-amino-1-(5-methyl- | Ex. 71 | | | | | 421.1 (M + 1) | (500 MHz, methanol-d₄) 8.07 (s, 1H), | |
| | 7H-pyrrolo[2,3-d]pyrimidin-4- | | | | | | | 7.54 (m, 2H), 6.94 (s, 1H), | |
| | yl)pyrrolidin-3-yl]methyl}-2- | | | | | | | 4.03 (m, 1H), 3.93 (m, 2H), | |
| | chloro-4,5-difluorobenzamide | | | | | | | 3.63 (m, 3H), 2.45 (s, 3H), 2.19 (m, 1H), | |
| | (144) | | | | | | | 1.99 (m, 1H) | |
| 145 | N-{[(3S)-3-amino-1-(5-cyano- | Ex. 71 | LCMS | 2.0 | | 414.5 | | (500 MHz, methanol-d₄) 8.23 (s, 1H), | |
| | 7H-pyrrolo[2,3-d]pyrimidin-4- | | polar | | | | | 7.99 (s, 1H), 7.45-7.48 (m, 1H), | |
| | yl)pyrrolidin-3-yl]methyl}-2- | | | | | | | 7.34 (d, J = 7.78 Hz, 1H), | |
| | chloro-6-fluorobenzamide (145) | | | | | | | 7.21 (t, J = 8.81 Hz, 1H), 4.07-4.24 (m, | |
| | | | | | | | | 2H), 3.93 (dd, 2H), | |
| | | | | | | | | 3.63-3.78 (m, 2H), 2.27-2.36 (m, 1H), | |
| | | | | | | | | 2.08-2.14 (m, 1H) | |
| 146 | N-{[(3S)-3-amino-1-(5-cyano- | Ex. 71 | LCMS | 2.0 | | 416.5 | | (500 MHz, methanol-d₄) 8.23 (s, 1H), | |
| | 7H-pyrrolo[2,3-d]pyrimidin-4- | | polar | | | | | 7.99 (s, 1H), 7.40-7.49 (m, 1H), | |
| | yl)pyrrolidin-3-yl]methyl}-2,3,6- | | | | | | | 7.06-7.12 (m, 1H), | |
| | trifluorobenzamide (146) | | | | | | | 4.08-4.25 (m, 2H), 3.93-4.01 (m, 1H), | |
| | | | | | | | | 3.68-3.76 (m, 2H), | |
| | | | | | | | | 3.66-3.75 (m, 1H), 2.25-2.33 (m, 1H), | |
| | | | | | | | | 2.06-2.16 (m, 1H) | |
| 147 | N-{[(3S)-3-amino-1-(5-chloro- | Ex. 71 | LCMS | 2.2 | | 441.4 | | (500 MHz, methanol-d₄) 8.13 (s, 1H), | |
| | 7H-pyrrolo[2,3-d]pyrimidin-4- | | polar | | | | | 7.48-7.60 (m, 2H), 7.20 (s, 1H), | |
| | yl)pyrrolidin-3-yl]methyl}-2- | | | | | | | 3.97-4.14 (m, 3H), 3.79 (d, | |
| | chloro-4,5-difluorobenzamide | | | | | | | J = 11.92 Hz, 1H), 3.61 (s, 2H), | |
| | (147) | | | | | | | 2.14-2.25 (m, 1H), | |
| | | | | | | | | 1.92-2.02 (m, 1H) | |
| 148 | N-{[(3S)-3-amino-1-(5-chloro- | Ex. 71 | LCMS | 2.1 | | 403.4 | | (500 MHz, methanol-d₄) 8.13 (s, 1H), | |
| | 7H-pyrrolo[2,3-d]pyrimidin-4- | | polar | | | | | 7.08-7.30 (m, 4H), | |
| | yl)pyrrolidin-3-yl]methyl}-3- | | | | | | | 3.99-4.19 (m, 3H), 3.79 (d, J = 11.40 Hz, | |
| | fluoro-2-methylbenzamide | | | | | | | 1H), 3.62 (s, 2H), 2.30 (s, 3H), | |
| | (148) | | | | | | | 2.13-2.18 (m, 1H), | |
| | | | | | | | | 1.96-2.03 (m, 1H) | |
| 149 | N-{[(3S)-3-amino-1-(5-cyano- | Ex. 71 | LCMS | 2.1 | | 394.5 | | (500 MHz, methanol-d₄) 8.23 (s, 1H), | |
| | 7H-pyrrolo[2,3-d]pyrimidin-4- | | polar | | | | | 7.99 (s, 1H), 7.24-7.30 (m, 2H), | |
| | yl)pyrrolidin-3-yl]methyl}-3- | | | | | | | 7.12-7.19 (m, 1H), | |
| | fluoro-2-methylbenzamide | | | | | | | 4.07-4.26 (m, 2H), 4.01 (d, J = 11.40 Hz, | |
| | (149) | | | | | | | 1H), 3.85 (d, 1H), 3.58-3.72 (m, | |
| | | | | | | | | 2H), 2.31 (s, 3H), 2.22-2.27 (m, | |
| | | | | | | | | 1H), 2.06-2.13 (m, 1H) | |
| 150 | N-{[(3S)-3-amino-1-(5-cyano- | Ex. 71 | | | | | 432.4 | (500 MHz, methanol-d₄) 8.22 (s, 1H), | |
| | 7H-pyrrolo[2,3-d]pyrimidin-4- | | | | | | | 7.98 (s, 1H), 7.49-7.62 (m, 2H), | |
| | yl)pyrrolidin-3-yl]methyl}-2- | | | | | | | 4.07-4.22 (m, 2H), 4.00 (d, | |
| | chloro-4,5-difluorobenzamide | | | | | | | J = 11.40 Hz, 1H), 3.84 (d, J = 11.40 Hz, | |
| | (150) | | | | | | | 1H), 3.57-3.75 (m, 3H), | |
| | | | | | | | | 2.21-2.30 (m, 1H), 2.04-2.09 (m, 1H) | |
| 151 | N-{[(3S)-3-amino-1-(5-cyano- | Ex. 71 | | | | | 396.4 | (500 MHz, methanol-d₄) 8.22 (s, 1H), | |
| | 7H-pyrrolo[2,3-d]pyrimidin-4- | | | | | | | 7.99 (s, 1H), 7.89 (s, 1H), | |
| | yl)pyrrolidin-3-yl]methyl}-3- | | | | | | | 7.80 (d, J = 7.78 Hz, 1H), 7.57 (d, | |
| | chlorobenzamide (151) | | | | | | | J = 8.29 Hz, 1H), 7.47 (t, J = 8.03 Hz, | |
| | | | | | | | | 1H), 4.10-4.21 (m, 2H), | |
| | | | | | | | | 4.06 (d, J = 11.92 Hz, 1H), 3.89 (d, | |
| | | | | | | | | J = 11.92 Hz, 1H), 3.71 (s, 2H), | |
| | | | | | | | | 2.25-2.36 (m, 1H), | |
| | | | | | | | | 2.10-2.19 (m, 1H) | |
| 152 | N-{[(3S)-3-amino-1-(5-ethyl-7H- | Ex. 71 | | | | | 435.4 | (500 MHz, methanol-d₄) 8.09 (s, 1H), | |
| | pyrrolo[2,3-d]pyrimidin-4- | | | | | | | 7.42-7.62 (m, 2H), 6.96 (s, 1H), | |
| | yl)pyrrolidin-3-yl]methyl}-2- | | | | | | | 3.90-4.06 (m, 3H), | |
| | chloro-4,5-difluorobenzamide | | | | | | | 3.53-3.75 (m, 3H), 2.89 (q, J = 7.26 Hz, | |
| | (152) | | | | | | | 2H), 2.19-2.26 (m, 1H), | |
| | | | | | | | | 1.94-2.08 (m, 1H), 1.32 (t, J = 7.26 Hz, 3H) | |
| 153 | N-{[(3S)-3-amino-1-(5-ethyl-7H- | Ex. 71 | | | | | 397.4 | (500 MHz, methanol-d₄) 8.08 (s, 1H), | |
| | pyrrolo[2,3-d]pyrimidin-4- | | | | | | | 7.11-7.30 (m, 3H), 6.96 (s, 1H), | |
| | yl)pyrrolidin-3-yl]methyl}-3- | | | | | | | 3.89-4.07 (m, 3H), | |
| | fluoro-2-methylbenzamide | | | | | | | 3.55-3.71 (m, 3H), 2.89 (q, J = 7.60 Hz, | |
| | (153) | | | | | | | 2H), 2.29 (s, 3H), 2.15-2.24 (m, | |
| | | | | | | | | 1H), 1.94-2.04 (m, 1H), 1.31 (t, | |
| | | | | | | | | J = 7.26 Hz, 3H) | |
| 154 | N-{[(3S)-3-amino-1-(5-ethyl-7H- | Ex. 71 | LCMS | 1.96 | | 357.2 | | (400 MHz, methanol-d₄) | |
| | pyrrolo[2,3-d]pyrimidin-4- | | Polar | | | | | 1.24-1.28 (3H, t), 1.51-1.60 (2H, m), | |
| | yl)pyrrolidin-3- | | | | | | | 1.61-1.70 (4H, m), 1.79-1.87 (3H, m), | |
| | yl]methyl}cyclopentane- | | | | | | | 1.97-2.04 (1H, m), 2.59-2.67 (1H, | |
| | carboxamide (154) | | | | | | | m), 278-2.83 (2H, q), | |
| | | | | | | | | 3.32-3.42 (2H, q), 3.53 (1H, d), 3.77 (1H, d), | |
| | | | | | | | | 3.82-3.86 (1H, m), 3.87-3.97 (1H, | |
| | | | | | | | | m), 6.89 (1H, s), 8.03 (1H, s) | |
| 155 | N-{[(3S)-3-amino-1-(5-chloro- | Ex. 71 | LCMS | 1.99 | | 349.4 | | (500 MHz, methanol-d₄) 0.57 (2H, | |
| | 7H-pyrrolo[2,3-d]pyrimidin-4- | | Polar | | | | | t), 1.03-1.05 (2H, m), 1.32 (3H, s), | |
| | yl)pyrrolidin-3-yl]methyl}-1- | | | | | | | 1.86-1.93 (1H, m), 2.03-2.10 (1H, | |
| | methylcyclopropane- | | | | | | | m), 3.42 (2H, s), 3.67 (1H, d), | |
| | carboxamide (155) | | | | | | | 3.92 (1H, d), 3.97-4.02 (2H, m), | |
| | | | | | | | | 7.16 (1H, s), 8.07 (1H, s) | |
| 156 | N-{[(3S)-3-amino-1-(5-cyano- | Ex. 87 | LCMS | 2.3 | | 414.4 | | (400 MHz, DMSO-d₆) 8.57 (t, 1H), | |
| | 7H-pyrrolo[2,3-d]pyrimidin-4- | | Polar | | | | | 8.17 (d, 2H), 7.67 (m, 1H), | |
| | yl)pyrrolidin-3-yl]methyl}-3- | | | | | | | 7.52 (m, 1H), 7.26 (t, 1H), 3.95 (m, 2H), | |
| | chloro-2-fluorobenzamide (156) | | | | | | | 3.66 (d, 1H), 3.55 (d, 1H), 3.42 (m, | |
| | | | | | | | | 2H), 1.97 (m, 1H), 1.82 (m, 1H). | |
| 157 | N-{[(3S)-3-amino-1-(5-methyl- | Ex. 87 | LCMS | 2.0 | | 381.5 | | (400 MHz, DMSO-d₆) 11.31 (s, | |
| | 7H-pyrrolo[2,3-d]pyrimidin-4- | | Polar | | | | | 1H), 8.38 (t, 1H), 8.04 (s, 1H), | |
| | yl)pyrrolidin-3-yl]methyl}-4- | | | | | | | 7.85 (d, 2H), 6.99 (d, 2H), 6.93 (s, 1H), | |
| | methoxybenzamide (157) | | | | | | | 3.83 (m, 4H), 3.70 (m, 2H), | |
| | | | | | | | | 3.42 (m, 3H), 2.30 (s, 3H), 1.94 (m, | |
| | | | | | | | | 1H), 1.74 (m, 1H). | |
| 158 | N-{[(3S)-3-amino-1-(5-ethyl-7H- | Ex. 87 | LCMS | 2.0 | | 381.5 | | (400 MHz, DMSO-d₆) 11.37 (s, | |
| | pyrrolo[2,3-d]pyrimidin-4- | | Polar | | | | | 1H), 8.38 (t, 1H), 8.03 (s, 1H), | |
| | yl)pyrrolidin-3-yl]methyl}-4- | | | | | | | 7.85 (d, 2H), 7.00 (d, 2H), 6.89 (s, 1H), | |
| | methoxybenzamide (158) | | | | | | | 3.95 (m, 4H), 3.68 (d, 2H), | |
| | | | | | | | | 3.40 (m, 3H), 2.72 (m 2H), 1.90 (m, | |
| | | | | | | | | 3H), 1.71 (m, 1H). | |
| 159 | N-{[(3S)-3-amino-1-(5-chloro- | Ex. 87 | LCMS | 2.0 | | 395.5 | | (400 MHz, DMSO-d₆) 11.92 (s, | |
| | 7H-pyrrolo[2,3-d]pyrimidin-4- | | Polar | | | | | 1H), 8.36 (t, 1H), 8.07 (s, 1H), | |
| | yl)pyrrolidin-3-yl]methyl}-4- | | | | | | | 7.81 (d, 2H), 7.31 (s, 1H), 6.96 (d, 2H), | |
| | methoxybenzamide (159) | | | | | | | 3.90 (m, 6H), 3.48 (m, 3H), | |
| | | | | | | | | 1.92 (m, 1H), 1.73 (m, 1H). | |
| 160 | N-{[(3S)-3-amino-1-(5-cyano- | Ex. 87 | LCMS | 2.1 | | 392.5 | | (400 MHz, DMSO-d₆) 8.31 (t, 1H), | |
| | 7H-pyrrolo[2,3-d]pyrimidin-4- | | Polar | | | | | 8.86 (d, 2H), 7.81 (d, 2H), 3.92 (m, | |
| | yl)pyrrolidin-3-yl]methyl}-4- | | | | | | | 2H), 3.77 (s, 3H), 3.66 (d, 1H), | |
| | methoxybenzamide (160) | | | | | | | 3.52 (d, 1H), 3.41 (d, 2H), 1.97 (m, | |
| | | | | | | | | 1H), 1.79 (m, 1H). | |
| 161 | N-{[(3S)-3-amino-1-(5-methyl- | Ex. 71 | LCMS | | 6.61 | | 453.0 (M + 1) | (DMSO-d₆) 12.58 (bs, 1H), 9.00 (t, | |
| | 7H-pyrrolo[2,3-d]pyrimidin-4- | | Polar | | | | 455.1 (M + 1) | J = 6.2 Hz., 1H), 8.90 (bs, 2H), | |
| | yl)pyrrolidin-3-yl]methyl}-2- | | | | | | | 8.38 (s, 1H), 7.99 (d, J = 7.8 Hz., | |
| | chloro-3- | | | | | | | 1H), 7.96 (d, J = 7.8 Hz., 1H), | |
| | (trifluoromethyl)benzamide | | | | | | | 7.65 (t, J = 7.8 Hz., 1H), 7.33 (s, 1H), | |
| | (161) | | | | | | | 4.22-4.16 (m, 2H), 4.09-4.06 (m, | |
| | | | | | | | | 1H), 4.01-3.97 (m, 1H), | |
| | | | | | | | | 3.87-3.83 (m, 1H), 3.79-3.75 (m, 1H), | |
| | | | | | | | | 2.46-2.36 (m, 2H), 2.42 (s, 3H); ¹⁹F | |
| | | | | | | | | NMR (DMSO-d₆) δ, ppm: −61.39. | |
| 162 | N-{[(3S)-3-amino-1-(5-chloro- | Ex. 71 | LCMS | | 8.01 | | 473.0 (M + 1) | (DMSO-d₆) 12.72 (bs, 1H), 9.21 (t, | |
| | 7H-pyrrolo[2,3-d]pyrimidin-4- | | Polar | | | | 476.1 (M + 1) | J = 6.2 Hz., 1H), 8.90 (bs, 2H), | |
| | yl)pyrrolidin-3-yl]methyl}-2- | | | | | | | 8.38 (s, 1H), 7.99 (d, J = 7.8 Hz., 1H), | |
| | chloro-3- | | | | | | | 7.96 (d, J = 7.8 Hz., 1H), 7.65 (t, J = 7.8 Hz., | |
| | (trifluoromethyl)benzamide | | | | | | | 1H), 7.61 (s, 1H), | |
| | (162) | | | | | | | 4.16-4.01 (m, 4H), 3.82-3.72 (m, 2H), | |
| | | | | | | | | 2.43-2.31 (m, 2H); ¹⁹F | |
| | | | | | | | | NMR (DMSO-d₆) δ, ppm: −61.39. | |
| 163 | N-{[(3S)-3-amino-1-(5-ethyl-7H- | Ex. 71 | LCMS | | 8.4 | | 467.0 (M + 1) | (DMSO-d₆) 12.56 (bs, 1H), 9.20 (t, | |
| | pyrrolo[2,3-d]pyrimidin-4- | | Polar | | | | 469.1 (M + 1) | J = 6.2 Hz., 1H), 8.85 (bs, 2H), | |
| | yl)pyrrolidin-3-yl]methyl}-2- | | | | | | | 8.36 (s, 1H), 7.96 (d, J = 7.7 Hz., | |
| | chloro-3- | | | | | | | 1H), 7.95 (d, J = 7.7 Hz., 1H), | |
| | (trifluoromethyl)benzamide | | | | | | | 7.65 (t, J = 7.7 Hz., 1H), 7.16 (s, 1H), | |
| | (163) | | | | | | | 4.18-3.97 (m, 4H), 3.84-3.80 (m, | |
| | | | | | | | | 1H), 3.76-3.72 (m, 1H), 2.83 (q, J = 7.3 Hz., | |
| | | | | | | | | 2H), 2.44-2.34 (m, 2H), | |
| | | | | | | | | 1.25 (t, J = 7.3 Hz., 3H); ¹⁹F | |
| | | | | | | | | NMR (DMSO-d₆) δ, ppm: −61.39. | |
| 164 | N-{[(3S)-3-amino-1-(5-methyl- | Ex. 71 | LCMS | | 5.07 | | 383.2 (M + 1) | (DMSO-d₆) 12.52 (bs, 1H), | |
| | 7H-pyrrolo[2,3-d]pyrimidin-4- | | Polar | | | | | 8.73 (bs, 3H), 8.35 (s, 1H), 7.65 (, J = 8.3, | |
| | yl)pyrrolidin-3-yl]methyl}-2- | | | | | | | 8.3 Hz., 1H), 7.31 (s, 1H), | |
| | fluoro-4-methylbenzamide | | | | | | | 7.14 (d, J = 8.3 Hz., 1H), 7.12 (d, J = 8.3 Hz., | |
| | (164) | | | | | | | 1H), 4.15-3.95 (m, 4H), | |
| | | | | | | | | 3.78-3.77 (m, 2H), 2.43-2.34 (m, | |
| | | | | | | | | 2H), 2.40 (s, 3H), 2.36 (s, 3H). | |
| 165 | N-{[(3S)-3-amino-1-(5-chloro- | Ex. 71 | LCMS | | 6.46 | | 403.1 (M + 1) | (DMSO-d₆) 12.61 (bs, 1H), | |
| | 7H-pyrrolo[2,3-d]pyrimidin-4- | | Polar | | | | 405.1 (M + 1) | 8.69 (bs, 1H), 8.56 (bs, 2H), 8.30 (s, | |
| | yl)pyrrolidin-3-yl]methyl}-2- | | | | | | | 1H), 7.64 (dd, J = 7.7, 7.7 Hz., | |
| | fluoro-4-methylbenzamide | | | | | | | 1H), 7.58 (s, 1H), 7.16-7.09 (m, | |
| | (165) | | | | | | | 2H), 4.10-4.01 (m, 4H), | |
| | | | | | | | | 3.74-3.73 (m, 2H), 2.36-2.30 (m, 2H), | |
| | | | | | | | | 2.40 (s, 3H); ¹⁹F NMR (DMSO-d₆) δ, | |
| | | | | | | | | ppm: −114.43. | |
| 166 | N-{[(3S)-3-amino-1-(5-ethyl-7H- | Ex. 71 | LCMS | | 6.95 | | | (DMSO-d₆) 12.54 (bs, 1H), | |
| | pyrrolo[2,3-d]pyrimidin-4- | | Polar | | | | | 8.68 (bs, 3H), 8.34 (s, 1H), 7.64 (dd, J = 7.8, | |
| | yl)pyrrolidin-3-yl]methyl}-2- | | | | | | | 7.8 Hz., 1H), 7.30-7.09 (m, | |
| | fluoro-4-methylbenzamide | | | | | | | 3H), 4.09-3.96 (m, 4H), | |
| | (166) | | | | | | | 3.79-3.71 (m, 2H), 2.80 (q, J = 7.3 Hz., 2H), | |
| | | | | | | | | 2.36-2.24 (m, 2H), 2.36 (s, 3H), | |
| | | | | | | | | 1.25 (t, J = 7.3 Hz., 3H); ¹⁹F | |
| | | | | | | | | NMR (DMSO-d₆) δ, ppm: −114.43. | |
| 167 | N-{[(3S)-3-amino-1-(5-methyl- | Ex. 87 | LCMS | 2.3 | | 437.5 | | (400 MHz, DMSO-d₆) 11.28 (s, | |
| | 7H-pyrrolo[2,3-d]pyrimidin-4- | | Polar | | | | | 1H), 8.79 (t, 1H), 8.26 (m, 2H), | |
| | yl)pyrrolidin-3-yl]methyl}-4- | | | | | | | 8.03 (s, 1H), 7.64 (m, 1H), 6.93 (s, 1H), | |
| | fluoro-3- | | | | | | | 3.84 (m, 1H), 3.70 (m, 2H), | |
| | (trifluoromethyl)benzamide | | | | | | | 3.42 (m, 3H), 2.31 (s, 3H), 1.85 (m, | |
| | (167) | | | | | | | 4H). | |
| 168 | N-{[(3S)-3-amino-1-(5-ethyl-7H- | Ex. 87 | LCMS | 2.5 | | 451.1 | | (400 MHz, DMSO-d₆) 11.35 (s, | |
| | pyrrolo[2,3-d]pyrimidin-4- | | Polar | | | | | 1H), 8.80 (t, 1H), 8.25 (m, 2H), | |
| | yl)pyrrolidin-3-yl]methyl}-4- | | | | | | | 8.02 (s, 1H), 7.64 (m, 1H), 6.90 (s, | |
| | fluoro-3- | | | | | | | 1H), 3.87 (m, 1H), 3.70 (m, 2H), | |
| | (trifluoromethyl)benzamide | | | | | | | 3.44 (m, 3H), 2.69 (m, 2H), | |
| | (168) | | | | | | | 1.84 (m, 4H) 1.17 (t, 3H). | |
| 169 | N-{[(3S)-3-amino-1-(5-cyano- | Ex. 87 | LCMS | 2.5 | | 448.1 | | (400 MHz, DMSO-d₆) 8.78 (m, | |
| | 7H-pyrrolo[2,3-d]pyrimidin-4- | | Polar | | | | | 1H), 8.24 (m, 4H), 7.64 (m, 1H), | |
| | yl)pyrrolidin-3-yl]methyl}-4- | | | | | | | 3.94 (m, 2H), 3.73 (d, 1H), 3.59 (d, | |
| | fluoro-3- | | | | | | | 1H), 3.50 (m, 2H), 2.04 (m, 1H), | |
| | (trifluoromethyl)benzamide | | | | | | | 1.87 (m, 1H). | |
| | (169) | | | | | | | | |
| 170 | N-{[(3S)-3-amino-1-(5-chloro- | Ex. 71 | LCMS | 2.5 | | 457.1 | | (400 MHz, DMSO-d₆) 11.97 (s, | |
| | 7H-pyrrolo[2,3-d]pyrimidin-4- | | Polar | | | | | 1H), 8.80 (t, 1H), 8.25 (m, 2H), | |
| | yl)pyrrolidin-3-yl]methyl}-4- | | | | | | | 8.09 (s, 1H), 7.64 (m, 1H), 7.34 (s, | |
| | fluoro-3- | | | | | | | 1H), 3.94 (m, 1H), 3.80 (m, 2H), | |
| | (trifluoromethyl)benzamide | | | | | | | 3.48 (m, 3H), 1.86 (m, 4H). | |
| | (170) | | | | | | | | |
| 171 | N-{[(3S)-3-amino-1-thieno[2,3- | Ex. 71 | LCMS | 2.13 | | | 390.0 (M + 1) | (DMSO-d₆) 8.44 (t, J = 5.4 Hz., | |
| | d]pyrimidin-4-ylpyrrolidin-3- | | Polar | | | | | 1H), 8.28 (s, 1H), 7.71-7.65 (m, | |
| | yl]methyl}-2,4- | | | | | | | 1H), 7.57 (bs, 1H), 7.49 (d, J = 6.2 Hz., | |
| | difluorobenzamide (171) | | | | | | | 1H), 7.33 (td, J = 10.0, 2.5 Hz., | |
| | | | | | | | | 1H), 7.14 (td, J = 8.4, 2.5 Hz., | |
| | | | | | | | | 1H), 3.98-3.42 (m, 6H), | |
| | | | | | | | | 1.95-1.77 (m, 4H). | |
| 172 | N-{[(3S)-3-amino-1-(5-ethyl-7H- | Ex. 72 | LCMS | 1.3 | | 343.2 | | | |
| | pyrrolo[2,3-d]pyrimidin-4- | | Basic | | | | | | |
| | yl)pyrrolidin-3-yl]methyl}-2- | | | | | | | | |
| | cyclopropylacetamide (172) | | | | | | | | |
| 173 | N-{[(3S)-3-amino-1-(5-ethyl-7H- | Ex. 72 | LCMS | 1.2 | | 329.2 | | | |
| | pyrrolo[2,3-d]pyrimidin-4- | | Basic | | | | | | |
| | yl)pyrrolidin-3- | | | | | | | | |
| | yl]methyl}cyclopropane- | | | | | | | | |
| | carboxamide (173) | | | | | | | | |
| 174 | N-{[(3S)-3-amino-1-(5-ethyl-7H- | Ex. 72 | LCMS | 1.8 | | 417.1 | | | |
| | pyrrolo[2,3-d]pyrimidin-4- | | Basic | | | | | | |
| | yl)pyrrolidin-3-yl]methyl}-3- | | | | | | | | |
| | chloro-2-fluorobenzamide (174) | | | | | | | | |
| 175 | N-{[(3S)-3-amino-1-(5-ethyl-7H- | Ex. 72 | LCMS | 1.6 | | 397.2 | | | |
| | pyrrolo[2,3-d]pyrimidin-4- | | Basic | | | | | | |
| | yl)pyrrolidin-3-yl]methyl}-2- | | | | | | | | |
| | fluoro-6-methylbenzamide | | | | | | | | |
| | (175) | | | | | | | | |
| 176 | N-{[(3S)-3-amino-1-(5-ethyl-7H- | Ex. 72 | LCMS | 1.1 | | 361.2 | | | |
| | pyrrolo[2,3-d]pyrimidin-4- | | Basic | | | | | | |
| | yl)pyrrolidin-3-yl]methyl}-4- | | | | | | | | |
| | methoxybutanamide (176) | | | | | | | | |
| 177 | N-{[(3S)-3-amino-1-(5-ethyl-7H- | Ex. 72 | LCMS | 1.4 | | 345.2 | | | |
| | pyrrolo[2,3-d]pyrimidin-4- | | Basic | | | | | | |
| | yl)pyrrolidin-3-yl]methyl}-3- | | | | | | | | |
| | methylbutanamide (177) | | | | | | | | |
| 178 | N-{[(3S)-3-amino-1-(5-ethyl-7H- | Ex. 72 | LCMS | 1.4 | | 397.2 | | | |
| | pyrrolo[2,3-d]pyrimidin-4- | | Basic | | | | | | |
| | yl)pyrrolidin-3-yl]methyl}-3- | | | | | | | | |
| | ethyl-1-methyl-1H-pyrazole-5- | | | | | | | | |
| | carboxamide (178) | | | | | | | | |
| 179 | N-{[(3S)-3-amino-1-(5-ethyl-7H- | Ex. 72 | LCMS | 1.7 | | 397.2 | | | |
| | pyrrolo[2,3-d]pyrimidin-4- | | Basic | | | | | | |
| | yl)pyrrolidin-3-yl]methyl}-5- | | | | | | | | |
| | fluoro-2-methylbenzamide | | | | | | | | |
| | (179) | | | | | | | | |
| 180 | N-{[(3S)-3-amino-1-(5-ethyl-7H- | Ex. 72 | LCMS | 1.6 | | 413.2 | | | |
| | pyrrolo[2,3-d]pyrimidin-4- | | Basic | | | | | | |
| | yl)pyrrolidin-3-yl]methyl}-3- | | | | | | | | |
| | fluoro-4-methoxybenzamide | | | | | | | | |
| | (180) | | | | | | | | |
| 181 | N-{[(3S)-3-amino-1-(5-ethyl-7H- | Ex. 72 | LCMS | 0.9 | | 359.2 | | | |
| | pyrrolo[2,3-d]pyrimidin-4- | | Basic | | | | | | |
| | yl)pyrrolidin-3- | | | | | | | | |
| | yl]methyl}tetrahydro-furan-3- | | | | | | | | |
| | carboxamide (181) | | | | | | | | |
| 182 | N-{[(3S)-3-amino-1-(5-ethyl-7H- | Ex. 72 | LCMS | 1.5 | | 409.2 | | | |
| | pyrrolo[2,3-d]pyrimidin-4- | | Basic | | | | | | |
| | yl)pyrrolidin-3-yl]methyl}-2-(4- | | | | | | | | |
| | methoxyphenyl)acetamide | | | | | | | | |
| | (182) | | | | | | | | |
| 183 | N-{[(3S)-3-amino-1-(5-ethyl-7H- | Ex. 72 | LCMS | 1.3 | | 343.2 | | | |
| | pyrrolo[2,3-d]pyrimidin-4- | | Basic | | | | | | |
| | yl)pyrrolidin-3- | | | | | | | | |
| | yl]methyl}cyclobutane- | | | | | | | | |
| | carboxamide (183) | | | | | | | | |
| 184 | N-{[(3S)-3-amino-1-(5-ethyl-7H- | Ex. 72 | LCMS | 1.4 | | 397.2 | | | |
| | pyrrolo[2,3-d]pyrimidin-4- | | Basic | | | | | | |
| | yl)pyrrolidin-3-yl]methyl}-1- | | | | | | | | |
| | ethyl-3-methyl-1H-pyrazole-5- | | | | | | | | |
| | carboxamide (184) | | | | | | | | |
| 185 | N-{[(3S)-3-amino-1-(5-ethyl-7H- | Ex. 72 | LCMS | 1.1 | | 359.2 | | | |
| | pyrrolo[2,3-d]pyrimidin-4- | | Basic | | | | | | |
| | yl)pyrrolidin-3- | | | | | | | | |
| | yl]methyl}tetrahydro-furan-2- | | | | | | | | |
| | carboxamide (185) | | | | | | | | |
| 186 | N-{[(3S)-3-amino-1-(5-ethyl-7H- | Ex. 72 | LCMS | 1.6 | | 358.2 | | | |
| | pyrrolo[2,3-d]pyrimidin-4- | | Basic | | | | | | |
| | yl)pyrrolidin-3-yl]methyl}-3,3- | | | | | | | | |
| | dimethylbutanamide (186) | | | | | | | | |
| 187 | N-{[(3S)-3-amino-1-(5-ethyl-7H- | Ex. 72 | LCMS | 1.6 | | 395.2 | | | |
| | pyrrolo[2,3-d]pyrimidin-4- | | Basic | | | | | | |
| | yl)pyrrolidin-3-yl]methyl}-3- | | | | | | | | |
| | methoxybenzamide (187) | | | | | | | | |
| 188 | N-{[(3S)-3-amino-1-(5-ethyl-7H- | Ex. 72 | LCMS | 1.7 | | 397.2 | | | |
| | pyrrolo[2,3-d]pyrimidin-4- | | Basic | | | | | | |
| | yl)pyrrolidin-3-yl]methyl}-3,5- | | | | | | | | |
| | difluorobenzamide (188) | | | | | | | | |
| 189 | N-{[(3S)-3-amino-1-(5-ethyl-7H- | Ex. 72 | LCMS | 1.8 | | 451.1 | | | |
| | pyrrolo[2,3-d]pyrimidin-4- | | Basic | | | | | | |
| | yl)pyrrolidin-3-yl]methyl}-4- | | | | | | | | |
| | fluoro-2- | | | | | | | | |
| | (trifluoromethyl)benzamide | | | | | | | | |
| | (189) | | | | | | | | |
| 190 | N-{[(3S)-3-amino-1-(5-ethyl-7H- | Ex. 72 | LCMS | 1.6 | | 411.2 | | | |
| | pyrrolo[2,3-d]pyrimidin-4- | | Basic | | | | | | |
| | yl)pyrrolidin-3-yl]methyl}-3- | | | | | | | | |
| | isopropyl-1-methyl-1H- | | | | | | | | |
| | pyrazole-5-carboxamide (190) | | | | | | | | |
| 191 | N-{[(3S)-3-amino-1-(5-ethyl-7H- | Ex. 72 | LCMS | 1.2 | | 347.2 | | | |
| | pyrrolo[2,3-d]pyrimidin-4- | | Basic | | | | | | |
| | yl)pyrrolidin-3-yl]methyl}-2- | | | | | | | | |
| | ethoxyacetamide (191) | | | | | | | | |
| 192 | N-{[(3S)-3-amino-1-(5-ethyl-7H- | Ex. 72 | LCMS | 1.0 | | 373.2 | | | |
| | pyrrolo[2,3-d]pyrimidin-4- | | Basic | | | | | | |
| | yl)pyrrolidin-3- | | | | | | | | |
| | yl]methyl}tetrahydro-2H-pyran- | | | | | | | | |
| | 4-carboxamide (192) | | | | | | | | |
| 193 | N-{[(3S)-3-amino-1-(5-ethyl-7H- | Ex. 72 | LCMS | 1.6 | | 397.2 | | | |
| | pyrrolo[2,3-d]pyrimidin-4- | | Basic | | | | | | |
| | yl)pyrrolidin-3-yl]methyl}-2-(4- | | | | | | | | |
| | fluorophenyl)acetamide (193) | | | | | | | | |
| 194 | N-{[1-(5-chloro-7H-pyrrolo[2,3- | Ex. 69 | | | | | | (Methanol-d₄) 8.25 (s, 1H), 7.9 (d, | 0.0897 |
| | d]pyrimidin-4-yl)-3- | | | | | | | 2H), 7.55-7.62 (m, 1H), | |
| | (methylamino)pyrrolidin-3- | | | | | | | 7.45-7.53 (m, 2H), 7.35 (s, 1H), | |
| | yl]methyl}benzamide (194) | | | | | | | 4.16-4.36 (m, 4H), 3.9 (d, 2H), 2.96 (s, 3H), | |
| | | | | | | | | 2.38-2.58 (m, 2H). | |
| 195 | 1-{[3-amino-1-(5-methyl-7H- | Ex. 76 | LCMS | 2.1 | | 437.4 | | (500 MHz, methanol-d₄) | 0.141 |
| | pyrrolo[2,3-d]pyrimidin-4- | | Polar | | | | | 1.87-1.90 (1H, m), 2.02-2.08 (1H, m), | |
| | yl)pyrrolidin-3-yl]methyl}-1-[2- | | | | | | | 2.40 (3H, s), 2.48 (3H, s), | |
| | (dimethylamino)ethyl]-3- | | | | | | | 2.63-2.70 (2H, m), 2.75-2.77 (2H, t), | |
| | phenylurea (195) | | | | | | | 3.06 (3H, s), 3.50-3.53 (2H, t), | |
| | | | | | | | | 3.59 (1H, d), 3.75-3.80 (2H, m), | |
| | | | | | | | | 3.97-4.02 (1H, m), 6.91 (1H, s), | |
| | | | | | | | | 6.96-6.99 (1H, t), 7.19-7.22 (2H, t), | |
| | | | | | | | | 7.32 (1H, s), 7.34 (1H, s), | |
| | | | | | | | | 8.05 (1H, s) | |
| 196 | N-((3-amino-1-(5-methyl-7H- | Ex. 76 | LCMS | 1.02 | | 399.1 | | (500 MHz, methanol-d₄) | 0.123 |
| | pyrrolo[2,3-d]pyrimidin-4- | | STD | | | | | 2.48-2.52 (1H, m), 2.53 (3H, s), | |
| | yl)pyrrolidin-3-yl)methyl)-4- | | | | | | | 2.80 (3H, s), 2.82-2.86 (1H, m), | |
| | chloro-N-methylbenzamide | | | | | | | 3.62 (1H, d), 3.86 (1H, d), | |
| | (196) | | | | | | | 4.11-4.17 (2H, m), 4.35 (1H, d), 4.57 (1H, d), | |
| | | | | | | | | 7.25 (1H, s), 7.48 (1H, s), | |
| | | | | | | | | 7.49 (1H, s), 7.89 (1H, s), 7.91 (1H, s), | |
| | | | | | | | | 8.30 (1H, s) | |
| 197 | N-((3-amino-1-(5-methyl-7H- | Ex. 76 | LCMS | 1.94 | | 383.1 | | (500 MHz, methanol-d₄) | 0.148 |
| | pyrrolo[2,3-d]pyrimidin-4- | | Polar | | | | | 2.00-2.05 (1H, m), 2.18-2.23 (1H, m), | |
| | yl)pyrrolidin-3-yl)methyl)-2- | | | | | | | 2.45 (3H, s), 3.10 (3H, s), 3.70 (1H, d), | |
| | fluoro-N-methylbenzamide | | | | | | | 3.78 (1H, d), 3.92-3.96 (3H, m), | |
| | (197) | | | | | | | 4.03-4.08 (1H, m), 6.91 (1H, d), | |
| | | | | | | | | 7.23-7.26 (1H, t), 7.30-7.33 (1H, t), | |
| | | | | | | | | 7.45-7.48 (1H, m), 7.50-7.54 (1H, | |
| | | | | | | | | m), 8.09 (1H, s) | |
| 198 | N-{[3-amino-1-(5-methyl-7H- | Ex. 77 | LCMS | 1.52 | | 423.3 | | | 0.00267 |
| | pyrrolo[2,3-d]pyrimidin-4- | | Polar | | | | | | |
| | yl)pyrrolidin-3-yl]methyl}-2,6- | | | | | | | | |
| | difluorobenzenesulfonamide | | | | | | | | |
| | (198) | | | | | | | | |

What is claimed is:

1. A compound of formula I: 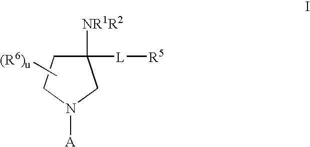
or a pharmaceutically acceptable salt thereof, wherein:
A is a moiety of formula: 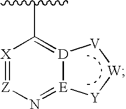
u is an integer from 0 to 3;
V is selected from the group consisting of N and CR⁷;
W and X are each independently selected from the group consisting of N and CR⁸;
Y is selected from the group consisting of CH, N and NH;
Z is selected from the group consisting of CH and N;
D and E are each selected from the group consisting of C and N, and wherein at least one of D and E is C;
L is a linker selected from the group consisting of —(CR³R⁴)ₘ— and —C(O)—, wherein one of said —(CR³R⁴)— moieties may optionally be replaced by a —CR³═CR⁴— group;
m is an integer from 1 to 6;
R¹ and R² are each independently selected from the group consisting of —H, —(C₁-C₆)alkyl, —(C₃-C₁₀)cycloalkyl, —(C₅-C₁₀)cycloalkenyl, —(C₂-C₉)heterocycloalkyl and —(C₄-C₉)heterocycloalkenyl; wherein each of the foregoing —(C₁-C₆)alkyl, —(C₃-C₁₀)cycloalkyl, —(C₅-C₁₀)cycloalkenyl, —(C₂-C₉)heterocycloalkyl and —(C₄-C₉)heterocycloalkenyl moieties is optionally substituted with one to five substituents independently selected from the group consisting of -halo, -cyano, —CF₃, —OR⁹, —C(O)R¹⁰, —NR¹¹R¹², —(C₁-C₆)alkyl and —(C₃-C₁₀)cycloalkyl;
R³ and R⁴ are each independently selected from the group consisting of —H, —(C₁-C₆)alkyl and —CF₃;
R⁵ is selected from the group consisting of:
(a) —OR¹³, —NR₁₄R¹⁵, —NR¹¹C(O)R¹⁰, —NR¹¹C(O)OR⁹, —NR¹¹C(O)NR¹¹R¹², —NR¹¹S(O)ⱼR¹⁶, —NR¹¹C(═N—R¹⁷)NR¹¹R¹², —C(O)OR¹⁸, —OC(O)OR⁹, —OC(O)R¹⁹, and —S(O)ⱼR²⁰;
(b) —(C₁-C₆)alkyl substituted with one to five R²¹ groups, —(C₂-C₆)alkenyl, —(C₂-C₆)alkynyl, —(C₃-C₁₀)cycloalkyl substituted with one to five R²¹ groups, —(C₅-C₁₀)cycloalkenyl, —(C₆-C₁₀)bicycloalkyl, and —(C₆-C₁₀)bicycloalkenyl;
(c) —(C₂-C₉)heterocycloalkyl substituted with one to five R²² groups, —(C₄-C₉)heterocycloalkenyl, —(C₆-C₉)heterobicycloalkyl, and —(C₆-C₉)heterobicycloalkenyl;
(d) —(C₆-C₁₀)aryl substituted with one to five R²³ groups; wherein two R²³ groups when attached to adjacent carbon atoms may be taken together with the carbon atoms to which they are attached to form a moiety selected from the group consisting of —(C₃-C₇)cycloalkyl, —(C₅-C₁₀)cycloalkenyl, —(C₂-C₉)heterocyclyl, and —(C₂-C₁₀)heterocycloalkenyl; and wherein each of the foregoing —(C₃-C₇)cycloalkyl, —(C₅-C₁₀)cycloalkenyl, —(C₂-C₉)heterocyclyl, and —(C₂-C₁₀)heterocycloalkenyl moieties formed by the joinder of two R²³ groups may optionally be fused to a —(C₆-C₁₀)aryl or —(C₁-C₉)heteroaryl moiety;
(e) —(C₁-C₉)heteroaryl substituted with one to five R²⁴ groups; wherein two R²⁴ groups when attached to adjacent carbon atoms may be taken together with the carbon atoms to which they are attached to form a moiety selected from the group consisting of —(C₃-C₇)cycloalkyl, —(C₅-C₁₀)cycloalkenyl, —(C₂-C₉)heterocyclyl and —(C₂-C₁₀)heterocycloalkenyl; and wherein each of the foregoing —(C₃-C₇)cycloalkyl, —(C₅-C₁₀)cycloalkenyl, —(C₂-C₉)heterocyclyl, and —(C₂-C₁₀)heterocycloalkenyl moieties formed by the joinder of two R²⁴ groups is optionally fused to a —(C₆-C₁₀)aryl or —(C₁-C₉)heteroaryl moiety;
wherein each of the foregoing —(C₂-C₆)alkenyl, —(C₂-C₆)alkynyl, —(C₅-C₁₀)cycloalkenyl, —(C₆-C₁₀)bicycloalkyl, —(C₆-C₁₀)bicycloalkenyl, —(C₂-C₉)heterocycloalkenyl, —(C6-C₉)heterobicycloalkyl and —(C₆-C₉)heterobicycloalkenyl R⁵ moieties in (b), (c), (d) and (e) above may optionally be substituted with one to five substituents independently selected from the group consisting of -halo, —OH, -cyano, —CF₃, —OCF₃, —OR⁹, —C(O)R¹⁰, —C(O)OR⁹, —OC(O)R¹⁰, —NR¹¹R¹², —NR¹¹C(O)R¹⁰, —C(O)NR¹¹R¹², —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C4-C₉)heterocycloalkenyl, —(C₆-C₁₀)aryl and —(C₁-C₉)heteroaryl;
R⁶ is selected from the group consisting of —(C₁-C₆)alkyl and —(C₃-C₁₀)cycloalkyl;
R⁷ is selected from the group consisting of -halo, —OH, —CF₃, —NR¹¹R¹², -cyano, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R⁸ is independently selected from the group consisting of —H, -halo, -cyano, —OH and —(C₁-C₆)alkyl;
each R⁹ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R¹⁰ is independently selected from the group consisting of —H, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₆-C₁₀)bicycloalkyl, —(C₆-C₁₀)bicycloalkenyl, —(C₂-C₉)heterocycloalkyl, —(C₄-C₉)heterocycloalkenyl, —(C₆-C₉)heterobicycloalkyl, —(C₆-C₉)heterobicycloalkenyl, —(C₆-C₁₀)aryl, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₆-C₁₀)bicycloalkyl, —(C₆-C₁₀)bicycloalkenyl, —(C₂-C₉)heterocycloalkyl, —(C₄-C₉)heterocycloalkenyl, —(C₆-C₉)heterobicycloalkyl, —(C₆-C₉)heterobicycloalkenyl, —(C₆-C₁₀)aryl, and —(C₁-C₉)heteroaryl moieties is optionally substituted with one to five R²⁴ groups;
R¹¹ and R¹² are each independently selected from the group consisting of —H, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
R¹¹ and R¹² when attached to the same N atom may be taken together with the N atom to which they are attached to form a 3- to 11-membered mono or bicyclic ring optionally containing one to two additional heteroatoms independently selected from the group consisting of N, O and S(O)ⱼ; wherein said 3- to 11-membered mono or bicyclic ring may be saturated, unsaturated or aromatic; wherein each ring carbon atom of said 3- to 11-membered mono or bicyclic ring is optionally substituted with an oxo moiety; and wherein each N atom of said 3- to 11-membered mono or bicyclic ring is optionally substituted with a —(C₁-C₆)alkyl;
each R¹³ is independently selected from the group consisting of —(C₁-C₆)alkyl substituted with one to five R²⁵ groups, —(C₃-C₁₀)cycloalkyl substituted with one to five R²⁵ groups, —(C₅-C₁₀)cycloalkenyl, —(C₂-C₆)alkenyl, —(C₂-C₆)alkynyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl moieties is optionally substituted with one to five R²⁴ groups;
each R¹⁴ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R¹⁵ is independently selected from the group consisting of —(C₁-C₆)alkyl substituted with one to five R²² groups, —(C₂-C₆)alkenyl, —(C₂-C₆)alkynyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₂-C₆)alkenyl, —(C₂-C₆)alkynyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R¹⁶ is independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R¹⁷ is independently selected from the group consisting of —H, —CF₃, -nitro, -cyano, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R¹⁸ is independently selected from the group consisting of —(C₁-C₆)alkyl substituted with one to five R²⁶ groups, —(C₂-C₆)alkenyl, —(C₂-C₆)alkynyl, —(C₃-C₁₀)cycloalkyl substituted with substituted with one to five R²⁶ groups, —(C₅-C₁₀)cycloalkenyl, —(C₆-C₁₀)aryl and —(C₁-C₉)heteroaryl; wherein each of the foregoing —(C₂-C₆)alkenyl, —(C₂-C₆)alkynyl, —(C₅-C₁₀)cycloalkenyl, —(C₆-C₁₀)aryl and —(C₁-C₉)heteroaryl moieties is optionally substituted with one to five R²⁴ groups;
each R¹⁹ is independently selected from the group consisting of —H, —NR²³R²⁹, —(C₁-C₆)alkyl substituted with one to five R²⁴ groups, —(C₂-C₆)alkenyl, —(C₂-C₆)alkynyl, —(C₃-C₁₀)cycloalkyl substituted with one to five R²⁴ groups, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, —(C₆-C₁₀)aryl, and —(C₁-C₉)heteroaryl moieties is optionally substituted with one to five R²⁴ groups;
each R²⁰ is independently selected from the group consisting of —H, —NR²⁸, R²⁹C₆)alkyl substituted with one to five R²² groups, —(C₂-C₆)alkenyl, —(C₂-C₆)alkynyl, —(C₃-C₁₀)cycloalkyl substituted with one to five R²² groups, —(C₅-C₁₀)cycloalkenyl, —(C₂-C₉)heterocycloalkyl, —(C₄-C₉)heterocycloalkenyl, —(C₆-C₁₀)aryl substituted with one to five R²² groups, and —(C₁-C₉)heteroaryl; wherein each of the foregoing —(C₂-C₆)alkenyl, —(C₂-C₆)alkynyl, —(C₅-C₁₀)cycloalkenyl, —(C₂-C₉)heterocycloalkyl, —(C₄-C₉)heterocycloalkenyl, and —(C₁-C₉)heteroaryl moieties is optionally substituted with one to five R²⁴ groups;
each R²¹ is independently selected from the group consisting of —CN, —NO₂, —SO₂NR²⁸R²⁹, —(C₂-C₆)alkenyl, and —(C₂-C₆)alkynyl; wherein each of the foregoing —(C₂-C₆)alkenyl and —(C₂-C₆)alkynyl moieties is optionally substituted with one to five R²⁴ groups;
each R²² is independently selected from the group consisting of -halo, —CF₃, —CN, —NO₂, —OR²⁸, —C(O)OR²⁸—OC(O)R²⁸—OC(O)OR²⁸, —NR²⁸R²⁹, —NR²⁸C(O)R²⁹, —S(O)₂R²⁸, —SO₂NR²⁸R²⁹ and —NR²⁸SO₂R²⁹, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl, wherein each of the foregoing —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl moieties is optionally substituted with one to five R²⁴ groups;
each R²³ is independently selected from the group consisting of -halo, —CF₃, —CN, —NO₂, —OR²⁸, —C(O)R²⁸, —C(O)OR²⁸—OC(O)R²⁸, —OC(O)OR²⁸, —NR²⁸R²⁹, —NR²⁸C(O)R²⁹—S(O)₂R², —SO₂NR²⁸R²⁹, —NR²⁸SO₂R²⁹, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl substituted with one to five R²⁷ groups, and —(C₁-C₉)heteroaryl; wherein each of the foregoing —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl and —(C₁-C₉)heteroaryl moieties is optionally substituted with one to five R²⁴ groups;
each R²⁴ is independently selected from the group consisting of -halo, —OH, —CF₃, —CN, —OCF₃, —NR²⁸R²⁹, —NR²⁸C(O)R²⁹, —C(O)R²⁸, —C(O)OR²⁸, —C(O)NR²⁸R²⁹, —C(O)NR²⁸C(O)R²⁹—C(O)NR²⁸C(O)NR²⁹, —SO₂R²⁸, —SO₂NR²⁸R²⁹, —(C₁-C₆)alkyl, —O(C₁-C₆)alkyl, —(C₃-C₁₀)cycloalkyl, —O(C₃-C₁₀)cycloalkyl, —(C₅-C₁₀)cycloalkenyl, —O(C₅-C₁₀)cycloalkenyl, —(C₂-C₉)heterocycloalkyl, —O(C₂-C₉)heterocycloalkyl, —C₄-C₉)heterocycloalkenyl, —O(C₄-C₉)heterocycloalkenyl, —(C₆-C₁₀)aryl, —O(C₆-C₁₀)aryl, —(C₁-C₉)heteroaryl and —O(C₁-C₉)heteroaryl; wherein each of the foregoing —(C₁-C₆)alkyl, —O(C₁-C₆)alkyl, —(C₃-C₁₀)cycloalkyl, —O(C₃-C₁₀)cycloalkyl, —(C₅-C₁₀)cycloalkenyl, —O(C₅-C₁₀)cycloalkenyl, —(C₂-C₉)heterocycloalkyl, —O(C₂-C₉)heterocycloalkyl, —(C₄-C₉)heterocycloalkenyl, —O(C₄-C₉)heterocycloalkenyl, —(C₆-C₁₀)aryl, —O(C₆-C₁₀)aryl, —(C₁-C₉)heteroaryl and —O(C₁-C₉)heteroaryl moieties is optionally substituted by one to three moieties independently selected from the group consisting of -halo, —CF₃, —CN, —NO₂, —OR²⁸, —C(O)R²⁸, —OC(O)R²⁹, —OC(O)OR²⁸, —NR²⁸R²⁹, —NR²⁸C(O)R²⁹, —S(O)₂R²⁸, —SO₂NR²⁸R²⁹, —NR²⁸SO₂R²⁹, —(C₁-C₆)alkyl, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl and —(C₁-C₉)heteroaryl;
each R²⁵ is independently selected from the group consisting of -halo, —CF₃, —CN, —NO₂, —OR²⁸, —C(O)OR²⁸—OC(O)R²⁸—OC(O)OR²⁸, —NR²⁸R²⁹, —NR²⁸C(O)R²⁹, —S(O)₂R²⁸, —SO₂NR²⁸R²⁹ and —NR²⁸SO₂R²⁸, —(C₂-C₆)alkenyl, —(C₂-C₆)alkynyl, —(C₃-C₁₀)cycloalkyl, —(C₂-C₉)heterocycloalkyl, —(C₄-C₉)heterocycloalkenyl, —(C₆-C₁₀)aryl substituted with one to five R²⁷ groups, and —(C₁-C₉)heteroaryl substituted with one to five R²⁶ groups;
each R²⁶ is independently selected from the group consisting of -halo, —CN, —NO₂, —OC(O)R²⁸—OC(O)OR²⁸, —NR²⁸C(O)R²⁹, —SO₂NR²⁸R²⁹, —(C₂-C₆)alkenyl, —(C₂-C₆)alkynyl, —(C₃-C₁₀)cycloalkyl, —(C₆-C₁₀)aryl substituted with one to five R²⁷ groups, and —(C₁-C₉)heteroaryl substituted with one to five R²⁷ groups;
each R²⁷ is independently selected from the group consisting of -halo, —CF₃, —CN, —NO₂, —C(O)OR²⁸—OC(O)OR²⁸, —SO₂NR²⁸R²⁹, —NR²⁸SO₂R²⁹, —(C₁-C₆)alkyl, —(C₂-C₆)alkenyl, and —(C₂-C₆)alkynyl;
R²⁸ and R²⁹ are each independently selected from the group consisting of —H, —CF₃, —(C₁-C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl; and
each j is independently an integer from 0 to 2.

2. The compound of claim 1, or a pharmaceutically acceptable salt thereof, wherein u is 1 and R⁶ is —(C₁-C₆)alkyl

3. The compound of claim 1, or a pharmaceutically acceptable salt thereof, wherein u is 0.

4. The compound of claim 1, or a pharmaceutically acceptable salt thereof, wherein the moiety A is selected from the group consisting of:

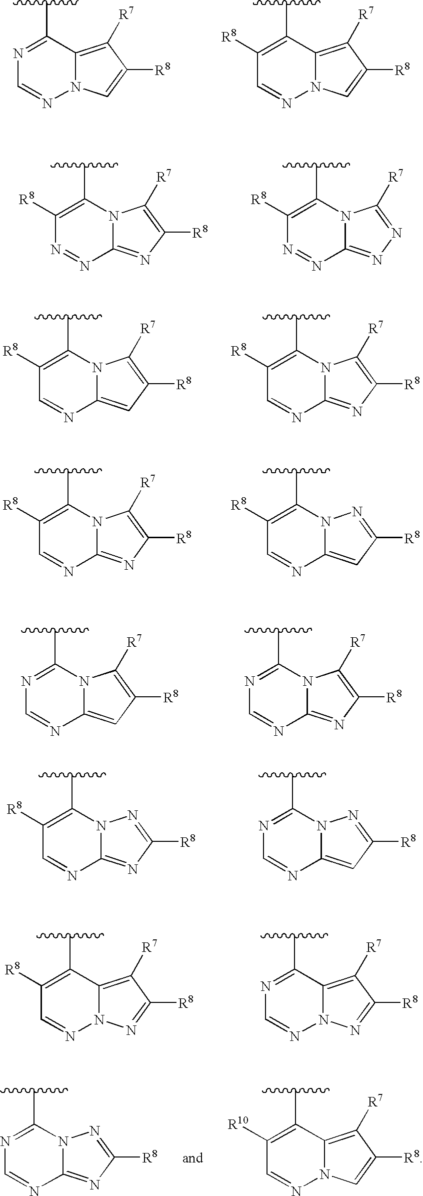

5. The compound of claim 1, or a pharmaceutically acceptable salt thereof, wherein R⁷ is selected from the group consisting of —(C₁-C₆)alkyl, —(C₂-C₆)alkenyl and —(C₂-C₆)alkynyl; wherein each of the foregoing —(C₁-C₆)alkyl, —(C₂-C₆)alkenyl and —(C₂-C₆)alkynyl moieties is optionally substituted with one to five R²⁴ groups.

6. The compound of claim 1, or a pharmaceutically acceptable salt thereof, wherein R⁷ is selected from the group consisting of —(C₃-C₁₀)cycloalkyl, —(C₅-C₁₀)cycloalkenyl, —(C₂-C₉)heterocycloalkyl, and —(C₄-C₉)heterocycloalkenyl; wherein each of the foregoing —(C₃-C₁₀)cycloalkyl, —(C₅-C₁₀)cycloalkenyl, —(C₂-C₉)heterocycloalkyl and —(C₄-C₉)heterocycloalkenyl moieties is optionally substituted with one to five R²⁴ groups.

7. The compound of claim 1, or a pharmaceutically acceptable salt thereof, wherein R³ is independently selected from the group consisting of —H and —(C₁-C₆)alkyl.

8. The compound of claim 1, or a pharmaceutically acceptable salt thereof, wherein R¹ and R² are each independently selected from the group consisting of —H and —(C₁-C₆)alkyl; wherein said —(C₁-C₆)alkyl may optionally be substituted with one to five substituents independently selected from the group consisting of -halo, -cyano, —CF₃, —OR⁹, —C(O)R¹⁰, —NR¹¹R¹², —(C₁-C₆)alkyl and —(C₃-C₁₀)cycloalkyl.

9. The compound of claim 8, or a pharmaceutically acceptable salt thereof, wherein R¹ and R² are each —H.

10. The compound of claim 1, or a pharmaceutically acceptable salt thereof, wherein L is —(CR³R⁴)ₘ—, and wherein one of said —(CR³R⁴)— moieties may optionally be replaced by a —CR³═CR⁴— moiety.

11. The compound of claim 10, or a pharmaceutically acceptable salt thereof, wherein m is an integer from 1 to 3.

12. The compound of claim 11, or a pharmaceutically acceptable salt thereof, wherein R³ and R₄ are each —H.

13. The compound of claim 1, or a pharmaceutically acceptable salt thereof, wherein L is —C(O)—.

14. The compound of claim 1, or a pharmaceutically acceptable salt thereof, wherein R⁵ is selected from the group consisting —(C₁-C₆)alkyl substituted with one to five R²¹ groups, —(C₂-C₆)alkenyl, —(C₂-C₆)alkynyl, —(C₃-C₁₀)cycloalkyl substituted with one to five R²¹ groups, —(C₅-C₁₀)cycloalkenyl, —(C₆-C₁₀)bicycloalkyl and —(C₆-C₁₀)bicycloalkenyl; wherein each of the foregoing —(C₂-C₆)alkenyl, —(C₂-C₆)alkynyl, —(C₅-C₁₀)cycloalkenyl, —(C₆-C₁₀)bicycloalkyl and —(C₆-C₁₀)bicycloalkenyl moieties may optionally be substituted with one to five substituents independently selected from the group consisting of -halo, —OH, -cyano, —CF₃, —OCF₃, —OR⁹, —C(O)R¹⁰, —C(O)OR⁹, —OC(O)R¹⁰, —NR¹¹R¹², —NR¹¹C(O)R¹⁰, —C(O)NR¹¹R¹², C₆)alkyl, —(C₂-C₆)alkenyl, —(C₂-C₆)alkynyl, —(C₃-C₁₀)cycloalkyl, —(C₅-C₁₀)cycloalkenyl, —(C₂-C₉)heterocycloalkyl, —(C₄-C₉)heterocycloalkenyl, —(C₆-C₁₀)aryl and —(C₁-C₉)heteroaryl.

15. The compound of claim 1, or a pharmaceutically acceptable salt thereof, wherein R⁵ is selected from the group consisting of —OR¹³, —NR¹⁴R¹⁵, —NR¹¹C(O)R¹⁰, —NR¹¹C(O)OR⁹, —NR¹¹C(O)NR¹¹R¹², —NR¹¹S(O)ⱼR¹⁶, —NR¹¹C(═N—R¹⁷)NR¹¹R¹², —C(O)OR¹⁸—OC(O)OR⁹, —OC(O)R¹⁹, and —S(O)ⱼR²⁰; and wherein

16. The compound of claim 15, or a pharmaceutically acceptable salt thereof, wherein R¹⁰ and R¹¹ are each independently selected from the group consisting of —H, —(C₁-C₆)alkyl, —(C₃-C₁₀)cycloalkyl, —(C₂-C₉)heterocycloalkyl, —(C₆-C₁₀)aryl, and —(C₁-C₉)heteroaryl, wherein each of the foregoing —(C₁-C₆)alkyl, —(C₃-C₁₀)cycloalkyl, —(C₂-C₉)heterocycloalkyl, —(C₆-C₁₀)aryl and —(C₁-C₉)heteroaryl moieties of said R¹⁰ and R¹¹ may each optionally be independently substituted with one to five R²⁴ groups.

17. The compound of claim 16, or a pharmaceutically acceptable salt thereof, wherein R¹⁰ is —(C₆-C₁₀)aryl and R¹¹ is —H; wherein said —(C₆-C₁₀)aryl of said R¹⁰ group is substituted with one to five groups selected from the group consisting of -halo, —OH, —CF₃, —CN, —OCF₃, —(C₁-C₆)alkyl and —(C₃-C₁₀)cycloalkyl.

18. The compound of claim 1 selected from the group consisting of:
(3S)-3-{[(4-chlorophenyl)amino]methyl}-1-(5-methyl-7H-pyrrolo[2,3-d]pyrimidin-4-yl)pyrrolidin-3-amine;
3-({[2-fluoro-3-(trifluoromethyl)phenyl]amino}methyl)-1-(5-methyl-7H-pyrrolo[2,3-d]pyrimidin-4-yl)pyrrolidin-3-amine;
(3S)-1-(5-methyl-7H-pyrrolo[2,3-d]pyrimidin-4-yl)-3-({[3-(trifluoromethyl)phenyl]amino}methyl)pyrrolidin-3-amine;
N-{[(3S)-3-amino-1-(5-ethyl-7H-pyrrolo[2,3-d]pyrimidin-4-yl)pyrrolidin-3-yl]methyl}-2,4-difluorobenzamide];
(3S)-3-({[2-fluoro-3-(trifluoromethyl)phenyl]amino}methyl)-1-(5-methyl-7H-pyrrolo[2,3-d]pyrimidin-4-yl)pyrrolidin-3-amine;
N-{[(3S)-3-amino-1-(5-methyl-7H-pyrrolo[2,3-d]pyrimidin-4-yl)pyrrolidin-3-yl]methyl}-5-tert-butylisoxazol-3-amine;
(3S)-3-{[(3-fluorophenyl)amino]methyl}-1-(5-methyl-7H-pyrrolo[2,3-d]pyrimidin-4-yl)pyrrolidin-3-amine;
N-{[(3S)-3-amino-1-(5-chloro-7H-pyrrolo[2,3-d]pyrimidin-4-yl)pyrrolidin-3-yl]methyl}-4-methylpyridin-3-amine;
N-{[(3S)-3-amino-1-(5-chloro-7H-pyrrolo[2,3-d]pyrimidin-4-yl)pyrrolidin-3-yl]methyl}-5-isopropyl-1H-pyrazol-3-amine;
N-{[(3S)-3-amino-1-(5-methyl-7H-pyrrolo[2,3-d]pyrimidin-4-yl)pyrrolidin-3-yl]methyl}-6-(trifluoromethyl)pyridin-2-amine;
N-{[(3S)-3-amino-1-(5-chloro-7H-pyrrolo[2,3-d]pyrimidin-4-yl)pyrrolidin-3-yl]methyl}pyridin-3-amine;
N-{[(3S)-3-amino-1-(5-chloro-7H-pyrrolo[2,3-d]pyrimidin-4-yl)pyrrolidin-3-yl]methyl}-5-methylisoxazol-3-amine;
N-{[(3S)-3-amino-1-(5-chloro-7H-pyrrolo[2,3-d]pyrimidin-4-yl)pyrrolidin-3-yl]methyl}-4-chloropyridin-2-amine;
N-{[(3S)-3-amino-1-(5-chloro-7H-pyrrolo[2,3-d]pyrimidin-4-yl)pyrrolidin-3-yl]methyl}-5-chloropyridin-2-amine;
N-{[(3S)-3-amino-1-(5-chloro-7H-pyrrolo[2,3-d]pyrimidin-4-yl)pyrrolidin-3-yl]methyl}-2,5-difluorobenzamide;
N-{[(3S)-3-amino-1-(5-chloro-7H-pyrrolo[2,3-d]pyrimidin-4-yl)pyrrolidin-3-yl]methyl}-2-chloro-4-fluorobenzamide;
N-{[(3S)-3-amino-1-(5-chloro-7H-pyrrolo[2,3-d]pyrimidin-4-yl)pyrrolidin-3-yl]methyl}benzamide;
N-{[(3S)-3-amino-1-(5-chloro-7H-pyrrolo[2,3-d]pyrimidin-4-yl)pyrrolidin-3-yl]methyl}-3-chlorobenzamide;
N-{[(3S)-3-amino-1-(5-chloro-7H-pyrrolo[2,3-d]pyrimidin-4-yl)pyrrolidin-3-yl]methyl}-3,4-difluorobenzamide; and
N-{[(3S)-3-amino-1-(5-ethyl-7H-pyrrolo[2,3-d]pyrimidin-4-yl)pyrrolidin-3-yl]methyl}-3-chlorobenzamide,
or a pharmaceutically acceptable salt of each of the foregoing compounds.

19. A composition comprising the compound of claim 1, or a pharmaceutically acceptable salt thereof, and at least one additional ingredient.

20. A method for the treatment of abnormal cell growth in a mammal comprising administering to said mammal an amount of the compound of claim 1, or a pharmaceutically acceptable salt thereof, that is effective in treating abnormal cell growth.

21. A method for making a compound of formula I, or a pharmaceutically acceptable salt thereof, comprising, reacting a cyclic amine of formula: 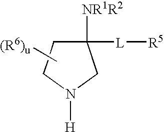
with a heterobicyclic compound of formula: 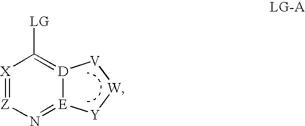
to provide a compound of formula I, wherein
D, E, V, W, X, Y, Z, u, R¹, R², R⁵ and R⁶ are as defined above in claim 1; and
LG is a leaving group.
